SEQUENCE LISTING (1 sequence)

SEQ ID NO: 1 - protein, 234 aa, Artificial Sequence
  TAG72-binding diabody derived from the CC49 antibody
SVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSDIVMTQSCSSCPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKR

(19) United States
(12) Patent Application Publication
(10) Pub. No.: US 2021/0299286 A1
(43) Pub. Date: Sep. 30, 2021

(54) TETRAZINES FOR HIGH CLICK CONJUGATION YIELD IN VIVO AND HIGH CLICK RELEASE YIELD

(71) Applicant: Tagworks Pharmaceuticals B.V., Eindhoven (NL)

(72) Inventors: Marc Stefan ROBILLARD, Eindhoven (NL); Ronny Mathieu VERSTEEGEN, Eindhoven (NL); Raffaella ROSSIN, Eindhoven (NL); Freek Johannes Maria HOEBEN, Eindhoven (NL); Sander Izaak VAN KASTEREN, Eindhoven (NL); Michel Johan VAN DE GRAAFF, Eindhoven (NL)

(73) Assignee: Tagworks Pharmaceuticals B.V., Eindhoven (NL)

(21) Appl. No.: 17/052,928

(22) PCT Filed: May 6, 2019
(86) PCT No.: PCT/NL2019/050271

§ 371 Date: Nov. 4, 2020

(30) Foreign Application Priority Data
May 4, 2018 (EP) 18170944.5

(51) Int. Cl. A61K 51/04

(52) U.S. Cl. CPC A61K 51/0461; A61K 51/0497; A61K 51/0495; A61K 51/0482

(57) ABSTRACT

Disclosed herein are tetrazines substituted with groups that result in a high click conjugation yield in vivo and high click release yields. In one aspect, the invention relates to kits having the tetrazines and a dienophile, preferably a trans-cyclooctene. In another aspect, the kits of the invention are for use as a medicament.

### FIELD OF THE INVENTION

The invention disclosed herein relates to tetrazines for high click conjugation yields in vivo and high click release yields.

### BACKGROUND OF THE INVENTION

Selective chemical reactions that are orthogonal to the diverse functionality of biological systems are called bio-orthogonal reactions and occur between two abiotic groups with exclusive mutual reactivity. These can be used to selectively modify biochemical structures, such as proteins or nucleic acids, which typically proceed in water and at near-ambient temperature, and may be applied in complex chemical environments, such as those found in living organisms.

Bio-orthogonal reactions are broadly useful tools with applications that span synthesis, materials science, chemical biology, diagnostics, and medicine.

Especially prominent application areas for bioorthogonal reactions include drug delivery agents and prodrugs for pharmaceutical applications, as well as various reversible bioconjugates and sophisticated spectroscopic bioprobes for applications in the field of biological analysis.

One prominent bioorthogonal reaction is the inverse-electron-demand Diels Alder (IEDDA) reaction between a trans-cyclooctene (TCO) and a tetrazine (TZ). In previous studies the IEDDA reaction was used for pretargeted radioimmunoimaging, treating tumor-bearing mice with trans-cyclooctene (TCO)-tagged antibody or antibody fragments, followed one or more days later by administration and selective conjugation of a radiolabeled tetrazine probe to the TCO tag of the tumor-bound antibody [R. Rossin, M. S. Robillard, Curr. Opin. Chem. Biol. 2014, 21, 161-169].

Based on the IEDDA conjugation a release reaction has been developed, which was termed the IEDDA pyridazine elimination, a “click-to-release” approach that affords instantaneous and selective release upon conjugation [R. M. Versteegen, R. Rossin, W. ten Hoeve, H. M. Janssen, M. S. Robillard, Angew. Chem. Int. Ed. 2013, 52, 14112-14116]. IEDDA reactions between tetrazines (i.e. diene) and alkenes (i.e. dienophile) afford 4,5-dihydropyridazines, which usually tautomerize to 1,4- and 2,5-dihydropyridazines. It was demonstrated that the 1,4-dihydropyridazine product derived from a TCO containing a carbamate-linked doxorubicin (Dox) at the allylic position and tetrazine is prone to eliminate CO₂ and Dox via an electron cascade mechanism eventually affording aromatic pyridazine. The triggered release has been demonstrated in PBS (phosphate buffered saline), serum, cell culture and in mice and holds promise for a range of applications in medicine, chemical biology, and synthetic chemistry, including triggered drug release, biomolecule uncaging and capture&release strategies.

The IEDDA pyridazine elimination has been applied in triggered drug release from antibody-drug conjugates (ADCs) capable of participating in an IEDDA reaction. ADCs are a promising class of biopharmaceuticals that combine the target-specificity of monoclonal antibodies (mAbs) or mAb fragments with the potency of small molecule toxins. Classical ADCs are designed to bind to an internalizing cancer cell receptor leading to uptake of the ADC and subsequent intracellular release of the drug by enzymes, thiols, or lysosomal pH. Routing the toxin to the tumour, while minimizing the peripheral damage to healthy tissue, allows the use of highly potent drugs resulting in improved therapeutic outcomes. The use of the IEDDA pyridazine elimination for ADC activation allows the targeting of non-internalizing receptors, as the drug is cleaved chemically instead of biologically.

In general prodrugs, which may comprise ADCs, are an interesting application for the IEDDA pyridazine elimination reaction, in which a drug is deactivated, bound or masked by a moiety, and is reactivated, released or unmasked after an IEDDA reaction has taken place.

Background art for the aforementioned technology further includes WO2012/156919, WO2012156918A1, WO 2014/081303, and US20150297741. Herein a dienophile is used as a chemically cleavable group. The group is attached to a Construct in such a way that the release of the dienophile from the Construct can be provoked by allowing the dienophile to react with a diene. The dienophile is an eight-membered non-aromatic cyclic alkenylene or alkenyl group, particularly a TCO group.

In some applications, the TCO is part of prodrug which is first injected in the blood stream of a subject and may be targeted to a certain part of the body, e.g. a tumor. Then, a certain percentage of the prodrug is immobilized at the targeted spot, while another percentage is cleared by the body. After several hours or days, an activator comprising a tetrazine is added to release a drug from the prodrug, preferably only at the targeted spot. The tetrazine itself is also subject to clearance by the body at a certain clearance rate.

In an initial step, the tetrazine reacts with a dienophile-containing prodrug to form a conjugate. This is referred to as the click conjugation step. Next, via one or multiple mechanisms, the drug is preferably released from the prodrug. It will be understood that a high yield in the click conjugation step, i.e. a high click conjugation yield, does not necessarily result in a high yield of released drug, i.e. a high drug release yield.

From the viewpoint of bio-orthogonality the chemistry works well.

However, it is desired that better IEDDA reactions are developed, preferably in vivo.

In one aspect, achieving high drug release yields in IEDDA reactions remains a challenge both in vivo and in vitro, in particular in vivo. In particular, the reaction between a drug-bearing dienophile and a tetrazine preferably results in a high drug release yield in vitro and/or in vivo.

In another aspect, the tetrazine motives that typically give high release are less reactive than the tetrazines that have successfully been used for click conjugations in vivo. These more reactive tetrazines give a good click conjugation yield, but result in a lower click release yield. Therefore, it is desired to improve the click conjugation yield of tetrazine motifs with relatively low reactivity towards dienophiles in vitro and/or in vivo.

In another aspect, a combination of a high click conjugation yield between the drug-bearing dienophile and the tetrazine and a high drug release yield is preferred both in vitro and in vivo. Preferably, this combination of a high click conjugation yield between the drug-bearing dienophile and the tetrazine and a high drug release yield is achieved in vivo.

In yet another aspect, it is desired to provide an increased reaction rate between the tetrazine and the dienophile.

A previous study ([R. Rossin, S. M. J. van Duijnhoven, W. ten Hoeve, H. M. Janssen, L. H. J. Kleijn, F. J. M. Hoeben, R. M. Versteegen, M. S. Robillard, Bioconj. Chem., 2016, 27, 1697-1706]) has shown that linking a 10 kDa dextran to a 3-methyl-6-(2-pyridyl)-tetrazine or a 3-methyl-6-(methylene)-tetrazine resulted in high click conjugation yields with a TCO in vivo, but to suboptimal drug release yields both in vitro and vivo.

Another publication ([X. Fan, Y. Ge, F. Lin, Y. Yang, G. Zhang, W. S. C. Ngai, Z. Lin, J. Wang, S. Zheng, J. Zhao, J. Li, P. R. Chen, Angew. Chem. Int. Ed., 2016, 55, 14046-14050]) aimed at in vitro reactions, showed that high release yields are obtained with small, asymmetrical tetrazines and TCOs.

In still another aspect, it is preferred that low doses of tetrazines are administered to subjects.

It is desired that compounds are developed that address one or more of the abovementioned problems and/or desires.

### SUMMARY OF THE INVENTION

In one aspect, the invention pertains to a kit comprising a tetrazine and a dienophile, wherein the tetrazine satisfies any one of the Formulae (1), (2), (3), (4), (5), (6), (7), or (8):

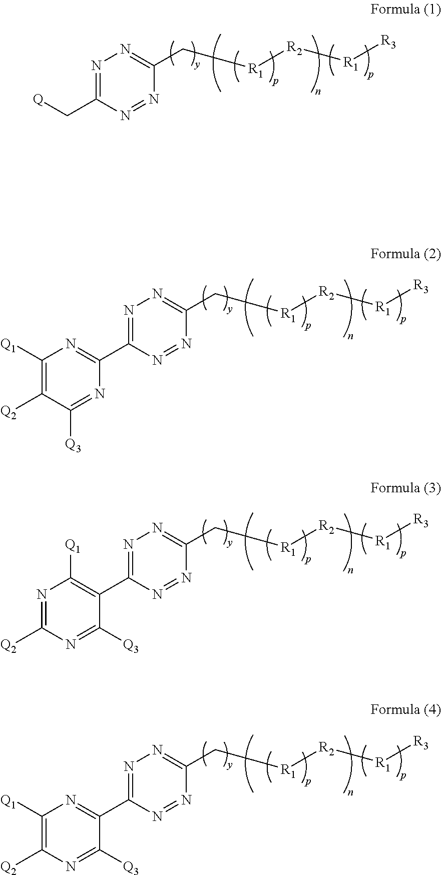

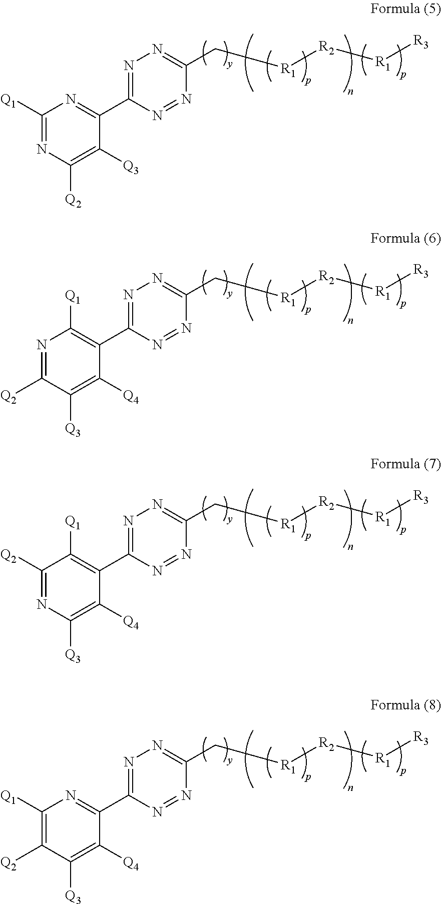

wherein each moiety Q, Q₁, Q₂, Q₃, and Q₄ is independently selected from the group consisting of hydrogen, and moieties according to Formula (9):

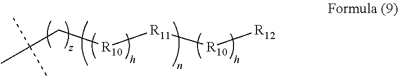

wherein the dashed line indicates a bond to the remaining part of the molecules satisfying any of the Formulae (1), (2), (3), (4), (5), (6), (7), or (8),
wherein in Formulae (1), (2), (3), (4), (5), (6), (7) and (8) at least one moiety selected from the group consisting of Q, Q₁, Q₂, Q₃, Q₄, and —(CH₂)_{y}—((R₁)ₚ—R₂)ₙ—(R₁)ₚ)—R₃ has a molecular weight in a range of from 100 Da to 3000 Da,
wherein in Formulae (1), (2), (3), (4), (5), (6), (7) and (8) moieties selected from the group consisting of Q, Q₁, Q₂, Q₃, Q₄, and —(CH₂)_{y}—((R₁)ₚ—R₂)ₙ—(R₁)ₚ)—R₃ have a molecular weight of at most 3000 Da,
wherein in Formula (1) when Q is not H, z is 0, n belonging to Q is at least 1, and at least one h is 1, then y is at least 2,
wherein in Formula (1) when Q is not H, y is 1, n belonging to —(CH₂)_{y}—((R₁)ₚ—R₂)ₙ—(R₁)ₚ)—R₃ is at least 1, and at least one p is 1, then z is at least 1, wherein in Formula (8) when Q₁, Q₂, Q₃, and Q₄ are hydrogen, then y is not 1,
wherein in Formula (8) when y is 1, all p are 0, n belonging to —(CH₂)_{y}—((R₁)ₚ—R₂)ₙ—(R₁)ₚ)—R₃ is 0, R₃ is hydrogen, Q₁ is hydrogen, Q₃ is hydrogen, Q₄ is hydrogen, and Q₂ is not hydrogen, then z is at least 1.

In another aspect, the invention pertains to a kit comprising a tetrazine and a dienophile, wherein the tetrazine satisfies any one of Formulae (11), (12), (13), (14), (15), (16), (17), or (18):

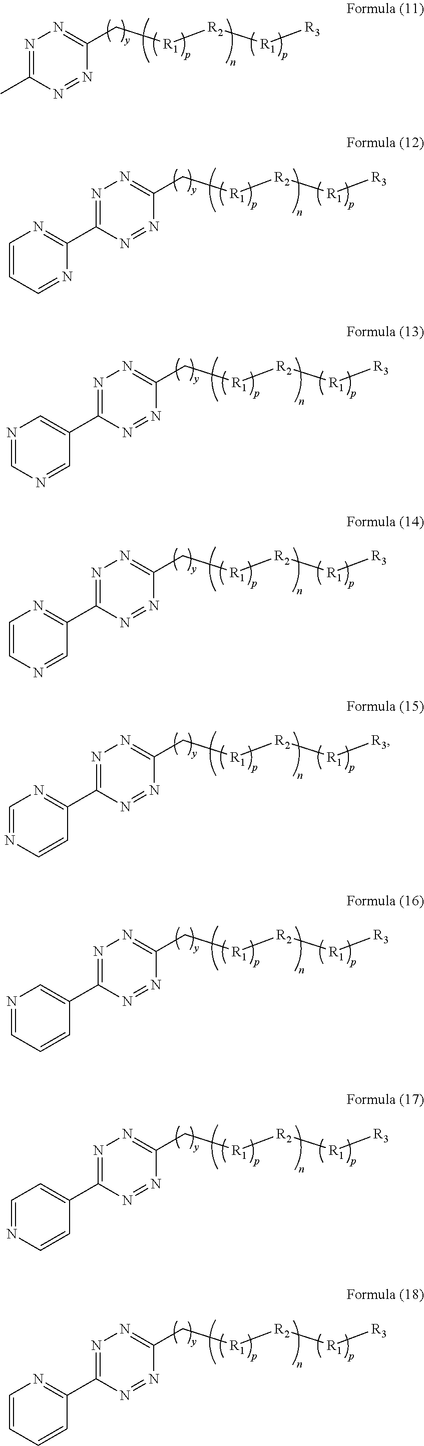

wherein in Formulae (11), (12), (13), (14), (15), (16), (17), and (18) the moiety —(CH₂)_{y}—((R₁)ₚ—R₂)ₙ—(R₁)ₚ)—R₃ has a molecular weight in a range of from 100 Da to 3000 Da,
wherein in Formula (18) y is not 1.

In yet another aspect, the invention pertains to a kit comprising a tetrazine and a dienophile, wherein the dienophile satisfies Formula (19a):

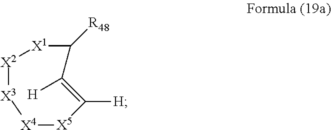

wherein the dienophile preferably satisfies Formula (19):

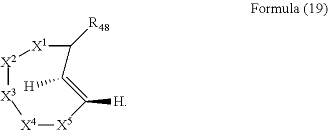

In a still further aspect, the invention pertains to kits comprising a tetrazine and a dienophile, wherein the dienophile satisfies Formula (20):

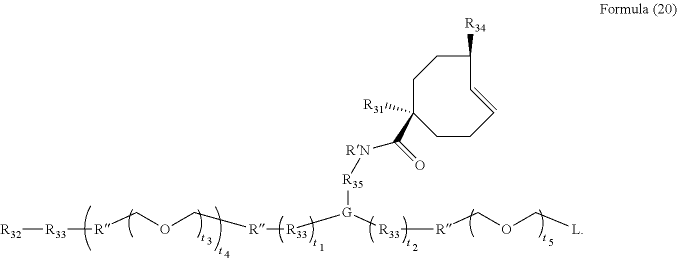

In a still further aspect, the invention pertains to kits comprising a tetrazine and a dienophile, wherein said kit comprises a Construct-B (C^{B}), preferably a targeting agent, preferably a compound selected from the group consisting of proteins, antibodies, peptoids and peptides, modified with at least one compound according to Formula (20).

In a still further aspect, the invention pertains to kits comprising a tetrazine and a dienophile, wherein said kit comprises a Construct-B (C^{B}), preferably a targeting agent, preferably a compound selected from the group consisting of proteins, antibodies, peptoids and peptides, modified with at least one compound according to Formula (20) so as to satisfy Formula (21):

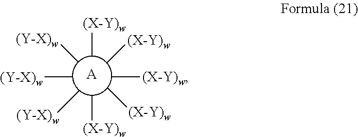

wherein moiety A is Construct-B (C^{B}), preferably a targeting agent, preferably selected from the group consisting of proteins, antibodies, peptoids and peptides,
wherein each moiety Y is independently selected from moieties according to Formula (22), wherein at least one moiety Y satisfies said Formula (22):

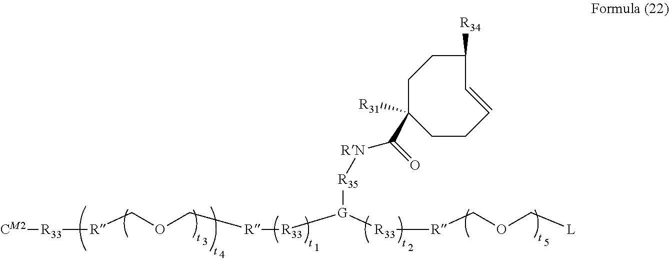

In a still further aspect, the invention pertains to kits comprising a tetrazine according to any one of Formulae (1) to (18) for use in the treatment of patients. In another aspect, the invention pertains to methods for treating patients, said methods comprising administering to a subject the compounds comprised in the kits disclosed herein.

### BRIEF DESCRIPTION OF THE FIGURES

FIG. 1 depicts HPLC-QTOF-MS analysis of AVP0458-TCO-MMAE activation mixture. Diabody conjugate with activator 2.12 in PBS; top: HPLC chromatogram (peak at 2.46 min is excess activator and at 3.90 min is free MMAE); middle: HPLC chromatogram filtered for m/z=718.51 Da (free MMAE); bottom: MS spectrum of the diabody conjugate after summation of the range from 3.2-4.2 min and subsequent deconvolution, showing fully reacted ADC with 2×MMAE release (31720 Da) and a minor amount of fully reacted ADC with 1×MMAE release (32481 Da).

FIG. 2 depicts the results from in vivo reactivity studies in LS174T tumor bearing mice pretreated with (A) an IgG-based ADC (CC49-TCO-Dox; ca. 5 mg/kg) or with (B) a diabody-based ADC (AVP0458-TCO-MMAE; ca. 2 mg/kg) followed by a series of TZ activators at different doses (dose 1×: ca. 3.35 μmol/kg; dose 2.5×: ca. 8.4 μmol/kg; close 5×: ca. 16.7 μmol/kg; dose 10×: ca. 0.033 mmol/kg; dose 100×: ca. 0.335 mmol/kg) and, finally, by the highly reactive probe [¹⁷⁷Lu]Lu-5.1 (ca. 0.335 μmol/kg). High tumor blocking signifies low probe binding in tumor and, therefore, high reaction yield between tumor-bound TCO and the TZ activator at the administered dose.

FIG. 3 depicts the MMAE concentration in (A) tumors, (B) plasma, and (C) livers of mice injected with diabody-based ADC (AVP0458-TCO-MMAE; ca. 2 mg/kg) followed by activator 2.12 (ca. 0.335 mmol/kg) or vehicle and euthanized 24 or 48 h after the activator/vehicle administration, or in mice euthanized 24 h after the administration of enzymatically-cleavable vc-ADC. (D) MMAE concentration in tumor of mice injected with diabody-based ADC followed by a low dose (ca. 3.35 μmol/kg) of activators 2.12, 4.26 or 4.28 and euthanized 24 h after activator administration.

FIG. 4 depicts the results of a proliferation assay on LS174T cells treated with a combination of diabody-based ADC (AVP0458-TCO-MMAE) or IgG-based ADC (CC49-TCO-Dox) and activators 2.12, 3.4, 4.12, 4.26, 4.33, 4.35; ADCs and activators alone and free drugs are used as controls.

FIG. 5 depicts single-tumor growth curves and combined survival results from a therapy study in mice bearing LS174T xenografts and injected with 4 cycles of combined diabody-based ADC (AVP0458-TCO-MMAE, ca. 3 mg/kg) and activator 4.12 (ca. 16.7 μmol/kg), ADC and activator alone, or vehicle.

FIG. 6 depicts the results of an activation assay in THP1-Dual cells treated with a TLR ADC (TA99-TCO-R848, 1.5 μM) reacted with activator 4.12 (1.5 μM) or treated with ADC and activator alone; TLR7/8 agonist (R848) and PBS are used as controls.

FIG. 7 depicts the results of an in vivo activation study in C57BL/6 mice bearing B16-F10 melanoma: (A) biodistribution of ¹²⁵I-labeled native TA99 and TA99-TCO-R848 (ca. 5 mg/kg), 48 h post-mAb injection, and biodistribution of TA99-TCO-R848 followed by a clearing agent (CA, 48 h post-mAb injection), TA99-TCO-R848 followed by clearing agent and activator 4.12 (ca. 3.35 μmol/kg, 50 h post-mAb injection) 54 h post-mAb injection. (B) Biodistribution of [¹¹¹In]In-5.1 probe in the mice injected with TA99-TCO-R848 followed by clearing agent alone or clearing agent and activator 4.12; the probe (ca. 0.335 mol/kg) was injected 51 h post-mAb and the mice were euthanized 54 h post-mAb injection. The decreased probe uptake in tumor, skin, blood and non-target tissues signifies that in vivo reaction between TCO linker and activator 4.12 has occurred.

FIG. 8 depicts the results from a therapy study in mice bearing OVCAR-3 xenografts. (A, B) Mean tumor volumes (with SEM) in mice injected with 4 cycles of ADC (AVP0458-TCO-MMAE) followed by 2.12 (ca. 0.335 mmol/kg), non-binding nb-ADC followed by 2.12, enzymatically cleavable vc-ADC followed by vehicle; control mice received vehicle, 2.12 or AVP0458-TCO-MMAE alone; the bars below the x axis indicate the treatment period. (C) Mean body weight of the mice during the therapy study (error bars omitted for clarity). (D) Survival curves for the therapy groups in A, B.

FIG. 9 depicts a preferred embodiment of this invention. In both panels an ADC is administered to a cancer patient, and is allowed to circulate and bind to a target on the cancer cell. After the freely circulating ADC has sufficiently cleared from circulation, for example after 2 days post injection, the Activator, is administered and distributes systemically, allowing the reaction with the Trigger of cancer-bound Prodrug or ADC, releasing the Drug, after which the Drug can penetrate and kill neighbouring cancer cells. Panel A depicts the cleavage of a carbamate-linked Drug and Panel B depicts the cleavage of an ether-linked Drug.

FIG. 10 depicts a preferred embodiment of this invention. An antibody construct comprising a bi-specific (anti-tumor and anti-CD3) antibody and a masking moiety (blocking protein) is administered to a cancer patient, and is allowed to circulate and bind to a target on the cancer cell. After the freely circulating construct has sufficiently cleared from circulation, for example after 2 days post injection, the Activator, is administered and distributes systemically, allowing the reaction with the Trigger of cancer-bound Prodrug, releasing the mask, after which T-cells bind the bi-specific antibody resulting in tumor killing.

### DETAILED DESCRIPTION OF THE INVENTION

The invention, in a broad sense, is based on the judicious insight to provide 3,6-bis-alkyl-tetrazine, 3-alkyl-6-pyridyl-tetrazine, and 3-alkyl-6-pyrimidyl-tetrazine motifs with a substituent group of a certain molecular weight, for example in a range of from 100 Da to 3000 Da. Particularly, this is believed to be useful in obtaining improved reactions, such as in vivo, with dienophile-containing prodrugs, in particular drug-bearing TCOs.

In one aspect, the kits of the invention achieve high click conjugation yields both in vitro and in vivo, in particular in vivo. In another aspect, the kits of the invention achieve high drug release yields both in vitro and in vivo, in particular in vivo. Without wishing to be bound by theory, it is believed that the bulky group of a certain size on the tetrazine improves the in vivo, on-site reaction time of the tetrazine that reacts with a drug-bearing dienophile, preferably a drug-bearing TCO, that may be directed to a certain site within the subject, for example a tumor.

In another aspect, the tetrazines of the invention are desirably used in vivo at doses that are lower than expected based on their reactivity. Particularly favorable embodiments of the invention in this particular aspect are 3-alkyl-6-pyridyl-tetrazines. Other particularly favorable embodiments of the invention in this particular aspect are 3-alkyl-6-pyrimidyl-tetrazines.

In particularly favorable embodiments of the invention, the kit comprises a 3-alkyl-6-pyridyl-tetrazine or a 3-alkyl-6-pyrimidyl-tetrazine that are substituted with a bulky group of a certain size on the pyridyl or the pyrimidyl group, respectively. It is believed that this may result in an even better drug release yield than when the bulky group is present on the alkyl group of the 3-alkyl-6-pyridyl-tetrazine or the 3-alkyl-6-pyrimidyl-tetrazine. Without wishing to be bound by theory, it is believed that in these embodiments, one mechanism via which this results in an increased drug release yield is based on the bulky group causing the formation a favorable regioisomer with a TCO in the click conjugation step. Again without wishing to be bound by theory, it is believed that another mechanism to achieve an increased drug release yield with these embodiments, is based on the steric hinder from the bulky group causing more out-of-plane rotation, increasing the drug release yield. Still without wishing to be bound by theory, it is believed that at least one, both or yet still other mechanisms contribute to the increased drug release yield when 3-alkyl-6-pyridyl-tetrazine or a 3-alkyl-6-pyrimidyl-tetrazine are used that are substituted with a bulky group of a certain size on the pyridyl or the pyrimidyl group, respectively.

Without wishing to be bound by theory, it is believed that decreasing the clearance rate of the tetrazine may help to increase the on-tumor reaction time, and thereby may help to achieve optimal drug release yields in vivo.

In yet another aspect, the click conjugation yield of tetrazine motifs with relatively low reactivity towards dienophiles in vitro and/or in vivo is increased in some embodiments of the invention.

In some embodiments of the invention, the drug is released extracellularly as the ADC binds a non-internalizing receptor. Without wishing to be bound by theory, extracellular release is believed to be beneficial in the treatment of solid tumors, wherein there is a lack of specific, suitable and internalizing receptors, while these tumors do have tumor-specific non-internalizing receptors that can be targeted for treatments.

### Definitions

The present invention will further be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where an indefinite or definite article is used when referring to a singular noun e.g. “a” or “an”, “the”, this includes a plural of that noun unless something else is specifically stated.

The verb “to comprise”, and its conjugations, as used in this description and in the claims is used in its non-limiting sense to mean that items following the word are included, but items not specifically mentioned are not excluded. Thus, the scope of the expression “a device comprising means A and B” should not be limited to devices consisting only of components A and B. It means that with respect to the present invention, the only relevant components of the device are A and B.

In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer, unless stated otherwise. When the structure of a compound is depicted as a specific diastereomer, it is to be understood that the invention of the present application is not limited to that specific diastereomer, unless stated otherwise.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer, unless stated otherwise.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer, unless stated otherwise.

Unless stated otherwise, the compounds of the invention and/or groups thereof may be protonated or deprotonated. It will be understood that it is possible that a compound may bear multiple charges which may be of opposite sign. For example, in a compound containing an amine and a carboxylic acid, the amine may be protonated while simultaneously the carboxylic acid is deprotonated.

In several formulae, groups or substituents are indicated with reference to letters such as “A”, “B”, “X”, “Y”, and various (numbered) “R” groups. In addition, the number of repeating units may be referred to with a letter, e.g. n in —(CH₂)ₙ—. The definitions of these letters are to be read with reference to each formula, i.e. in different formulae these letters, each independently, can have different meanings unless indicated otherwise.

In several chemical formulae and texts below reference is made to “alkyl”, “heteroalkyl”, “aryl”, “heteroaryl”, “alkenyl”, “alkynyl”, “alkylene”, “alkenylene”, “alkynylene”, “arylene”, “cycloalkyl”, “cycloalkenyl”, “cycloakynyl”, arenetriyl, and the like. The number of carbon atoms that these groups have, excluding the carbon atoms comprised in any optional substituents as defined below, can be indicated by a designation preceding such terms (e.g. “C₁-C₈ alkyl” means that said alkyl may have from 1 to 8 carbon atoms). For the avoidance of doubt, a butyl group substituted with a —OCH₃ group is designated as a C₄ alkyl, because the carbon atom in the substituent is not included in the carbon count.

Unsubstituted alkyl groups have the general formula CₙH₂ₙ₊₁ and may be linear or branched. Optionally, the alkyl groups are substituted by one or more substituents further specified in this document. Examples of alkyl groups include methyl, ethyl, propyl, 2-propyl, t-butyl, 1-hexyl, 1-dodecyl, etc. Unless stated otherwise, an alkyl group optionally contains one or more heteroatoms independently selected from the group consisting of O, NR₅, S, P, and Si, wherein the N, S, and P atoms are optionally oxidized and the N atoms are optionally quaternized. In preferred embodiments, up to two heteroatoms may be consecutive, such as in for example —CH₂—NH—OCH₃ and —CH₂—O—Si(CH₃)₃. In some preferred embodiments the heteroatoms are not directly bound to one another. Examples of heteroalkyls include —CH₂CH₂O—CH₃, —CH₂CH₂—NH—CH₃, —CH₂CH₂—S(O)—CH₃, —CH═CH—O—CH₃, —Si(CH₃)₃. In preferred embodiments, a C₁-C₄ alkyl contains at most 2 heteroatoms.

A cycloalkyl group is a cyclic alkyl group. Unsubstituted cycloalkyl groups comprise at least three carbon atoms and have the general formula CₙH₂ₙ₊₁. Optionally, the cycloalkyl groups are substituted by one or more substituents further specified in this document. Examples of cycloalkyl groups include cyclopropyl, cyclobutyl, cyclopentyl and cyclohexyl. Unless stated otherwise, a cycloalkyl group optionally contains one or more heteroatoms independently selected from the group consisting of O, NR₅, S, P, and Si, wherein the N, S, and P atoms are optionally oxidized and the N atoms are optionally quaternized.

An alkenyl group comprises one or more carbon-carbon double bonds, and may be linear or branched. Unsubstituted alkenyl groups comprising one C—C double bond have the general formula CₙH₂ₙ₋₁. Unsubstituted alkenyl groups comprising two C—C double bonds have the general formula CₙH₂ₙ₋₃. An alkenyl group may comprise a terminal carbon-carbon double bond and/or an internal carbon-carbon double bond. A terminal alkenyl group is an alkenyl group wherein a carbon-carbon double bond is located at a terminal position of a carbon chain. An alkenyl group may also comprise two or more carbon-carbon double bonds. Examples of an alkenyl group include ethenyl, propenyl, isopropenyl, t-butenyl, 1,3-butadienyl, 1,3-pentadienyl, etc. Unless stated otherwise, an alkenyl group may optionally be substituted with one or more, independently selected, substituents as defined below. Unless stated otherwise, an alkenyl group optionally contains one or more heteroatoms independently selected from the group consisting of O, NR₅, S, P, and Si, wherein the N, S, and P atoms are optionally oxidized and the N atoms are optionally quaternized.

An alkynyl group comprises one or more carbon-carbon triple bonds, and may be linear or branched. Unsubstituted alkynyl groups comprising one C—C triple bond have the general formula CₙH₂ₙ₋₃. An alkynyl group may comprise a terminal carbon-carbon triple bond and/or an internal carbon-carbon triple bond. A terminal alkynyl group is an alkynyl group wherein a carbon-carbon triple bond is located at a terminal position of a carbon chain. An alkynyl group may also comprise two or more carbon-carbon triple bonds. Unless stated otherwise, an alkynyl group may optionally be substituted with one or more, independently selected, substituents as defined below. Examples of an alkynyl group include ethynyl, propynyl, isopropynyl, t-butynyl, etc. Unless stated otherwise, an alkynyl group optionally contains one or more heteroatoms independently selected from the group consisting of O, NR₅, S, P, and Si, wherein the N, S, and P atoms are optionally oxidized and the N atoms are optionally quaternized.

An aryl group refers to an aromatic hydrocarbon ring system that comprises six to twenty-four carbon atoms, more preferably six to twelve carbon atoms, and may include monocyclic and polycyclic structures. When the aryl group is a polycyclic structure, it is preferably a bicyclic structure. Optionally, the aryl group may be substituted by one or more substituents further specified in this document. Examples of aryl groups are phenyl and naphthyl.

Arylalkyl groups and alkylaryl groups comprise at least seven carbon atoms and may include monocyclic and bicyclic structures. Optionally, the arylalkyl groups and alkylaryl may be substituted by one or more substituents further specified in this document. An arylalkyl group is for example benzyl. An alkylaryl group is for example 4-tert-butylphenyl.

Heteroaryl groups comprise at least two carbon atoms (i.e. at least C₂) and one or more heteroatoms N, O, P or S. A heteroaryl group may have a monocyclic or a bicyclic structure. Optionally, the heteroaryl group may be substituted by one or more substituents further specified in this document. Examples of suitable heteroaryl groups include pyridinyl, quinolinyl, pyrimidinyl, pyrazinyl, pyrazolyl, imidazolyl, thiazolyl, pyrrolyl, furanyl, triazolyl, benzofuranyl, indolyl, purinyl, benzoxazolyl, thienyl, phosphonyl and oxazolyl. Heteroaryl groups preferably comprise five to sixteen carbon atoms and contain between one to five heteroatoms.

Heteroarylalkyl groups and alkylheteroaryl groups comprise at least three carbon atoms (i.e. at least C₃) and may include monocyclic and bicyclic structures. Optionally, the heteroaryl groups may be substituted by one or more substituents further specified in this document.

Where an aryl group is denoted as a (hetero)aryl group, the notation is meant to include an aryl group and a heteroaryl group. Similarly, an alkyl(hetero)aryl group is meant to include an alkylaryl group and an alkylheteroaryl group, and (hetero)arylalkyl is meant to include an arylalkyl group and a heteroarylalkyl group. A C₂-C₂₄ (hetero)aryl group is thus to be interpreted as including a C₂-C₂₄ heteroaryl group and a C₆-C₂₄ aryl group. Similarly, a C₃-C₂₄ alkyl(hetero)aryl group is meant to include a C₇-C₂₄ alkylaryl group and a C₃-C₂₄ alkylheteroaryl group, and a C₃-C₂₄ (hetero)arylalkyl is meant to include a C₇-C₂₄ arylalkyl group and a C₃-C₂₄ heteroarylalkyl group.

A cycloalkenyl group is a cyclic alkenyl group. An unsubstituted cycloalkenyl group comprising one double bond has the general formula CₙH₂ₙ₋₃. Optionally, a cycloalkenyl group is substituted by one or more substituents further specified in this document. An example of a cycloalkenyl group is cyclopentenyl. Unless stated otherwise, a cycloalkenyl group optionally contains one or more heteroatoms independently selected from the group consisting of O, NR₅, S, P, and Si, wherein the N, S, and P atoms are optionally oxidized and the N atoms are optionally quaternized.

A cycloalkynyl group is a cyclic alkynyl group. An unsubstituted cycloalkynyl group comprising one triple bond has the general formula CₙH₂ₙ₋₅. Optionally, a cycloalkynyl group is substituted by one or more substituents further specified in this document. An example of a cycloalkynyl group is cyclooctynyl. Unless stated otherwise, a cycloalkynyl group optionally contains one or more heteroatoms independently selected from the group consisting of O, NR₅, S, P, and Si, wherein the N, S, and P atoms are optionally oxidized and the N atoms are optionally quaternized.

In general, when (hetero) is placed before a group, it refers to both the variant of the group without the prefix hetero- as well as the group with the prefix hetero-. Herein, the prefix hetero- denotes that the group contains one or more heteroatoms selected from the group consisting of O, N, S, P, and Si. It will be understood that groups with the prefix hetero- by definition contain heteroatoms. Hence, it will be understood that if a group with the prefix hetero- is part of a list of groups that is defined as optionally containing heteroatoms, that for the groups with the prefix hetero- it is not optional to contain heteroatoms, but is the case by definition.

Herein, it will be understood that when the prefix hetero- is used for combinations of groups, the prefix hetero- only refers to the one group before it is directly placed. For example, heteroarylalkyl denotes the combination of a heteroaryl group and an alkyl group, not the combination of a heteroaryl and a heteroalkyl group. As such, it will be understood that when the prefix hetero- is used for a combination of groups that is part of a list of groups that are indicated to optionally contain heteroatoms, it is only optional for the group within the combination without the prefix hetero- to contain a heteroatom, as it is not optional for the group within the combination with the prefix hetero- by definition (see above). For example, if heteroarylalkyl is part of a list of groups indicated to optionally contain heteroatoms, the heteroaryl part is considered to contain heteroatoms by definition, while for the alkyl part it is optional to contain heteroatoms.

Herein, the prefix cyclo- denotes that groups are cyclic. It will be understood that when the prefix cyclo- is used for combinations of groups, the prefix cyclo- only refers to the one group before it is directly placed. For example, cycloalkylalkenylene denotes the combination of a cycloalkylene group (see the definition of the suffix -ene below) and an alkenylene group, not the combination of a cycloalkylene and a cycloalkenylene group.

In general, when (cyclo) is placed before a group, it refers to both the variant of the group without the prefix cyclo- as well as the group with the prefix cyclo-.

Herein, the suffix -ene denotes divalent groups, i.e. that the group is linked to at least two other moieties. An example of an alkylene is propylene (—CH₂—CH₂—CH₂—), which is linked to another moiety at both termini. It is understood that if a group with the suffix -ene is substituted at one position with —H, then this group is identical to a group without the suffix. For example, an alkylene substituted with —H is identical to an alkyl group. I.e. propylene, —CH₂—CH₂—CH₂—, substituted with —H at one terminus, —CH₂—CH₂—CH₂—H, is logically identical to propyl, —CH₂—CH₂—CH₃.

Herein, when combinations of groups are listed with the suffix -ene, it refers to a divalent group, i.e. that the group is linked to at least two other moieties, wherein each group of the combination contains one linkage to one of these two moieties. As such, for example alkylarylene is understood as a combination of an arylene group and an alkylene group. An example of an alkylarylene group is -phenyl-CH₂—, and an example of an arylalkylene group is —CH₂-phenyl-.

Herein, the suffix -triyl denotes trivalent groups, i.e. that the group is linked to at least three other moieties. An example of an arenetriyl is depicted below:

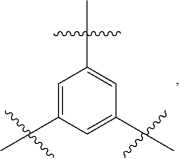 [SMILES: *c1cc(*)cc(*)c1]

wherein the wiggly lines denote bonds to different groups of the main compound.

It is understood that if a group with the suffix -triyl is substituted at one position with —H, then this group is identical to a divalent group with the suffix -ene. For example, an arenetriyl substituted with —H is identical to an arylene group. Similarly, it is understood that if a group with the suffix -triyl is substituted at two positions with —H, then this group is identical to a monovalent group. For example, an arenetriyl substituted with two —H is identical to an aryl group.

It is understood that if a group, for example an alkyl group, contains a heteroatom, then this group is identical to a hetero-variant of this group. For example, if an alkyl group contains a heteroatom, this group is identical to a heteroalkyl group. Similarly, if an aryl group contains a heteroatom, this group is identical to a heteroaryl group. It is understood that “contain” and its conjugations mean herein that when a group contains a heteroatom, this heteroatom is part of the backbone of the group. For example, a C₂ alkylene containing an N refers to —NH—CH₂—CH₂—, —CH₂—NH—CH₂—, and —CH₂—CH₂—NH—.

Unless indicated otherwise, a group may contain a heteroatom at non-terminal positions or at one or more terminal positions. In this case, “terminal” refers to the terminal position within the group, and not necessarily to the terminal position of the entire compound. For example, if an ethylene group contains a nitrogen atom, this may refer to —NH—CH₂—CH₂—, —CH₂—NH—CH₂—, and —CH₂—CH₂—NH—. For example, if an ethyl group contains a nitrogen atom, this may refer to —NH—CH₂—CH₃, —CH₂—NH—CH₃, and —CH₂—CH₂—NH₂.

Herein, it is understood that cyclic compounds (i.e. aryl, cycloalkyl, cycloalkenyl, etc.) are understood to be monocyclic, polycyclic or branched. It is understood that the number of carbon atoms for cyclic compounds not only refers to the number of carbon atoms in one ring, but that the carbon atoms may be comprised in multiple rings. These rings may be fused to the main ring or substituted onto the main ring. For example, C₁₀ aryl optionally containing heteroatoms may refer to inter alia a naphthyl group (fused rings) or to e.g. a bipyridyl group (substituted rings, both containing an N atom).

Unless stated otherwise, (hetero)alkyl groups, (hetero)alkenyl groups, (hetero)alkynyl groups, (hetero)cycloalkyl groups, (hetero)cycloalkenyl groups, (hetero)cycloalkynyl groups, (hetero)alkylcycloalkyl groups, (hetero)alkylcycloalkenyl groups, (hetero)alkylcycloalkynyl groups, (hetero)cycloalkylalkyl groups, (hetero)cycloalkenylalkyl groups, (hetero)cycloalkynylalkyl groups, (hetero)alkenylcycloalkyl groups, (hetero)alkenylcycloalkenyl groups, (hetero)alkenylcycloalkynyl groups, (hetero)cycloalkylalkenyl groups, (hetero)cycloalkenylalkenyl groups, (hetero)cycloalkynylalkenyl groups, (hetero)alkynylcycloalkyl groups, (hetero)alkynylcycloalkenyl groups, (hetero)alkynylcycloalkynyl groups, (hetero)cycloalkylalkynyl groups, (hetero)cycloalkenylalkynyl groups, (hetero)cycloalkynylalkynyl groups, (hetero)aryl groups, (hetero)arylalkyl groups, (hetero)arylalkenyl groups, (hetero)arylalkynyl groups, alkyl(hetero)aryl groups, alkenyl(hetero)aryl groups, alkenyl(hetero)aryl groups, cycloalkyl(hetero)aryl groups, cycloalkenyl(hetero)aryl groups, cycloalkynyl(hetero)aryl groups, (hetero)arylcycloalkyl groups, (hetero)arylcycloalkenyl groups, (hetero)arylcycloalkynyl groups, (hetero)alkylene groups, (hetero)alkenylene groups, (hetero)alkynylene groups, (hetero)cycloalkylene groups, (hetero)cycloalkenylene groups, (hetero)cycloalkynylene groups, (hetero)arylene groups, alkyl(hetero)arylene groups, (hetero)arylalkylene groups, (hetero)arylalkenylene groups, (hetero)arylalkynylene groups, alkenyl(hetero)arylene, alkynyl(hetero)arylene, (hetero)arenetriyl groups, (hetero)cycloalkanetriyl groups, (hetero)cycloalkenetriyl and (hetero)cycloalkynetriyl groups are optionally substituted with one or more substituents independently selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₆-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups. Unless stated otherwise, the substituents disclosed herein optionally contain one or more heteroatoms selected from the group consisting of O, S, NR₅, P, and Si, wherein the N, S, and P atoms are optionally oxidized, wherein the N atoms are optionally quaternized. Preferably, these substituents optionally contain one or more heteroatoms selected from the group consisting of O, S and NR₅.

In some embodiments, the substituents are selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₄-C₁₂ (hetero)arylalkenyl groups, C₄-C₁₂ (hetero)arylalkynyl groups, C₄-C₁₂ alkenyl(hetero)aryl groups, C₄-C₁₂ alkynyl(hetero)aryl groups, C₄-C₁₂ alkylcycloalkyl groups, C₆-C₁₂ alkylcycloalkenyl groups, C₁₃-C₁₆ alkylcycloalkynyl groups, C₄-C₁₂ cycloalkylalkyl groups, C₆-C₁₂ cycloalkenylalkyl groups, C₁₃-C₁₆ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₅-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₁₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero)arylcycloalkynyl groups.

In some embodiments, the substituents are selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₇ alkyl groups, C₂-C₇ alkenyl groups, C₂-C₇ alkynyl groups, C₆-C₇ aryl groups, C₂-C₇ heteroaryl groups, C₃-C₇ cycloalkyl groups, C₅-C₇ cycloalkenyl groups, C₁₂ cycloalkynyl groups, C₃-C₇ alkyl(hetero)aryl groups, C₃-C₇ (hetero)arylalkyl groups, C₄-C₇ (hetero)arylalkenyl groups, C₄-C₇ (hetero)arylalkynyl groups, C₄-C₇ alkenyl(hetero)aryl groups, C₄-C₇ alkynyl(hetero)aryl groups, C₄-C₇ alkylcycloalkyl groups, C₆-C₇ alkylcycloalkenyl groups, C₁₃-C₁₆ alkylcycloalkynyl groups, C₄-C₇ cycloalkylalkyl groups, C₆-C₇ cycloalkenylalkyl groups, C₁₃-C₁₆ cycloalkynylalkyl groups, C₅-C₇ alkenylcycloalkyl groups, C₅-C₇ alkenylcycloalkenyl groups, C₁₄-C₁₆ alkenylcycloalkynyl groups, C₅-C₇ cycloalkylalkenyl groups, C₇-C₈ cycloalkenylalkenyl groups, C₁₄-C₁₆ cycloalkynylalkenyl groups, C₅-C₇ alkynylcycloalkyl groups, C₇-C₈ alkynylcycloalkenyl groups, C₁₄-C₁₆ alkynylcycloalkynyl groups, C₅-C₇ cycloalkylalkynyl groups, C₇-C₈ cycloalkenylalkynyl groups, C₁₄-C₁₆ cycloalkynylalkynyl groups, C₅-C₇ cycloalkyl(hetero)aryl groups, C₇-C₈ cycloalkenyl(hetero)aryl groups, C₁₄-C₁₆ cycloalkynyl(hetero)aryl groups, C₅-C₇ (hetero)arylcycloalkyl groups, C₇-C₈ (hetero)arylcycloalkenyl groups, and C₁₄-C₁₆ (hetero)arylcycloalkynyl groups, C₁—C₈ (hetero)arylalkenyl groups, C₄-C₈ (hetero)arylalkynyl groups, C₄-C₈ alkenyl(hetero)aryl groups, C₄-C₈ alkynyl(hetero)aryl groups, C₅-C₉ cycloalkyl(hetero)aryl groups, C₇-C₁₁ cycloalkenyl(hetero)aryl groups, C₁₄-C₁₈ cycloalkynyl(hetero)aryl groups, C₅-C₉ (hetero)arylcycloalkyl groups, C₇-C₁₁ (hetero)arylcycloalkenyl groups, and C₁₄-C₁₈ (hetero)arylcycloalkynyl groups.

Unless stated otherwise, any group disclosed herein that is not cyclic is understood to be linear or branched. In particular, (hetero)alkyl groups, (hetero)alkenyl groups, (hetero)alkynyl groups, (hetero)alkylene groups, (hetero)alkenylene groups, (hetero)alkynylene groups, and the like are linear or branched, unless stated otherwise.

The general term “sugar” is herein used to indicate a monosaccharide, for example glucose (Glc), galactose (Gal), mannose (Man) and fucose (Fuc). The term “sugar derivative” is herein used to indicate a derivative of a monosaccharide sugar, i.e. a monosaccharide sugar comprising substituents and/or functional groups. Examples of a sugar derivative include amino sugars and sugar acids, e.g. glucosamine (GlcNH₂), galactosamine (GalNH₂), N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (IdoA).

A sugar may be without further substitution, and then it is understood to be a monosaccharide. A sugar may be further substituted with at one or more of its hydroxyl groups, and then it is understood to be a disaccharide or an oligosaccharide. A disaccharide contains two monosaccharide moieties linked together. An oligosaccharide chain may be linear or branched, and may contain from 3 to 10 monosaccharide moieties.

The term “protein” is herein used in its normal scientific meaning. Herein, polypeptides comprising about 10 or more amino acids are considered proteins. A protein may comprise natural, but also unnatural amino acids. The term “protein” herein is understood to comprise antibodies and antibody fragments.

The term “peptide” is herein used in its normal scientific meaning. Herein, peptides are considered to comprise a number of amino acids in a range of from 2 to 9.

The term “peptoids” is herein used in its normal scientific meaning.

An antibody is a protein generated by the immune system that is capable of recognizing and binding to a specific antigen. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as Drug or Targeting Agent (T^{T}), such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. For the avoidance of doubt, in the context of this invention the term “antibody” is meant to encompass all of the antibody variations, fragments, derivatives, fusions, analogs and mimetics outlined in this paragraph, unless specified otherwise.

A linker is herein defined as a moiety that connects two or more elements of a compound. For example in a bioconjugate, a biomolecule and a targeting moiety are covalently connected to each other via a linker.

A biomolecule is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “salt thereof” means a compound formed when an acidic proton, typically a proton of an acid, is replaced by a cation, such as a metal cation or an organic cation and the like. The term “salt thereof” also means a compound formed when an amine is protonated. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter-ions having acceptable mammalian safety for a given dosage regime). Such salts may be derived from pharmaceutically acceptable inorganic or organic bases and from pharmaceutically acceptable inorganic or organic acids.

“Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The logarithm of the partition-coefficient, i.e. Log P, is herein used as a measure of the hydrophobicity of a compound. Typically, the Log P is defined as

$$log ( \frac{{[ Solute ]}_{octanol}^{un \text{-} ionized}}{{[ Solute ]}_{water}^{un \text{-} ionized}} )$$

The skilled person is aware of methods to determine the partition-coefficient of compounds without undue experimentation. Alternatively, the skilled person knows that software is available to reliably estimate the Log P value, for example as a function within ChemDraw® software or online available tools.

The unified atomic mass unit or Dalton is herein abbreviated to Da. The skilled person is aware that Dalton is a regular unit for molecular weight and that 1 Da is equivalent to 1 g/mol (grams per mole).

It will be understood that herein, the terms “moiety” and “group” are used interchangeably when referring to a part of a molecule.

It will be understood that when a heteroatom is denoted as —X(R′)₂—, wherein X is the heteroatom and R′ is a certain moiety, then this denotes that two moieties R′ are attached to the heteroatom.

It will be understood that when a group is denoted as, for example, —((R₅₁)₂—R₅₂)₂— or a similar notation, in which R₅₁ and R₅₂ are certain moieties, then this denotes that first, it should be written as —R₅₁—R₅₁—R₅₂—R₅₁—R₅₁—R₅₂— before the individual R₅₁ and R₅₂ moieties are selected, rather than first selecting moieties R₅₁ and R₅₂ and then writing out the formula.

### The Inverse Electron-Demand Diels-Alder Reaction (IEDDA)

The established IEDDA conjugation chemistry generally involves a pair of reactants that comprise, as one reactant (i.e. one Bio-orthogonal Reactive Group), a suitable diene, such as a derivative of tetrazine (TZ), e.g. an electron-deficient tetrazine and, as the other reactant (i.e. the other Bio-orthogonal Reactive Group), a suitable dienophile, such as a trans-cyclooctene (WO). The exceptionally fast reaction of (substituted) tetrazines, in particular electron-deficient tetrazines, with a TCO moiety results in an intermediate that rearranges to a dihydropyridazine Diels-Alder adduct by eliminating N₂ as the sole by-product. The initially formed 4,5-dihydropyridazine product may tautomerize to a 1,4- or a 2,5-dihydropyridazine product, especially in aqueous environments. Below a reaction scheme is given for a [4+2] IEDDA reaction between (3,6)-di-(2-pyridyl)-s-tetrazine diene and a trans-cyclooctene dienophile, followed by a retro Diels Alder reaction in which the product and dinitrogen is formed. Because the trans-cyclooctene derivative does not contain electron withdrawing groups as in the classical Diels Alder reaction, this type of Diels Alder reaction is distinguished from the classical one, and frequently referred to as an “inverse-electron-demand Diels Alder (IEDDA) reaction”. In the following text the sequence of both reaction steps, i.e. the initial Diels-Alder cyclo-addition (typically an inverse electron-demand Diels Alder cyclo-addition) and the subsequent retro Diels Alder reaction will be referred to in shorthand as the “inverse electron-demand Diels Alder reaction” or “inverse electron-demand Diels Alder conjugation” or “IEDDA”. The product of the reaction is then the IEDDA adduct or conjugate. This is illustrated in Scheme 1 below.

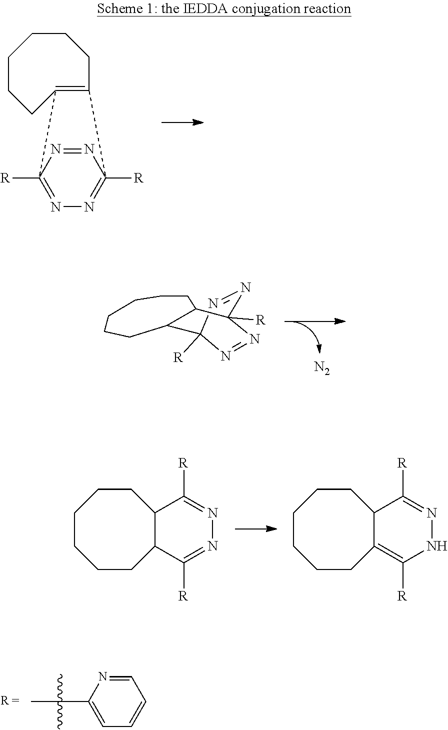

The two reactive species are abiotic and do not undergo fast metabolism or side reactions in vitro or in vivo. They are bio-orthogonal, e.g. they selectively react with each other in physiologic media. Thus, the compounds and the method of the invention can be used in a living organism. Moreover, the reactive groups are relatively small and can be introduced in biological samples or living organisms without significantly altering the size of biomolecules therein. References on the inverse electron demand Diels Alder reaction, and the behavior of the pair of reactive species include: [Thalhammer et at, Tetrahedron Lett., 1990, 31, 47, 6851-6854], [Wijnen et al., J. Org. Chem., 1996, 61, 2001-2005], [Blackman et at, J. Am. Chem. Soc., 2008, 130, 41, 13518-19], [Rossin et al., Angew. Chem. Int. Ed. 2010, 49, 3375], [Devaraj et al., Angew. Chem. Int. Ed. 2009, 48, 7013], [Devaraj et al., Angew. Chem. Int. Ed., 2009, 48, 1-5].

### The IEDDA Pyridazine Elimination Reaction

Below, the dienophile, a TCO, that is comprised in kits of the invention may be referred to as a “Trigger”. The dienophile is connected at the allylic position to a Construct-A. Moreover, tetrazines that are used in the IEDDA pyridazine elimination reaction may be referred to as “Activators”. The term Construct-A in this invention is used to indicate any substance, carrier, biological or chemical group, of which it is desired to have it first in a bound (or masked) state, and being able to provoke release from that state.

The inventors previously demonstrated that the dihydropyridazine product derived from a tetrazine (the Activator) and a TCO containing a carbamate-linked drug (doxorubicin, the Construct-A) at the allylic position is prone to eliminate CO₂ and the amine-containing drug, eventually affording aromatic pyridazine.

Without wishing to be bound by theory, the inventors believe that the Activator provokes Construct-A release via a cascade mechanism within the IEDDA adduct, i.e. the dihydropyridazine. The cascade mechanism can be a simple one step reaction, or it can be comprised in multiple steps that involves one or more intermediate structures. These intermediates may be stable for some time or may immediately degrade to the thermodynamic end-product or to the next intermediate structure. In any case, whether it be a simple or a multistep process, the result of the cascade mechanism is that the Construct-A gets released from the IEDDA adduct. Without wishing to be bound by theory, the design of the diene is such that the distribution of electrons within the IEDDA adduct is unfavorable, so that a rearrangement of these electrons must occur. This situation initiates the cascade mechanism, and it therefore induces the release of the Construct-A. Specifically, and without wishing to be bound by theory, the inventors believe that the NH moiety comprised in the various dihydropyridazine tautomers, such as the 1,4-dihydropyridazine tautomer, of the IEDDA adduct can initiate an electron cascade reaction, a concerted or consecutive shift of electrons over several bonds, leading to release of the Construct-A. Occurrence of the cascade reaction in and/or Construct-A release from the Trigger is not efficient or cannot take place prior to the IEDDA reaction, as the Trigger-Construct-A conjugate itself is relatively stable as such. The cascade can only take place after the Activator and the Trigger-Construct conjugate have reacted and have been assembled in the IEDDA adduct.

With reference to Scheme 2 below, and without wishing to be bound by theory, the inventors believe that the pyridazine elimination occurs from the 1,4-dihydropyridazine tautomer 4. Upon formation of the 4,5-dihydropyridazine 3, tautomerization affords intermediates 4 and 7, of which the 2,5-dihydropyridazine 7 cannot eliminate the Construct-A (C^{A}). Instead it can slowly convert into aromatic 8, which also cannot eliminate C^{A} or it can tautomerize back to intermediate 3. Upon formation of 4 the C^{A} is eliminated near instantaneously, affording free C^{A} 8 as an amine, and pyridazine elimination products 5 and 6. This elimination reaction has been shown to work equally well in the cleavage of carbonates, esters and ethers from the TCO trigger. The Trigger in Scheme 2 is also optionally bound to a Construct-B (C^{B}), which in this case cannot release from the Trigger. Thereby Construct A can be separated from Construct B by means of the IEDDA pyridazine elimination.

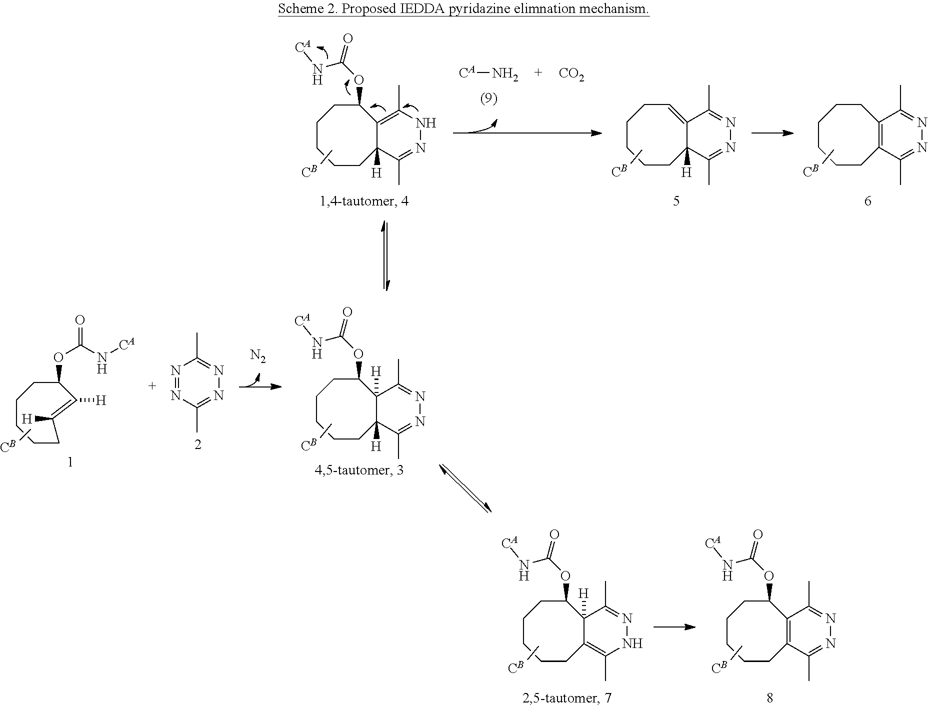

In some embodiments, the dienophile trigger moiety used in the present invention comprises a trans-cyclooctene ring. Herein, this eight-membered ring moiety will be defined as a trans-cyclooctene moiety, for the sake of legibility, or abbreviated as “TCO” moiety. It will be understood that the essence resides in the possibility of the eight-membered ring to act as a dienophile and to be released from its conjugated Construct-A upon reaction.

The tetrazines of the kits of the invention and dienophiles are capable of reacting in an inverse electron-demand Diels-Alder reaction (IEDDA). IEDDA reaction of the Trigger with the Activator leads to release of the Construct-A through an electron-cascade-based elimination, termed the “pyridazine elimination”. When an Activator reacts with a Trigger capable of eliminating Construct-A, the combined process of reaction and Construct-A elimination is termed the “IEDDA pyridazine elimination”.

This invention provides an Activator that reacts with a Construct-A-conjugated Trigger, resulting in the cleavage of the Trigger from the Construct-A and optionally the cleavage of one or more Construct-A from one or more Construct-B. In some embodiments, the Trigger is used as a reversible covalent bond between two molecular species.

Scheme 3 below is a general scheme of Construct release according to this invention, wherein the Construct being released is termed Construct-A (C^{A}), and wherein another Construct, Construct-B (C^{B}) can be bound to the dienophile, wherein Construct-B may or may not be able to be released from the dienophile. Typically, only Construct-A can be released from the dienenophile.

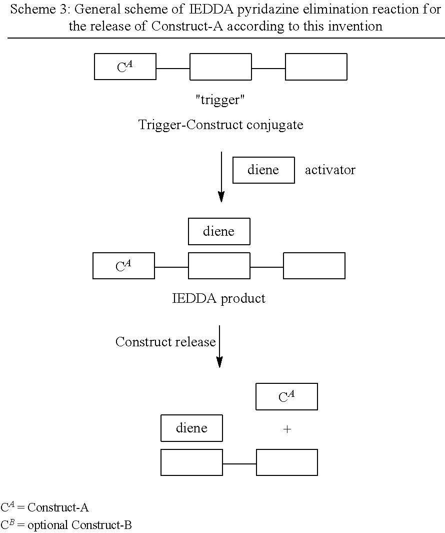

The Construct release occurs through a powerful, abiotic, bio-orthogonal reaction of the dienenophile (Trigger) with the diene (Activator), viz. the aforementioned IEDDA. The masked or bound Construct is a Construct-dienenophile conjugate. Possibly the Construct-A is linked to one or more additional Constructs A linked via a self-immolative linker. It will be understood that in Scheme 3 in the IEDDA adduct as well as in the end product after release, the indicated dienophile group and the indicated diene group are the residues of, respectively, the dienophile and diene groups after these groups have been converted in the IEDDA reaction.

The invention provides, in one aspect, the use of a tetrazine as an Activator for the release, in a chemical, biological, or physiological environment, of a Construct linked to a TCO. In connection herewith, the invention also pertains to a tetrazine as an Activator for the release, in a chemical, biological, or physiological environment, of a substance linked to a TCO. The fact that the reaction is bio-orthogonal, and that many structural options exist for the reaction pairs, will be clear to the skilled person. E.g., the IEDDA reaction is known in the art of bioconjugation, diagnostics, pre-targeted medicine. Reference is made to, e.g., WO 2010/119382, WO 2010/119389, and WO 2010/051530. Whilst the invention presents an entirely different use of the reaction, it will be understood that the various structural possibilities available for the IEDDA reaction pairs as used in e.g. pre-targeting, are also available in the field of the present invention.

Other than is the case with e.g. medicinally active substances, where the in vitro or in vivo action is often changed with minor structural changes, the present invention first and foremost requires the right chemical reactivity combined with sufficient stability for the intended application. Thus, the possible structures extend to those of which the skilled person is familiar with that these are reactive as dienes or dienophiles.

### Tetrazine

The compound comprising a tetrazine used to activate the dienophile is herein referred to as “Activator”. The tetrazine reacts with the other Bio-orthogonal Reactive Group, that is a dienophile (vide supra). The diene of the Activator is selected so as to be capable of reacting with the dienophile, e.g. the TCO, by undergoing a Diels-Alder cycloaddition followed by a retro Diels-Alder reaction, giving the IEDDA adduct. This intermediate adduct then releases the Construct-A, where this release can be caused by various circumstances or conditions that relate to the specific molecular structure of the IEDDA adduct.

Synthesis routes to tetrazines in general are readily available to the skilled person, based on standard knowledge in the art. References to tetrazine synthesis routes include for example Lions et al, J. Org. Chem., 1965, 30, 318-319; Horwitz et al, J. Am. Chem. Soc., 1958, 80, 3155-3159; Hapiot et al, New J. Chem., 2004, 28, 387-392, Kahn et al, Z. Naturforsch, 1995, 50b, 123-127; Yang et al., Angew. Chem. 2012, 124, 5312-5315; Mao et al., Angew. Chem. Int. Ed. 2019, 58, 1106-1109; Qu et al. Angew. Chem. Int. Ed. 2018, 57, 12057-12061; Selvaraj et al., Tetrahedron Lett. 2014, 55, 4795-4797; Fan et al., Angew. Chem. Int. Ed. 2016, 55, 14046-14050.

In one aspect, the invention pertains to a kit comprising a tetrazine and a dienophile, wherein the tetrazine satisfies any one of Formulae (1), (2), (3), (4), (5), (6), (7), or (8):

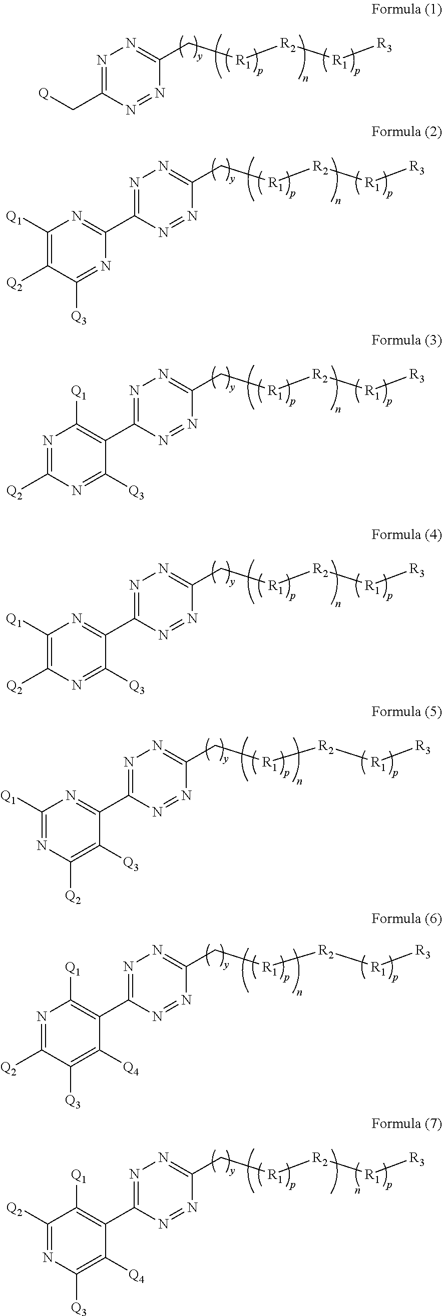

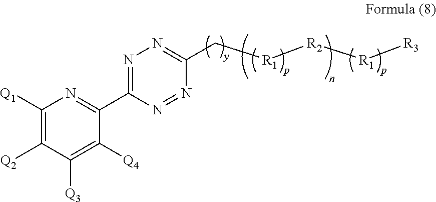

and preferably including pharmaceutically acceptable salts thereof,
wherein each moiety Q, Q₁, Q₂, Q₃, and Q₄ is independently selected from the group consisting of hydrogen, and moieties according to Formula (9):

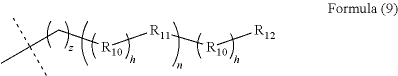

wherein the dashed line indicates a bond to the remaining part of the molecules satisfying any of the Formulae (1), (2), (3), (4), (5), (6), (7), or (8),
wherein each n is an integer independently selected from a range of from 0 to 24,
wherein each p is independently 0 or 1,
wherein y is an integer in a range of from 1 to 12,
wherein z is an integer in a range of from 0 to 12,
wherein each h is independently 0 or 1,
wherein each R₁ and R₁₀ are independently selected from the group consisting of —O—, —S—, —SS—, —NR₄—, —N(R₄)₂_{⁺}—, —N═N—, —C(O)—, —C(S)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, SC(S)—, —C(S)S—, —SC(S)NR₄—, —NR₄C(S)S—, —C(S)O—, —OC(S)O—, —OC(S)NR₄—, —NR₄C(S)—, —NR₄C(S)O—, —NR₄C(S)—, —C(S)NR₄—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —S(O)₂NR₄—, NR₄S(O)₂—, —OP(O)(R₄)₂—, —SP(O)(R₄)₂—, —NR₄P(O)(R₄)₂—,
wherein R₂ and R₁₁ are independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups,
wherein R₄ and R₁₂ are independently selected from the group consisting of hydrogen, —OH, —NH₂, —N₃, —Cl, —Br, —F, —I, and a chelating moiety,
wherein each R₄ is independently selected from the group consisting of hydrogen, C₁-C₂₄ alkyl groups, C₂-C₂₄ alkenyl groups, C₂-C₂₄ alkynyl groups, C₆-C₂₄ aryl, C₂-C₂₄ heteroaryl, C₃-C₂₄ cycloalkyl groups, C₅-C₂₄ cycloalkenyl groups, C₁₂-C₂₄ cycloalkynyl groups,
wherein in Formulae (1), (2), (3), (4), (5), (6), (7) and (8) at least one moiety selected from the group consisting of Q, Q₁, Q₂, Q₃, Q₄, and —(CH₂)_{y}—((R₁)ₚ—R₂)ₙ—(R₁)ₚ)—R₃ has a molecular weight in a range of from 100 Da to 3000 Da,
wherein in Formulae (1), (2), (3), (4), (5), (6), (7) and (8) moieties selected from the group consisting of Q, Q₁, Q₂, Q₃, Q₄, and —(CH₂)_{y}—((R₁)ₚ—R₂)ₙ—(R₁)ₚ)—R₃ have a molecular weight of at most 3000 Da,
wherein in Formula (1) when Q is not H, z is 0, n belonging to Q is at least 1, and at least one h is 1, then y is at least 2,
wherein in Formula (1) when Q is not H, y is 1, n belonging to —(CH₂)_{y}—((R₁)ₚ—R₂)ₙ—(R₁)ₚ)—R₃ is at least 1, and at least one p is 1, then z is at least 1,
wherein in Formula (8) when Q₁, Q₂, Q₃, and Q₄ are hydrogen, then y is not 1,
wherein in Formula (8) when y is 1, all p are 0, n belonging to —(CH₂)_{y}—((R₁)ₚ—R₂)ₙ—(R₁)ₚ)—R₃ is 0, R₃ is hydrogen, Q₁ is hydrogen, Q₃ is hydrogen, Q₄ is hydrogen, and Q₂ is not hydrogen, then z is at least 1,
wherein the R₂ groups, the R₁₁ groups, and the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized,
wherein the R₂ groups, the R₁₁ groups, and the R₄ groups not being hydrogen, are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkenylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized,
wherein each R₅ is independently selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero) arylcycloalkyl groups,
wherein the R₅ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, 5, and P atoms are optionally oxidized, wherein the N atoms are optionally quaternized.

In another aspect, the invention pertains to a kit comprising a tetrazine and a dienophile, wherein the tetrazine satisfies any one of Formulae (11), (12), (13), (14), (15), (16), (17), or (18):

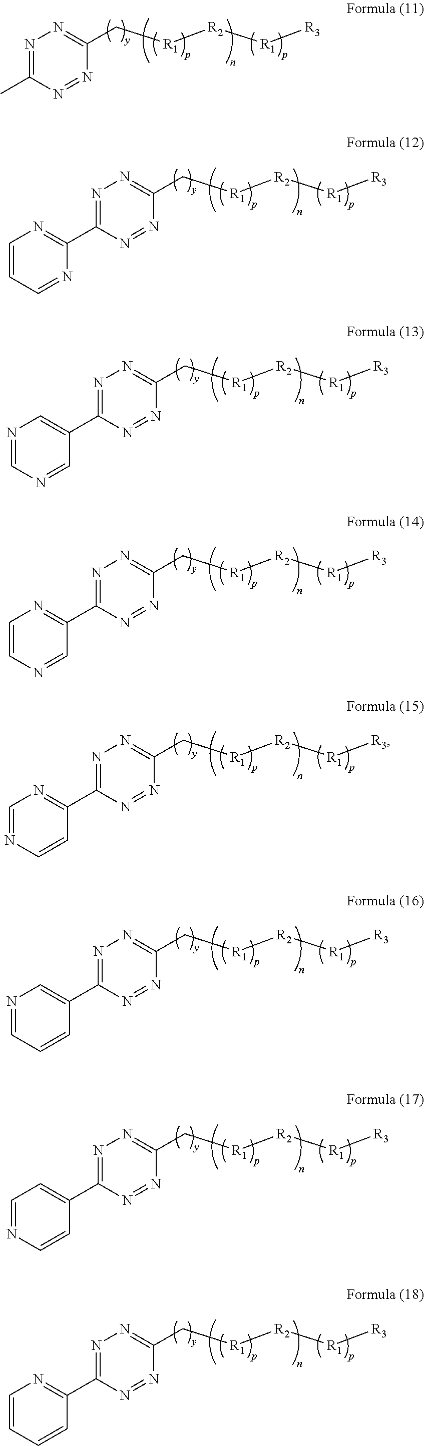

wherein n, p, y, R₁, R₂, and R₃ are as defined for Formulae (1), (2), (3), (4), (5), (6), (7), and (8),
wherein in Formulae (11), (12), (13), (14), (15), (16), (17), and (18) the moiety —(CH₂)_{y}—((R₁)ₚ—R₂)ₙ—(R₁)ₚ—R₃ has a molecular weight in a range of from 100 Da to 3000 Da,
wherein in Formula (18) y is not 1.

In some embodiments R₃ is a chelator moiety selected from the group consisting of

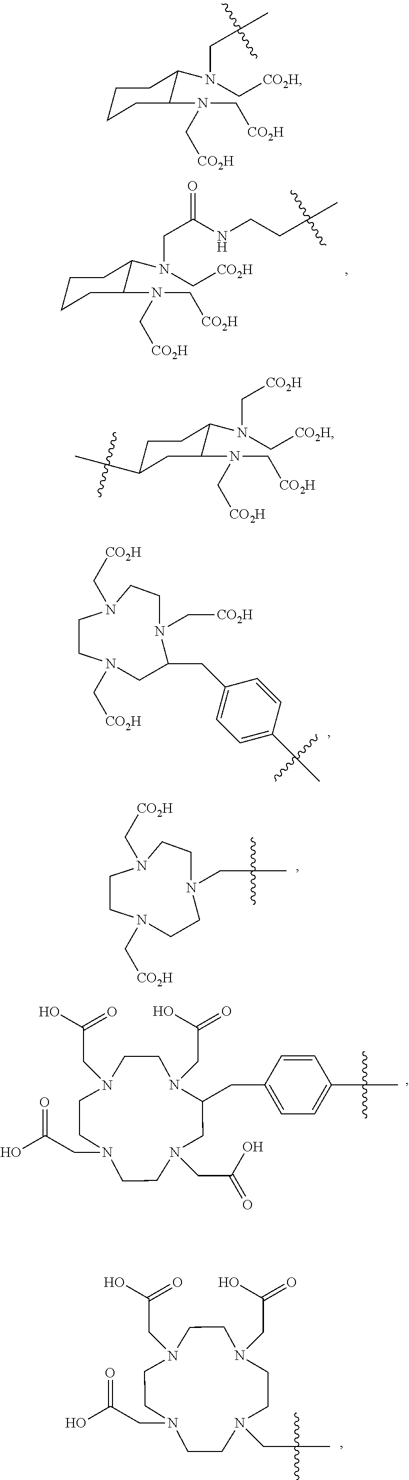

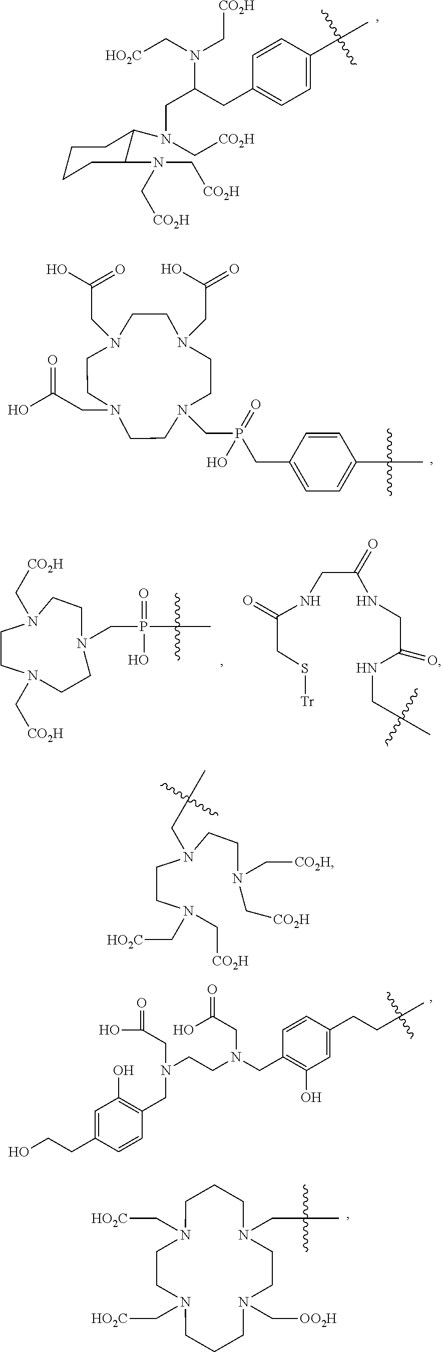

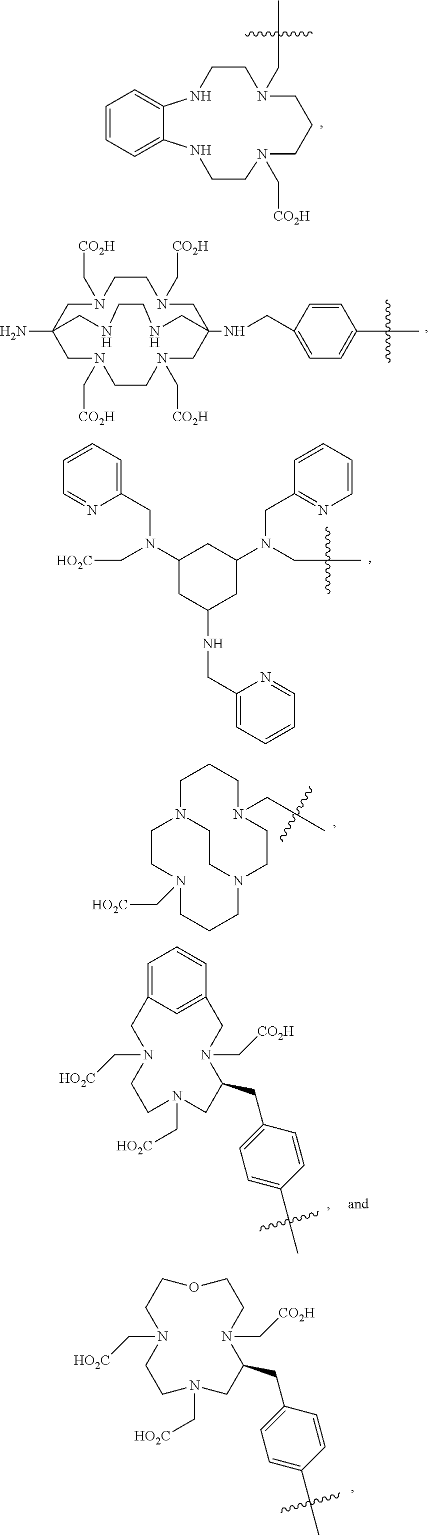

wherein the wiggly line denotes a bond to the remaining part of the molecule, optionally bound via —C(O)NH—, wherein the chelator moieties according to said group optionally chelate a metal ion.

In some embodiments the chelator moiety chelates an isotope selected from the group consisting of ⁶²Cu, ⁶⁴Cu, ⁶⁶Ga, ⁶⁷Ga, ⁶⁷Cu, ⁶⁸Ga, ⁸⁶Y, ⁸⁹Zr, ⁹⁰Y, ^{99m}Tc, ¹¹¹In, ¹⁶⁶Ho, ¹⁷⁷Lu, ¹⁸⁶Re, ¹⁸⁸Re, ²¹¹Bi, ²¹²Bi, ²¹²Pb, ²¹³Bi, ²¹⁴Bi, and ²²⁵Ac.

### The TCO Trigger:

In a preferred embodiment, the invention pertains to a kit as defined herein wherein the dienophile satisfies Formula (19a):

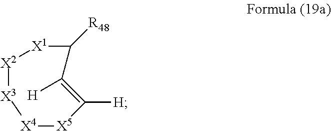

and preferably including pharmaceutically acceptable salts thereof,
wherein preferably the dienophile satisfies Formula (19):

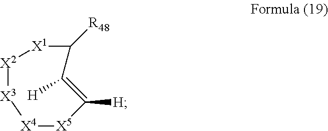

wherein R₄₈ is selected from the group consisting of —OH,
—OC(O)Cl, —OC(O)O—N-succinimidyl, —OC(O)O-4-nitrophenyl, —OC(O)O— tetrafluorophenyl, —OC(O)O-pentafluorophenyl, —OC(O)—C^{A}, —OC(S)—C^{A}, —O-(L^{C}(C^{A})ₛ(C^{A})ₛ((S^{P})ᵢ—C^{B})ⱼ)ᵣ—C^{A}, and —C^{A},
wherein r is an integer in range of from 0 to 2,
wherein each s is independently 0 or 1,
wherein i is an integer in a range of from 0 to 4,
wherein j is 0 or 1,
wherein L^{C} is a self-immolative linker,
wherein C_{A} denotes a Construct A, wherein said Construct A is selected from the group consisting of drugs, targeting agents and masking moieties,
wherein C^{B} denotes a Construct B, wherein said Construct B is selected from the group consisting of masking moieties, drugs and targeting agents,
wherein, when C^{B} is a targeting agent or a masking moiety, then C^{A} is a drug,
wherein, when C^{B} is a drug, then C^{A} is a masking moiety or a targeting agent,
wherein, when R₄₈ is —OC(O)—C^{A} or —OC(S)—C^{A}, C^{A} is bound to the —OC(O)— or —OC(S)— of R₁₅ via an atom selected from the group consisting of O, C, S, and N, preferably a secondary or a tertiary N, wherein this atom is part of C^{A},
wherein, when R₄₈ is —O-(L^{C}(C^{A})ₛ(C^{A})ₛ((S^{P})ᵢ—C^{B})ⱼ)ᵣ—C^{A} and r is 0, C^{A} is bound to the —O— moiety of R₄₈ on the allylic position of the trans-cyclooctene ring of Formula (19) via a group selected from the group consisting of —C(O)—, and —C(S)—, wherein this group is part of C^{A},
wherein, when R₄₈ is —O-(L^{C}(C^{A})ₛ(C^{A})ₛ((S^{P})ᵢ—C^{B})ⱼ)ᵣ—C^{A} and r is 1, L^{C} is bound to the —O— moiety on the allylic position of the trans-cyclooctene ring of Formula (19) via a group selected from the group consisting of —C(Y^{C2})Y^{C1}—, and a carbon atom, preferably an aromatic carbon, wherein this group is part of L^{C},
wherein Y^{C1} is selected from the group consisting of —O—, —S—, and —NR₃₆—,
wherein Y^{C2} is selected from the group consisting of O and S,
wherein, when R₄₈ is —O-(L^{C}(C^{A})ₛ(C^{A})ₛ((S^{P})ᵢ—C⁸)ⱼ)ᵣ—C^{A}, and r is 1, then C^{A} is bound to L^{C} via a moiety selected from the group consisting of —O—, —S—, and —N—, preferably a secondary or a tertiary N, wherein said moiety is part of C^{A},
wherein, when R₄₈ is —C^{A}, then C^{A} is bound to the allylic position of the trans-cyclooctene of Formula (19) via an —O— atom, wherein this atom is part of C^{A},
wherein R₃₆ is selected from the group consisting of hydrogen and C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein X⁵ is —C(R⁴⁷)₂— or —CHR₄₈, preferably X⁵ is —C(R₄₇)₂—,
wherein each X¹, X², X³, X⁴ is independently selected from the group consisting of —C(R₄₇)₂—, —C(O)—, —O—, such that at most two of X¹, X², X³, X⁴ are not —C(R₄₇)₂—, and with the proviso that no sets consisting of adjacent atoms are present selected from the group consisting of —O—O—, —O—N—, —C(O)—O—, N—N—, and —C(O)—C(O)—,
wherein each R₄₇ is independently selected from the group consisting of hydrogen, —(S^{P})ᵢ—C^{B} with i being an integer in a range of from 0 to 4, —F, —Cl, —Br, —I, —OR₃₇, —N(R₃₇)₂, —SO₃, —PO₃_{^{−}}, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(═S)O—R₃₇, C(═S)S—R₃₇, C₄-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups; wherein preferably each R₄₇ is independently selected from the group consisting of hydrogen, —F, —Cl, —Br, —I, —OH, —NH₂, —SO₃_{^{−}}, —PO₃_{^{−}}, —NO₂, —CF₃, —SH, —(S^{P})ᵢ—C^{B}, C₁-C₈ alkyl groups, C₂-C₈ alkenyl groups, C₂-C₈ alkynyl groups, C₆-C₁₂ aryl groups, C₂-C₁₂ heteroaryl groups, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₁-C₁₂ cycloalkylalkyl groups, C₅-C₁₂ cycloalkyl(hetero)aryl groups, and C₅-C₁₂ (hetero)arylcycloalkyl groups;
wherein preferably i is an integer ranging from 0 to 1,
wherein the alkyl groups, alkenyl groups, alkynyl groups, aryl, heteroaryl,
cycloalkyl groups, cycloalkenyl groups, cycloalkynyl groups,
(cyclo)alkyl(hetero)aryl groups, (hetero)aryl(cyclo)alkyl groups,
(cyclo)alkenyl(hetero)aryl groups, (hetero)aryl(cyclo)alkenyl groups,
(cyclo)alkynyl(hetero)aryl groups, (hetero)aryl(cyclo)alkynyl groups,
alkylcycloalkyl groups, cycloalkylalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized,
wherein two R₄₇ are optionally comprised in a ring,
wherein two R₄₇ are optionally comprised in a ring so as to form a ring fused to the eight-membered trans-ring,
wherein each R₃₇ is independently selected from the group consisting of hydrogen, —(S^{P})ᵢ—C^{B} with i being an integer in a range of from 0 to 4, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups;
wherein preferably each R₃₇ is independently selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₁-C₁₂ alkylcycloalkyl groups, C₄-C₁₂ cycloalkylalkyl groups, C₅-C₁₂ cycloalkyl(hetero)aryl groups, and C₅-C₁₂ (hetero)arylcycloalkyl groups;
wherein preferably i is an integer ranging from 0 to 1,
wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized,
wherein S^{P} is a spacer,
wherein preferably at most one C^{B} is comprised in the structure of Formula (19).

When C^{B} is present in a structure according to any one of Formulae (19), in some embodiments C¹³ is bound to the remainder of the molecule via a residue of R₃₂ as defined herein, wherein preferably said residue of R₃₂ equals or is comprised in a Spacer.

In other embodiments, when C^{B} is present in a structure according to any one of Formulae (19) C^{B} is bound to the remainder of the molecule via C^{M2} as defined herein, wherein preferably C^{M2} equals or is comprised in a Spacer.

In yet other embodiments, when C^{B} is present in a structure according to any one of Formulae (19) C^{B} is bound to the remainder of the molecule via C^{X} as defined herein, wherein preferably C^{X} equals or is comprised in a Spacer.

In preferred embodiments, C^{M2} is:

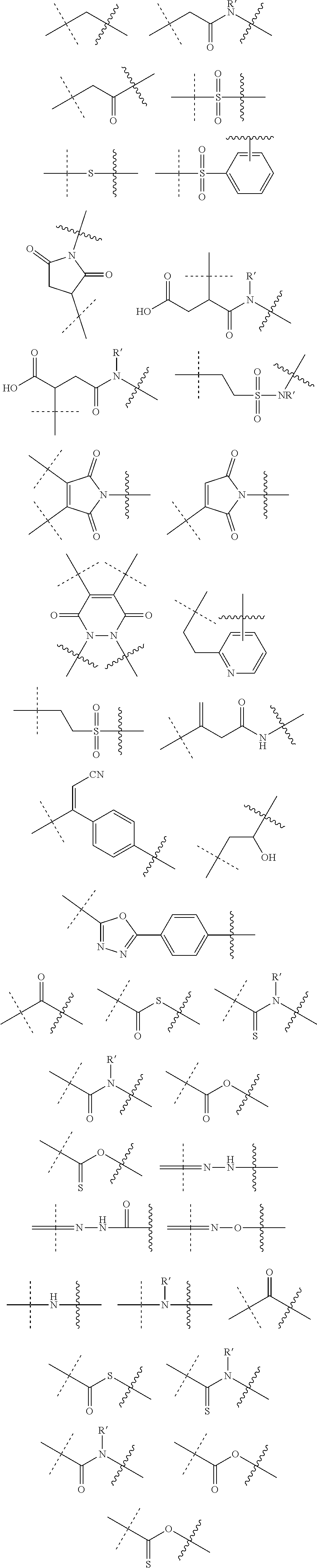

wherein the dashed line denotes a bond to C^{B} and the wiggly line denotes a bond to the remaining part of the dienophile.

In preferred embodiments, C^{X} is:

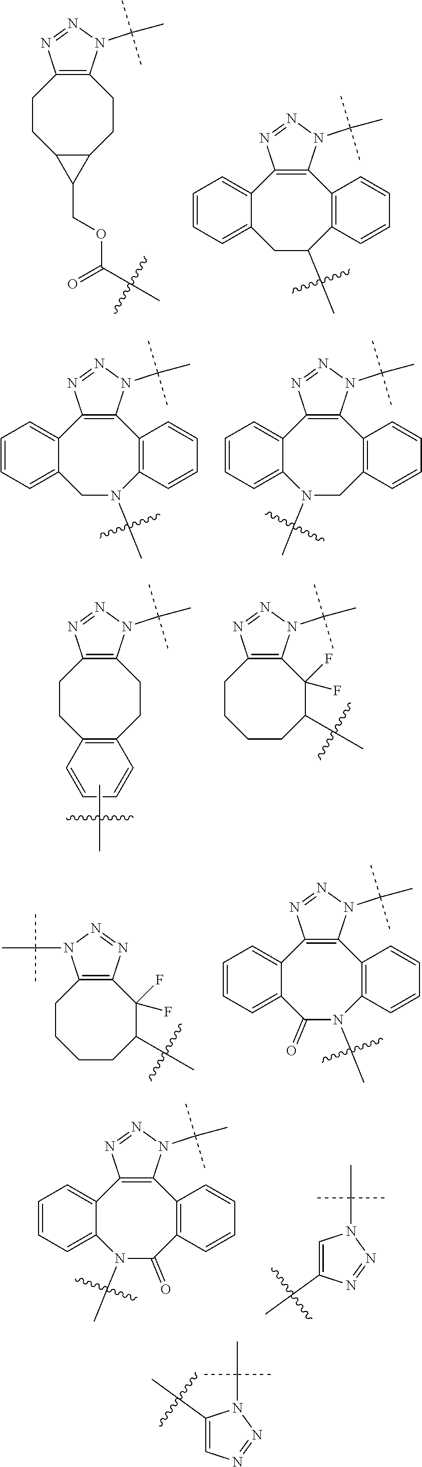

wherein the dashed line denotes a bond to C^{B} and the wiggly line denotes a bond to the remaining part of the dienophile.

In some embodiments, it is preferred when i is 0, that C^{B} is linked to the remaining part of Formulae 19 via a moiety selected from the group consisting of —O—, —C(R⁶)₂—, —NR⁶—, —C(O)—, and —S—, wherein said moieties are part of C^{B},

In some embodiments, when i is at least 1, then C^{B} is linked to S^{P} via a moiety selected from the group consisting of —O—, —C(R⁶)₂—, —NR⁶—, C(O), and —S—, wherein said moieties are part of C^{B}, and S^{P} is linked to the remaining part of Formulae 19 via a moiety selected from the group consisting of —O—, —C(R⁶)₂—, —NR⁶—, —C(O)— and —S—, wherein said moieties are part of S.

In some embodiments, it is preferred that at most one C^{B} is comprised in the structure of Formulae (19).

In some embodiments, two R₄₇ are comprised in a ring so as to form a ring fused to the eight-membered trans-ring,

In a preferred embodiment, X¹, X², X³, X⁴ are all —C(R₄₇)₂— and at most 3 of R₄₇ are not H, more preferably at most 2 R₄₇ are not H.

In a preferred embodiment, at most one of X¹, X², X³, X⁴ is not —C(R₄₇)₂— and at most 3 of R₄₇ are not H, more preferably at most 2 R₄₇ are not H.

In a preferred embodiment, two of X², X³, X⁴ together form an amide and at most 3 of R₄₇ are not H, more preferably at most 2 R₄₇ are not H.

In a preferred embodiment, X¹ is C(R₄₇)₂.

In particularly favorable embodiments, RN is in the axial position.

It is preferred that when two R₄₇ groups are comprised in a ring so as to form a ring fused to the eight-membered trans-ring, that these rings fused to the eight-membered trans-ring are C₃-C₇ cycloalkylene groups and C₄-C₇ cycloalkenylene groups, optionally substituted and containing heteroatoms as described for R₄₇.

In some embodiments the dienophile satisfies any one of the Formulae (20)-(20m) below

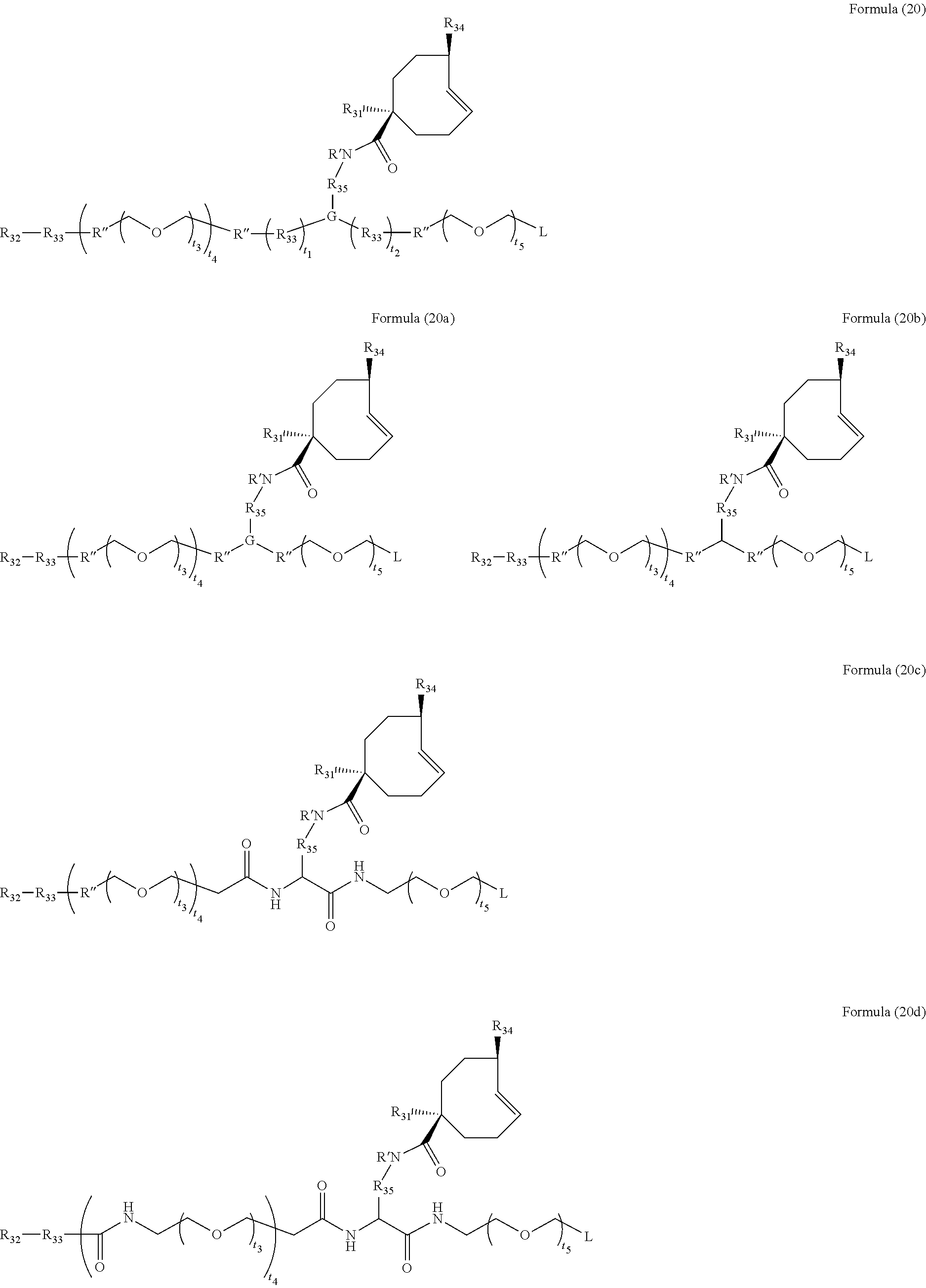

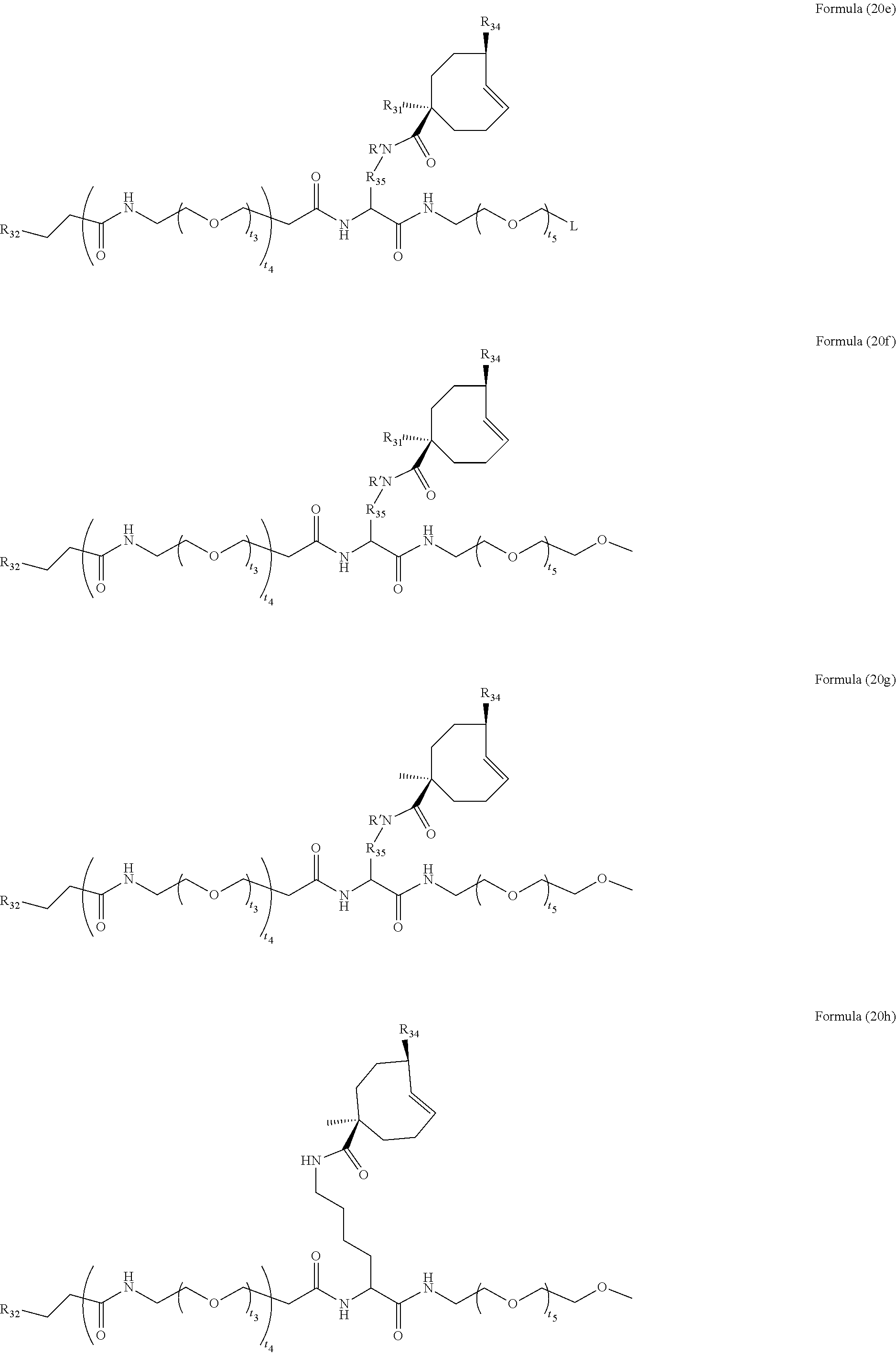

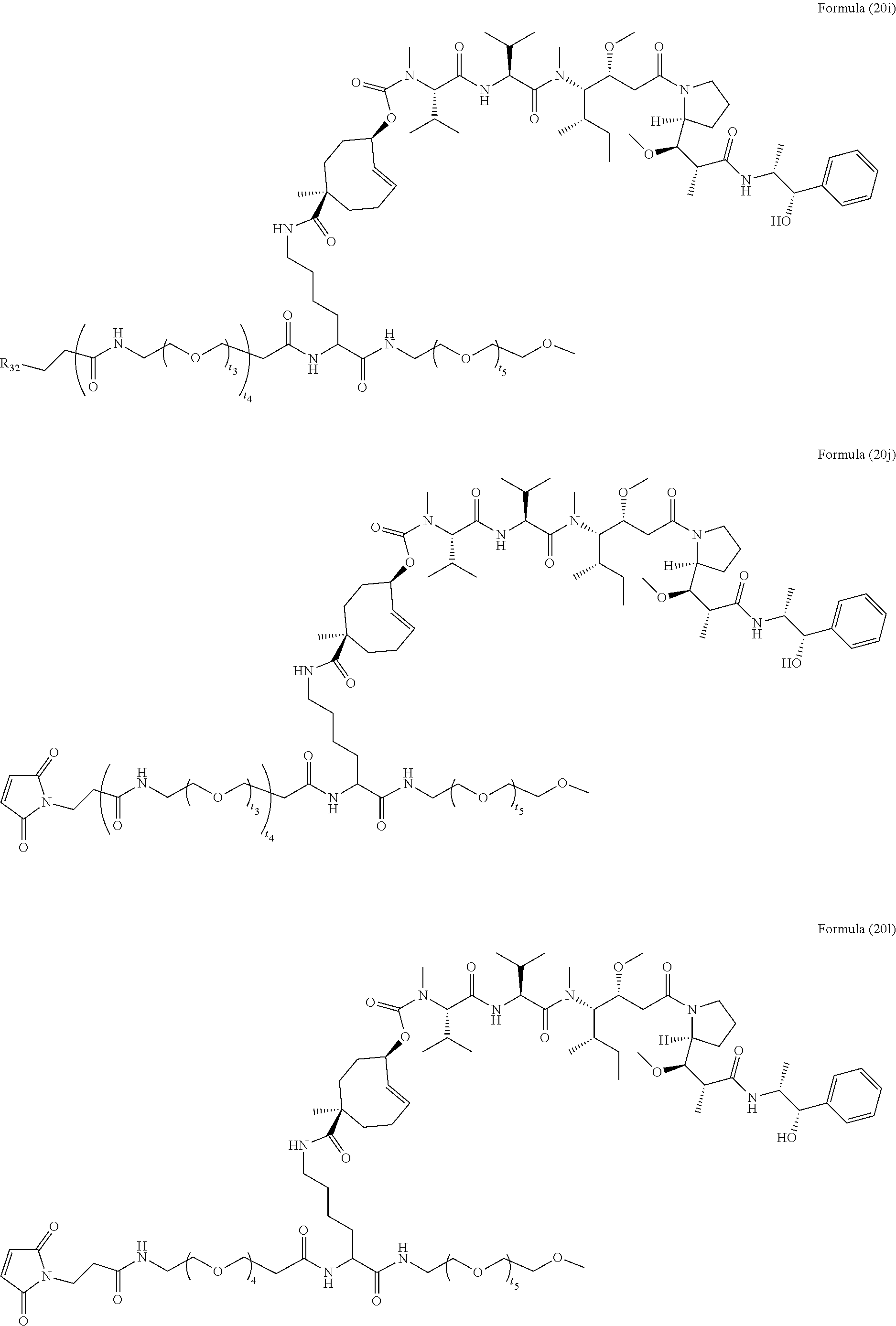

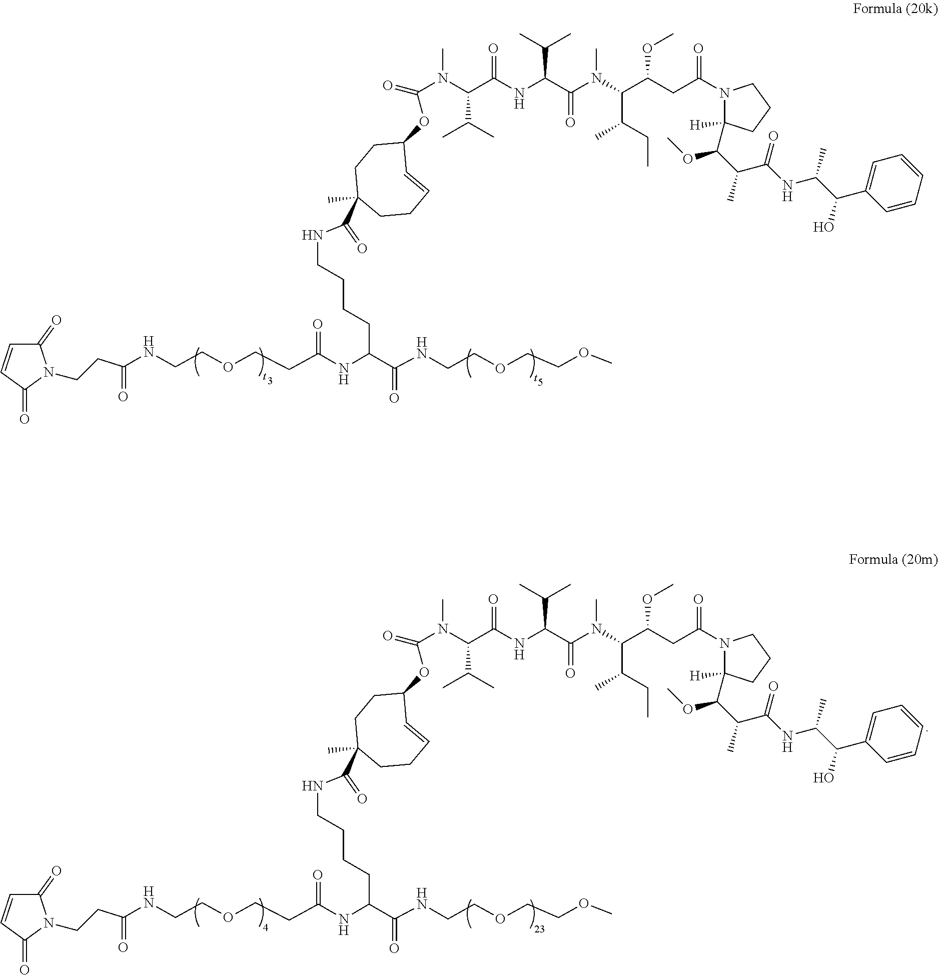

In some embodiments the dienophile satisfies Formula 21 below

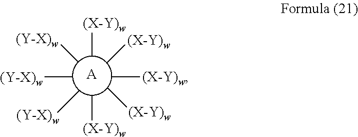

wherein moiety A is Construct-B (C^{B}), preferably a targeting agent, preferably selected from the group consisting of proteins, antibodies, peptoids and peptides, wherein C^{B} comprises at least one moiety M preferably selected from the group consisting of —OH, —NHR′, —CO₂H, —SH, —S—S—, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrile, (oxa)norbornene before modification with a compound according to Formula 20, wherein C^{B} satisfies Formula (21) after modification with at least one compound according to Formula 20:
wherein each individual w is 0 or 1, wherein at least one w is 1,
wherein each moiety Y is independently selected from moieties according to Formula (22), wherein at least one moiety Y satisfies said Formula (22):

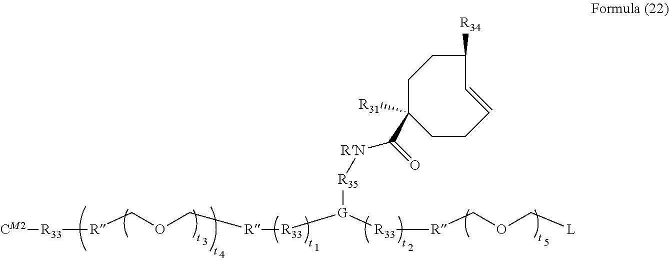

wherein moiety X is part of moiety A and was a moiety M before modification of moiety A,
wherein moiety C^{M2} is part of moiety Y and was a moiety R₃₂ before modification of moiety A,
wherein when moiety X is —S—, then C^{M2} is selected from the group consisting of

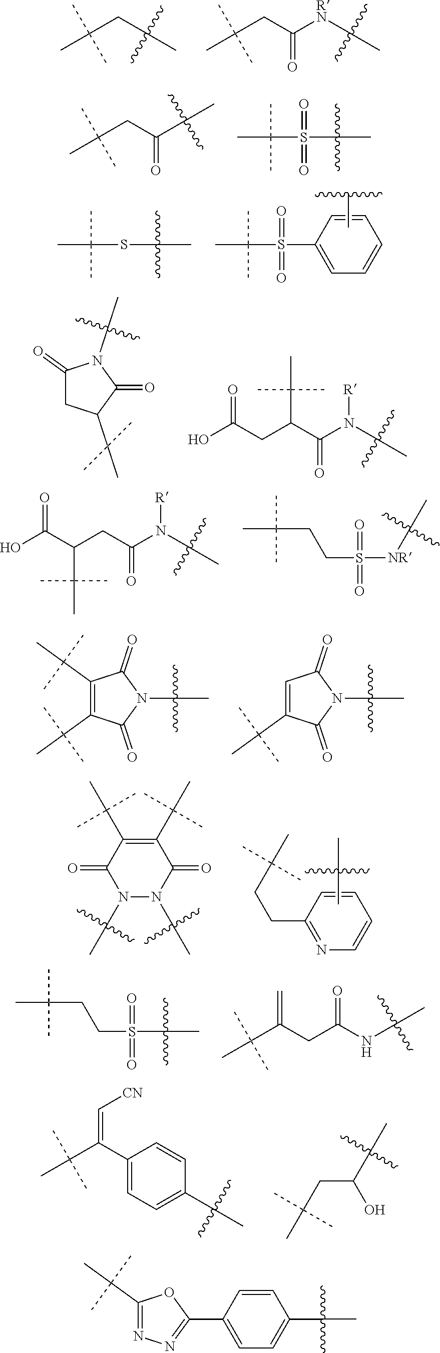

wherein the wiggly line denotes a bond to the remaining part of moiety Y, and wherein the dashed line denotes a bond to moiety X,
wherein when moiety X is —NR′—, then C^{M2} is selected from the group consisting of

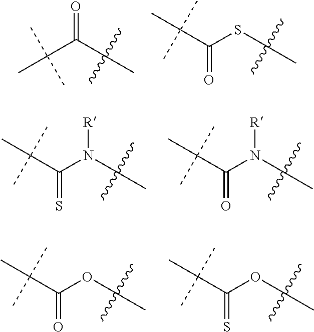

wherein the wiggly line denotes a bond to the remaining part of moiety Y, and wherein the dashed line denotes a bond to moiety X,
wherein when moiety X is —C— derived from a moiety M that was —C(O)R′ or —C(O)R′—, then C^{M2} is selected from the group consisting of

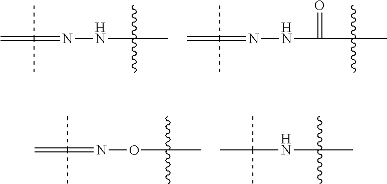

wherein the wiggly line denotes a bond to the remaining part of moiety Y, and wherein the dashed line denotes a bond to moiety X,
wherein when moiety X is —C(O)— derived from a moiety M that was —C(O)OH, then C^{M2} is selected from the group consisting of

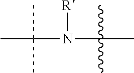

wherein the wiggly line denotes a bond to the remaining part of moiety Y, and wherein the dashed line denotes a bond to moiety X,
wherein when moiety X is —O—, then C^{M2} is selected from the group consisting of

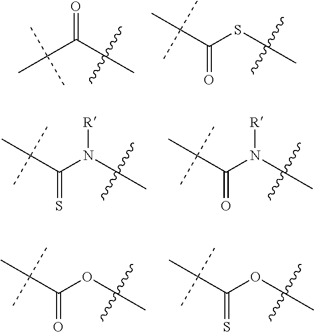

wherein the wiggly line denotes a bond to the remaining part of moiety Y, and wherein the dashed line denotes a bond to moiety X,
wherein when moiety X is derived from a moiety M that was —N₃ and that was reacted with an R₃₂ that comprised an alkyne group, then X and C^{M2} together form a moiety C^{X}, wherein C^{X} comprises a triazole ring,
wherein each C^{X} is independently selected from the group consisting of

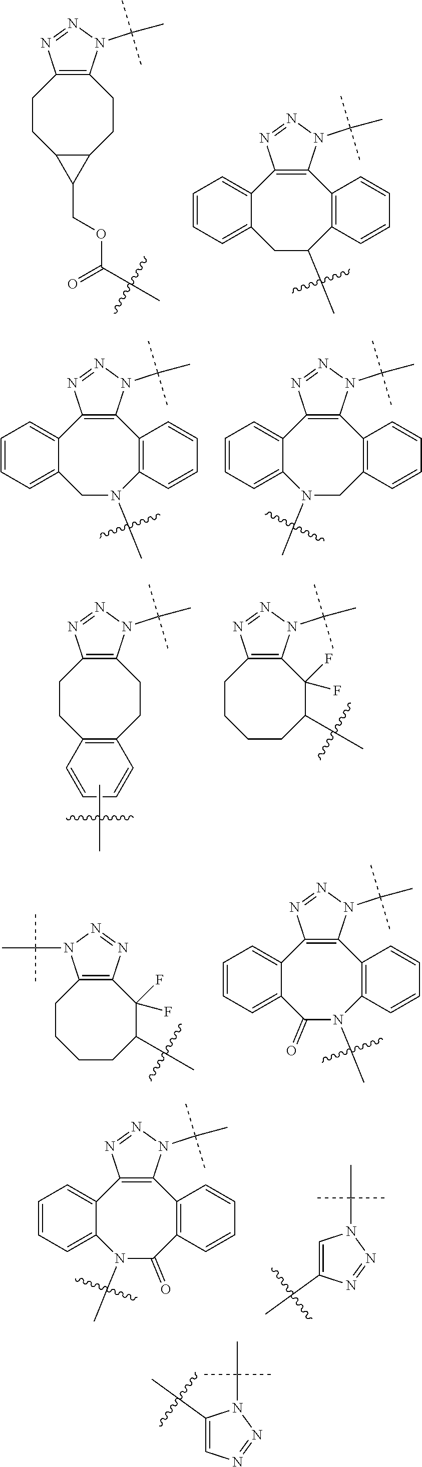

wherein the wiggly line denotes a bond to the remaining part of moiety Y, and wherein the dashed line denotes a bond to moiety X.

In preferred embodiments, moiety A is selected from the group consisting of antibodies, proteins, peptoids and peptides.

In some embodiments, moiety A can be modified with a group according to any one of Formulae (20a), (20b), (20c), (20d), (20e), (20f), (20g), (20h), (20i), (20j), (20k), (201), and (20m) as disclosed herein.

Preferably, moiety A is modified at 1 to 8 positions, more preferably from 1 to 6 positions, even more preferably at 1 to 4 positions.

In particularly favourable embodiments, moiety A is a diabody according to the sequence listed below in Table 1 as SEQ ID NO:1.

TABLE 1

| Diabody | Diabody sequence (SEQ ID NO: 1) |
|---|---|
| TAG72-binding diabody derived | SVQLQQSDAELVKPGASVKISCKASGYTFTD |
| from the CC49 antibody | HAIHWVKQNPEQGLEWIGYFSPGNDDFKY |
| | NERFKGKATLTADKSSSTAYLQLNSLTSEDS |
| | AVYFCTRSLNMAYWGQGTSVTVSSGGGGSD |
| | IVMTQSCSSCPVSVGEKVTLSCKSSQSLLYS |
| | GNQKNYLAWYQQKPGQSPKLLIYWASTRES |
| | GVPDRFTGSGSGTDFTLSISSVETEDLAVYY |
| | CQQYYSYPLTFGAGTKLVLKR |

### Formula (22)

In some embodiments of the invention, the compounds pertaining to Formula (22) can be further specified by any one of the Formulae (22a), (22b), (22c), (22d), (22e), (22f), (22g), (22h), (22i), (22j), (22k), (22l), and (22m) depicted below:

In some embodiments, the dienophile satisfies a compound according to Formula (21), wherein moiety A is modified with any one of the compounds depicted in the Formulae below:

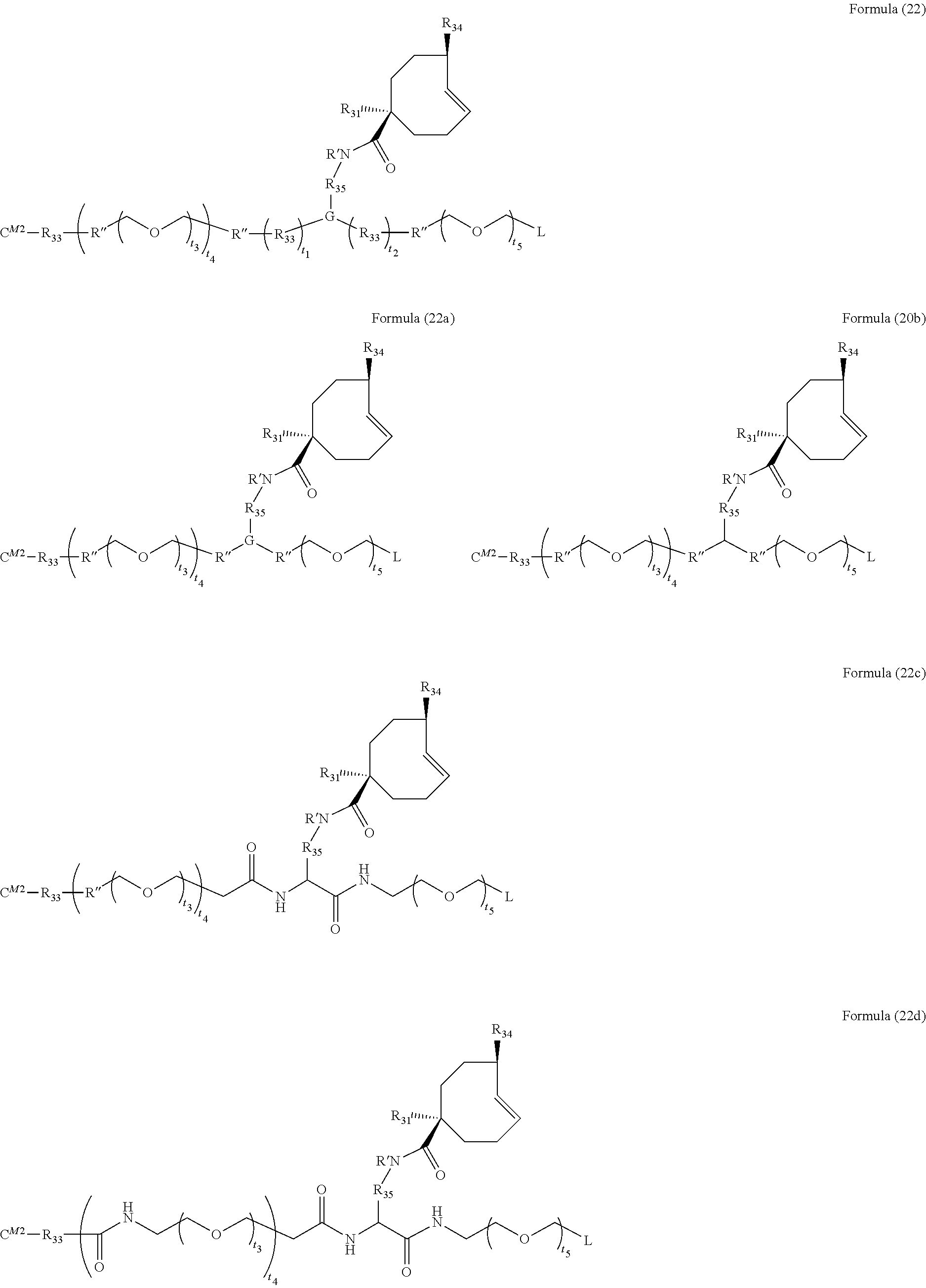

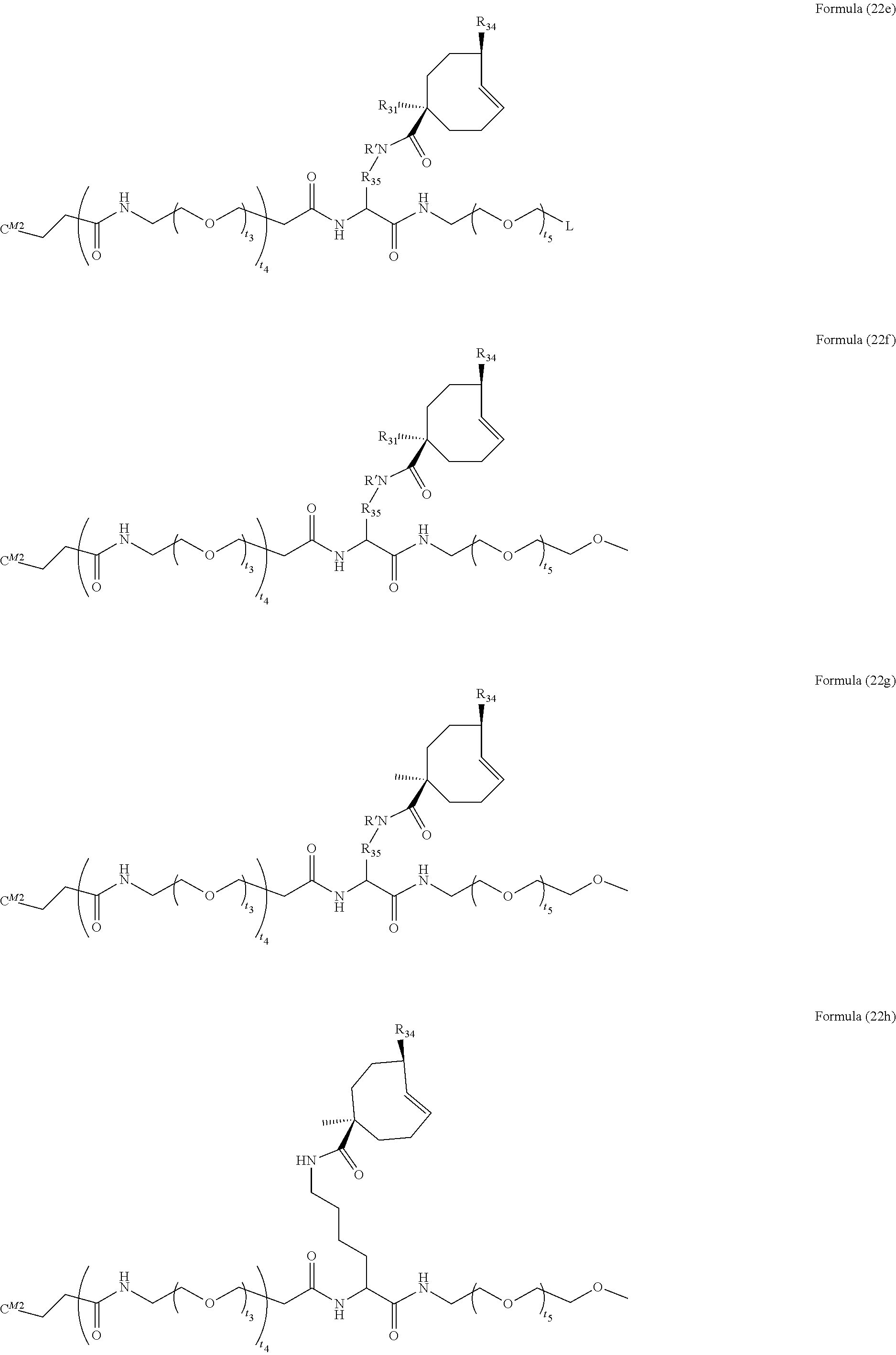

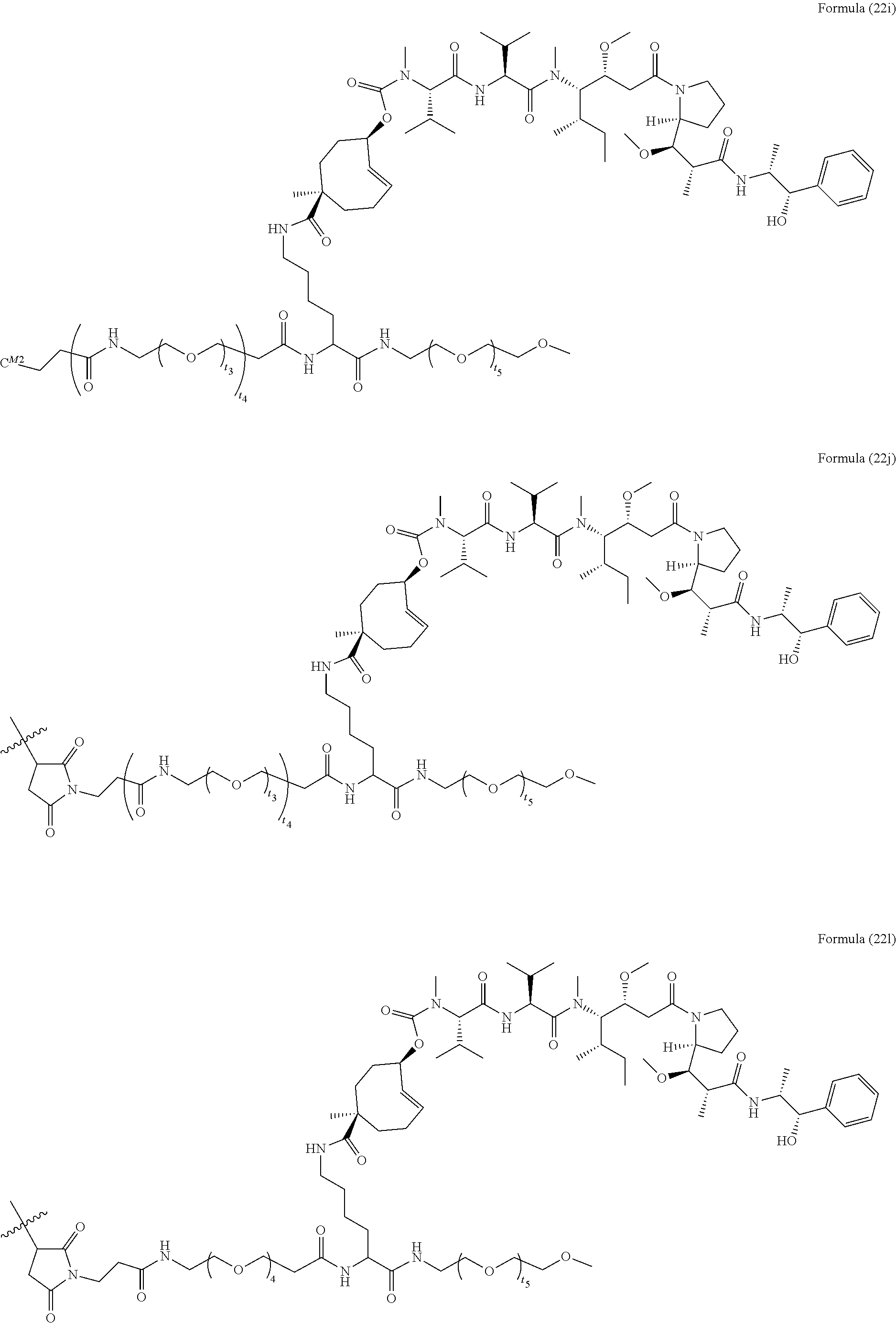

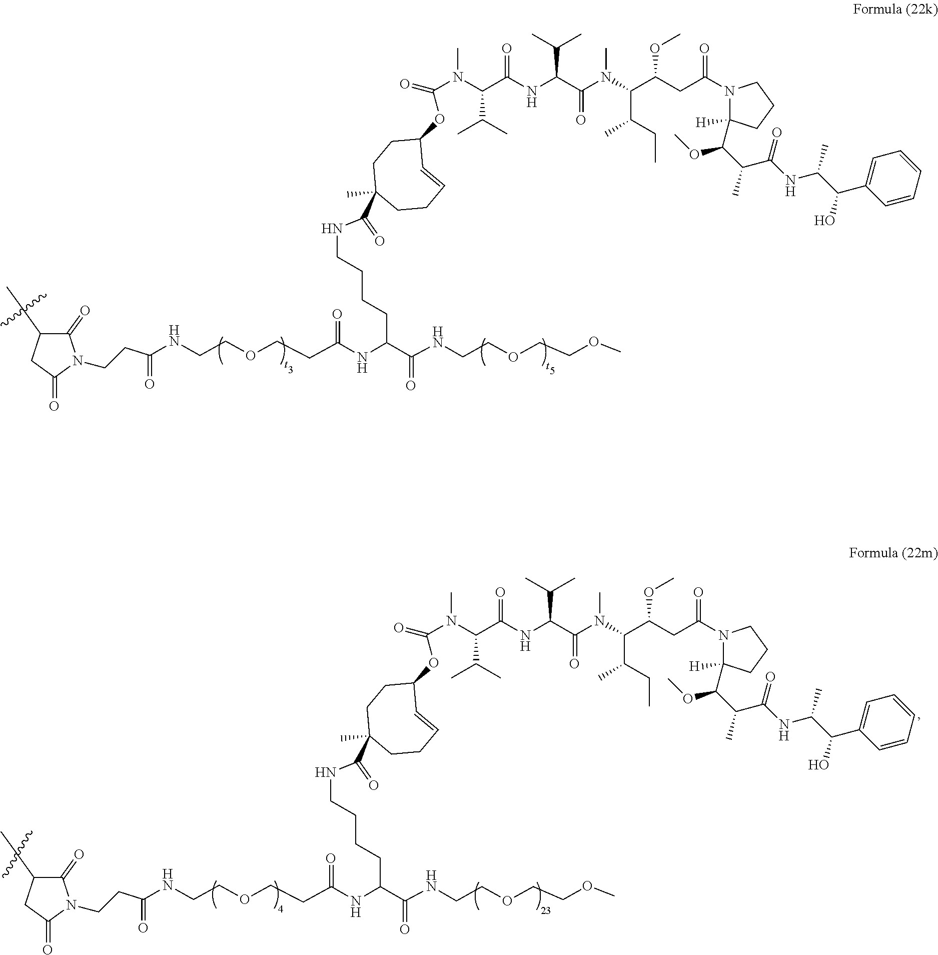

wherein in Formulae (22j), (22k), (22l), (22m), the wiggly line denotes a bond to moiety X of moiety A in Formula (21).

It will be understood that the imide moiety (22j), (22k), (22l), and (22m) may hydrolyze in aqueous environments. The hydrolysis products of these compounds, which comprise regioisomers, are understood to be disclosed herein as well.

In a particularly favourable embodiment, in Formula (21) moiety A is a diabody according to SEQ ID NO:1 as disclosed herein, and Y is the compound according to any one of the Formulae (22a), (22b), (22c), (22d), (22e), (22f), (22g), (22h), (22i), (22j), (22k), (22l), and (22m).

Preferably, in Formula (21) moiety A is a diabody according to SEQ ID NO:1 as disclosed herein, and Y is the compound according to the Formula (22m).

More preferably, in Formula (21) moiety A is a diabody according to SEQ ID NO:1 as disclosed herein, and Y is the compound according to the Formula (22m), and in four moieties —(X-Y)_{w} of Formula (21) w is 1, i.e. the diabody according to SEQ ID NO:1 is modified at four positions.

Even more preferably, in Formula (21) moiety A is a diabody according to SEQ ID NO:1 as disclosed herein, and Y is the compound according to the Formula (22m), and in four moieties —(X-Y)_{w} of Formula (21) w is 1, and X in these four moieties —(X-Y)_{w} is a sulphur atom, i.e. S, that is part of a cysteine that is part of the diabody according to SEQ ID NO:1.

The following structures are non limiting examples of suitable dienophiles:

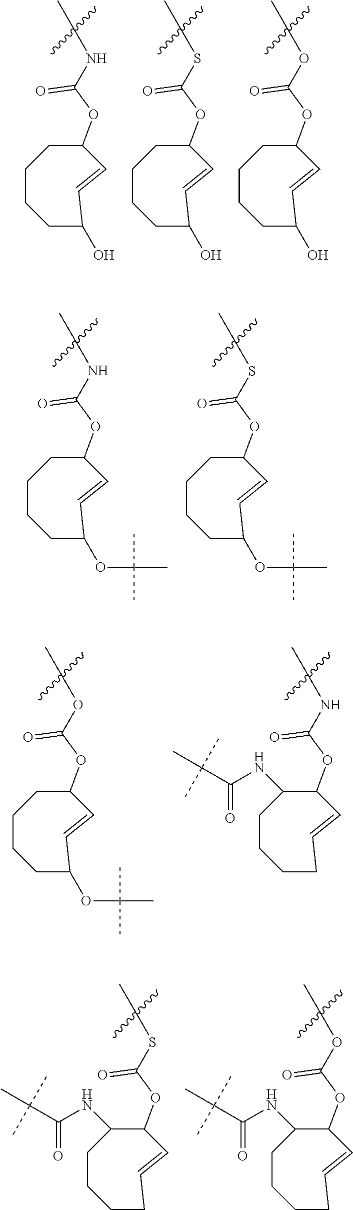

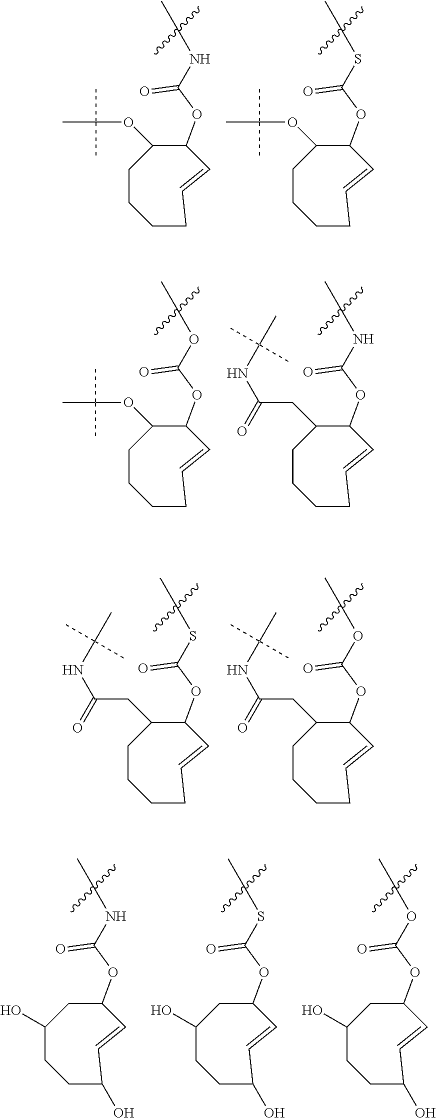

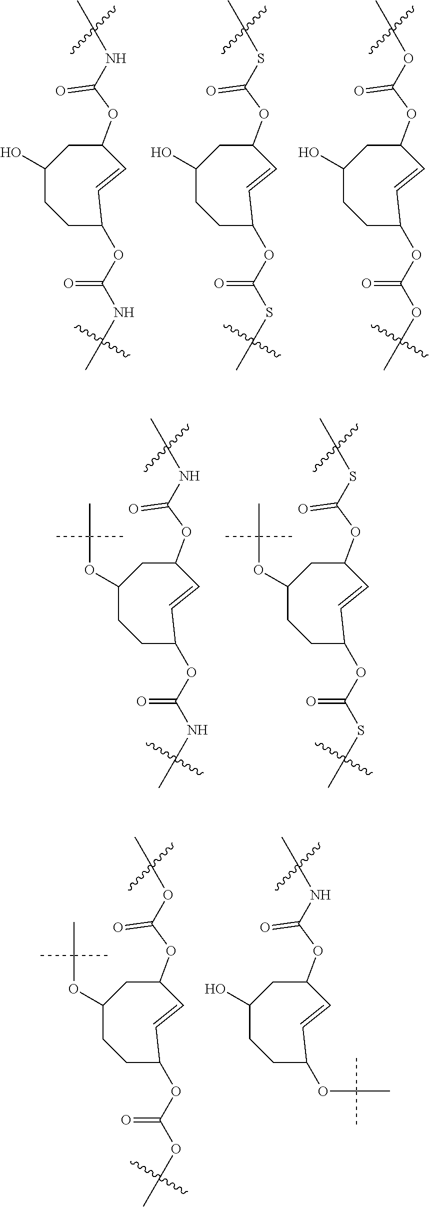

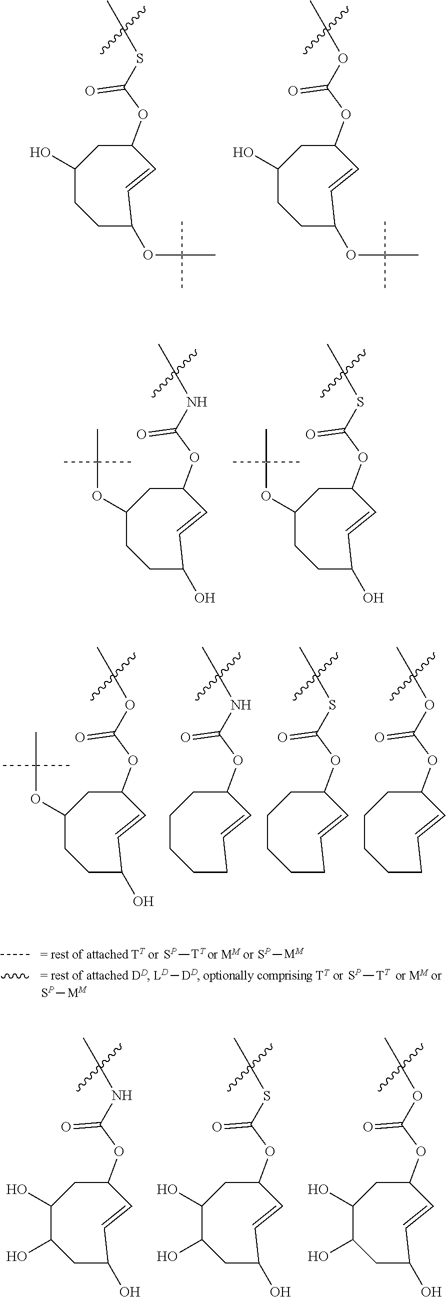

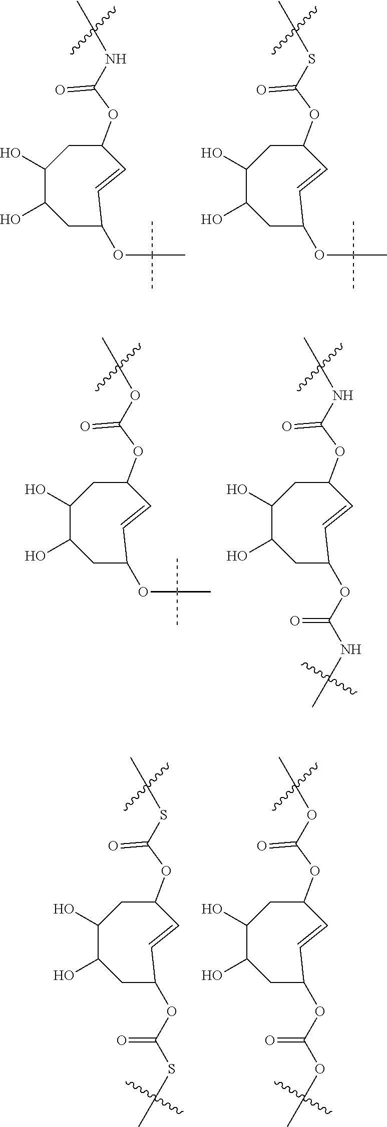

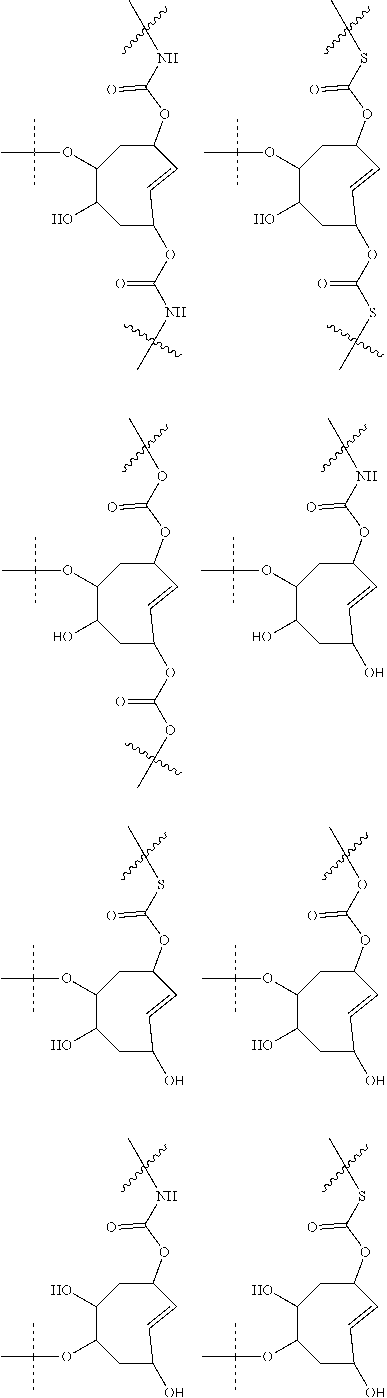

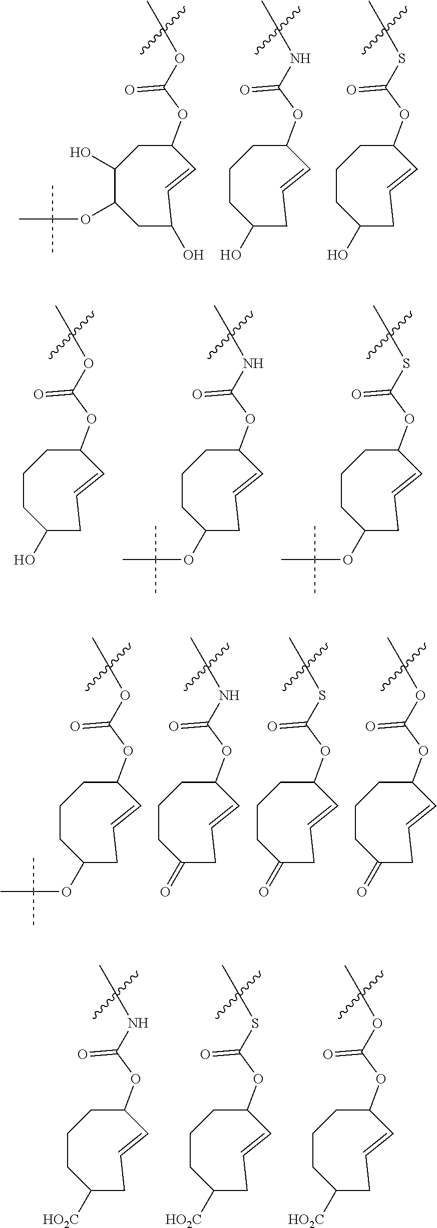

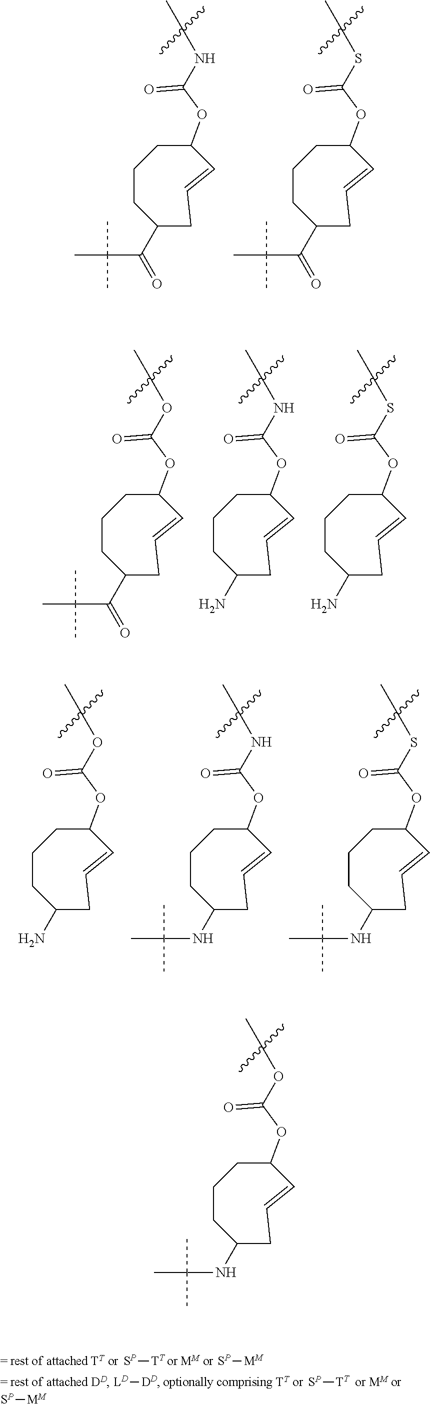

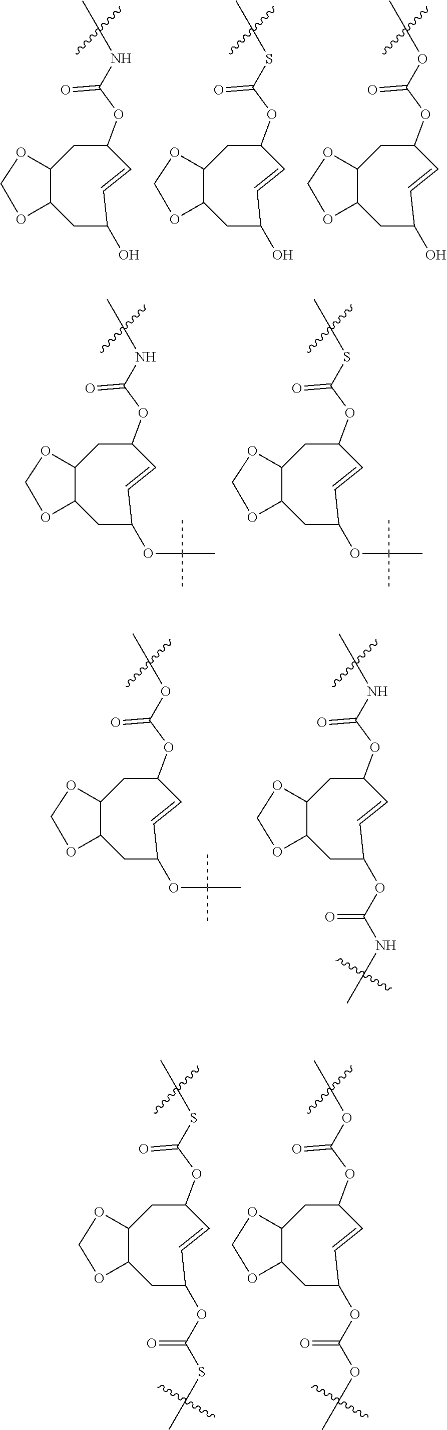

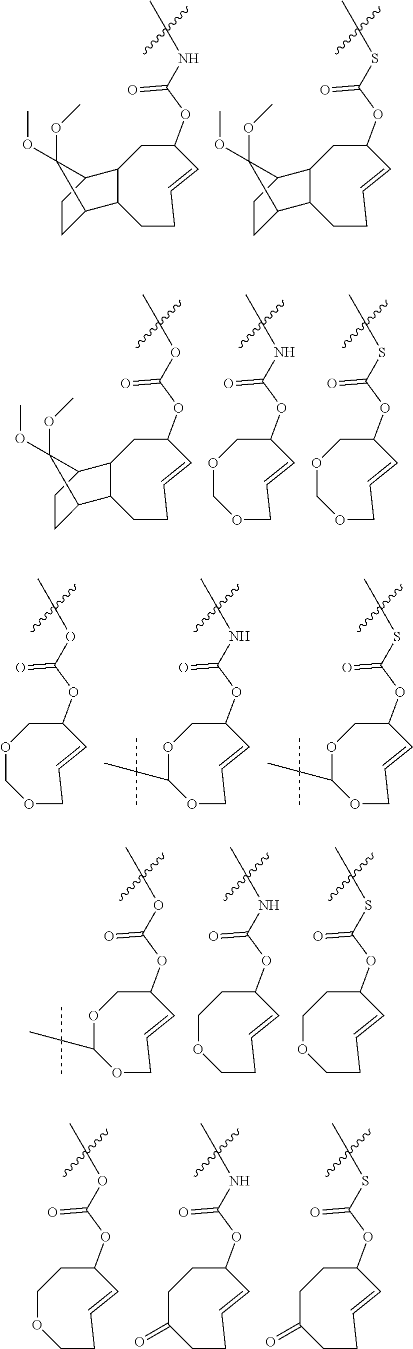

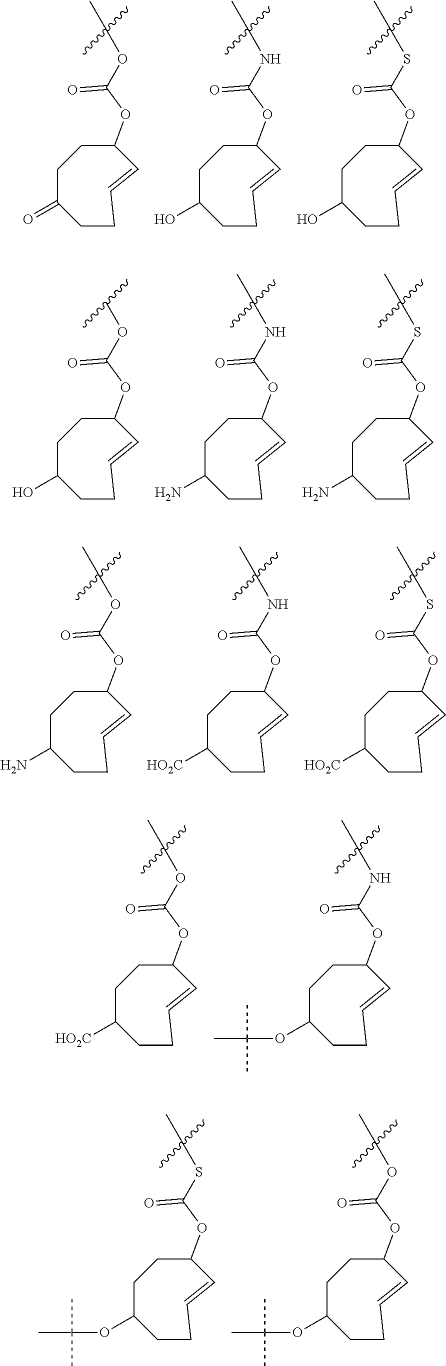

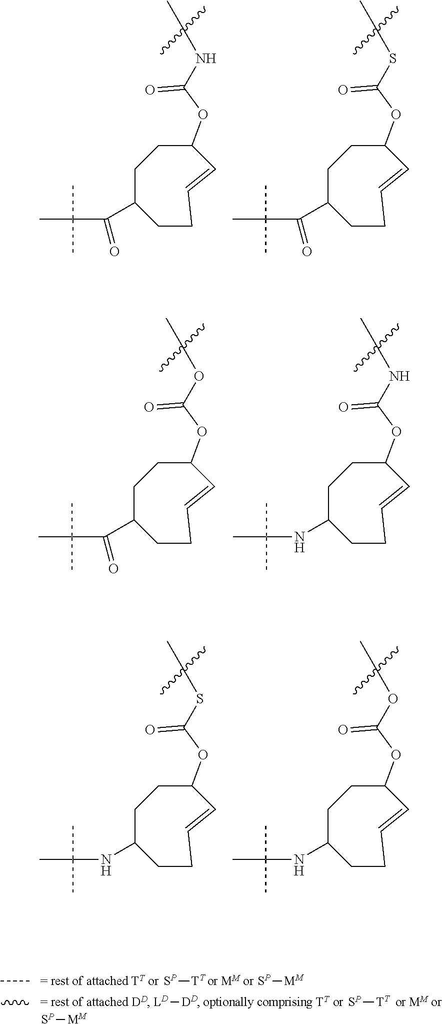

Preferred TCO compounds according to this invention are the racemic and enantiomerically pure compounds listed below:

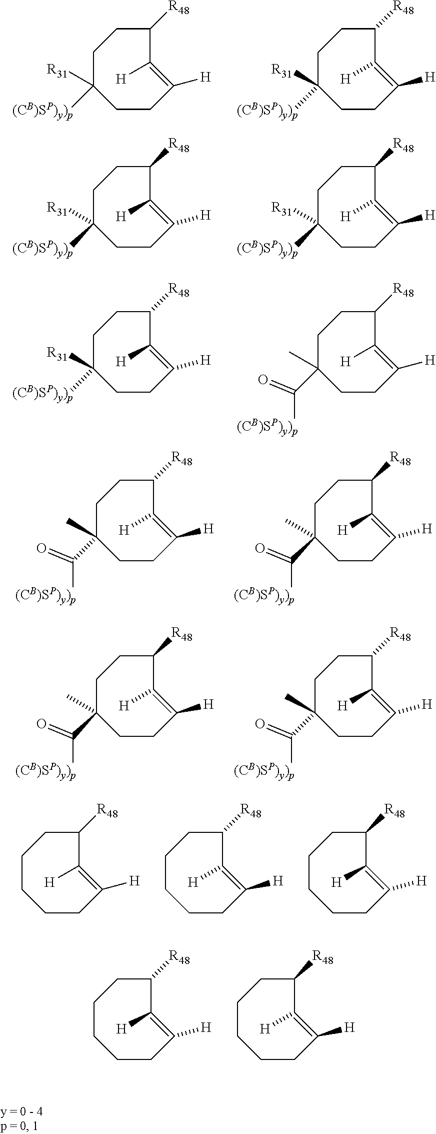

Especially preferred TCO compounds according to this invention are the enantiomerically pure compounds listed below:

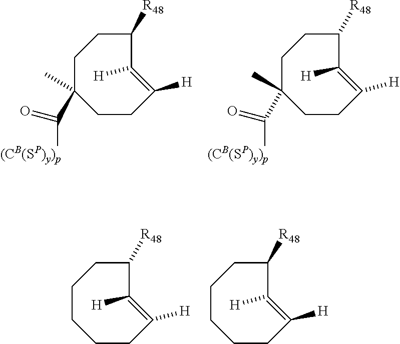

Other preferred TCO compounds are:

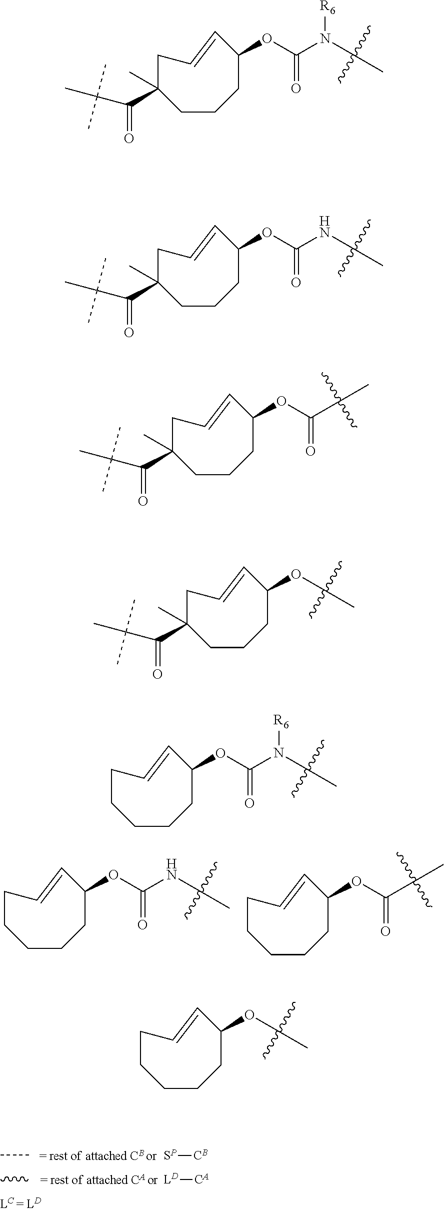

Where reference is made to L^{D} is the schemes above, L^{D} equals L^{C}.

Preferred TCO intermediates to prepare the TCO prodrugs of the invention are listed below. Particularly preferred intermediates from the below are enantiomerically pure compounds A-F, in particular A, D, E, F. A person skilled in the art will understand that compounds E and F still need to be isomerized to E-cyclooctenes, after which the enantiomer with the axial OH can be separated from the enantiomer with the equatorial OH as described by Rossin et al Bioconj. Chem., 2016 27(7):1697-1706.

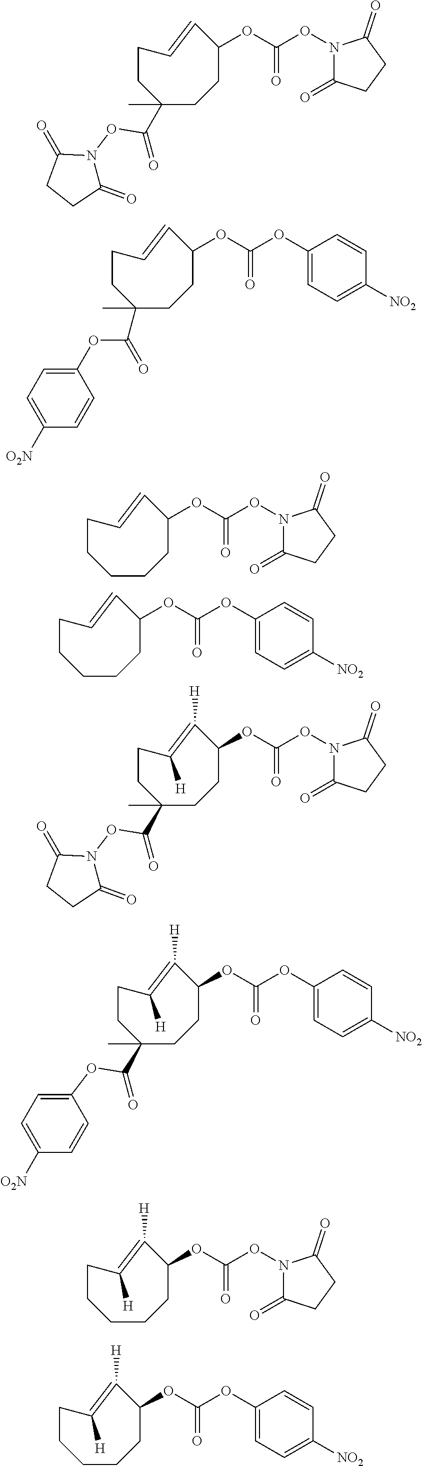

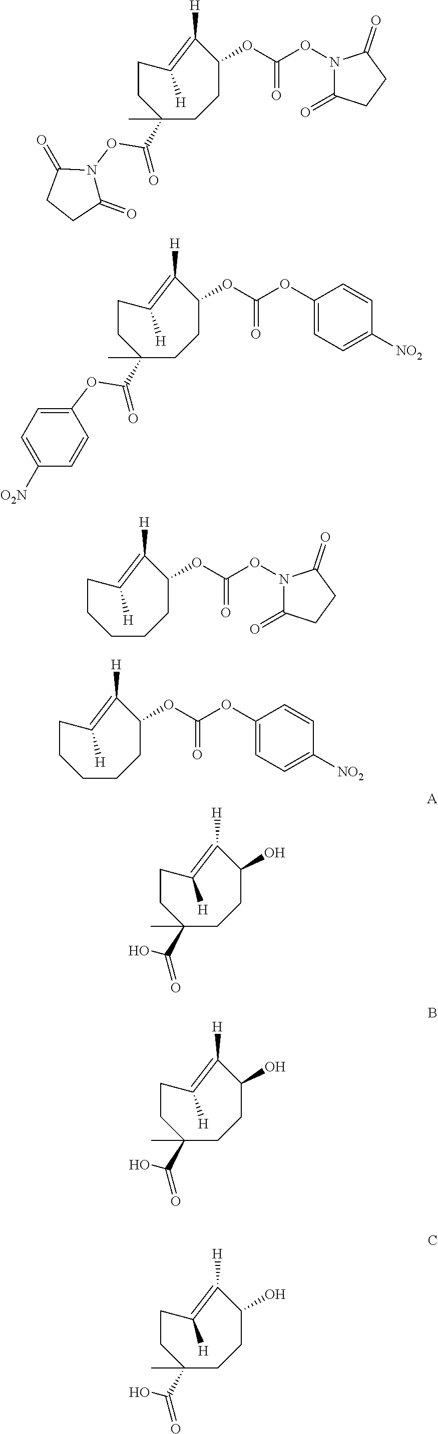

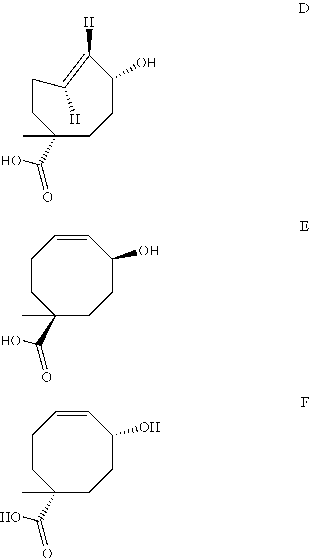

A general synthesis method of a TCO trigger and its corresponding prodrugs is shown directly below. The synthesis method is as reported in Rossin et al Nature Communications 2018, 9, 1484 and Rossin et al Bioconj. Chem., 2016 27(7):1697-1706 with the exception of the conversion of D to F, which now is conducted by mixing D with hydroxide solution in methanol, followed by evaporation and reaction with iodomethane. Please note that for sake of clarity only one of the two enantiomers of E-K is shown. A person skilled in the art will understand that the enantiomers can be separated at various stages in the synthesis using established chiral resolution methods to obtain enantiomerically pure B, E, F, H, for example, such as chiral salts.

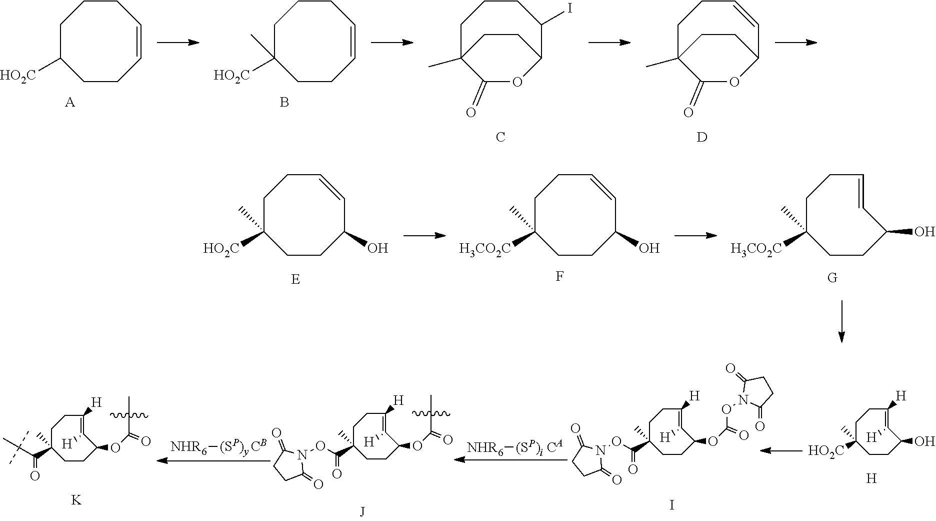

wherein the wiggly line denotes the bond to C^{A} or to a moiety comprising C^{A}, and the dashed line denotes the bond to the remainder of the molecule.

The skilled person is familiar with the fact that the dienophile activity is not necessarily dependent on the presence of all carbon atoms in the ring, since also heterocyclic monoalkenylene eight-membered rings are known to possess dienophile activity.

Thus, in general, the invention is not limited to strictly trans-cyclooctene. The person skilled in organic chemistry will be aware that other eight-membered ring-based dienophiles exist, which comprise the same endocyclic double bond as the trans-cyclooctene, but which may have one or more heteroatoms elsewhere in the ring. I.e., the invention generally pertains to eight-membered non-aromatic cyclic alkenylene moieties, preferably a cyclooctene moiety, and more preferably a trans-cyclooctene moiety.

Trans-cyclooctene or E-cyclooctene derivatives are very suitable as Triggers, especially considering their high reactivity. Optionally, the trans-cyclooctene (TCO) moiety comprises at least two exocyclic bonds fixed in substantially the same plane, and/or it optionally comprises at least one substituent in the axial position, and not the equatorial position. The person skilled in organic chemistry will understand that the term “fixed in substantially the same plane” refers to bonding theory according to which bonds are normally considered to be fixed in the same plane. Typical examples of such fixations in the same plane include double bonds and strained fused rings. E.g., the at least two exocyclic bonds can be the two bonds of a double bond to an oxygen (i.e. C═O). The at least two exocyclic bonds can also be single bonds on two adjacent carbon atoms, provided that these bonds together are part of a fused ring (i.e. fused to the TCO ring) that assumes a substantially flat structure, therewith fixing said two single bonds in substantially one and the same plane. Examples of the latter include strained rings such as cyclopropyl and cyclobutyl. Without wishing to be bound by theory, the inventors believe that the presence of at least two exocyclic bonds in the same plane will result in an at least partial flattening of the TCO ring, which can lead to higher reactivity in the IEDDA reaction. A background reference providing further guidance is WO 2013/153254.

TCO moieties may consist of multiple isomers, also comprising the equatorial vs. axial positioning of substituents on the TCO. In this respect, reference is made to Whitham et al. J. Chem. Soc. (C), 1971, 883-896, describing the synthesis and characterization of the equatorial and axial isomers of trans-cyclo-oct-2-en-ol, identified as (1RS, 2RS) and (1SR, 2RS), respectively. In these isomers the OH substituent is either in the equatorial or axial position. Without wishing to be bound by theory, the inventors believe that the presence of an axial substituent increases the TCO ring strain resulting in higher reactivity in the IEDDA reaction. A background reference providing further guidance is WO 2012/049624.

Furthermore, in case of allylic substituents on the TCO in some embodiments it is preferred that these are positioned axially and not equatorially.

It should be noted that, depending on the choice of nomenclature, the TCO dienophile may also be denoted E-cyclooctene. With reference to the conventional nomenclature, it will be understood that, as a result of substitution on the cyclooctene ring, depending on the location and molecular weight of the substituent, the same cyclooctene isomer may formally become denoted as a Z-isomer. In the present invention, any substituted variants of the invention, whether or not formally “E” or “Z,” or “cis” or “trans” isomers, will be considered derivatives of unsubstituted trans-cyclooctene, or unsubstituted E-cyclooctene. The terms “trans-cyclooctene” (TCO) as well as E-cyclooctene are used interchangeably and are maintained for all dienophiles according to the present invention, also in the event that substituents would formally require the opposite nomenclature. I.e., the invention relates to cyclooctene in which carbon atoms 1 and 6 as numbered below are in the E (entgegen) or trans position.

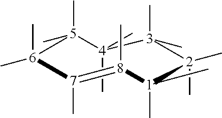

The dienophiles for use in the invention can be synthesized by the skilled person, on the basis of known synthesis routes to cyclooctenes and corresponding hetero atom(s)-containing rings. The skilled person further is aware of the wealth of cyclooctene derivatives that can be synthesized via the ring closing metathesis reaction using Grubbs catalysts. As mentioned above, the TCO possibly includes one or more heteroatoms in the ring. This is as such sufficiently accessible to the skilled person. Reference is made, e.g., to the presence of a thioether in TCO: [Cere et al. J. Org. Chem. 1980, 45, 261]. Also, e.g., an —O—SiR₂—O moiety in TCO: [Prevost et al. J. Am. Chem. Soc. 2009, 131, 14182]. Exemplary TCOs include the following structures, indicated below with literature references. Where a cyclooctene derivative is depicted as a Z-cyclooctene it is conceived that this can be converted to the E-cyclooctene analog.

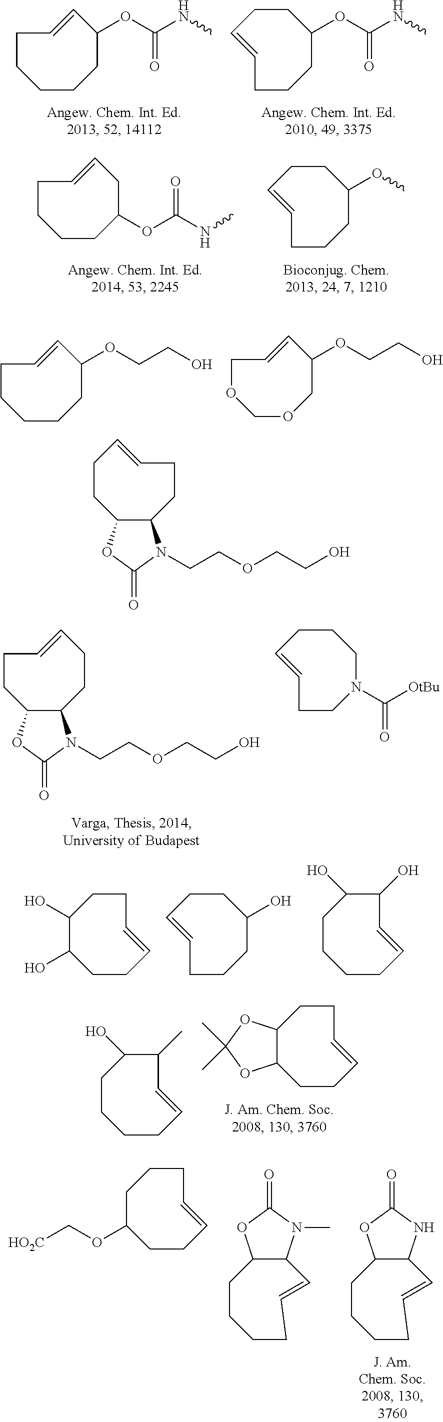

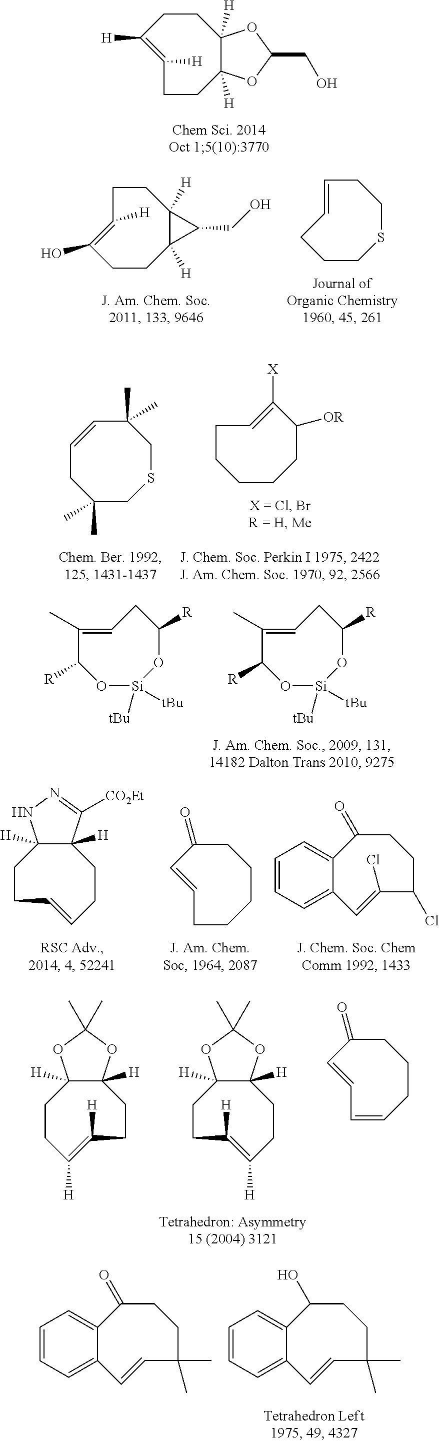

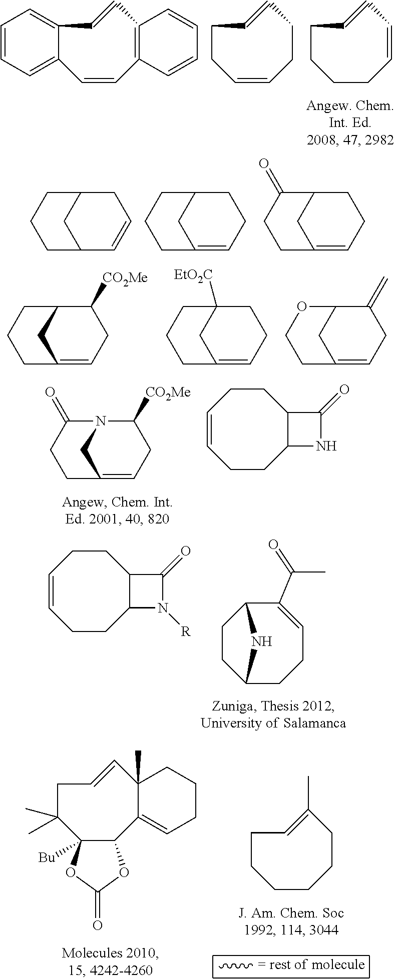

### Prodrug

A Prodrug is a conjugate of the Drug and the TCO and comprises a Drug that is capable of increased therapeutic action after release of Construct-A from the TCO. Such a Prodrug may optionally have specificity for disease targets. In a preferred embodiment Construct A is a Drug.

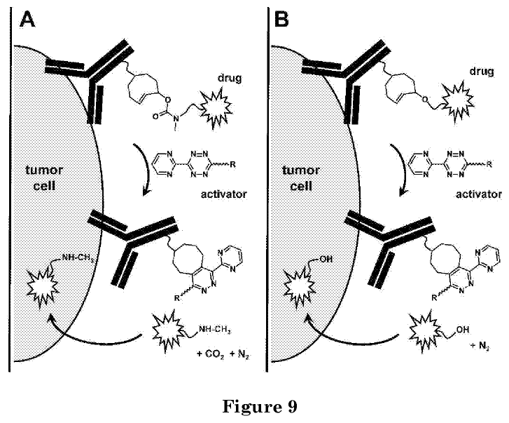

In a preferred embodiment the targeted Prodrug is an Antibody-Drug Conjugate (ADC). Activation of the Prodrug by the IEDDA pyridazine elimination of the TCO with the Activator leads to release of the Drug (FIG. 9).

It is desirable to be able to activate targeted Prodrugs such as ADCs selectively and predictably at the target site without being dependent on homogenous penetration and targeting, and on endogenous activation parameters (e.g. pH, enzymes) which may vary en route to and within the target, and from indication to indication and from patient to patient. The use of a biocompatible chemical reaction that does not rely on endogenous activation mechanisms for selective Prodrug activation would represent a powerful new tool in cancer therapy. It would expand the scope to cancer-related receptors and extracellular matrix targets that do not afford efficient internalization of the ADC and therefore cannot be addressed with the current ADC approaches. In addition, extraneous and selective activation of Prodrugs when and where required leads to enhanced control over Prodrug activation, intracellularly and extracellularly. Finally this approach would maximize the bystander effect, allowing more efficient Drug permeation throughout the tumor tissue.

Other areas that would benefit from an effective prodrug approach are protein-based therapies and immunotherapy, for example bispecific T-cell engaging antibody constructs, which act on cancer by binding cancer cells and by engaging the immune system [Trends in Biotechnology 2015, 33, 2, 65]. Antibody constructs containing an active T-cell binding site suffer from peripheral T-cell binding. This not only prevents the conjugate from getting to the tumor but can also lead to cytokine storms and T-cell depletion. Photo-activatable anti-T-cell antibodies i.e. T-cell directed Prodrugs, in which the anti-T-cell activity is only restored when and where it is required (i.e. after tumor localization via the tumor binding arm), following irradiation with UV light, has been used to overcome these problems [Thompson et al., Biochem. Biophys. Res. Commun. 366 (2008) 526-531]. However, light based activation is limited to regions in the body where light can penetrate, and is not easily amendable to treating systemic disease such as metastatic cancer.

Other proteins that could benefit from a Prodrug approach are immunotoxins and immunocytokines which suffer from respectively immunogenicity and general toxicity.

Hydrophilic polymers (such as polyethylene glycol, peptide and proteins have been used as cleavable masking moieties of various substrates, such as proteins, drugs and liposomes, in order to reduce their systemic activity. However, the used cleavage strategies were biological (pH, thiol, enzyme), as used in the ADC field, with the same drawbacks

In order to avoid the drawbacks of current prodrug activation, this invention makes use of an abiotic, bio-orthogonal chemical reaction to provoke release of the Drug from the Prodrug, such as an ADC. In this type of ADC, in a preferred embodiment, the Drug is attached to the antibody (or another type of Targeting Agent) via a Trigger, and this Trigger is not activated endogeneously by e.g. an enzyme or a specific pH, but by a controlled administration of the Activator, i.e. a species that reacts with the Trigger moiety in the ADC, to induce release of the Drug from the Trigger (or vice versa, release of the Trigger from the Drug, however one may view this release process) (FIG. 9).

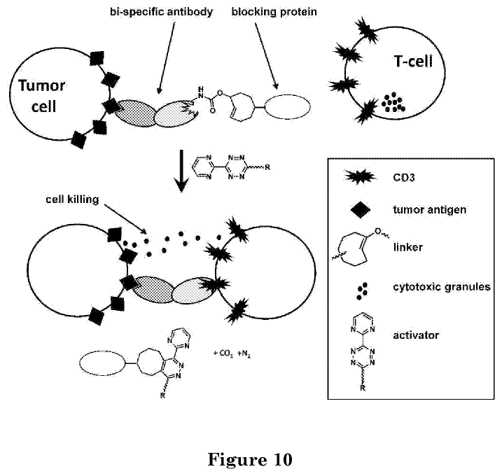

In another preferred embodiment, the Prodrug comprises a Drug bound via the trigger to a Masking Moiety. Administration of the Activator, induces release of the Drug from the Masking Moiety, resulting in activation of the Drug. In a particular embodiment, a protein with specificity for a tumor target is fused to a protein with specificity for the CD3 receptor on T-cells, wherein the CD3 binding domain is masked by conjugation of a cysteine near the domain to a Trigger comprising a Masking Moiety. Following tumor binding of the masked bispecific protein, the Activator is administered leading to unmasking of the CD3 domain and the binding to T-cells (FIG. 10).

In a preferred embodiment, the present invention provides a kit for the administration and activation of a Prodrug, the kit comprising a Drug, denoted as C^{A}, linked directly, or indirectly through a linker L^{C}, to a Trigger moiety T^{R}, wherein T^{R} or L^{C} is bound to a Construct-B, C^{B}, that is Targeting Agent T^{T} or a Masking Moiety M^{M}, and an Activator for the Trigger moiety, wherein the Trigger moiety comprises a dienophile and the Activator comprises a diene, the dienophile satisfying Formulae (19).

In other embodiments, C^{B} is the Drug and C^{A} is a targeting agent or a masking moiety.

In yet another aspect, the invention provides a method of modifying a Drug compound into a Prodrug that can be triggered by an abiotic, bio-orthogonal reaction, the method comprising the steps of providing a Drug and chemically linking the Drug to a TCO moiety satisfying Formulae (19).

In a still further aspect, the invention provides a method of treatment wherein a patient suffering from a disease that can be modulated by a Drug, is treated by administering, to said patient, a Prodrug comprising a Drug, a Trigger moiety and a Targeting agent after activation of which by administration of an Activator the Drug will be released, wherein the Trigger moiety comprises a structure satisfying Formulae (19).

In a still further aspect, the invention is a compound comprising a TCO moiety, said moiety comprising a linkage to a Drug, for use in Prodrug therapy in an animal or a human being.

In another aspect, the invention is the use of a tetrazine as an

Activator for the release, in a physiological environment, of a substance covalently linked to a compound satisfying Formulae (19). In connection herewith, the invention also pertains to a tetrazine for use as an Activator for the release, in a physiological environment, of a substance linked to a compound satisfying Formulae (19), and to a method for activating, in a physiological environment, the release of a substance linked to a compound satisfying Formulae (19), wherein a tetrazine is used as an Activator.

In preferred embodiments a Prodrug is a conjugate of the Drug and the Trigger and thus comprises a Drug that is capable of increased therapeutic action after its release from the Trigger. In embodiments where the Prodrug is targeted to a Primary Target, as is the case with for example Antibody Drug Conjugates, the Prodrug can comprise a Targeting agent T^{T}, which is bound to either the Trigger or the L^{C}.

According to a further particular embodiment of the invention, the Prodrug is selected so as to target and or address a disease, such as cancer, an inflammation, an autoimmune disease, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme.

According to one embodiment, the Prodrug and/or the Activator can be, but are not limited to, multimeric compounds, comprising a plurality of Drugs and/or bioorthogonal reactive moieties. These multimeric compounds can be polymers, dendrimers, liposomes, polymer particles, or other polymeric constructs.

It is preferred that the optional L^{C} comprised in the Prodrug is self-immolative, affording traceless release of the C^{A}, preferably a Drug.

A Construct-Trigger comprises a conjugate of the Construct or Constructs C^{A} and the Trigger T^{R}. Optionally the Trigger is further linked to Construct or Constructs C^{B}.

The general formula of the Construct-Trigger is shown below in Formula (10a) and (10b). For the avoidance of doubt, as Y^{C} is part of L^{C} and C^{A}, Y^{C} is not separately denoted in Formula (10a) and (10b).

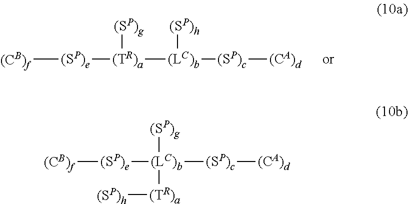

C^{A} is Construct A, C^{B} is Construct B, S^{P} is Spacer; T^{R} is Trigger, and L^{C} is Linker.

b,c,e,f,g,h≥0;a,d≥1. Formula (10a):

c,e,f,g,h≥0;a,b,d≥1. Formula (10b):

In the Trigger-Construct conjugate, the Construct C^{A} and the Trigger T^{R}—the TCO derivative- can be directly linked to each other. They can also be bound to each other via a self-immolative linker L^{C}, which may consist of multiple (self-immolative, or non immolative) units. With reference to Formula 10a and 10b, when L^{C} contains a non immolative unit, this unit equals a Spacer S^{P} and c≥1. It will be understood that the invention encompasses any conceivable manner in which the diene Trigger is attached to the one or more Construct C^{A}. The same holds for the attachment of one or more Construct C^{B} to the Trigger or the linker L^{C}. The same holds for the optional attachment of one or more Spacer S^{P} to the Trigger or the linker L^{C}. Methods of affecting conjugation, e.g. through reactive amino acids such as lysine or cysteine in the case of proteins, are known to the skilled person. Exemplary conjugation methods are outlined in the section on Conjugation herein below.

It will be understood that the Construct C^{A} is preferably linked to the TCO in such a way that the Construct C^{A} is eventually capable of being released after formation of the IEDDA adduct. Generally, this means that the bond between the Construct C^{A} and the TCO, or in the event of a self-immolative Linker L^{C}, the bond between the Linker and the TCO and between the Construct C^{A} and the Linker, should be cleavable. Predominantly, the Construct C^{A} and the optional Linker is linked via a hetero-atom, preferably via O, N, NH, or S. The cleavable bond is preferably selected from the group consisting of carbamate, thiocarbamate, carbonate, ester, amide, thioester, sulfoxide, and sulfonamide bonds.

It shall be understood that one C^{B} can be modified with more than one Trigger. For example, an antibody can be modified with four TCO-drug constructs by conjugation to four amino acid residues, wherein C^{A} is drug.

Likewise, it shall be understood that one C^{A} can be modified with more than one Trigger. For example, a protein drug can be masked by conjugation of four amino acid residues to four TCO-polyethylene glycol constructs, wherein polyethylene glycol is C^{B}.

Furthermore, it shall be understood that one C^{A} can be modified with more than one Trigger, wherein at least one Trigger links to a Targeting Agent, being C^{B}, and at least one Trigger links to a Masking Moiety being C^{B}.

### Drugs:

Drugs that can be used in a Prodrug, e.g. an ADC, relevant to this invention are pharmaceutically active compounds, in particular low to medium molecular weight compounds, preferably organic compounds, (e.g. about 200 to about 2500 Da, preferably about 300 to about 1750 Da, more preferably about 300 to about 1000 Da).

In a preferred embodiment the pharmaceutically active compound is selected from the group consisting of cytotoxins, antiproliferative/antitumor agents, antiviral agents, antibiotics, anti-inflammatory agents, chemosensitizing agents, radiosensitizing agents, immunomodulators, immunosuppressants, immunostimulants, anti-angiogenic factors, and enzyme inhibitors.

In some embodiments these pharmaceutically active compounds are selected from the group consisting of antibodies, antibody derivatives, antibody fragments, proteins, aptamers, oligopeptides, oligonucleotides, oligosaccharides, carbohydrates, as well as peptides, peptoids, steroids, toxins, hormones, cytokines, and chemokines.

In some embodiments these drugs are low to medium molecular weight compounds, preferably organic compounds, (e.g. about 200 to about 2500 Da, preferably about 300 to about 1750 Da, more preferably about 300 to about 1000 Da).

Exemplary cytotoxic drug types for use as conjugates to the TCO and to be released upon IEDDA reaction with the Activator, for example for use in cancer therapy, include but are not limited to DNA damaging agents, DNA crosslinkers, DNA binders, DNA alkylators, DNA intercalators, DNA cleavers, microtubule stabilizing and destabilizing agents, topoisomerases inhibitors, radiation sensitizers, anti-metabolites, natural products and their analogs, peptides, oligonucleotides, enzyme inhibitors such as dihydrofolate reductase inhibitors and thymidylate synthase inhibitors.

Examples include but are not limited to colchinine, vinca alkaloids, anthracyclines (e.g. doxorubicin, epirubicin, idarubicin, daunorubicin), camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, deBouganin, dactinomycines, CC1065 and its analogs, duocarmycins, maytansines, maytansinoids, dolastatins, auristatins, pyrrolobenzodiazepines and dimers (PBDs), indolinobenzodiazepines and dimers, pyridinobenzodiazepines and dimers, mitomycins (e.g. mitomycin C, mitomycin A, caminomycin), melphalan, leurosine, leurosideine, actinomycin, tallysomycin, lexitropsins, bleomycins, podophyllotoxins, etoposide, etoposide phosphate, staurosporin, esperamicin, the pteridine family of drugs, SN-38 and its analogs, platinum-based drugs, cytotoxic nucleosides.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, and RNA polymerase inhibitors.

Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. Suitable auristatins are also described in U.S. Publication Nos. 2003/0083263, 2011/0020343, and 2011/0070248; PCT Application Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety.

Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497. Other examples include mertansine and ansamitocin.

Pyrrolobenzodiazepines (PBDs), which expressly include dimers and analogs, include but are not limited to those described in [Denny, Exp. Opin. Ther. Patents, 10(4):459-474 (2000)], [Hartley et al., Expert Opin Investig Drugs. 2011, 20(6):733-44], Antonow et al., Chem Rev. 2011, 111(4), 2815-64]. Exemplary indolinobenzodiazepines are described in literature. Exemplary pyridinobenzodiazepines are described in literature.

Calicheamicins include, e.g. enediynes, esperamicin, and those described in U.S. Pat. Nos. 5,714,586 and 5,739,116

Examples of duocarmycins and analogs include CC1065, duocarmycin SA, duocarmycin A, duocarmycin B1, duocarmycin B2, duocarmycin C1, duocarmycin C2, duocarmycin D, DU-86, KW-2189, adozelesin, bizelesin, carzelesin, seco-adozelesin, CPI, CBI. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety.

Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety.

Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety.

Exemplary cryptophycin compounds are described in U.S. Pat. Nos. 6,680,311 and 6,747,021; the disclosures of which are incorporated herein by reference in their entirety.

Exemplary platinum compounds include cisplatin, carboplatin, oxaliplatin, iproplatin, ormaplatin, tetraplatin.

Exemplary DNA binding or alkylating drugs include CC-1065 and its analogs, anthracyclines, calicheamicins, dactinomycines, mitromycines, pyrrolobenzodiazepines, indolinobenzodiazepines, pyridinobenzodiazepines and the like.

Exemplary microtubule stabilizing and destabilizing agents include taxane compounds, such as paclitaxel, docetaxel, tesetaxel, and carbazitaxel;

maytansinoids, auristatins and analogs thereof, vinca alkaloid derivatives, epothilones and cryptophycins.

Exemplary topoisomerase inhibitors include camptothecin and camptothecin derivatives, camptothecin analogs and non-natural camptothecins, such as, for example, CPT-11, SN-38, topotecan, 9-aminocamptothecin, rubitecan, gimatecan, karenitecin, silatecan, lurtotecan, exatecan, diflometotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med Chem., 30:1774 (1987).

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure.

Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MLN8237; PLK inhibitors such as, for example, BI 2536, BI6727, GSK461364, ON-01910; and KSP inhibitors such as, for example, SB 743921, SB 715992, MK-0731, AZD8477, AZ3146 and ARRY-520.

Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors.

Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,944 and include GDC-0973, GSK1120212, MSC1936369B, AS703026, RO5126766 and RO4987655, PD0325901, AZD6244, AZD8330 and GDC-0973.

Exemplary B-raf inhibitors include CDC-0879, PLX-4032, and SB590885.

Exemplary B p38 MAPK inhibitors include BIRB 796, LY2228820 and SB 202190.

Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib.

Exemplary protein chaperon inhibitors include HSP90 inhibitors. Exemplary inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA).

Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461.

Exemplary Wnt/Hedgehog signalling pathway inhibitors include vismodegib, cyclopamine and XAV-939.

Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin.

Exemplary cytokines include IL-2, IL-7, IL-10, IL-12, IL-15, IL-21, TNF.

Exemplary immunomodulators are APRIL, cytokines, including IL-2, IL-7, IL-10, IL-12, IL-15, IL-21, TNF, interferon gamma, GMCSF, NDV-GMCSF, and agonists and antagonists of STING, agonists and antagonists of TLRs including TLR1/2, TLR3, TLR4, TLR7/8, TLR9, TLR12, agonists and antagonists of GITR, CD3, CD28, CD40, CD74, CTLA4, OX40, PD1, PDL1, RIG, MDA-5, NLRP1, NLRP3, AIM2, IDO, MEK, cGAS, and CD25, NKG2A.

Other exemplary drugs include puromycins, topetecan, rhizoxin, echinomycin, combretastatin, netropsin, estramustine, cemadotin, discodermolide, eleutherobin, mitoxantrone, pyrrolobenzimidazoles (PSI), gamma-interferon, Thialanostatin (A) and analogs, CDK11, immunotoxins, comprising e.g. ricin A, diphtheria toxin, cholera toxin.

In exemplary embodiments of the invention, the drug moiety is a mytomycin compound, a vinca alkaloid compound, taxol or an analogue, an anthracycline compound, a calicheamicin compound, a maytansinoid compound, an auristatin compound, a duocarmycin compound, SN38 or an analogue, a pyrrolobenzodiazepine compound, a indolinobenzodiazepine compound, a pyridinobenzodiazepine compound, a tubulysin compound, a non-natural camptothecin compound, a DNA binding drug, a kinase inhibitor, a MEK inhibitor, a KSP inhibitor, a P13 kinase inhibitor, a topoisomerase inhibitor, or analogues thereof.

In one preferred embodiment the drug is a non-natural camptothecin compound, vinca alkaloid, kinase inhibitor, (e.g. P13 kinase inhibitor: GDC-0941 and PI-103), MEK inhibitor, KSP inhibitor, RNA polymerase inhibitor, PARP inhibitor, docetaxel, paclitaxel, doxorubicin, dolastatin, calicheamicins, SN38, pyrrolobenzodiazepines, pyridinobenzodiazepines, indolinobenzodiazepines, DNA binding drugs, maytansinoids DM1 and DM4, auristatin MMAE, CC1065 and its analogs, camptothecin and its analogs, SN-38 and its analogs.

In another preferred embodiment the drug is selected from DNA binding drugs and microtubule agents, including pyrrolobenzodiazepines, indolinobenzodiazepines, pyridinobenzodiazepines, maytansinoids, maytansines, auristatins, tubulysins, duocarmycins, anthracyclines, taxanes.

In another preferred embodiment the drug is selected from colchinine, vinca alkaloids, tubulysins, irinotecans, an inhibitory peptide, amanitin and deBouganin.

In another embodiment, a combination of two or more different drugs are used.

In other embodiments the released Drug is itself a prodrug designed to release a further drug.

Drugs optionally include a membrane translocation moiety (e.g. adamantine, poly-lysine/arginine, TAT, human lactoferrin) and/or a targeting agent (against e.g. a tumor cell receptor) optionally linked through a stable or labile linker. Exemplary references include: Trends in Biochemical Sciences, 2015, 40, 12, 749; J. Am. Chem. Soc. 2015, 137, 12153-12160; Pharmaceutical Research, 2007, 24, 11, 1977.

It will further be understood that, in addition to a targeting agent or one or more targeting agents that may be attached to the Trigger or Linker L^{C} a targeting agent may optionally be attached to a drug, optionally via a spacer S^{P}.

Alternatively, it will be further understood that the targeting agent may comprise one or more additional drugs which are bound to the targeting agent by other types of linkers, e.g. cleavable by proteases, pH, thiols, or by catabolism.

It will further be understood that, in addition to a Construct-B (C^{B}) or one or more Constructs-B that may be attached to the Trigger or Linker L^{C} a C^{B} may optionally be attached to a drug, optionally via a spacer S^{P}.

Alternatively, it will be further understood that the C^{B} may comprise one or more additional drugs which are bound to the C^{B} by other types of linkers, e.g. cleavable by proteases, pH, thiols, or by catabolism.

The invention further contemplates that when a targeting agent is a suitably chosen antibody or antibody derivative that such targeting agent can induce antibody-dependent cellular toxicity (ADCC) or complement dependent cytotoxicity (CDC).

Several drugs may be replaced by an imagable label to measure drug targeting and release.

It will be understood that chemical modifications may also be made to the desired compound in order to make reactions of that compound more convenient for purposes of preparing conjugates of the invention.

Drugs containing an amine functional group for coupling to the TCO include mitomycin-C, mitomycin-A, daunorubicin, doxorubicin, aminopterin, actinomycin, bleomycin, 9-amino camptothecin, N8-acetyl spermidine, 1-(2 chloroethyl)1,2-dimethanesulfonyl hydrazide, tallysomycin, cytarabine, dolastatins (including auristatins) and derivatives thereof.

Drugs containing a hydroxyl function group for coupling to the TCO include etoposide, camptothecin, taxol, esperamicin, 1,8-dihydroxy-bicyclo[7.3.1]trideca-4-9-diene-2,6-diyne-13-one (U.S. Pat. No. 5,198,560), podophyllotoxin, anguidine, vincristine, vinblastine, morpholine-doxorubicin, n-(5,5-diacetoxy-pentyl)doxorubicin, and derivatives thereof.

Drugs containing a sulfhydryl functional group for coupling to the TCO include esperamicin and 6-mercaptopurine, and derivatives thereof.

It will be understood that the drugs can optionally be attached to the TCO derivative through a self-immolative linker L^{C}, or a combination thereof, and which may consist of multiple (self-immolative, or non immolative) units.

Several drugs may be replaced by an imagable label to measure chug targeting and release.

According to a further particular embodiment of the invention, the Prodrug is selected so as to target and or address a disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme.

In the Prodrug, the Construct-A, preferably a Drug, and the TCO derivative can be directly linked to each other. They can also be bound to each other via a linker or a self-immolative linker L^{C}. It will be understood that the invention encompasses any conceivable manner in which the dienophile TCO is attached to the Construct-A, preferably a Drug. In preferred embodiments Construct-A is a Drug. Methods of affecting conjugation to these drugs, e.g. through reactive amino acids such as lysine or cysteine in the case of proteins, are known to the skilled person.

### Log P

In some embodiments, compounds disclosed herein comprising a tetrazine group have a Log P value of 3.0 or lower, preferably 2.0 or lower, more preferably 1.0 or lower, most preferably 0.0 or lower.

In another preferred embodiment the Log P of compounds disclosed herein comprising a tetrazine group have a value in a range of from 2.0 and −2.0, more preferably in a range of from 1.0 and −1.0.

### Molecular Weight

For all compounds disclosed herein comprising a group Q, Q₁, Q₂, Q₃, Q₄ or —(CH₂)_{y}—((R₁)ₚ—R₂)ₙ—(R₁)ₚ—R₃, at least one of these groups has a molecular weight in a range of from 100 Da to 3000 Da. Preferably, at least one of these groups has a molecular weight in a range of from 100 Da to 2000 Da. More preferably, at least one of these groups has a molecular weight in a range of from 100 Da to 1500 Da, even more preferably in a range of from 150 Da to 1500 Da. Even more preferably still, at least one of these groups has a molecular weight in a range of from 150 Da to 1000 Da, most preferably in a range of from 200 Da to 1000 Da.

For all compounds disclosed herein comprising a group Q, Q₁, Q₂, Q₃, Q₄ or —(CH₂)_{y}—((R₁)ₚ—R₂)ₙ—(R₁)ₚ—R₃, none of these groups has a molecular weight of more than 3000 Da.

Group —(CH₂)_{y}—((R₁)ₚ—R₂)ₙ—(R₁)ₚ—R₃

In some embodiments, y is an integer in a range of from 1 to 12, preferably from 1 to 10, more preferably from 1 to 8, even more preferably from 2 to 6, most preferably from 2 to 4. In some embodiments, y is at least 2, preferably y is at least 3.

In some embodiments, p is 0 or 1, wherein each p is independently selected.

In some embodiments, each n is an integer independently selected from a range of from 0 to 24, preferably from 1 to 12, more preferably from 1 to 6, even more preferably from 1 to 3, most preferably n is 0 or 1. In other embodiments n is preferably an integer from 12 to 24.

In some embodiments, the entire group —((R₁)ₚ—R₂)ₙ—(R₁)ₚ—R₃ has a molecular weight in a range of from 100 Da to 3000 Da. Preferably, the entire group —((R₁)ₚ—R₂)ₙ—(R₁)ₚ—R₃ has a molecular weight in a range of from 100 Da to 2000 Da. More preferably, the entire group —((R₁)ₚ—R₂)ₙ—(R₁)ₚ—R₃ has a molecular weight in a range of from 100 Da to 1500 Da, even more preferably in a range of from 150 Da to 1500 Da. Even more preferably still, the entire group —((R₁)ₚ—R₂)ₙ—(R₁)ₚ—R₃ has a molecular weight in a range of from 150 Da to 1000 Da, most preferably in a range of from 200 Da to 1000 Da.

In some embodiments, the entire group —((R₁)ₚ—R₂)ₙ—(R₁)ₚ—R₃ satisfies molecules from Group R^{M} shown below:

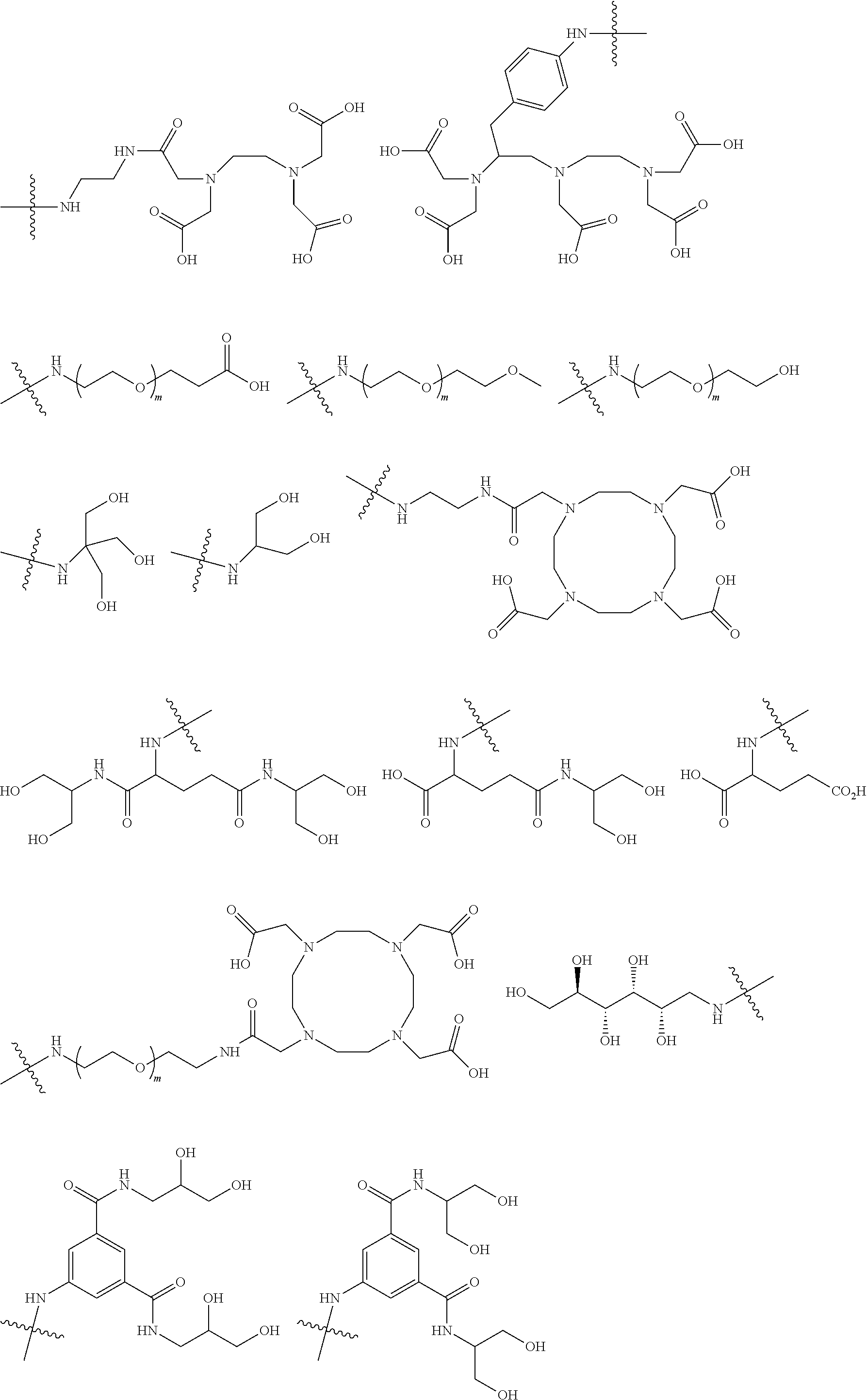

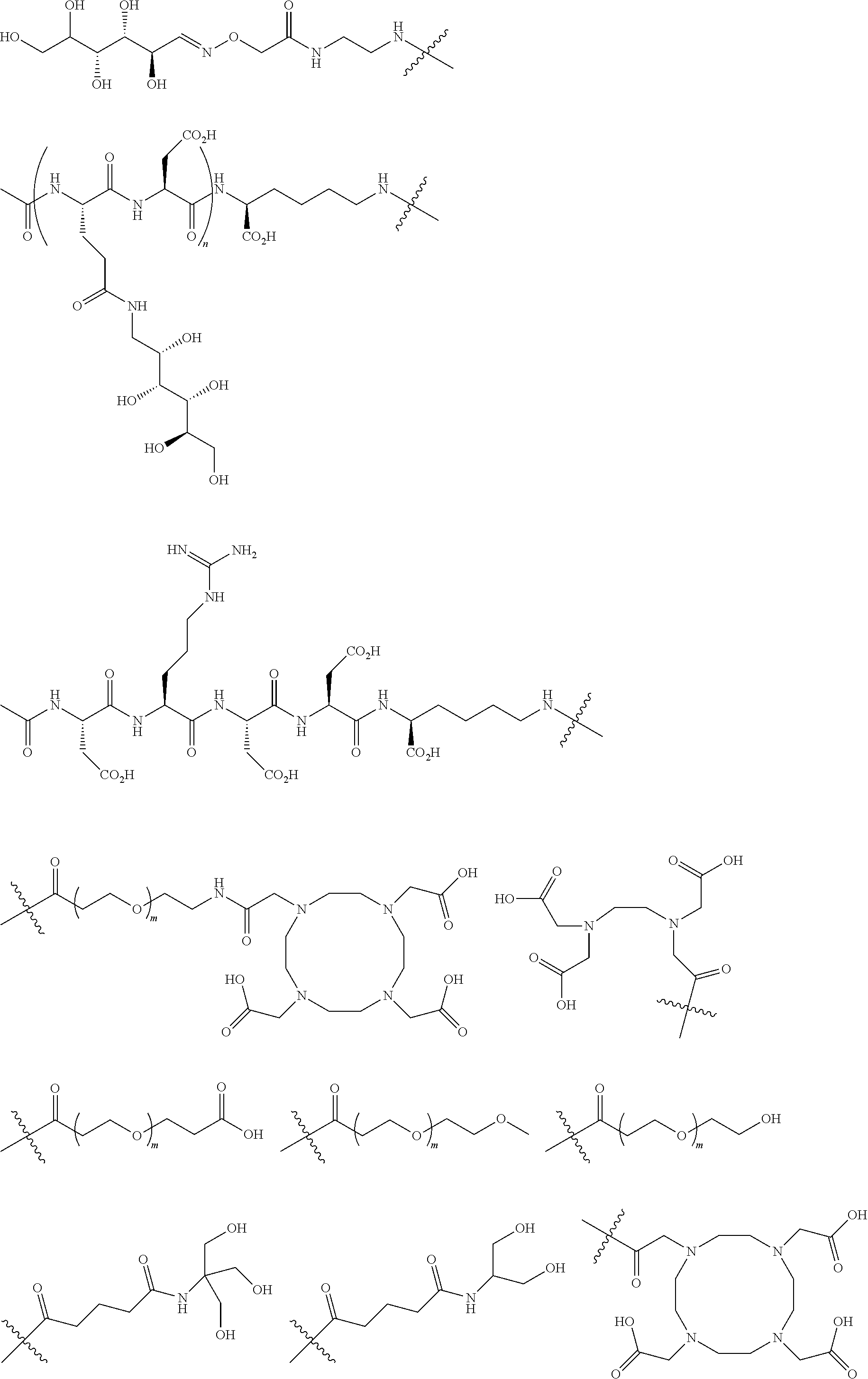

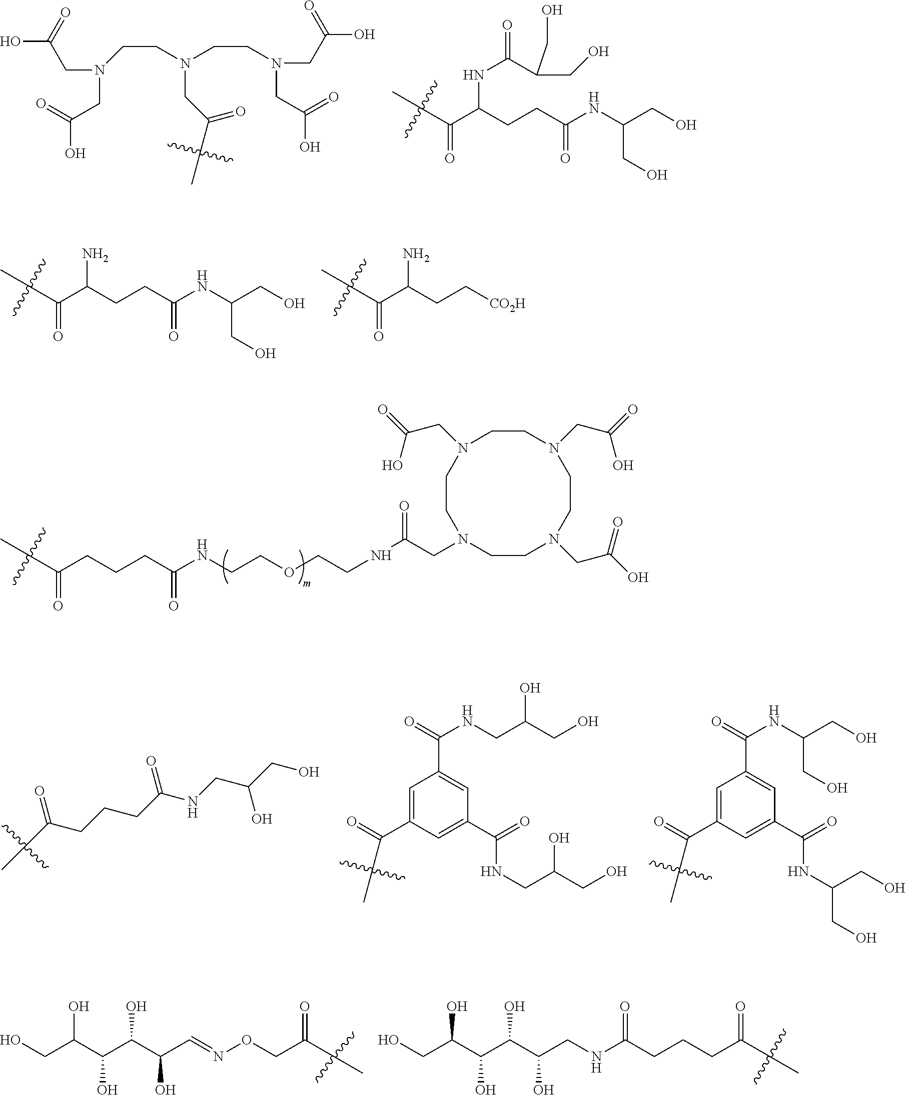

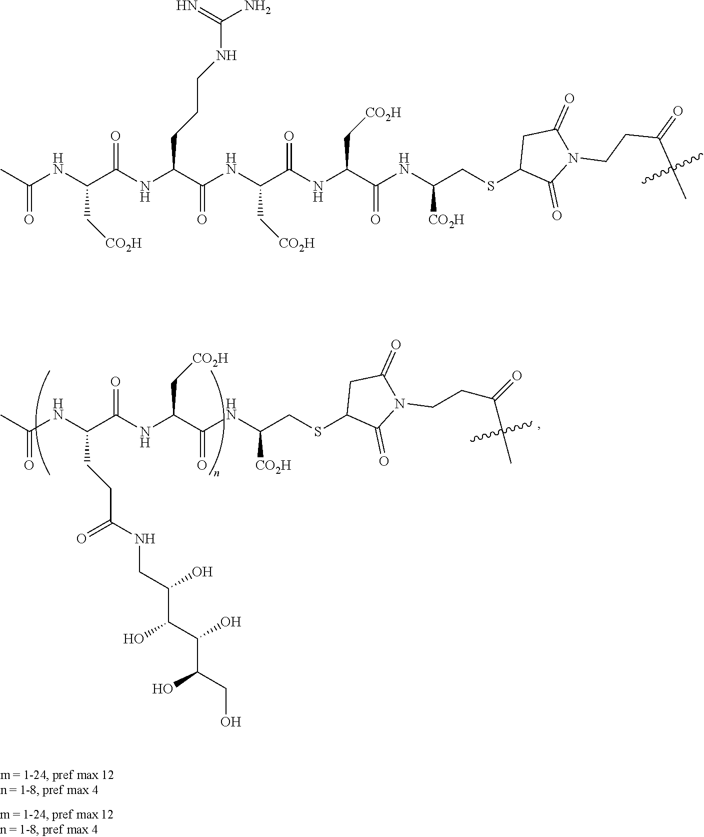

wherein the wiggly line denotes a bond to a tetrazine group as disclosed herein or to a group R₁ or R₂.

In some embodiments, the group —((R₁)ₚ—R₂)ₙ—(R₄)ₚ—R₃ satisfies molecules from Group R^{M}, wherein it is understood that when n is more than 1, —((R₁)ₚ—R₂)ₙ—(R₁)ₚ—R₃ may be preceded by a group —((R₁)ₚ—R₂)— so as to form a group —((R₁)ₚ—R₂)—((R₁)ₚ—R₂)ₙ₋₁—(R₁)ₚ—R₃. It is understood that this follows from the definition of how to write out the repeating units, i.e. —((R₁)ₚ—R₂)₂— would first be written as —(R₁)ₚ—R₂—(R₁)ₚ—R₂— before R₁, p, and R₂ are independently selected.

### R₁

In some embodiments, each R₁ is independently selected from the group consisting of —O—, —S—, —SS—, —NR₄—, —N═N—, —C(O)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NH₄—, —NR₄OC(S)—, —NR₄OC(S)O—, —NR₄OC(S)NR₄—, —OC(S)—, —C(S)O—, —OC(S)O—, —OC(S)NR₄—, —NR₄C(S)—, —NR₄C(S)O—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —OP(O)(R₄)₂—, —SP(O)(R₄)₂—, —NR₄P(O)(R₄)₂—, and combinations thereof, wherein R₄ is defined as described herein.

### R₂

In some embodiments, each R₂ is independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups, which are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized; and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, each R₂ is independently selected from the group consisting of C₁-C₁₂ alkylene groups, C₂-C₁₂ alkenylene groups, C₂-C₁₂ alkynylene groups, C₆-C₁₂ arylene, C₂-C₁₂ heteroarylene, C₃-C₁₂ cycloalkylene groups, C₅-C₁₂ cycloalkenylene groups, and C₁₂ cycloalkynylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, each R₂ is independently selected from the group consisting of C₁-C₆ alkylene groups, C₂-C₆ alkenylene groups, C₂-C₆ alkynylene groups, C₆-C₆ arylene, C₂-C₆ heteroarylene, C₃-C₆ cycloalkylene groups, and C₅-C₆ cycloalkenylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, the R₂ groups are optionally further substituted with one or more substituents selected from the group consisting of —Cl, —F, —Br, —I, —OH, —NH₂, —SO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₁-C₁₂ (hetero)arylalkenyl groups, C₄-C₁₂ (hetero)arylalkynyl groups, C₄-C₁₂ alkenyl(hetero)aryl groups, C₁₂ alkynyl(hetero)aryl groups, C₄-C₁₂ alkylcycloalkyl groups, C₆-C₁₂ alkylcycloalkenyl groups, C₁₃-C₁₈ alkylcycloalkynyl groups, C₄-C₁₂ cycloalkylalkyl groups, C₆-C₁₂ cycloalkenylalkyl groups, C₁₃-C₁₈ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₄-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₁₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In some embodiments, the R₂ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₆ alkyl(hetero)aryl groups, C₃-C₆ (hetero)arylalkyl groups, C₄-C₆ (hetero)arylalkenyl groups, C₄-C₆ (hetero)arylalkynyl groups, C₄-C₆ alkenyl(hetero)aryl groups, C₄-C₆ alkynyl(hetero)aryl groups, C₄-C₆ alkylcycloalkyl groups, C₆ alkylcycloalkenyl groups, C₄-C₆ cycloalkylalkyl groups, C₆ cycloalkenylalkyl groups, C₃-C₆ alkenylcycloalkyl groups, C₇ alkenylcycloalkenyl groups, C₅-C₆ cycloalkylalkenyl groups, C₇ cycloalkenylalkenyl groups, C₅-C₆ alkynylcycloalkyl groups, C₇ alkynylcycloalkenyl groups, C₅-C₆ cycloalkylalkynyl groups, C₅-C₆ cycloalkyl(hetero)aryl groups, and C₅-C₆ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In preferred embodiments, the R₂ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₇ alkyl(hetero)aryl groups, C₃-C₇ (hetero)arylalkyl groups, C₄-C₈ (hetero)arylalkenyl groups, C₄-C₈ (hetero)arylalkynyl groups, C₄-C₈ alkenyl(hetero)aryl groups, C₄-C₈ alkynyl(hetero)aryl groups, C₄-C₆ alkylcycloalkyl groups, C₆-C₇ alkylcycloalkenyl groups, C₄-C₆ cycloalkylalkyl groups, C₆-C₇ cycloalkenylalkyl groups, C₅-C₆ alkenylcycloalkyl groups, C₇-C₈ alkenylcycloalkenyl groups, C₅-C₆ cycloalkylalkenyl groups, C₇-C₈ cycloalkenylalkenyl groups, C₅-C₆ alkynylcycloalkyl groups, C₇-C₈ alkynylcycloalkenyl groups, C₅-C₆ cycloalkylalkynyl groups, C₅-C₉ cycloalkyl(hetero)aryl groups, and C₅-C₉ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

### R₃

R₃ is selected from the group consisting of —H, —OH, —NH₂, —N₃, —Cl, —Br, —F, —I, and a chelating moiety.

Non-limiting examples of chelating moieties for use in R₃ are DTPA (diethylenetriaminepentaacetic acid), DOTA (1,4,7,10-tetraazacyclododecane-N,N′,N″,N″-tetraacetic acid), NOTA (1,4,7-triazacyclononane-N,N′,N″-triacetic acid), TETA (1,4,8,11-tetraazacyclotetradecane-N,N′,N″,N′-tetraacetic acid), OTTA (N1-(p-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide).

Moieties Q, Q₁, Q₂, Q₃, Q₄

In some embodiments, z is an integer in a range of from 0 to 12, preferably from 0 to 10, more preferably from 0 to 8, even more preferably from 1 to 6, most preferably from 2 to 4. In other preferred embodiments g is 0. In case more than one moiety selected from the group consisting of Q, Q₁, Q₂, Q₃, and Q₄ within one compound satisfies Formula (9), each z is independently selected.

In some embodiments, h is 0 or 1. In case more than one moiety selected from the group consisting of Q, Q₁, Q₂, Q₃, and Q₄ within one compound satisfies Formula (9), each h is independently selected.

In some embodiments, each n belonging to a moiety Q, Q₁, Q₂, Q₃, or Q₄ is an integer independently selected from a range of from 0 to 24, preferably from 1 to 12, more preferably from 1 to 6, even more preferably from 1 to 3, most preferably f is 0 or 1. In other embodiments f is preferably an integer from 12 to 24.

In some embodiments, the group —((R₁₀)ₕ—R₁₁)ₙ—(R₁₀)ₕ—R₁₂ satisfies molecules from Group R^{M} shown above.

In some embodiments, the group —((R₁₀)ₕ—R₁₁)ₙ—(R₁₀)ₕ—R₁₂ satisfies molecules from Group R^{M}, wherein it is understood that when n is more than 1, e.g. —((R¹⁰)ₕ—R₁₁)ₙ₋₁—(R₁₀)ₕ—R₁₂ may be preceded by a group —(R₁₀)ₕ—R₁₁— so as to form a group —(R₁₀)ₕ—R₁₁—((R₁₀)ₕ—R₁₁)ₙ₋₁—(R₁₀)ₕ—R₁₂. It is understood that this follows from the definition of how to write out the repeating units, i.e. —((R₁₀)ₕ—R₁₁)₂— would first be written as —(R₁₀)ₕ—R₁₁—(R₁₀)ₕ—R₁₁— before R₁₀, h, and R₁₁ are independently selected.

### R₁₀

In some embodiments, each R₁₀ is independently selected from the group consisting of —O—, —S—, —SS—, —NR₄—, —N═N—, —C(O)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NRAS(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, —C(S)O—, —OC(S)O—, —OC(S)NR₄—, —NR₄C(S)—, —NR₄C(S)O—, —SS(O)₂—, —S(O)₂S—, —OS(O₂)S—, —SS(O)₂O—, —NR₄OS(O)—, —NR₁₀S(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —OP(O)(R₄)₂—, —SP(O)(R₄)₂—, —NR₄P(O)(R₄)₂—, and combinations thereof, wherein R₄ is defined as described herein.

### R₁₁

In some embodiments, each R₁₁ is independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups, which are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkynylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized; and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, each R₁₁ is independently selected from the group consisting of C₁-C₁₂ alkylene groups, C₂-C₁₂ alkenylene groups, C₂-C₁₂ alkynylene groups, C₆-C₁₂ arylene, C₂-C₁₂ heteroarylene, C₃-C₁₂ cycloalkylene groups, C₅-C₁₂ cycloalkenylene groups, and C₁₂ cycloalkynylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, each R₁₁ is independently selected from the group consisting of C₁-C₆ alkylene groups, C₂-C₆ alkenylene groups, C₂-C₆ alkynylene groups, C₆-C₆ arylene, C₂-C₆ heteroarylene, C₃-C₆ cycloalkylene groups, and C₅-C₆ cycloalkenylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

In some embodiments, the R₁₁ groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₁₂ alkyl groups, C₂-C₁₂ alkenyl groups, C₂-C₁₂ alkynyl groups, C₆-C₁₂ aryl groups, C₂-C₁₂ heteroaryl groups, C₃-C₁₂ cycloalkyl groups, C₅-C₁₂ cycloalkenyl groups, C₁₂ cycloalkynyl groups, C₃-C₁₂ alkyl(hetero)aryl groups, C₃-C₁₂ (hetero)arylalkyl groups, C₄-C₁₂ (hetero)arylalkenyl groups, C₄-C₁₂ (hetero)arylalkynyl groups, C₄-C₁₂ alkenyl(hetero)aryl groups, C₄-C₁₂ alkynyl(hetero)aryl groups, C₄-C₁₂ alkylcycloalkyl groups, C₆-C₁₂ alkylcycloalkenyl groups, C₁₃-C₁₈ alkylcycloalkynyl groups, C₁-C₁₂ cycloalkylalkyl groups, C₆-C₁₂ cycloalkenylalkyl groups, C₁₃-C₁₈ cycloalkynylalkyl groups, C₅-C₁₂ alkenylcycloalkyl groups, C₇-C₁₂ alkenylcycloalkenyl groups, C₁₄-C₁₆ alkenylcycloalkynyl groups, C₅-C₁₂ cycloalkylalkenyl groups, C₇-C₁₂ cycloalkenylalkenyl groups, C₁₄-C₁₆ cycloalkynylalkenyl groups, C₅-C₁₂ alkynylcycloalkyl groups, C₇-C₁₂ alkynylcycloalkenyl groups, C₁₄-C₁₆ alkynylcycloalkynyl groups, C₅-C₁₂ cycloalkylalkynyl groups, C₇-C₁₂ cycloalkenylalkynyl groups, C₁₄-C₁₆ cycloalkynylalkynyl groups, C₅-C₁₂ cycloalkyl(hetero)aryl groups, C₇-C₁₂ cycloalkenyl(hetero)aryl groups, C₄-C₁₆ cycloalkynyl(hetero)aryl groups, C₅-C₁₂ (hetero)arylcycloalkyl groups, C₇-C₁₂ (hetero)arylcycloalkenyl groups, and C₁₄-C₁₆ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In some embodiments, the Ru groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₆ alkyl(hetero)aryl groups, C₃-C₆ (hetero)arylalkyl groups, C₄-C₆ (hetero)arylalkenyl groups, C₄-C₆ (hetero)arylalkynyl groups, C₄-C₆ alkenyl(hetero)aryl groups, C₁-C₆ alkynyl(hetero)aryl groups, C₄-C₆ alkylcycloalkyl groups, C₆ alkylcycloalkenyl groups, C₄-C₆ cycloalkylalkyl groups, C₆ cycloalkenylalkyl groups, C₅-C₆ alkenylcycloalkyl groups, C₇ alkenylcycloalkenyl groups, C₅-C₆ cycloalkylalkenyl groups, C₇ cycloalkenylalkenyl groups, C₅-C₆ alkynylcycloalkyl groups, C₇ alkynylcycloalkenyl groups, C₅-C₆ cycloalkylalkynyl groups, C₅-C₆ cycloalkyl(hetero)aryl groups, and C₅-C₆ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

In preferred embodiments, the Ru groups are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₆ alkyl groups, C₂-C₆ alkenyl groups, C₂-C₆ alkynyl groups, C₆ aryl groups, C₂-C₆ heteroaryl groups, C₃-C₆ cycloalkyl groups, C₅-C₆ cycloalkenyl groups, C₃-C₇ alkyl(hetero)aryl groups, C₃-C₇ (hetero)arylalkyl groups, C₄-C₈ (hetero)arylalkenyl groups, C₄-C₈ (hetero)arylalkynyl groups, C₄-C₈ alkenyl(hetero)aryl groups, C₁-C₈ alkynyl(hetero)aryl groups, C₄-C₆ alkylcycloalkyl groups, C₆-C₇ alkylcycloalkenyl groups, C₄-C₆ cycloalkylalkyl groups, C₆-C₇ cycloalkenylalkyl groups, C₃-C₆ alkenylcycloalkyl groups, C₇-C₈ alkenylcycloalkenyl groups, C₅-C₆ cycloalkylalkenyl groups, C₇-C₈ cycloalkenylalkenyl groups, C₅-C₆ alkynylcycloalkyl groups, C₇-C₈ alkynylcycloalkenyl groups, C₅-C₆ cycloalkylalkynyl groups, C₅-C₉ cycloalkyl(hetero)aryl groups, and C₅-C₆ (hetero)arylcycloalkyl groups, wherein the substituents optionally contain one or more heteroatoms selected from the group consisting of O, S, NR₅, P, and Si, wherein the N, S, and P atoms are optionally oxidized, wherein the N atoms are optionally quaternized.

### R₁₂

R₁₂ is selected from the group consisting of —H, —OH, —NH₂, —N₃, —Cl, —Br, —F, —I, and a chelating moiety.

Non-limiting examples of chelating moieties for use in R₁₂ are DTPA (diethylenetriaminepentaacetic acid), DOTA (1,4,7,10-tetraazacyclododecane-N,N′,N″,N″-tetraacetic acid), NOTA (1,4,7-triazacyclononane-N,N′,N″-triacetic acid), TETA (1,4,8,11-tetraazacyclotetradecane-N,N′,N″,N′-tetraacetic acid), OTTA (N1-(p-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide).

### Formula (2)

In some embodiments, the structures according to Formula (2) can be further specified by satisfying any one of Formulae (2a), (2b), (2c), (2d), (2e), (2f), (2g), (2h), (2i), (2j), (2k), or (2l):

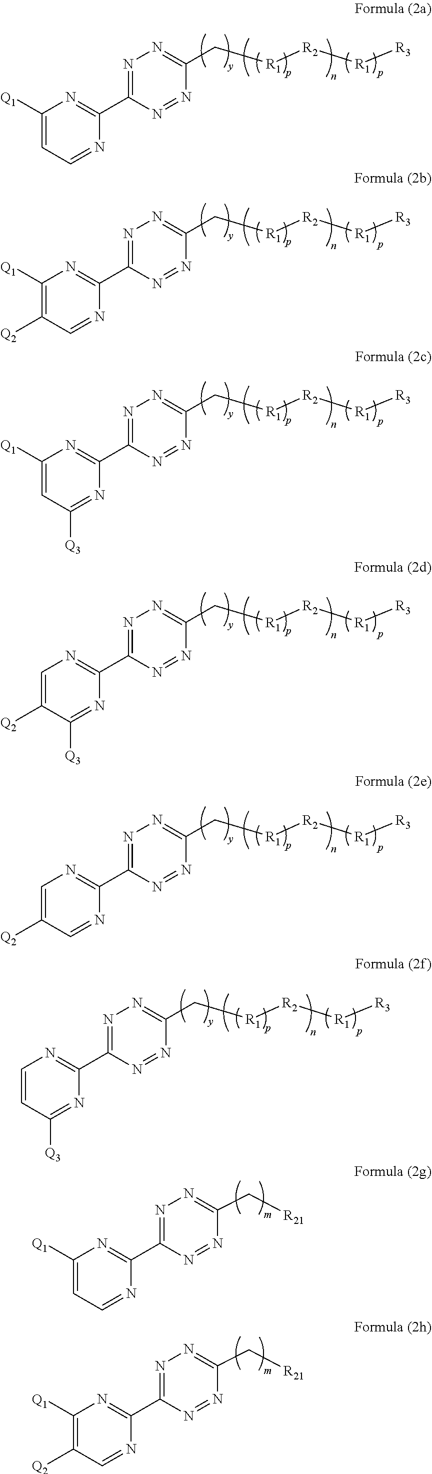

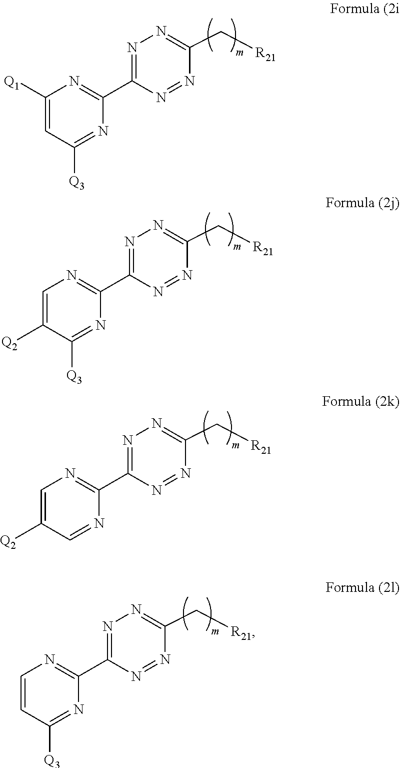

wherein y, n, p, R₁, R₂, R₃, Q₁, Q₂, and Q₃, are as defined above for Formula (2).

In Formulae (2a), (2b), (2c), (2d), (2e), and (2f), at least one moiety selected from the group consisting of Q₁, Q₂, Q₃, and —(CH₂)_{y}—((R₁)ₚ—R₂)ₙ—(R₁)ₚ—R₃ has a molecular weight in a range of from 100 Da to 3000 Da.

In Formulae (2g), (2h), (2i), (2j), (2k), and (2l), at least one moiety selected from the group consisting of Q₁, Q₂, and Q₃ has a molecular weight in a range of from 100 Da to 3000 Da.

In Formulae (2a), (2b), (2c), (2d), (2e), (2f), (2g), (2h), (2i), (2j), (2k), or (2l), the groups Q₁, Q₂, Q₃, and —(CH₂)_{y}—(R₁)ₚ—R₂)ₙ—(R₁)ₚ—R₃ have a molecular weight of at most 3000 Da.

In Formulae (2g), (2h), (2i), (2j), (2k), and (2l), m is an integer in a range of from 1 to 4, more preferably from 1 to 3.

In Formulae (2g), (2h), (2i), (2j), (2k), and (2l), R₂₁ is selected from the group consisting of —H, —OH, —C(O)OH, and —NH₂.

In some embodiments, in any one of Formulae (2g), (2h), (2i), (2j), (2k), and (2l), m is 1 and R₂₁ is —H, so as to form a methyl group.

In some embodiments, in any one of Formulae (2g), (2h), (2i), (2j), (2k), and (2l), m is 2 and R₂₁ is —OH.

In some embodiments, in any one of Formulae (2g), (2h), (2i), (2j), (2k), and (2l), in is 2 and R₂₁ is —NH₂.

In some embodiments, in any one of Formulae (2g), (2h), (2i), (2j), (2k), and (2l), m is 1 and R₂₁ is —C(O)OH.

In some embodiments, in any one of Formulae (2g), (2h), (2i), (2j), (2k), and (2l), in is 2 and R₂₁ is —C(O)OH.

### Formula (3)

In some embodiments, the structures according to Formula (3) can be further specified by satisfying any one of Formulae (3a), (3b), (3c), (3d), (3e), (3f), (3g), (3h), (3i), (3j), (3k), or (3l):

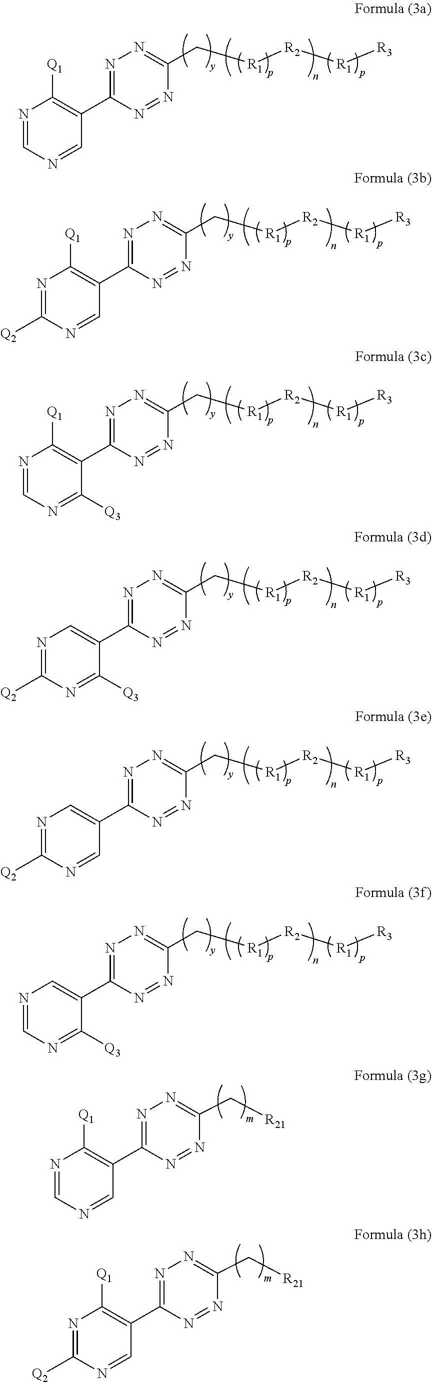

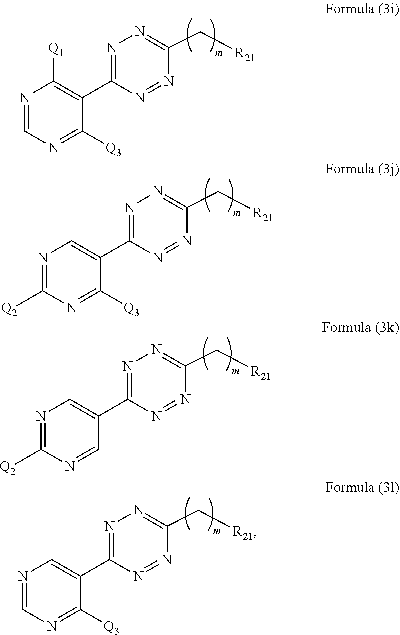

wherein y, n, p, R₁, R₂, R₃, Q₁, Q₂, and Q₃, are as defined above for Formula (3).

In Formulae (3a), (3b), (3c), (3d), (3e), and (3f), at least one moiety selected from the group consisting of Q₁, Q₂, Q₃, and —(CH₂)_{y}—((R₁)ₚ—R₂)ₙ—(R₁)ₚ—R₃ has a molecular weight in a range of from 100 Da to 3000 Da.

In Formulae (3g), (3h), (3i), (3j), (3k), and (3l), at least one moiety selected from the group consisting of Q₁, Q₂, and Q₃ has a molecular weight in a range of from 100 Da to 3000 Da.

In Formulae (3a), (3b), (3c), (3d), (3e), (3f), (3g), (3h), (3i), (3j), (3k), or (3l) the groups Q₁, Q₂, Q₃, and —(CH₂)_{y}—((R₁)ₚ—R₂)ₙ—(R₁)ₚ—R₃ have a molecular weight of at most 3000 Da.

In Formulae (3g), (3h), (3i), (3j), (3k), and (3l), in is an integer in a range of from 1 to 4, more preferably from 1 to 3.

In Formulae (3g), (3h), (3i), (3j), (3k), and (3l), R₂₁ is selected from the group consisting of —H, —OH, —C(O)OH, and —NH₂.

In some embodiments, in any one of Formulae (3g), (3h), (3i), (3j), (3k), and (3l), m is 1 and R₂₁ is —H, so as to form a methyl group.

In some embodiments, in any one of Formulae (3g), (3h), (3i), (3j), (3k), and (3l), m is 2 and R₂₁ is —OH.

In some embodiments, in any one of Formulae (3g), (3h), (3i), (3j), (3k), and (3l), m is 2 and R₂₁ is —NH₂.

In some embodiments, in any one of Formulae (3g), (3h), (3i), (3j), (3k), and (3l), m is 1 and R₂₁ is —C(O)OH.

In some embodiments, in any one of Formulae (3g), (3h), (3i), (3j), (3k), and (3l), m is 2 and R₂₁ is —C(O)OH.

### Formula (4)

In some embodiments, the structures according to Formula (4) can be further specified by satisfying any one of Formulae (4a), (4b), (4c), (4d), (4e), (4f), (4g), (4h), (4i), (4j), (4k), or (4l):

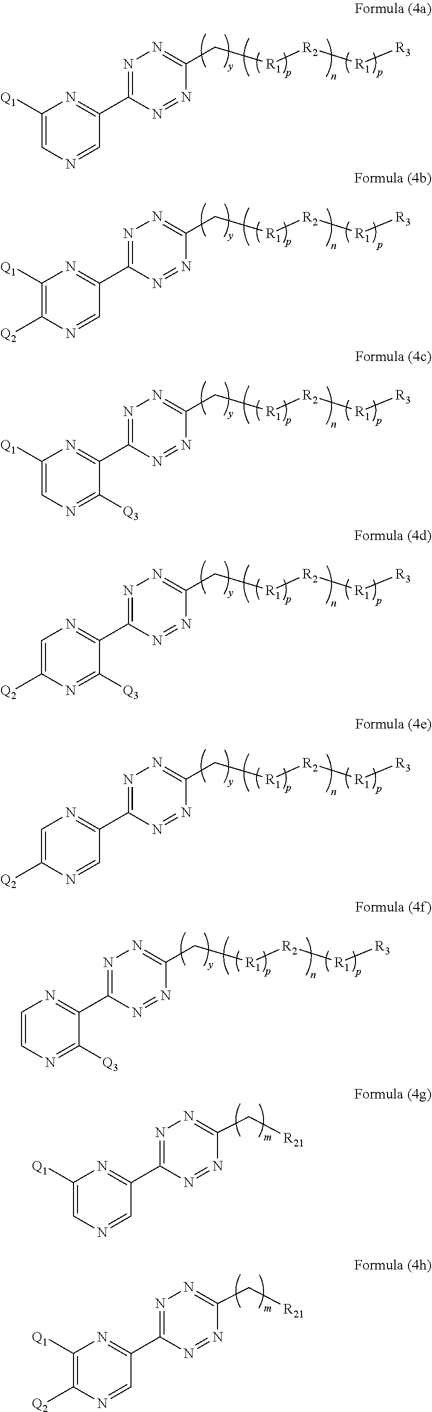

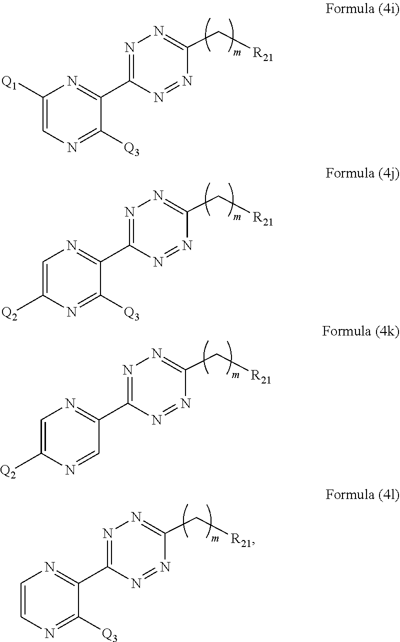

wherein y, n, p, R₁, R₂, R₃, Q₁, Q₂, and Q₃, are as defined above for Formula (2) or Formula (4).

In Formulae (4a), (4b), (4c), (4d), (4e), and (4f), at least one moiety selected from the group consisting of Q₁, Q₂, Q₃, and —(CH₂)_{y}—((R₁)ₚ—R₂)ₙ—(R₁)ₚ—R₃ has a molecular weight in a range of from 100 Da to 3000 Da.

In Formulae (4g), (4h), (4i), (4j), (4k), and (4l), at least one moiety selected from the group consisting of Q₁, Q₂, and Q₃ has a molecular weight in a range of from 100 Da to 3000 Da.

In Formulae (4a), (4b), (4c), (4d), (4e), (4f), (4g), (4h), (4i), (4j), (4k), or (4l) the groups Q₁, Q₂, Q₃, and —(CH₂)_{y}—((R₁)ₚ—R₂)ₙ—(R₁)ₚ—R₃ have a molecular weight of at most 3000 Da.

In Formulae (4g), (4h), (4i), (4j), (4k), and (4l), in is an integer in a range of from 1 to 4, more preferably from 1 to 3.

In Formulae (4g), (4h), (4i), (4j), (4k), and (4l), R₂₁ is selected from the group consisting of —H, —OH, —C(O)OH, and —NH₂.

In some embodiments, in any one of Formulae (4g), (4h), (4i), (4j), (4k), and (4l), m is 1 and R₂₁ is —H, so as to form a methyl group.

In some embodiments, in any one of Formulae (4g), (4h), (4i), (4j), (4k), and (4l), m is 2 and R₂₁ is —OH.

In some embodiments, in any one of Formulae (4g), (4h), (4i), (4j), (4k), and (4l), m is 2 and R₂₁ is —NH₂.

In some embodiments, in any one of Formulae (4g), (4h), (4i), (4j), (4k), and (4l), in is 1 and R₂₁ is —C(O)OH.

In some embodiments, in any one of Formulae (4g), (4h), (4i), (4j), (4k), and (4l), m is 2 and R₂₁ is —C(O)OH.

### Formula (5)

In some embodiments, the structures according to Formula (5) can be further specified by satisfying any one of Formulae (5a), (5b), (5c), (5d), (5e), (5f), (5g), (5h), (5i), (5j), (5k), or (5l):

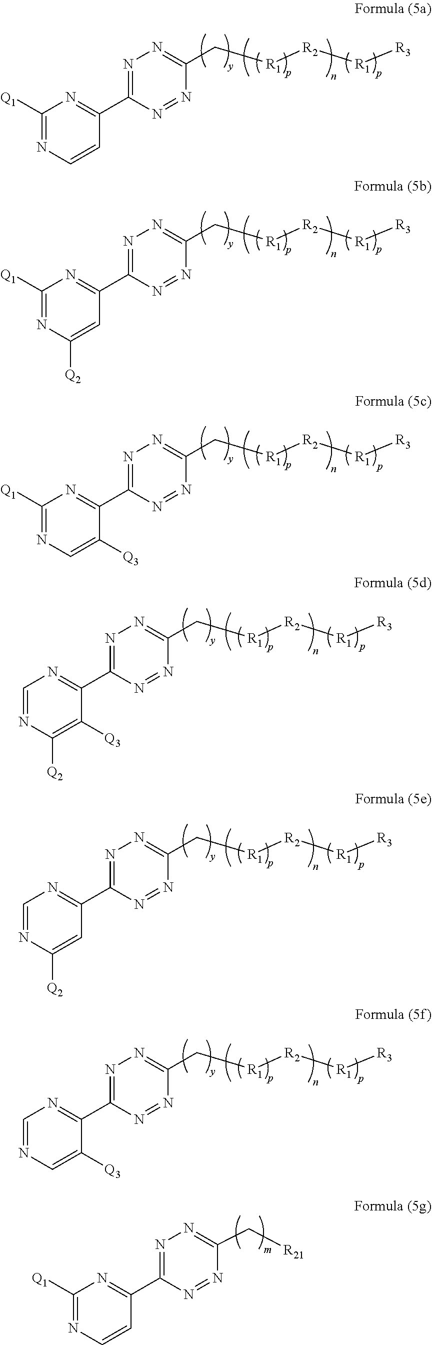

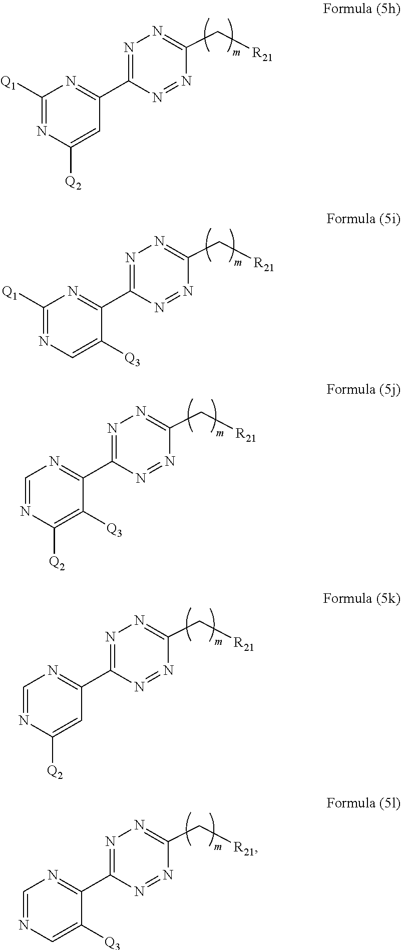

wherein y, n, p, R₁, R₂, R₃, Q₁, Q₂, and Q₃, are as defined above for Formula (5).

In Formulae (5a), (5b), (5c), (5d), (5e), and (5f), at least one moiety selected from the group consisting of Q₁, Q₂, Q₃, and —(CH₂)_{y}—((R₁)ₚ—R₂)ₙ—(R₁)ₚ—R₃ has a molecular weight in a range of from 100 Da to 3000 Da.

In Formulae (5g), (5h), (5i), (5j), (5k), and (5l), at least one moiety selected from the group consisting of Q₁, Q₂, and Q₃ has a molecular weight in a range of from 100 Da to 3000 Da.

In Formulae (5a), (5b), (5c), (5d), (5e), (5f), (5g), (5h), (5i), (5j), (5k), or (5l) the groups Q₁, Q₂, Q₃, and —(CH₂)_{y}—((R₁)ₚ—R₂)ₙ—(R₁)ₚ—R₃ have a molecular weight of at most 3000 Da.

In Formulae (5g), (5h), (5i), (5j), (5k), and (5l), m is an integer in a range of from 1 to 4, more preferably from 1 to 3.

In Formulae (5g), (5h), (5i), (5j), (5k), and (5l), R₂₁ is selected from the group consisting of —H, —OH, —C(O)OH, and —NH₂.

In some embodiments, in any one of Formulae (5g), (5h), (5i), (5j), (5k), and (5l), m is 1 and R₂₁ is —H, so as to form a methyl group.

In some embodiments, in any one of Formulae (5g), (5h), (5i), (5j), (5k), and (5l), m is 2 and R₂₁ is —OH.

In some embodiments, in any one of Formulae (5g), (5h), (5i), (5j), (5k), and (5l), m is 2 and R₂₁ is —NH₂.

In some embodiments, in any one of Formulae (5g), (5h), (5i), (5j), (5k), and (5l), m is 1 and R₂₁ is —C(O)OH.

In some embodiments, in any one of Formulae (5g), (5h), (5i), (5j), (5k), and (5l), m is 2 and R₂₁ is —C(O)OH.

### Formula (6)

In some embodiments, the structures according to Formula (6) can be further specified by satisfying any one of Formulae (6a), (6b), (6c), (6d), (6e), (6f), (6g), (6h), (6i), (6j), (6k), (6l), (6m), or (6n):

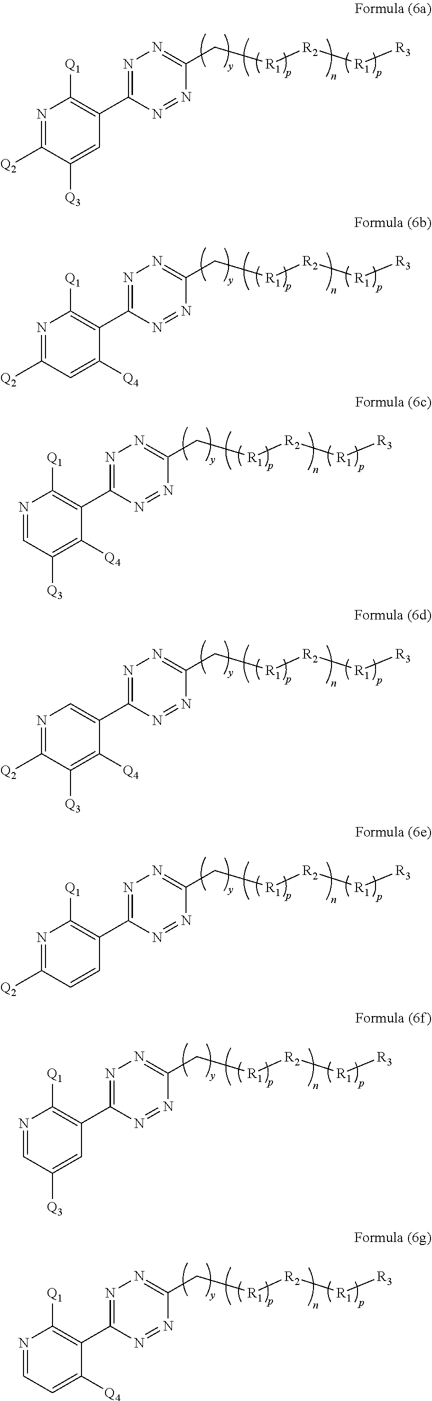

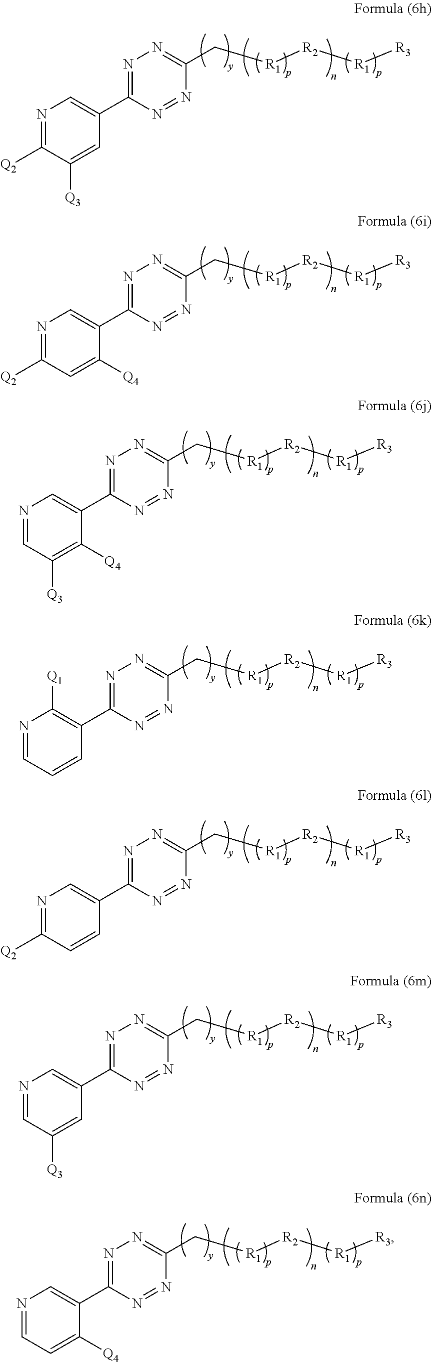

wherein y, n, p, R₁, R₂, R₃, Q₁, Q₂, Q₃, and Q₄ are as defined above for Formula (6).

In Formulae (6a), (6b), (6c), (6d), (6e), (6f), (6g), (6h), (6i), (6j), (6k), (6l), (6m), and (6n) at least one moiety selected from the group consisting of Q₁, Q₂, Q₃, Q₄, —(CH₂)_{y}—((R₁)ₚ—R₂)ₙ—(R₁)ₚ—R₃ has a molecular weight in a range of from 100 Da to 3000 Da.

In Formulae (6a), (6b), (6c), (6d), (6e), (6f), (6g), (6h), (6i), (6j), (6k), (6l), (6m), and (6n) moieties Q₁, Q₂, Q₃, Q₄, —(CH₂)_{y}—((R₁)ₚ—R₂)ₙ—(R₁)ₚ—R₃ have a molecular weight of at most 3000 Da.

In some embodiments, the structures according to Formula (6) can be further specified by satisfying any one of Formulae (6o), (6p), (6q), (6r), (6s), (6t), (6u), (6v), (6w), (6x), (6y), (6z), (6aa), or (6ab):

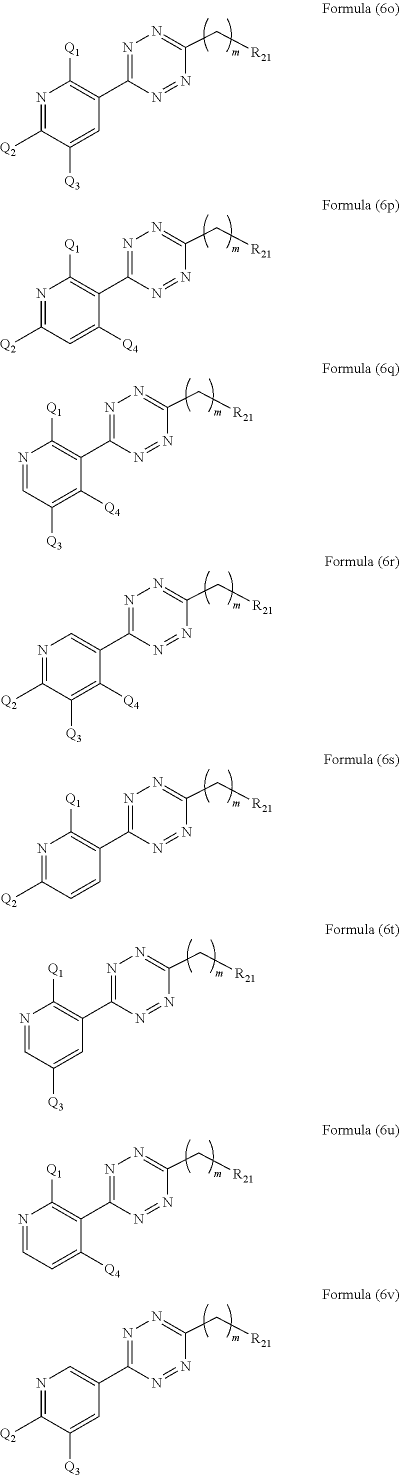

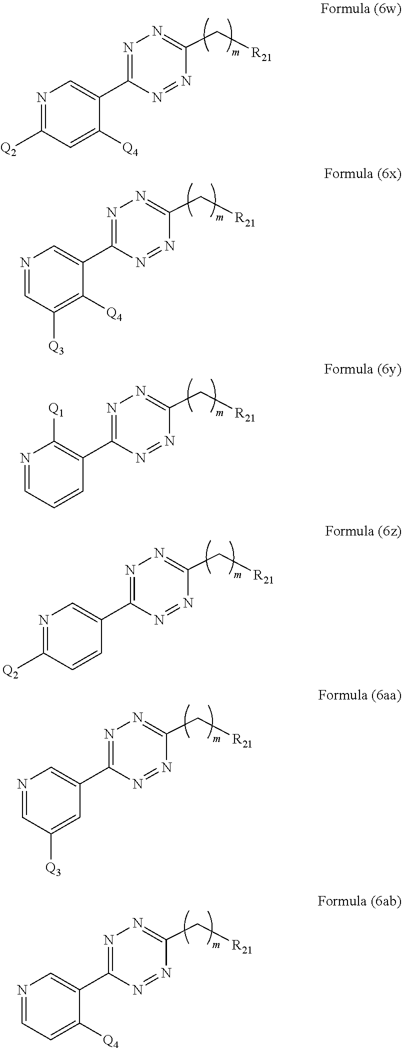

In Formulae (6o), (6p), (6q), (6r), (6s), (6t), (6u), (6v), (6w), (6x), (6y), (6z), (6aa), and (6ab), at least one moiety selected from the group consisting of Q₁, Q₂, Q₃, and Q₄ has a molecular weight in a range of from 100 Da to 3000 Da.

In Formulae (6o), (6p), (6q), (6r), (6s), (6t), (6u), (6v), (6w), (6x), (6y), (6z), (6aa), and (6ab), moieties Q₁, Q₂, Q₃, and Q₄ have a molecular weight of at most 3000 Da.

In Formulae (6o), (6p), (6q), (6r), (6s), (6t), (6u), (6v), (6w), (6x), (6y), (6z), (6aa), and (6ab), m is an integer in a range of from 1 to 4, more preferably from 1 to 3.

In Formulae (6o), (6p), (6q), (6r), (6s), (6t), (6u), (6v), (6w), (6x), (6y), (6z), (6aa), and (6ab), R₂₁ is selected from the group consisting of —H, —OH, —C(O)OH, and —NH₂.

In some embodiments, in any one of Formulae (6o), (6p), (6q), (6r), (6s), (6t), (6u), (6v), (6w), (6x), (6y), (6z) (6aa), and (6ab), in is 1 and R₂₁ is —H, so as to form a methyl group.

In some embodiments, in any one of Formulae (6o), (6p), (6q), (6r), (6s), (6t), (6u), (6v), (6w), (6x), (6y), (6z), (6aa), and (6ab), m is 2 and R₂₁ is —OH.

In some embodiments, in any one of Formulae (6o), (6p), (6q), (6r), (6s), (6t), (6u), (6v), (6w), (6x), (6y), (6z), (6aa), and (6ab), m is 2 and R₂₁ is —NH₂.

In some embodiments, in any one of Formulae (6o), (6p), (6q), (6r), (6s), (6t), (6u), (6v), (6w), (6x), (6y), (6z), (6aa), and (6ab), m is 1 and R₂₁ is —C(O)OH.

In some embodiments, in any one of Formulae (6o), (6p), (6q), (6r), (6s), (6t), (6u), (6v), (6w), (6x), (6y), (6z) (6aa), and (6ab), in is 2 and R₂₁ is —C(O)OH.

### Formula (7)

In some embodiments, the structures according to Formula (7) can be further specified by satisfying any one of Formulae (7a), (7b), (7c), (7d), (7e), (7f), (7g), (7h), (7i), (7j), (7k), (7l), (7m), or (7n):

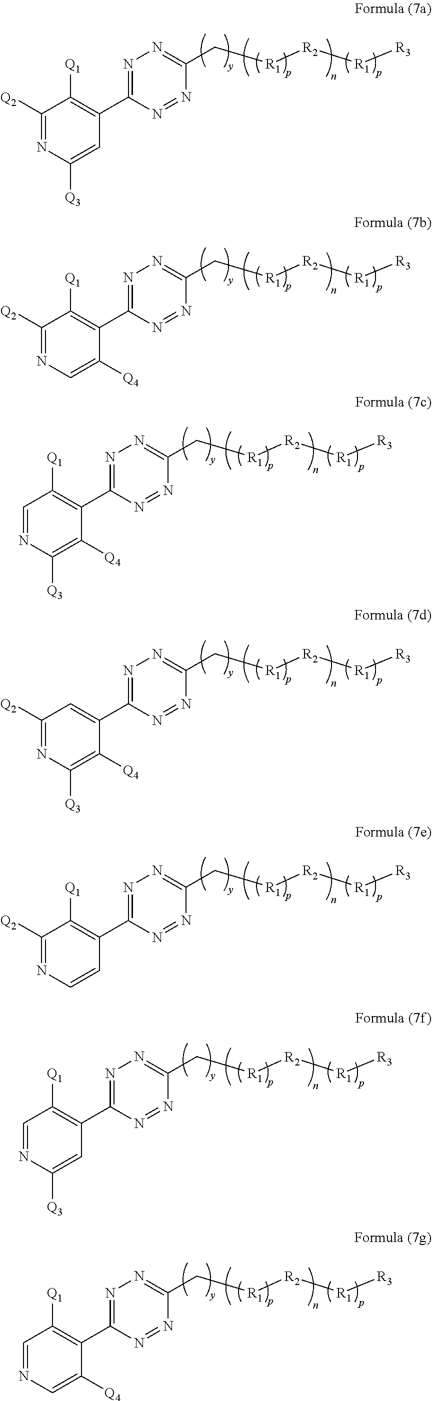

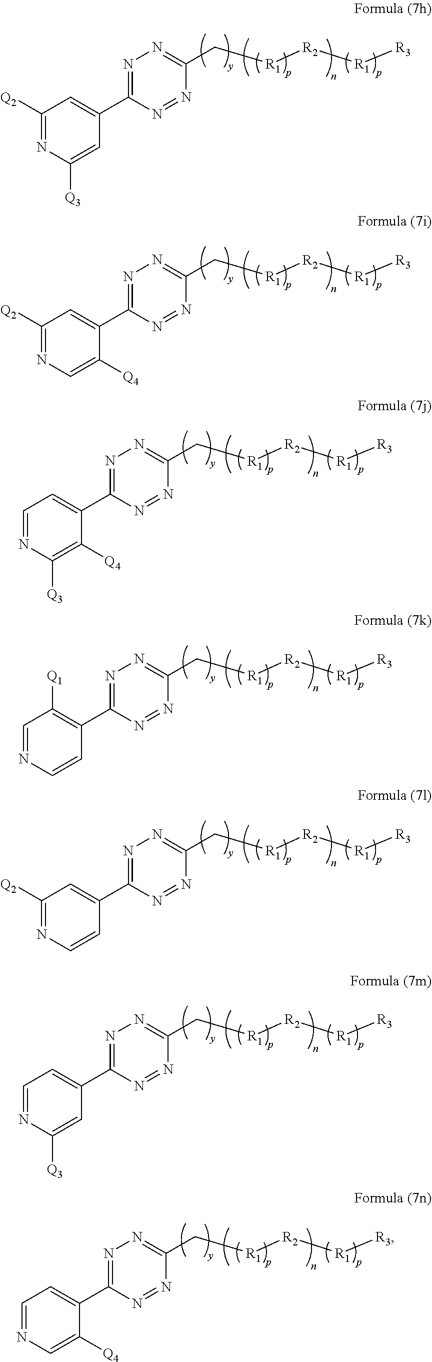

wherein y, n, p, R₁, R₂, R₃, Q₁, Q₂, Q₃, and Q₄ are as defined above for Formula (7).

In Formulae (7a), (7b), (7c), (7d), (7e), (7f), (7g), (7h), (7i), (7j), (7k), (7l), (7m), and (7n) at least one moiety selected from the group consisting of Q₁, Q₂, Q₃, Q₄, —(CH₂)_{y}—((R₁)ₚ—R₂)ₙ—(R₁)ₚ—R₃ has a molecular weight of in a range of from 100 Da to 3000 Da.

In Formulae (7a), (7b), (7c), (7d), (7e), (7f), (7g), (7h), (7i), (7j), (7k), (7l), (7m), and (7n) moieties Q₁, Q₂, Q₃, Q₄, —(CH₂)_{y}—((R₁)ₚ—R₂)ₙ—(R₁)ₚ—R₃ have a molecular weight of at most 3000 Da.

In some embodiments, the structures according to Formula (7) can be further specified by satisfying any one of Formulae (7o) (7p), (7q), (7r), (7s), (7t), (7u), (7v), (7w), (7x), (7y), (7z), (7aa), or (7ab):

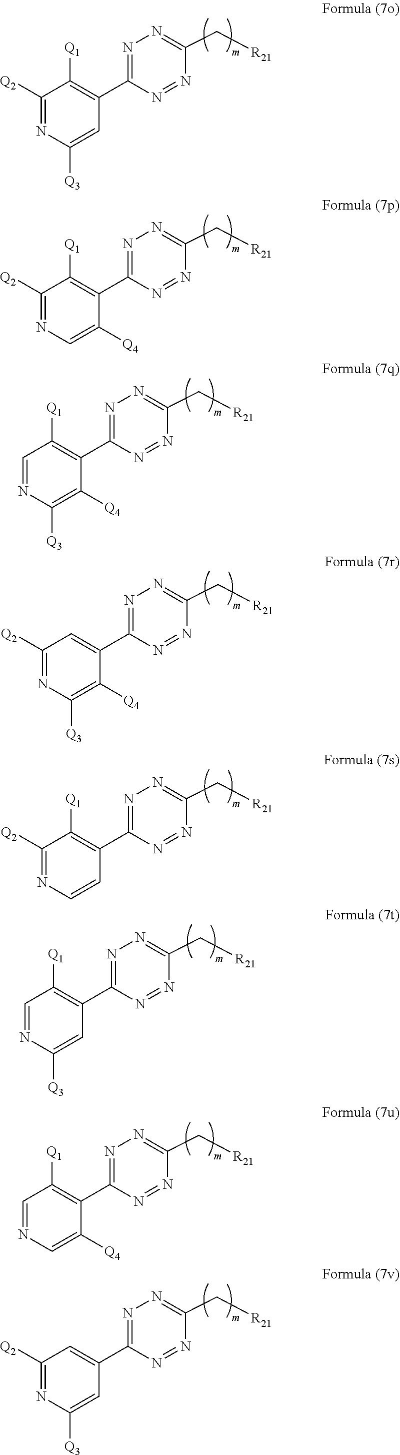

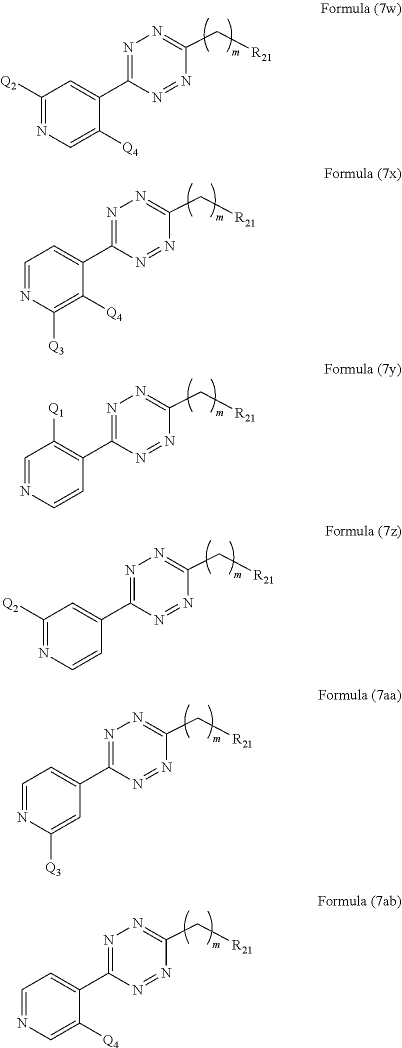

In Formulae (7o), (7p), (7q), (7r), (7s), (7t), (7u), (7v), (7w), (7x), (7y), (7z), (7aa), and (7ab), at least one moiety selected from the group consisting of Q₁, Q₂, Q₃, and Q₄ has a molecular weight in a range of from 100 Da to 3000 Da.

In Formulae (7o), (7p), (7q), (7r), (7s), (7t), (7u), (7v), (7w), (7x), (7y), (7z), (7aa), and (7ab), moieties Q₁, Q₂, Q₃, and Q₄ have a molecular weight of at most 3000 Da.

In Formulae (7o), (7p), (7q), (7r), (7s), (7t), (7u), (7v), (7w), (7x), (7y), (7z), (7aa), and (7ab), m is an integer in a range of from 1 to 4, more preferably from 1 to 3.

In Formulae (7o), (7p), (7q), (7r), (7s), (7t), (7u), (7v), (7w), (7x), (7y), (7z), (7aa), and (7ab), R₂₁ is selected from the group consisting of —H, —OH, —C(O)OH, and —NH₂.

In some embodiments, in any one of Formulae (7o), (7p), (7q), (7r), (7s), (7t), (7u), (7v), (7w), (7x), (7y), (7z), (7aa), and (7ab), m is 1 and R₂₁ is —H, so as to form a methyl group.

In some embodiments, in any one of Formulae (7o), (7p), (7q), (7r), (7s), (7t), (7u), (7v), (7w), (7x), (7y), (7z), (7aa), and (7ab), m is 2 and R₂₁ is —OH.

In some embodiments, in any one of Formulae (7o), (7p), (7q), (7r), (7s), (7t), (7u), (7v), (7w), (7x), (7y), (7z), (7aa), and (7ab), m is 2 and R₂₁ is —NH₂.

In some embodiments, in any one of Formulae (7o), (7p), (7q), (7r), (7s), (7t), (7u), (7v), (7w), (7x), (7y), (7z), (7aa), and (7ab), m is 1 and R₂₁ is —C(O)OH.

In some embodiments, in any one of Formulae (7o), (7p), (7q), (7r), (7s), (7t), (7u), (7v), (7w), (7x), (7y), (7z), (7aa), and (7ab), m is 2 and R₂₁ is —C(O)OH.

### Formula (8)

In some embodiments, the structures according to Formula (8) can be further specified by satisfying any one of Formulae (8a), (8b), (8c), (8d), (8e), (8f), (8g), (8h), (8i), (8j), (8k), (8l), (8m), or (8n):

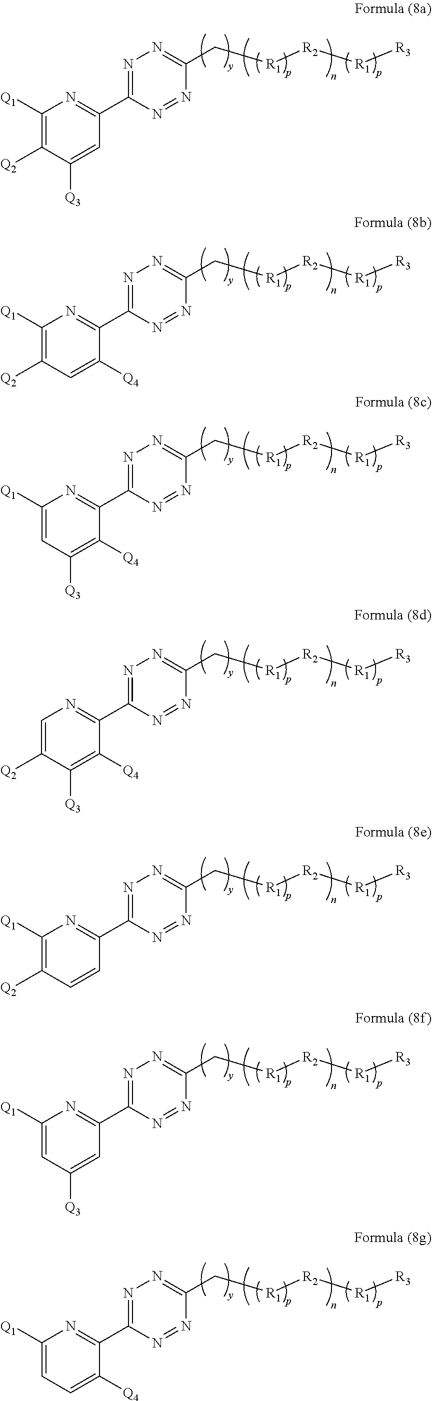

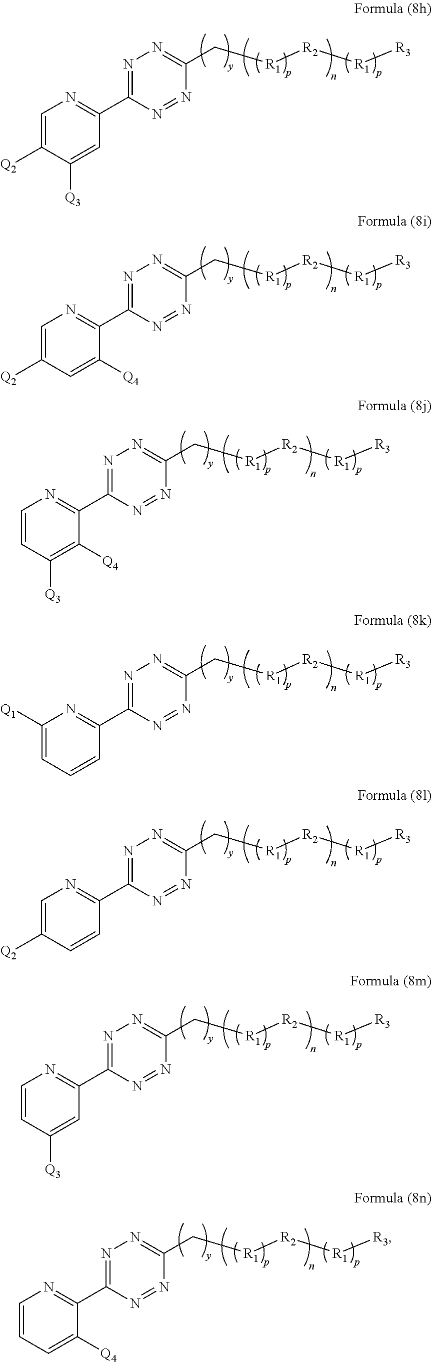

wherein y, n, p, R₁, R₂, R₃, Q₁, Q₂, Q₃, and Q₄ are as defined above for Formula (8).

In Formulae (8a), (8b), (8c), (8d), (8e), (8f), (8g), (8h), (8i), (8j), (8k), (8l), (8m), and (8n) at least one moiety selected from the group consisting of Q₁, Q₂, Q₃, Q₄, —(CH₂)_{y}—((R₁)ₚ—R₂)ₙ—(R₁)ₚ—R₃ has a molecular weight in a range of from 100 Da to 3000 Da.

In Formulae (8a), (8b), (8c), (8d), (8e), (8f), (8g), (8h), (8i), (8j), (8k), (8l), (8m), and (8n) moieties Q₁, Q₂, Q₃, Q₄, —(CH₂)_{y}—((R₁)ₚ—R₂)ₙ—(R₁)ₚ—R₃ have a molecular weight of at most 3000 Da.

In Formula (81), when the group —(CH₂)_{y}—((R₁)ₚ—R₂)ₙ—(R₁)ₚ—R₃ is a methyl group (i.e. y is 1, all p are 0, n is 0 and R₃ is —H), z in moiety Q₂ is at least 1.

In some embodiments, the structures according to Formula (8) can be further specified by satisfying any one of Formulae (8o), (8p), (8q), (8r), (8s), (8t), (8u), (8v), (8w), (8x), (8y), (8z), (8aa), or (8ab):

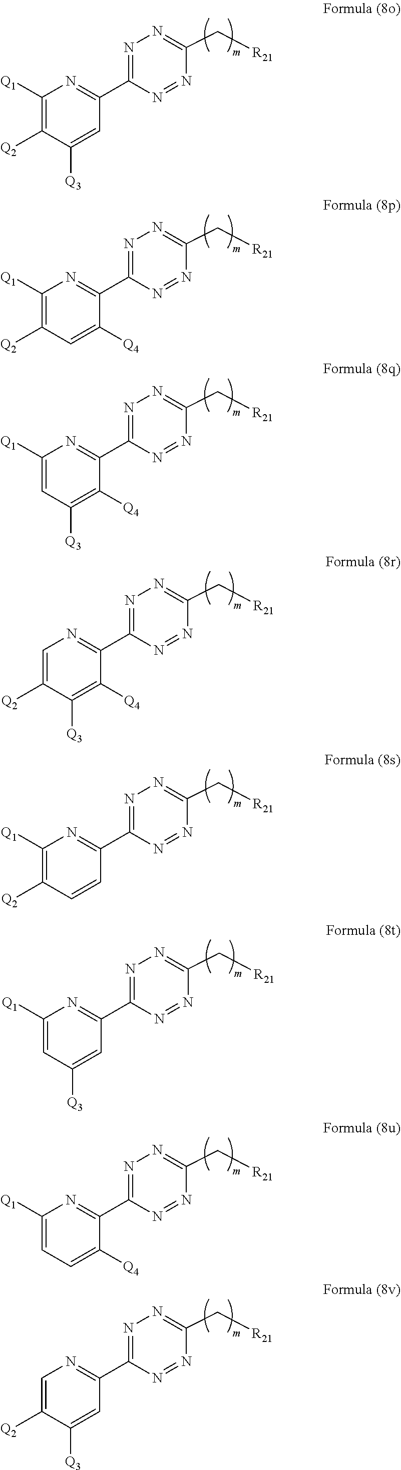

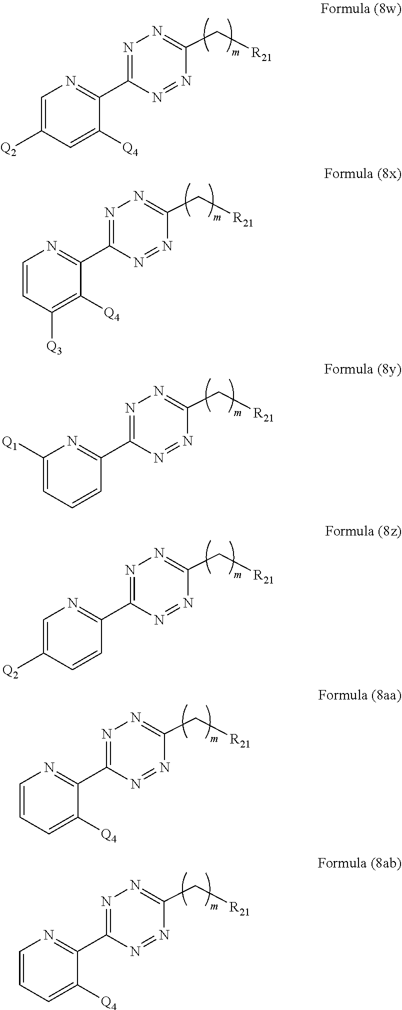

In Formulae (8o), (8p), (8q), (8r), (8s), (8t), (8u), (8v), (8w), (8x), (8y), (8z), (8aa), and (8ab), at least one moiety selected from the group consisting of Q₁, Q₂, Q₃, and Q₄ has a molecular weight in a range of from 100 Da to 3000 Da.

In Formulae (8o), (8p), (8q), (8r), (8s), (8t), (8u), (8v), (8w), (8x), (8y), (8z), (8aa), and (8ab), at least one moiety selected from the group consisting of Q₁, Q₂, Q₃, and Q₄ has a molecular weight of at most 3000 Da.

In Formulae (8o), (8p), (8q), (8r), (8s), (8t), (8u), (8v), (8w), (8x), (8y), (8z), (8aa), and (8ab), m is an integer in a range of from 1 to 4, more preferably from 1 to 3.

In Formulae (8o), (8p), (8q), (8r), (8s), (8t), (8u), (8v), (8w), (8x), (8y), (8z), (8aa), and (8ab), R₂₁ is selected from the group consisting of —H, —OH, —C(O)OH, and —NH₂.

In some embodiments, in any one of Formulae (8o), (8p), (8q), (8r), (8s), (8t), (8u), (8v), (8w), (8x), (8y), (8z), (8aa), and (8ab), m is 1 and R₂₁ is —H, so as to form a methyl group. In Formula (8z), when m is 1 and R₂ is —H, so as to form a methyl group, then z in moiety Q₂ is at least 1.

In some embodiments, in any one of Formulae (8o), (8p), (8q), (8r), (8s), (8t), (8u), (8v), (8w), (8x), (8y), (8z), (8aa), and (8ab), m is 2 and R₂₁ is —OH.

In some embodiments, in any one of Formulae (8o), (8p), (8q), (8r), (8s), (8t), (8u), (8v), (8w), (8x), (8y), (8z), (8aa), and (8ab), m is 2 and R₂₁ is —NH₂.

In some embodiments, in any one of Formulae (8o), (8p), (8q), (8r), (8s), (8t), (8u), (8v), (8w), (8x), (8y), (8z), (8aa), and (8ab), m is 1 and R₂₁ is —C(O)OH.

In some embodiments, in any one of Formulae (8o), (8p), (8q), (8r), (8s), (8t), (8u), (8v), (8w), (8x), (8y), (8z), (8aa), and (8ab), m is 2 and R₂₁ is —C(O)OH.

### Dienophile

Suitable dienophiles for use in kits disclosed herein are known to the skilled person.

In some embodiments, the dienophile satisfies Formula (19):

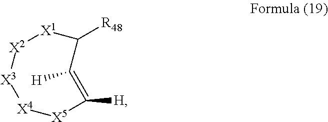

wherein each X¹, X², X³, X⁴ is independently selected from the group consisting of —C(R₄₇)₂—, —NR₃₇—, —C(O)—, —O—, such that at most two of X¹, X², X³, X⁴ are not —C(R₄₇)₂—, and with the proviso that no sets consisting of adjacent atoms are present selected from the group consisting of —O—O—, —O—N—, —C(O)—O—, N—N—, and —C(O)—C(O)—.

It is preferred that at most one CD is comprised in the structure of Formula (19).

In some embodiments, two R₄₇ are comprised in a ring so as to form a ring fused to the eight-membered trans-ring,

In a preferred embodiment, X¹, X², X³, X⁴ are all —C(R₄₇)₂— and at most 3 of R₄₇ are not H, more preferably at most 2 R₄₇ are not H.

In a preferred embodiment, at most one of X¹, X², X³, X⁴ is not —C(R₄₇)₂— and at most 3 of R₄₇ are not H, more preferably at most 2 R₄₇ are not H.

In a preferred embodiment, two of X², X³, X⁴ together form an amide and at most 3 of R₄₇ are not H, more preferably at most 2 R₄₇ are not H.

In a preferred embodiment, X¹ is C(R₄₇)₂.

In particularly favourable embodiments, R₄₈ is in the axial position.

It is preferred that when two R₄₇ groups are comprised in a ring so as to form a ring fused to the eight-membered trans-ring, that these rings fused to the eight-membered trans-ring are C₃-C₇ cycloalkylene groups and C₄-C₇ cycloalkenylene groups, optionally substituted and containing heteroatoms as described for R₄₇.

### R⁶

In some embodiments, R⁶ is selected from the group consisting of hydrogen, C₁-C₄ alkyl groups, C₂-C₄ alkenyl groups, and C₄₋₆ (hetero)aryl groups, wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In some embodiments, R⁶ is selected from the group consisting of hydrogen, C₁-C₃ alkyl groups, C₂-C₃ alkenyl groups, and C₄₋₆ (hetero)aryl groups, wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

### R⁷

In some embodiments, each R⁷ is independently selected from the group consisting of hydrogen and C₁-C₃ alkyl groups, C₂-C₃ alkenyl groups, and C₄₋₆ (hetero)aryl groups, wherein the alkyl groups, alkenyl groups, and (hetero)aryl groups are optionally substituted with a moiety selected from the group consisting of —Cl, —F, —Br, —I, —OH, —NH₂, ═O, ═NH, —N(CH₃)₂, —S(O)₂CH₃, and —SH, and are optionally interrupted by at most one heteroatom selected from the group consisting of —O—, —S—, —NH—, —P—, and —Si—, wherein the N, S, and P atoms are optionally oxidized, wherein the N atoms are optionally quaternized.

In preferred embodiments, R⁷ is preferably selected from the group consisting of hydrogen, methyl, —CH₂—CH₂—N(CH₃)₂, and —CH₂—CH₂—S(O)₂—CH₃,

R⁸ and R⁹

R⁸ and R⁹ are as defined for R⁶. In some embodiments, at least one or all R⁸ are —H. In some embodiments, at least one or all R⁸ are —CH₃. In some embodiments, at least one or all R⁹ are —H. In some embodiments, at least one or all R⁹ are —CH₃.

### R₃₁

In some embodiments, R₃₁ is selected from the group consisting of hydrogen, C₁-C₆ alkyl groups, C₆ aryl groups, C₄-C₅ heteroaryl groups, C₃-C₆ cycloalkyl groups, C₅-C₁₂ alkyl(hetero)aryl groups, C₅-C₁₂ (hetero)arylalkyl groups, C₁-C₁₂ alkylcycloalkyl groups, —N(R′)₂, —OR′, —SR′, —SO₃H, —C(O)OR′, and Si(R′)₃, wherein for R₃₁ the alkyl groups, (hetero)aryl groups, cycloalkyl groups, alkyl(hetero)aryl groups, (hetero)arylalkyl groups, alkylcycloalkyl groups are optionally substituted with a moiety selected from the group consisting of —Cl, —F, —Br, —I, NO₂, SO₃H, PO₃H, —PO₄H₂, —OR′, —N(R′)₂, —CF₃, ═O, ═NR′, —SR′, and optionally contain one or more heteroatoms selected from the group consisting of —O—, —S—, —NR′—, —P—, and —Si—, wherein the N, S, and P atoms are optionally oxidized, wherein the N atoms are optionally quaternized,

In preferred embodiments, R₃₁ is hydrogen. In other preferred embodiments, R₃₁ is —CH₃.

### R₃₂

Preferably, R₃₂ is a conjugation moiety, which is chemical group that can be used for binding, conjugation or coupling to a Construct-B. The person skilled in the art is aware of the myriad of strategies that are available for the chemoselective or -unselective coupling or conjugation of one molecule or construct to another. In some embodiments, R₃₂ is a moiety that allows conjugation to a protein comprising natural and/or non-natural amino acids. Moieties suitable for conjugation are known to the skilled person. Conjugation strategies are for example found in [O. Boutureira, G. J. L. Bernardes, Chem. Rev., 2015, 115, 2174-2195].

In particularly favourable embodiments, R₃₂ is selected from the group consisting of N-maleimidyl groups, halogenated N-alkylamido groups, sulfonyloxy N-alkylamido groups, vinyl sulfone groups, activated carboxylic acids, benzenesulfonyl halides, ester groups, carbonate groups, sulfonyl halide groups, thiol groups or derivatives thereof, C₂₋₆ alkenyl groups, C₂₋₈ alkynyl groups, C₇₋₁₈ cycloalkynyl groups, C₅₋₁₈ heterocycloalkynyl groups, bicyclo[6.1.0]non-4-yn-9-yl] groups, C₄₋₁₂ cycloalkenyl groups, azido groups, phosphine groups, nitrile oxide groups, nitrone groups, nitrile imine groups, isonitrile groups, diazo groups, ketone groups, (O-alkyl)hydroxylamino groups, hydrazine groups, halogenated N-maleimidyl groups, aryloxymaleimides, dithiophenolmaleimides, bromo- and dibromopyridazinediones, 2,5-dibromohexanediamide groups, alkynone groups, 3-arylpropionitrile groups, 1,1-bis(sulfonylmethyl)-methylcarbonyl groups or elimination derivatives thereof, carbonyl halide groups, allenamide groups, 1,2-quinone groups, isothiocyanate groups, aldehyde groups, triazine groups, squaric acids, 2-imino-2-methoxyethyl groups, (oxa)norbornene groups, (imino)sydnones, methylsulfonyl phenyloxadiazole groups, aminooxy groups, 2-amino benzamidoxime groups, groups reactive in the Pictet Spengler ligation and hydrazino-Pictet Spengler (HIPS) ligation.

In preferred embodiments, R₃₂ is an N-maleimidyl group connected to the remaining part of the compound according to Formula (20) via the N atom of the N-maleimidyl group.

### R₃₃

In some embodiments, each individual R₃₃ is selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized.

In particularly favourable embodiments, each individual R₃₃ is selected from the group consisting of C₁-C₆ alkylene groups, C₂-C₆ alkenylene groups, and C₂-C₆ alkynylene groups, more preferably from the group consisting of C₁-C₃ alkylene groups, C₂-C₃ alkenylene groups, and C₂-C₃ alkynylene groups;

and wherein preferably the alkylene groups, alkenylene groups, alkynylene groups, cycloalkylene groups, cycloalkenylene groups, and cycloalkynylene groups optionally contain one or more heteroatoms selected from the group consisting of O, S, NR₅, P, and Si, wherein the N, S, and P atoms are optionally oxidized, wherein the N atoms are optionally quaternized.

R₃₁

In some embodiments, each individual R₃₄ is selected from the group consisting of —OH, —OC(O)Cl, —OC(O)O—N-succinimidyl, —OC(O)O-4-nitrophenyl, —OC(O)O— tetrafluorophenyl, —OC(O)O-pentafluorophenyl, —OC(O)—C^{A}, —OC(S)—C^{A}, —O-(L^{C}(C^{A})ₛ(C^{A})ₛ)ᵣ—C^{A}, and —C^{A},

wherein preferably r is an integer in range of from 0 to 2,
wherein preferably each s is independently 0 or 1.

It is preferred that R₃₄ is an axial substituent on the TCO ring.

### R₃₅

In some embodiments, each individual R₃₅ is selected from the group consisting of C₁-C₈ alkylene groups, C₂-C₈ alkenylene groups, C₂-C₈ alkynylene groups, C₆ arylene groups, C₄-C₅ heteroarylene groups, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein for the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,

In some embodiments, each individual R₃₅ is selected from the group consisting of C₁-C₄ alkylene groups, C₂-C₄ alkenylene groups, C₂-C₄ alkynylene groups, C₆ arylene groups, C₄-C₅ heteroarylene groups, C₃-C₆ cycloalkylene groups, wherein the alkylene groups, alkenylene groups, alkynylene groups, (hetero)arylene groups, and cycloalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized.

### R₃₆

In some embodiments, R₃₆ is selected from the group consisting of hydrogen, C₁-C₄ alkyl groups, C₂-C₄ alkenyl groups, and C₄₋₆ (hetero)aryl groups, wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

In some embodiments, R₃₆ is selected from the group consisting of hydrogen, C₁-C₃ alkyl groups, C₂-C₃ alkenyl groups, and C₄₋₆ (hetero)aryl groups, wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized.

### R₃₇

In some embodiments, R₃₇ is selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups, wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

In some embodiments, R₃₇ is selected from the group consisting of hydrogen, —(S^{P})ᵢ—C^{B}, C₁-C₄ alkyl groups, C₂-C₄ alkenyl groups, C₂-C₄ alkynyl groups, C₆-C₈ aryl, C₂-C₈ heteroaryl, C₃-C₈ cycloalkyl groups, C₅-C₆ cycloalkenyl groups, C₃-C₁₀ alkyl(hetero)aryl groups, C₃-C₁₀ (hetero)arylalkyl groups, C₄-C₈ alkylcycloalkyl groups, C₄-C₈ cycloalkylalkyl groups, C₅-C₁₀ cycloalkyl(hetero)aryl groups and C₃-C₁₀ (hetero)arylcycloalkyl groups, wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

### R₄₇

In some embodiments, each R₄₇ is independently selected from the group consisting of hydrogen, —F, —Cl, —Br, —I, —OH, —NH₂, —SO₃_{^{−}}, —PO₃_{^{−}}, —NO₂, —CF₃, —SH, —(S^{P})ᵢ—C^{B}, C₁-C₈ alkyl groups, C₂-C₈ alkenyl groups, C₂-C₈ alkynyl groups, C₆-C₁₂ aryl groups, C₂-C₁₂ heteroaryl groups, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₃-C₁₂ (hetero)arylcycloalkyl groups, wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.

In some embodiments, each R₄₇ is independently selected from the group consisting of hydrogen, —F, —Cl, —Br, —I, —OH, —NH₂, —SO₃_{^{−}}, —PO₃_{^{−}}, —NO₂, —CF₃, —SH, —(S^{P})ᵢ—C^{B}, C₁-C₄ alkyl groups, C₂-C₄ alkenyl groups, C₂-C₄ alkynyl groups, C₆-C₈ aryl groups, C₂-C₈ heteroaryl groups, C₃-C₆ cycloalkyl groups, C₅-C₆ cycloalkenyl groups, C₃-C₁₀ alkyl(hetero)aryl groups, C₃-C₁₀ (hetero)arylalkyl groups, C₄-C₁₀ alkylcycloalkyl groups, C₄-C₁₀ cycloalkylalkyl groups, C₅-C₁₀ cycloalkyl(hetero)aryl groups and C₅-C₁₀ (hetero)arylcycloalkyl groups, wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized.

### R₄₈

In some embodiments, R₄₈ is selected from the group consisting of —OH, —OC(O)Cl, —OC(O)O—N-succinimidyl, —OC(O)O-4-nitrophenyl, —OC(O)O-tetrafluorophenyl, —OC(O)O-pentafluorophenyl, —OC(O)—C^{A}, —OC(S)—C^{A}, —O-(L^{C}(C^{A})ₛ(C^{A})ₛ((S^{P})ᵢ—C^{B})ⱼ)ᵣ—C^{A}, and —C^{A}.

In preferred embodiments, R₄₈ is an axial substituent on the trans-cyclooctene ring.

### R′

In some embodiments, each R′ is independently selected from the group consisting of hydrogen, C₁-C₆ alkylene groups, C₂-C₆ alkenylene groups, C₂-C₆ alkynylene groups, C₆ arylene, C₄-C₅ heteroarylene, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, and C₄-C₁₂ cycloalkylalkylene groups.

In some embodiments, each R′ is independently selected from the group consisting of hydrogen, C₁-C₄ alkylene groups, C₂-C₄ alkenylene groups, C₂-C₄ alkynylene groups, C₆ arylene, C₄-C₅ heteroarylene, C₃-C₆ cycloalkylene groups, C₅-C₈ cycloalkenylene groups, C₅-C₈ alkyl(hetero)arylene groups, C₅-C₈ (hetero)arylalkylene groups, C₄-C₁₂ alkylcycloalkylene groups, and C₄-C₈ cycloalkylalkylene groups.

Unless stated otherwise, for R′ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups are optionally substituted with a moiety selected from the group consisting of —Cl, —F, —Br, —I, —OH, —NH₂, ═O, —SH, —SO₃H, —PO₃H, —PO₄H₂, —NO₂, and optionally contain one or more heteroatoms selected from the group consisting of —O—, —S—, —NH—, —P—, and —Si, wherein the N, S, and P atoms are optionally oxidized.

### R″

In some embodiments, each R″ is independently selected from the group consisting of

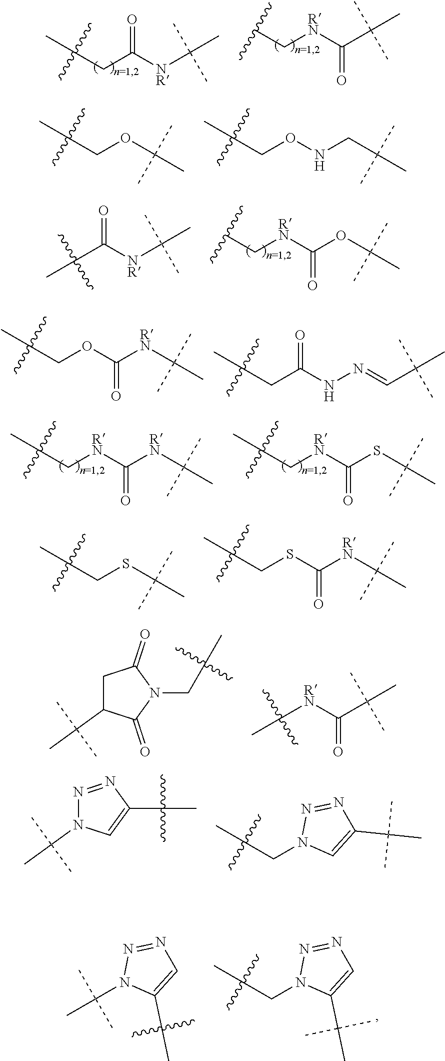

wherein the wiggly line depicts a bond to an ethylene glycol group or optionally to the R₃₃ adjacent to R₃₂ when t₄ is 0, and the dashed line depicts a bond to R₃₃ or G.

In preferred embodiments, R″ is —CH₂—C(O)NR′— or —CH₂—NR′C(O)—.

### G

In some embodiments, G is selected from the group consisting of CR′, N, C₅-C₅ arenetriyl, C₄-C₅ heteroarenetriyl, C₃-C₆ cycloalkanetriyl, and C₄-C₆ cycloalkenetriyl, wherein the arenetriyl, heteroarenetriyl, cycloalkanetriyl, and cycloalkenetriyl are optionally further substituted with groups selected from the group consisting of —Cl, —F, —Br, —I, —OR′, —N(R′)₂, —SR′, —SO₃H, —PO₃H, —PO₄H₂, —NO₂, —CF₃ and —R₃₁, and optionally contain one or more heteroatoms selected from the group consisting of —O—, —S—, —NR′—, —P—, and —Si—, wherein the N, S, and P atoms are optionally oxidized, wherein the N atoms are optionally quaternized. Preferably, G is CR′.

### L

In some embodiments, L is selected from the group consisting of —CH₂—OCH₃, —CH₂—OH, —CH₂—C(O)OH, —C(O)OH. In some embodiments, L is preferably —CH₂—OCH₃,

### Moieties M and X

It is understood that when moiety M is modified with a compound according to Formula (20), and M is —OH, —NHR′, or —SH, that it will loose a proton and will become a moiety X that is —O—, —NR′— or —S—, respectively. It is understood that when moiety M is —C(O)OH, that it will loose an —OH upon modification with a compound according to Formula (20), and that the resulting moiety X is —C(O)—. It is understood that when moiety M is —C(O)R′ or —C(O)R′— it will become a moiety X that is —C— upon modification with a compound according to Formula (20).

It is understood that a moiety M that is a —COOH may be derived from the C-terminus of the peptide, protein or peptoid, or from an acidic amino acid residue such as aspartic acid or glutamic acid.

It is understood that moiety M may be derived from non-natural amino acid residues containing —OH, —NHR′, —CO₂H, —SH, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrile, or a (oxa)norbornene.

It is understood that when moiety M is —OH it may be derived from an amino acid residue such as serine, threonine and tyrosine.

It is understood that when moiety M is —SH it may be derived from an amino acid residue such as cysteine.

It is understood that when moiety M is —NHR′ it may be derived from an amino acid residue such as lysine, homolysine, or ornithine.

t₁, t₂, t₃, t₄,

In some embodiments, t₁ is 0. In other embodiments, t₁ is 1.

In some embodiments, t₂ is 0. In other embodiments, t₂ is 1.

In some embodiments, t₃ is an integer in a range of from 0 to 12. Preferably, t₃ is an integer in a range of from 1 to 10, more preferably in a range of from 2 to 8. In particularly favourable embodiments, t₃ is 4 and y is 1.

In some embodiments, t₄ is 0. In other embodiments, t₄ is 1.

In some embodiments, t₅ is an integer in a range of from 6 to 48, preferably from 15 to 40, more preferably from 17 to 35, even more preferably from 20 to 30, most preferably from 22 to 28. In particularly preferred embodiments, t₅ is 23.

C^{A} and C^{B}

In some embodiments, C^{A} denotes a Construct A that is selected from the group consisting of drugs, targeting agents, and masking moieties. Preferably, Construct A is a drug, preferably a drug as defined herein.

In some embodiments, C^{B} denotes a Construct B, wherein said Construct B is selected from the group consisting of masking moieties, drugs, and targeting agents. Preferably, Construct B is selected from the group consisting of masking moieties, and targeting agents.

### Spacers S^{P}

It will be understood that when herein, it is stated that “each individual S^{P} is linked at all ends to the remainder of the structure” this refers to the fact that the spacer S^{P} connects multiple moieties within a structure, and therefore the spacer has multiple ends by definition. The spacer S^{P} may be linked to each individual moiety via different or identical moieties that may be each individually selected. Typically, these linking moieties are to be seen to be part of spacer S^{P} itself. In case the spacer S^{P} links two moieties within a structure, “all ends” should be interpreted as “both ends”. As an example, if the spacer connects a trans-cylooctene moiety to a Construct A, then “the remainder of the molecule” refers to the trans-cylooctene moiety and Construct A, while the connecting moieties between the spacer and the trans-cyclooctene moiety and Construct A (i.e. at both ends) may be individually selected.

Spacers S^{P} may consist of one or multiple Spacer Units S^{U} arranged linearly and/or branched and may be connected to one or more C^{B} moieties and/or one or more L^{C} or T^{R} moieties. The Spacer may be used to connect C^{B} to one T^{R} (Example A below; with reference to Formula 10a and 10b: f, e, a=1) or more T^{R} (Example B and C below; with reference to Formula 10a and 10b: f, e=1, a≥1), but it can also be used to modulate the properties, e.g. pharmacokinetic properties, of the C^{B}-T^{B}-C^{A} conjugate (Example D below; with reference to Formula 10a and 10b: one or more of c,e,g,h≥1). Thus a Spacer does not necessarily connect two entities together, it may also be bound to only one component, e.g. the T^{R} or L^{C}. Alternatively, the Spacer may comprise a Spacer Unit linking C^{B} to T^{R} and in addition may comprise another Spacer Unit that is only bound to the Spacer and serves to modulate the properties of the conjugate (Example F below; with reference to Formula 10a and 10b: e≥1). The Spacer may also consist of two different types of Su constructs, e.g. a PEG linked to a peptide, or a PEG linked to an alkylene moiety (Example E below; with reference to Formula 10a and 10b: e≥1). For the sake of clarity, Example B depicts a S^{U} that is branched by using a multivalent branched S^{U}. Example C depicts a S^{U} that is branched by using a linear S^{U} polymer, such as a peptide, whose side chain residues serve as conjugation groups.

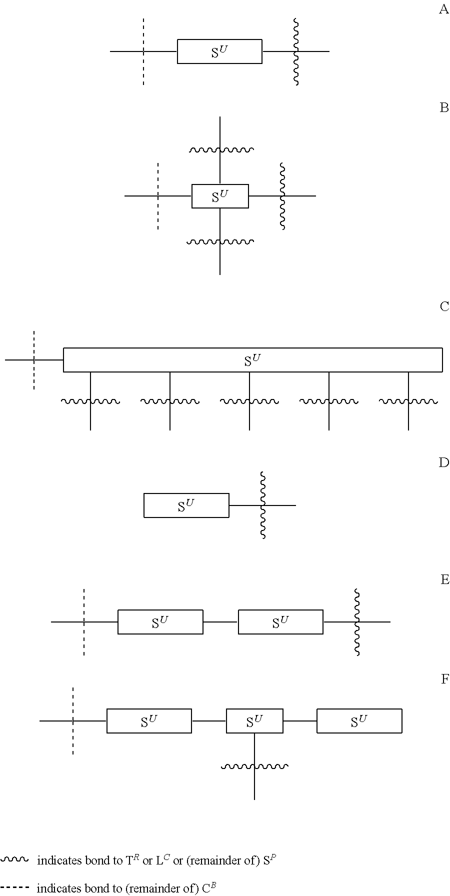

The Spacer may be bound to the Activator in similar designs such as depicted in above examples A-F.

The Spacer Units include but are not limited to amino acids, nucleosides, nucleotides, and biopolymer fragments, such as oligo- or polypeptides, oligo- or polypeptoids, or oligo- or polylactides, or oligo- or poly-carbohydrates, varying from 2 to 200, particularly 2 to 113, preferably 2 to 50, more preferably 2 to 24 and more preferably 2 to 12 repeating units. Exemplary preferred biopolymer S^{U} are peptides.

Yet other examples are alkyl, alkylene, alkenyl, alkenylene, alkynyl, alkynylene, cycloalkyl, cycloalkylene, cycloalkenyl, cycloalkenylene, cycloalkynyl, cycloalkynylene, aryl, arylene, alkylaryl, alkylarylene, arylalkyl, arylalkylene, arylalkenyl, arylalkynylene, arylalkynyl, arylalkynylene, polyethyleneamino, polyamine, which may be substituted or unsubstituted, linear or branched, may contain further cyclic moieties and/or heteroatoms, preferably O, N, and S, more preferably O; wherein in some embodiments these example S^{U} comprise at most 50 carbon atoms, more preferably at most 25 carbon atoms, more preferably at most 10 carbon atoms. In some embodiments the Su is independently selected from the group consisting of (CH₂)ᵣ, (C₃-C₈ carbocyclo), O—(CH₂)ᵣ, arylene, (CH₂)ᵣ-arylene, arylene-(CH₂)ᵣ, (CH₂)ᵣ—(C₃-C₈ carbocyclo), (C₃-C₈ carbocyclo)-(CH₂)ᵣ, (C₃-C₈ heterocyclo, (CH₂)ᵣ—(C₃-C₈ heterocyclo), (C₃-C₈ heterocyclo)-(CH₂)ᵣ, —(CH₂)ᵣC(O)NR₄(CH₂)ᵣ, (CH₂CH₂O)ᵣ, (CH₂CH₂O)ᵣCH₂, (CH₂)ᵣC(O)NR₄(CH₂CH₂O)ᵣ, (CH₂)ᵣC(O)NR₄(CH₂CH₂O)ᵣCH₂, (CH₂CH₂O)ᵣC(O)NR₄(CH₂CH₂O)ᵣ, (CH₂CH₂O)ᵣC(O)NR₄(CH₂CH₂O)ᵣCH₂, (CH₂CH₂O)ᵣC(O)NR₄CH₂, —(CH₂)ᵣC(O)NR₃₇(CH₂)ᵣ, (CH₂CH₂O)ᵣ, (CH₂CH₂O)ᵣCH₂, (CH₂)ᵣC(O)NR₃₇(CH₂CH₂O)ᵣ, (CH₂)ᵣC(O)NR₃₇(CH₂CH₂O)ᵣCH₂, (CH₂CH₂O)ᵣC(O)NR₃₇(CH₂CH₂O)ᵣ, (CH₂CH₂O)ᵣC(O)NR₃₇(CH₂CH₂O)ᵣCH₂, (CH₂CH₂O)ᵣC(O)NR₃₇CH₂; wherein r is independently an integer from 1-10, R₄ is as defined in Formula (1), and R₃₇ is as defined in Formula (19).

Other examples of Spacer Units Su are linear or branched polyalkylene glycols such as polyethylene glycol (PEG) or polypropylene glycol (PPG) chains varying from 2 to 200, particularly 2 to 113, preferably 2 to 50, more preferably 2 to 24 and more preferably 2 to 12 repeating units. It is preferred that when polyalkylene glycols such as PEG and PPG polymers are only bound via one end of the polymer chain, that the other end is terminated with —OCH₃, —OCH₂CH₃, OCH₂CH₂CO₂H.

Other polymeric Spacer Units are polymers and copolymers such as poly(N-(2-hydroxypropyl)methacrylamide) (HPMA), polylactic acid (PLA), polylactic-glycolic acid (PLGA), polyglutamic acid (PG), dextran, polyvinylpyrrolidone (PVP), poly(1-hydroxymethylethylene hydroxymethyl-formal (PHF). Other exemplary polymers are polysaccharides, glycopolysaccharides, glycolipids, polyglycoside, polyacetals, polyketals, polyamides, polyethers, polyesters. Examples of naturally occurring polysaccharides that can be used as Su are cellulose, amylose, dextran, dextrin, levan, fucoidan, carraginan, inulin, pectin, amylopectin, glycogen, lixenan, agarose, hyaluronan, chondroitinsulfate, dermatansulfate, keratansulfate, alginic acid and heparin. In yet other exemplary embodiments, the polymeric S^{U} comprises a copolymer of a polyacetal/polyketal and a hydrophilic polymer selected from the group consisting of polyacrylates, polyvinyl polymers, polyesters, polyorthoesters, polyamides, oligopeptides, polypeptides and derivatives thereof. Exemplary preferred polymeric S^{U} are PEG, HPMA, PLA, PLGA, PVP, PHF, dextran, oligopeptides, and polypeptides.

In some aspects of the invention polymers used in a SU have a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa.

Other exemplary S^{U} are dendrimers, such as poly(propylene imine) (PPI) dendrimers, PAMAM dendrimers, and glycol based dendrimers.

The Su of the invention expressly include but are not limited to conjugates prepared with commercially available cross-linker reagents such as BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB, DTME, BMB, BMDB, BMH, BMOE, BM(PEO)₃ and BM(PEO)₄.

To construct a branching Spacer one may use a S^{U} based on one or several natural or non-natural amino acids, amino alcohol, aminoaldehyde, or polyamine residues or combinations thereof that collectively provide the required functionality for branching. For example serine has three functional groups, i.e. acid, amino and hydroxyl groups and may be viewed as a combined amino acid an aminoalcohol residue for purpose of acting as a branching S^{U}. Other exemplary amino acids are lysine and tyrosine.

In some embodiments, the Spacer consist of one Spacer Unit, therefore in those cases S^{P} equals S^{U}. In other embodiments the Spacer consist of two, three or four Spacer Units.

In some aspects of the S^{P} has a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa. In some aspects of the invention, the S^{P} has a mass of no more than 5000 daltons, no more than 4000 daltons, no more than 3000 daltons, no more than 2000 daltons, no more than 1000 daltons, no more than 800 daltons, no more than 500 daltons, no more than 300 daltons, no more than 200 daltons. In some aspects the S^{P} has a mass from 100 daltons, from 200 daltons, from 300 daltons to 5000 daltons. In some aspects of the S^{P} has a mass from 30, 50, or 100 daltons to 1000 daltons, from about 30, 50, or 100 daltons to 500 daltons.

In some embodiments, S^{P} is a spacer selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein for S^{P} the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized.

In some embodiments, S^{P} comprises a moiety C^{M2} as described herein. When S^{P} comprises a moiety C^{M2}, it is coupled to a moiety C^{B} as indicated herein for how moieties according to Formula (22) are coupled to a moiety A according to Formula (21). In that case, C^{B} is equivalent to moiety A as defined for Formula (21), wherein X as defined for Formula (21) is part of C^{B}.

### Linker L^{C}

L^{C} is an optional self-immolative linker, which may consist of multiple units arranged linearly and/or branched and may release one or more C^{A} moieties. By way of further clarification, if r in R₄₈ is 0 the species C^{A} directly constitutes the leaving group of the release reaction, and if r in R₄₈ is 1, the self-immolative linker L^{C} constitutes the leaving group of the release reaction. The position and ways of attachment of linkers L^{C} and constructs C^{A} are known to the skilled person, see for example [Papot et al., Anticancer Agents Med. Chem., 2008, 8, 618-637]. Nevertheless, typical but non-limiting examples of self-immolative linkers L^{C} are benzyl-derivatives, such as those drawn below. There are two main self-immolation mechanisms: electron cascade elimination and cyclization-mediated elimination. The example below on the left functions by means of the cascade mechanism, wherein the bond to the Y^{C} between Trigger and L^{C}, here termed Y^{C1}, is cleaved, and an electron pair of Y^{C1}, for example an electron pair of NR⁶, shifts into the benzyl moiety resulting in an electron cascade and the formation of 4-hydroxybenzyl alcohol, CO₂ and the liberated C^{A} also comprising an Y^{C}, here termed Y^{C2}. The example in the middle functions by means of the cyclization mechanism, wherein cleavage of the bond to the amine of Y^{C1} leads to nucleophilic attack of the amine on the carbonyl, forming a 5-ring 1,3-dimethylimidazolidin-2-one and liberating the C^{A} including Y^{C2}. The example on the right combines both mechanisms, this linker will degrade not only into CO₂ and one unit of 4-hydroxybenzyl alcohol (when Y^{C1} is O), but also into one 1,3-dimethylimidazolidin-2-one unit.

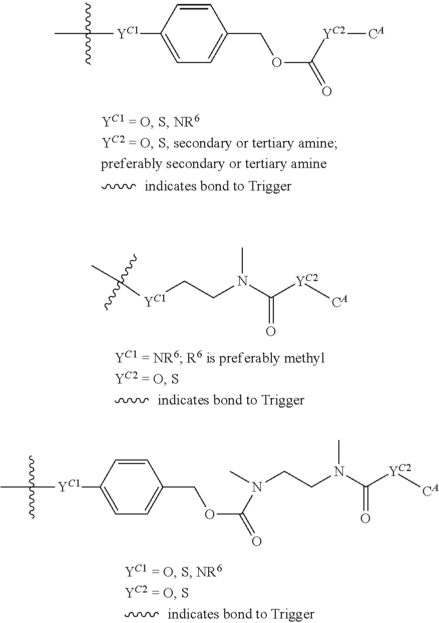

By substituting the benzyl groups of aforementioned self-immolative linkers L^{C}, it is possible to tune the rate of release of the construct C^{A}, caused by either steric and/or electronic effects on the cyclization and/or cascade release. Synthetic procedures to prepare such substituted benzyl-derivatives are known to the skilled person (see for example [Greenwald et al, J. Med. Chem., 1999, 42, 3657-3667] and [Thornthwaite et al, Polym. Chem., 2011, 2, 773-790]. Some examples of substituted benzyl-derivatives with different release rates are drawn below.

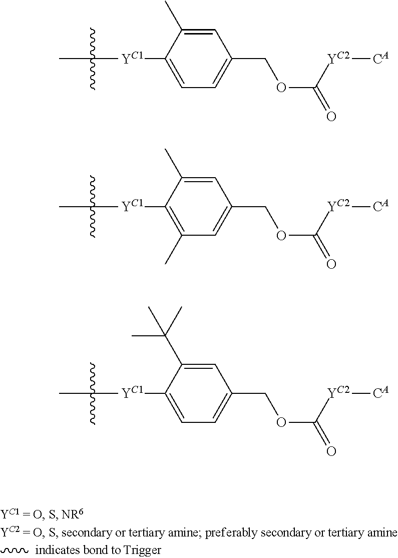

In some exemplary embodiments the L^{C} satisfies one of the following Formulae 23a-c

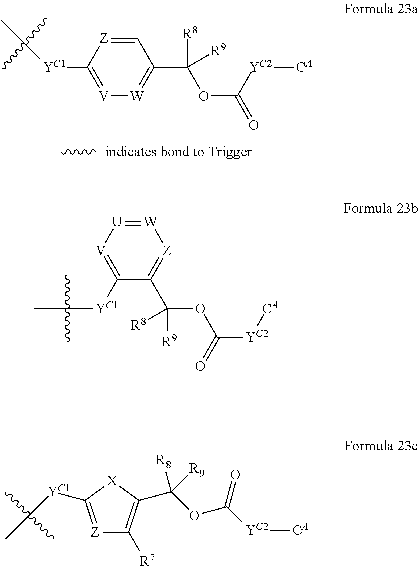

wherein Y^{C1} is O, S or NR⁶; V, U, W, Z are each independently CR⁷ or N; Y^{C2} is O, S, secondary amine or tertiary amine, wherein these Y^{C2} moieties are part of C^{A}; with R⁶, R⁷, R⁸, R⁹ as defined above. In some embodiments it is preferred that R⁶ is H or methyl, R⁷ is H, R⁸ is H or methyl and R⁹ is H. In some embodiments the R⁷ comprised in Formula 23c is CF₃ and Z is N.

In other embodiments the L^{C} satisfies the following Formula 23d

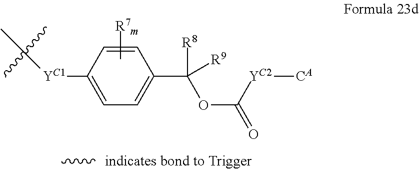

wherein Y^{C1} is O, S or NR⁶; Y^{C2} is O, S, secondary amine or tertiary amine, wherein these Y^{C2} moieties are part of C^{A}; with R⁶, R⁷, R⁸, R⁹ as defined above; preferably R⁷ is C₁-C₈ alkyl, C₆-C₁₂ aryl, C₁-C₈ O-alkyl, C₆-C₁₂ O-aryl, NO₂, F, Cl, Br, I, CN, with m being an integer from 0 to 4; each R⁸ and R⁹ are independently H, C₁-C₈ alkyl, C₆-C₁₂ aryl, C₁-C₈ O-alkyl, C₆-C₁₂ O-aryl, NO₂, F, Cl, Br, I, CN. Preferably R⁷ is electron donating and preferably m is an integer between 0 and 2, more preferably m is 0. Preferably R⁸ is H and R⁹ is H or methyl.

Self-immolative linkers that undergo cyclization include but are not limited to substituted and unsubstituted aminobutyric acid amide, appropriately substituted bicyclo[2.2.1] and bicyclo[2.2.2] ring system, 2-aminophenylpropionic acid amides, and trimethyl lock-based linkers, see e.g. [Chem. Biol. 1995, 2, 223], [J. Am. Chem. Soc. 1972, 94, 5815], [J. Org. Chem. 1990, 55, 5867], the contents of which are hereby incorporated by reference.

In other embodiments such cyclization L^{C} satisfies one of the following Formulae 3a-d.

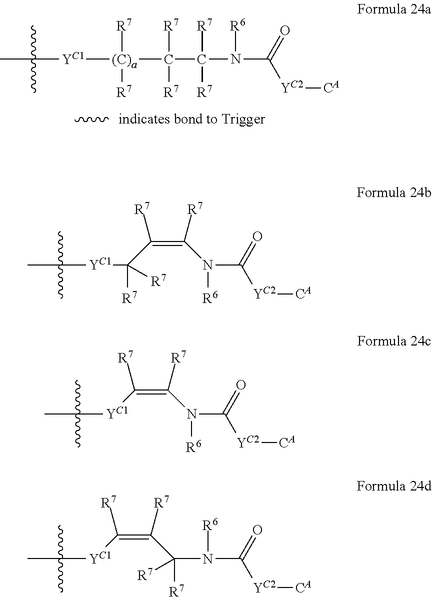

Wherein Y^{C1} is NR⁶; Y^{C2} is O or S, wherein these Y^{C2} moieties are part of C^{A}; a is independently 0 or 1; R⁶ and R⁷ are as defined above. Preferably R⁶ and R⁷ are H, unsubstituted C₁-C₈ alkyl, C₆ aryl, more preferably R⁶ is H or methyl and R⁷ is H.

Several non-limiting example structures of L^{C} are shown below. In these examples C^{A} is preferably bound to L^{C} via an Y^{C2} that is O or S, wherein O or S is part of C^{A}. For the avoidance of doubt, in these examples Y^{C1} is not denoted as such but is embodied by the relevant NH, NR⁶, S, O groups.

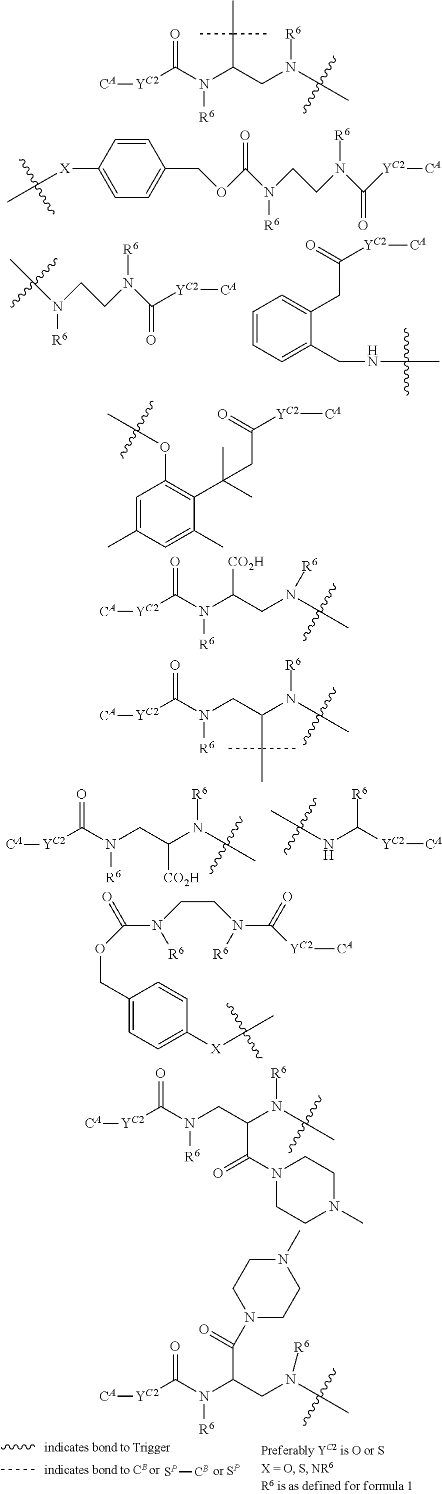

Several other non-limiting example structures of L^{C} are shown below. In these examples C^{A} is preferably bound to L^{C} via an Y^{C2} that is a secondary or primary amine, and wherein said Y^{C2} is part of C^{A}. For the avoidance of doubt, in these examples Y^{C1} is not denoted as such but is embodied by the relevant NH, NR⁶, S, O groups

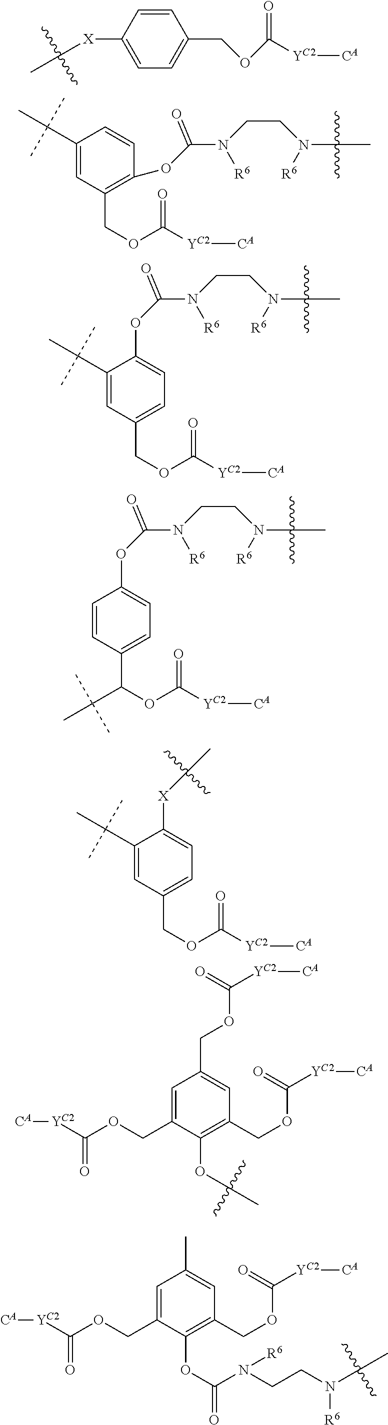

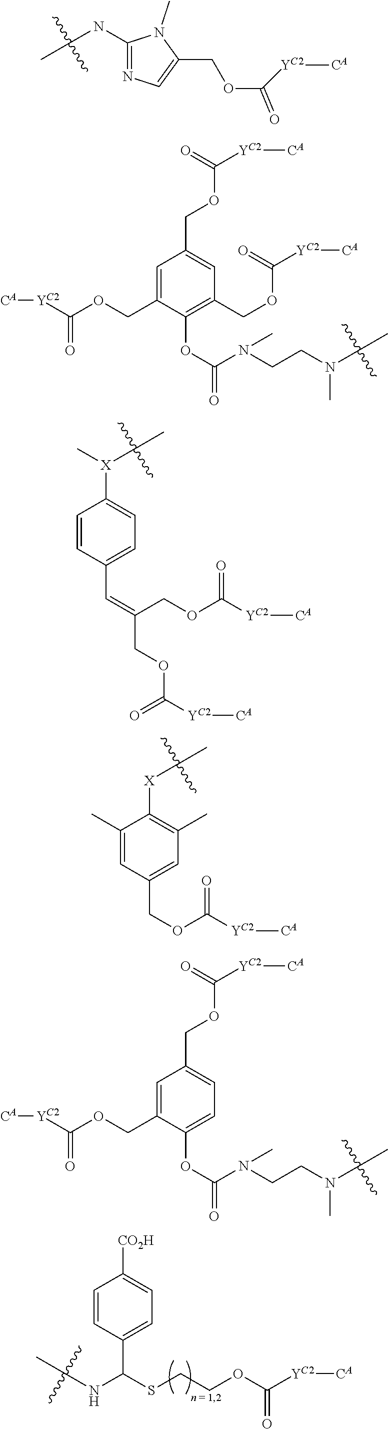

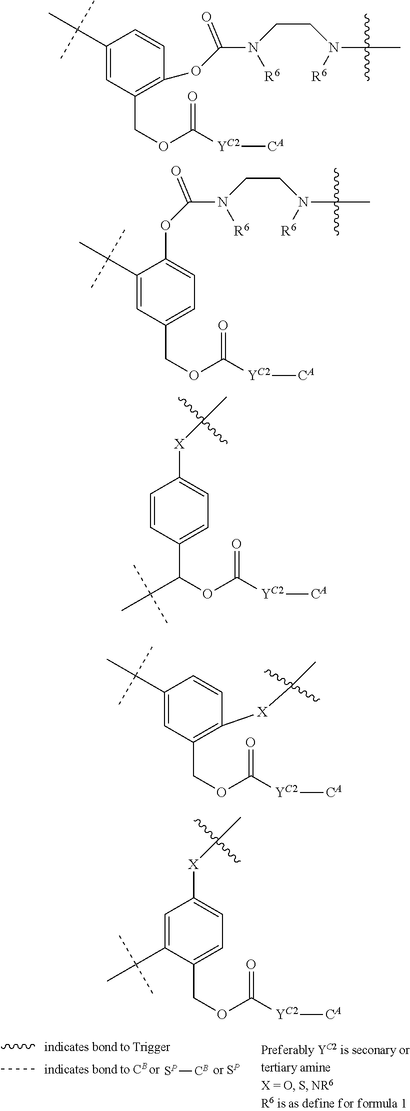

Further non-limiting examples of L^{C} can be found in WO2009017394(A1), U.S. Pat. No. 7,375,078, WO2015038426A1, WO2004043493, Angew. Chem. Int. Ed. 2015, 54, 7492-7509, the contents of which are hereby incorporated by reference.

In some aspects of the invention the L^{C} has a mass of no more than 1000 daltons, no more than 500 daltons, no more than 400 daltons, no more than 300 daltons, or from 10, 50 or 100 to 1000 daltons, from 10, 50, 100 to 400 daltons, from 10, 50, 100 to 300 daltons, from 10, 50, 100 to 200 daltons, e.g., 10-1000 daltons, such as 50-500 daltons, such as 100 to 400 daltons.

### Targeting

The kits of the invention are very suitable for use in targeted delivery of drugs.

A “primary target” as used in the present invention relates to a target for a targeting agent for therapy. For example, a primary target can be any molecule, which is present in an organism, tissue or cell. Targets include cell surface targets, e.g. receptors, glycoproteins; structural proteins, e.g. amyloid plaques; abundant extracellular targets such as stroma targets, tumor microenvironment targets, extracellular matrix targets such as growth factors, and proteases; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is up regulated or down-regulated in a certain disorder. According to a particular embodiment of the present invention, the primary target is a protein such as a (internalizing or non-internalizing) receptor.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosaccharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, fibronectin, VEGF/EGF and VEGF/EGF receptors, TAG72, CEA, CD19, CD20, CD22, CD40, CD45, CD74, CD79, CD105, CD138, CD174, CD227, CD326, CD340, MUC1, MUC16, GPNMB, PSMA, Cripto, Tenascin C, Melanocortin-1 receptor, CD44v6, G250, HLA DR, ED-A, ED-B, TMEFF2, EphB2, EphA2, FAP, Mesothelin, GD2, CAIX, 5T4, matrix metalloproteinase (MMP), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase, MSR1, FAP, CXCR, tumor endothelial marker (TEM), cMET, IGFR, FGFR, GPA33, hCG,

According to a further particular embodiment of the invention, the primary target and targeting agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

In some embodiments the Primary Target equals a therapeutic target. It shall be understood that a therapeutic target is the entity that is targeted by the Drug to afford a therapeutic effect.

### Targeting Agents T_{T}

A Targeting Agent, T_{T}, binds to a Primary Target. In order to allow specific targeting of the above-listed Primary Targets, the Targeting Agent T_{T} can comprise compounds including but not limited to antibodies, antibody derivatives, antibody fragments, antibody (fragment) fusions (e.g. bi-specific and tri-specific mAb fragments or derivatives), proteins, peptides, e.g. octreotide and derivatives, VIP, MSH, LHRH, chemotactic peptides, cell penetrating peptide, membrane translocation moiety, bombesin, elastin, peptide mimetics, organic compounds, inorganic compounds, carbohydrates, monosaccharides, oligosaccharides, polysaccharides, oligonucleotides, aptamers, viruses, whole cells, phage, drugs, polymers, liposomes, chemotherapeutic agents, receptor agonists and antagonists, cytokines, hormones, steroids, toxins. Examples of organic compounds envisaged within the context of the present invention are, or are derived from, estrogens, e.g. estradiol, androgens, progestins, corticosteroids, methotrexate, folic acid, and cholesterol.

According to a particular embodiment of the present invention, the Primary Target is a receptor and a Targeting Agent is employed, which is capable of specific binding to the Primary Target. Suitable Targeting Agents include but are not limited to, the ligand of such a receptor or a part thereof which still binds to the receptor, e.g. a receptor binding peptide in the case of receptor binding protein ligands. Other examples of Targeting Agents of protein nature include insulin, transferrin, fibrinogen-gamma fragment, thrombospondin, claudin, apolipoprotein E, Affibody molecules such as for example ABY-025, Ankyrin repeat proteins, ankyrin-like repeat proteins, interferons, e.g. alpha, beta, and gamma interferon, interleukins, lymphokines, colony stimulating factors and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin. Alternative examples of targeting agents include DNA, RNA, PNA and LNA which are e.g. complementary to the Primary Target.

Examples of peptides as targeting agents include LHRH receptor targeting peptides, EC-1 peptide, RGD peptides, HER2-targeting peptides, PSMA targeting peptides, somatostatin-targeting peptides, bombesin. Other examples of targeting agents include lipocalins, such as anticalins. One particular embodiment uses Affibodies™ and multimers and derivatives.

In one embodiment antibodies are used as the T^{T}. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodlies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as T^{T}, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. For the avoidance of doubt, in the context of this invention the term “antibody” is meant to encompass all of the antibody variations, fragments, derivatives, fusions, analogs and mimetics outlined in this paragraph, unless specified otherwise.

In a preferred embodiment the T^{T} is selected from antibodies and antibody derivatives such as antibody fragments, fragment fusions, proteins, peptides, peptide mimetics, organic molecules, dyes, fluorescent molecules, and enzyme substrates.

In a preferred embodiment the T^{T} being an organic molecule has a molecular weight of less than 2000 Da, more preferably less than 1500 Da, more preferably less than 1000 Da, even more preferably less than 500 Da.

In another preferred embodiment the T^{T} is selected from antibody fragments, fragment fusions, and other antibody derivatives that do not contain a Fc domain.

In another embodiment the T^{T} is a polymer and accumulates at the Primary Target by virtue of the EPR effect. Typical polymers used in this embodiment include but are not limited to polyethyleneglycol (PEG), poly(N-(2-hydroxypropyl)methacrylamide) (HPMA), polylactic acid (PLA), polylactic-glycolic acid (PLGA), polyglutamic acid (PG), polyvinylpyrrolidone (PVP), poly(l-hydroxymethylethylene hydroxymethyl-formal (PHF). Other examples are copolymers of a polyacetal/polyketal and a hydrophilic polymer selected from the group consisting of polyacrylates, polyvinyl polymers, polyesters, polyorthoesters, polyamides, oligopeptides, polypeptides and derivatives thereof. Other examples are oligopeptides, polypeptides, glycopolysaccharides, and polysaccharides such as dextran and hyaluronan,

In addition reference is made to [G. Pasut, F. M. Veronese, Prog. Polym. Sci. 2007, 32, 933-961].

According to a further particular embodiment of the invention, the Primary Target and Targeting Agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting Primary Targets with tissue-, cell- or disease-specific expression. For example, the CC49 antibody targets TAG72, the expression of which is limited in normal tissues, but receptors are overexpressed in various solid tumor cell types.

In one embodiment the Targeting Agent specifically binds or complexes with a cell surface molecule, such as a cell surface receptor or antigen, for a given cell population. Following specific binding or complexing of the T^{T} with the receptor, the cell is permissive for uptake of the Prodrug, which then internalizes into the cell. The subsequently administered Activator will then enter the cell and activate the Prodrug, releasing the Drug inside the cell. In another embodiment the Targeting Agent specifically binds or complexes with a cell surface molecule, such as a cell surface receptor or antigen, for a given cell population. Following specific binding or complexing of the T^{T} with the receptor, the cell is not permissive for uptake of the Prodrug. The subsequently administered Activator will then activate the Prodrug on the outside of the cell, after which the released Drug will enter the cell.

As used herein, a T^{T} that “specifically binds or complexes with” or “targets” a cell surface molecule, an extracellular matrix target, or another target, preferentially associates with the target via intermolecular forces. For example, the ligand can preferentially associate with the target with a dissociation constant (K_{d} or K_{D}) of less than about 50 nM, less than about 5 nM, or less than about 500 pM.

In another embodiment the targeting agent T^{T} localizes in the target tissue by means of the EPR effect. An exemplary T^{T} for use in with the EPR effect is a polymer.

It is preferred that when a T^{T} is comprised in an embodiment of the invention, it equals C^{B}.

### Masking Moieties

In order to avoid the drawbacks of current prodrug activation, it has been proposed to make use of an abiotic, bio-orthogonal chemical reaction to provoke release of the Masking Moiety from the masked Drug, preferably an antibody. In this type of Prodrug, the Masking Moiety is attached to the Drug, preferably an antibody, via a Trigger, and this Trigger is not activated endogeneously by e.g. an enzyme or a specific pH, but by a controlled administration of the Activator, i.e. a species that reacts with the Trigger moiety in the Prodrug, to induce release of the Masking Moiety or the Drug from the Trigger (or vice versa, release of the Trigger from the Masking Moiety or Drug, however one may view this release process), resulting in activation of the Drug. The previously presented Staudinger approach for this concept, as well as the earlier designs to use the IEDDA for this purpose, has turned out not to work well (vide supra).

In order to better address one or more of the foregoing desires, the present invention provides a kit for the administration and activation of a Prodrug, the kit comprising a Masking Moiety, denoted as M^{M}, linked covalently, directly or indirectly, to a Trigger moiety, which in turn is linked covalently, directly or indirectly, to a Drug, denoted as D^{D}, and an Activator for the Trigger moiety, wherein the Trigger moiety comprises a dienophile satisfying Formulae (19), (20) or (22) and the Activator comprises a tetrazine.

In another aspect, the invention presents a Prodrug comprising a Masking Moiety, M^{M}, linked, directly or indirectly, to dienophile moiety satisfying above Formulae (19), (20) or (22).

In yet another aspect, the invention provides a method of modifying a Drug, D^{D}, with a Masking Moiety M^{M} or one or more Masking Moieties M^{M} affording a Prodrug that can be activated by an abiotic, bio-orthogonal reaction, the method comprising the steps of providing a Masking Moiety and a Drug and chemically linking the Masking Moiety and a Drug to a dienophile moiety satisfying Formulae (19), (20) or (22).

In a still further aspect, the invention provides a method of treatment wherein a patient suffering from a disease that can be modulated by a drug, is treated by administering, to said patient, a Prodrug comprising a Trigger moiety linked to a Masking Moiety M^{M} and a Drug D^{D}, after activation of which by administration of an Activator the Masking Moiety will be released, activating the Drug, wherein the Trigger moiety comprises a dienophile structure satisfying Formulae (19), (20) or (22).

In a still further aspect, the invention is a compound comprising a dienophile moiety, said moiety comprising a linkage to a Masking Moiety M^{M}, for use in prodrug therapy in an animal or a human being.

In another aspect, the invention is the use of a diene as an Activator for the release, in a physiological environment, of a substance covalently linked to a compound satisfying Formulae (19), (20) or (22). In connection herewith, the invention also pertains to a diene, for use as an Activator for the release, in a physiological environment, of a substance linked to a compound satisfying Formulae (19), (20) or (22), and to a method for activating, in a physiological environment, the release of a substance linked to a compound satisfying Formulae (19), (20) or (22), wherein a tetrazine is used as an Activator.

In another aspect, the invention presents the use of the inverse electron-demand Diels-Alder reaction between a compound satisfying Formulae (19), (20) or (22) and a dienophile, preferably a trans-cyclooctene, as a chemical tool for the release, in a physiological environment, of a substance administered in a covalently bound form, wherein the substance is bound to a compound satisfying Formulae (19), (20) or (22).

For the avoidance of doubt, in the context of this invention wherein a M^{M} is removed from an antibody (i.e. Drug) the terms “activatable antibodies” and “Prodrug” mean the same.

For the avoidance of doubt, in the context of this invention wherein a M^{M} is removed from a Drug, the Drug itself can optionally bind to one or more Primary Targets without the use of an additional Targeting Agent T^{T}. In this context, the Primary Target is preferably the therapeutic target.

In a preferred embodiment, the Drug comprises a Targeting Agent T^{T} so that the Prodrug can bind a Primary Target. Following activation and M^{M} removal the Drug then binds another Primary Target, which can be a therapeutic target. In other embodiments, the Drug comprises one or more T^{T} moieties, against one or different Primary Targets.

For the avoidance of doubt, in the context of the use of Masking Moieties, Primary target and therapeutic target are used interchangeably.

For the avoidance of doubt, one Drug construct can be modified by more than one Masking Moieties.

In some embodiments the activatable antibodies or Prodrugs of this invention are used in the treatment of cancer. In some embodiments the activatable antibodies or Prodrugs of this invention are used in the treatment of an autoimmune disease or inflammatory disease such as rheumatoid arthritis. In some embodiments the activatable antibodies or Prodrugs of this invention are used in the treatment of a fibrotic disease such as idiopathic pulmonary fibrosis.

Exemplary classes of Primary Targets for activatable antibodies or Prodrugs of this invention include but are not limited to cell surface receptors and secreted proteins (e.g. growth factors), soluble enzymes, structural proteins (e.g. collagen, fibronectin) and the like. In preferred embodiments the Primary Target is an extracellular target. In other embodiments, the Primary Target is an intracellular target.

In another embodiment, the drug is a bi- or trispecific antibody derivative that serves to bind to tumor cells and recruit and activate immune effector cells (e.g. T-cells, NK cells), the immune effector cell binding function of which is masked and inactivated by being linked to a dienophile moiety as described above. The latter, again, serving to enable bio-orthogonal chemically activated drug activation.

When D^{D} is C^{B} it is preferred that D^{D} is not attached to remainder of the Prodrug through its antigen-binding domain. Preferably D^{D} is C^{A}.

Masking moieties M^{M} can for example be an antibody, protein, peptide, polymer, polyethylene glycol, polypropylene glycol carbohydrate, aptamers, oligopeptide, oligonucleotide, oligosaccharide, carbohydrate, as well as peptides, peptoids, steroids, organic molecule, or a combination thereof that further shield the bound drug D^{D} or Prodrug. This shielding can be based on e.g. steric hindrance, but it can also be based on a non covalent interaction with the drug D^{D}. Such Masking Moiety may also be used to affect the in vivo properties (e.g. blood clearance; biodistribution, recognition by the immune system) of the drug D^{D} or Prodrug.

In some embodiments, the Masking Moiety is an albumin binding moiety.

In some embodiments, the Masking Moiety equals a Targeting Agent.

In other embodiments, the Masking Moiety is bound to a Targeting Agent.

In some embodiments the T^{R} can itself act as a Masking Moiety, provided that C^{A} is D^{D}. For the sake of clarity, in these embodiments the size if the T^{R} without the attachment of a M^{M} is sufficient to shield the Drug D^{D} from its Primary Target, which, in this context, is preferably the therapeutic target.

The M^{M} of the modified D^{D} can reduce the D^{D}'s ability to bind its target allosterically or sterically.

In specific embodiments, the M^{M} is a peptide and does not comprise more than 50% amino acid sequence similarity to a natural protein-based binding partner of an antibody-based D^{D}.

In some embodiments M^{M} is a peptide between 2 and 40 amino acids in length.

In one embodiment the M^{M} reduces the ability of the D^{D} to bind its target such that the dissociation constant of the D^{D} when coupled to the M^{M} towards the target is at least 100 times greater than the dissociation constant towards the target of the D^{D} when not coupled to the M^{M}. In another embodiment, the coupling of the M^{M} to the D^{D} reduces the ability of the D^{D} to bind its target by at least 90%.

In some embodiments the M^{M} in the masked D^{D} reduces the ability of the D^{D} to bind the target by at least 50%, by at least 60 (N), by at least 70%, by at least 75%, by at least 80%, by at least 85%, by at least 90%, by at least 95%, by at least 96%, by at least 97%, by at least 98%, by at least 99 (N), or by 100%, as compared to the ability of the unmasked D^{D} to bind the target. The reduction in the ability of a D^{D} to bind the target can be determined, for example, by using an in vitro displacement assay, such as for example described for antibody D^{D} in WO2009/025846 and WO2010/081173.

In preferred embodiments the D^{D} comprised in the masked D^{D} is an antibody, which expressly includes full-length antibodies, antigen-binding fragments thereof, antibody derivatives antibody analogs, antibody mimics and fusions of antibodies or antibody derivatives.

In certain embodiments the M^{M} is not a natural binding partner of the antibody. In some embodiments, the M^{M} contains no or substantially no homology to any natural binding partner of the antibody. In other embodiments the M^{M} is no more than 5%, 10%, 15%, 20%, 25%, 30%, 35%, 40%, 45%, 50%, 55%, 60%, 65%, 70%, 75%, or 80% similar to any natural binding partner of the antibody. In some embodiments the M^{M} is no more than 5%, 10%, 15%, 20%, 25%, 30%, 35%, 40%, 45%, 50%, 55%, 60%, 65%, 70%, 75%, or 80% identical to any natural binding partner of the antibody. In some embodiments, the M^{M} is no more than 50% identical to any natural binding partner of the antibody. In some embodiments, the M^{M} is no more than 25% identical to any natural binding partner of the antibody. In some embodiments, the M^{M} is no more than 20% identical to any natural binding partner of the antibody. In some embodiments, the M^{M} is no more than 10% identical to any natural binding partner of the antibody.

In the Prodrug, the M^{M} and the Trigger T^{R}—the dienophile derivative- can be directly linked to each other. They can also be bound to each other via a spacer S^{P} or a self-immolative linker L^{C}. It will be understood that the invention encompasses any conceivable manner in which the diene Trigger is attached to the M^{M}. It will be understood that M^{M} is linked to the dienophile in such a way that the M^{M} is eventually capable of being released from the D^{D} after formation of the IEDDA adduct. Generally, this means that the bond between the D^{D} and the dienophile, or in the event of a self-immolative linker L^{C} the bond between the L^{C} and the dienophile and between the D^{D} and the L^{C} should be cleavable. Alternatively, this means that the bond between the M^{M} and the dienophile, or in the event of a self-immolative linker Le the bond between the L^{C} and the dienophile and between the M^{M} and the L^{C} should be cleavable.

In some embodiments, the antibody comprised in the masked antibody is a multi-antigen targeting antibody, comprising at least a first antibody or antigen-binding fragment or mimic thereof that binds a first Primary Target and a second antibody or antigen-binding fragment or mimic thereof that binds a second Primary Target. In some embodiments, the antibody comprised in the masked antibody is a multi-antigen targeting antibody, comprising a first antibody or antigen-binding fragment or mimic thereof that binds a first Primary Target, a second antibody or antigen-binding fragment or mimic thereof that binds a second Primary Target, and a third antibody or antigen-binding fragment or mimic thereof that binds a third Primary Target. In some embodiments, the multi-antigen targeting antibodies bind two or more different Primary Targets. In some embodiments, the multi-antigen targeting antibodies bind two or more different epitopes on the same Primary Target. In some embodiments the multi-antigen targeting antibodies bind a combination of two or more different targets and two or more different epitopes on the same Primary Target. In some embodiments the masked multi-antigen targeting antibodies comprise one M^{M} group, or two or more M^{M} groups. It shall be understood that preferably at least one of the Primary Targets is a therapeutic target.

In some embodiments of a multispecific activatable antibody, a scFv can be fused to the carboxyl terminus of the heavy chain of an IgG activatable antibody, to the carboxyl terminus of the light chain of an IgG activatable antibody, or to the carboxyl termini of both light and the heavy chain of an IgG activatable antibody. In some embodiments of a multispecific activatable antibody, a scFv can be fused to the amino terminus of the heavy chain of an IgG activatable antibody, to the amino terminus of the light chain of an IgG activatable antibody, or to the amino termini of both light and the heavy chain of an IgG activatable antibody. In some embodiments of a multispecific activatable antibody, a scFv can be fused to any combination of one or more carboxyl termini and one or more amino termini of an IgG activatable antibody. Methods of preparing multispecific antibodies are known to the person skilled in the art. In addition reference is made to [Weilde et at, Cancer Genomics & Proteomics 2013, 10, 1-18], [Weidle et al., Seminars in Oncology 2014, 41, 5, 653-660], [Jachimowicz et al., BioDrugs (2014) 28:331-343], the contents of which are hereby incorporated by reference.

In some embodiments, a M^{M} linked to a T^{R} is attached to and masks an antigen binding domain of the IgG. In some embodiments, a M^{M} linked to a T^{R} is attached to and masks an antigen binding domain of at least one scFv. In some embodiments, a M^{M} linked to a T^{R} is attached to and masks an antigen binding domain of the IgG and a M^{M} linked to a T^{R} is attached to and masks an antigen binding domain of at least one scFv.

In some embodiments, the M^{M} has a dissociation constant, i.e., dissociation constant at an equilibrium state, K_{d}, for binding to the antibody that is greater than the K_{d} for binding of the antibody to its Primary Target. In some embodiments, the M^{M} has a K_{d} for binding to the antibody that is approximately equal to the K_{d} for binding of the antibody to its Primary Target. In some embodiments, the M^{M} has a K_{d} for binding to the antibody that is less than the K_{d} for binding of the antibody to its Primary Target. In some embodiments, the M^{M} has a K_{d} for binding to the antibody that is no more than 2, 3, 4, 5, 10, 25, 50, 100, 250, 500, or 1,000 fold greater than the K_{d} for binding of the antibody to its Primary Target. In some embodiments, the M^{M} has a K_{d} for binding to the antibody that is between 1-5, 2-5, 2-10, 5-10, 5-20, 5-50, 5-100, 10-100, 10-1,000, 20-100, 20-1,000, or 100-1,000 fold greater than the Ki for binding of the antibody to its Primary Target.

In some embodiments, the M^{M} has an affinity for binding to the antibody that is greater than the affinity of binding of the antibody to its Primary Target. In some embodiments, the M^{M} has an affinity for binding to the antibody that is approximately equal to the affinity of binding of the antibody to its Primary Target. In some embodiments, the M^{M} has an affinity for binding to the antibody that is less than the affinity of binding of the antibody to its Primary Target. In some embodiments, the M^{M} has an affinity for binding to the antibody that is 2, 3, 4, 5, 10, 25, 50, 100, 250, 500, or 1,000 fold less than the affinity of binding of the antibody to its Primary Target. In some embodiments, the M^{M} has an affinity of binding to the antibody that is between 1-5, 2-5, 2-10, 5-10, 5-20, 5-50, 5-100, 10-100, 10-1,000, 20-100, 20-1,000, or 100-1,000 fold less than the affinity of binding of the antibody to its Primary Target. In some embodiments, the M^{M} has an affinity of binding to the antibody that is 2 to 20 fold less than the affinity of binding of the antibody to its Primary Target.

In some embodiments, a M^{M} not covalently linked to the antibody and at equimolar concentration to the antibody does not inhibit the binding of the antibody to its Primary Target. In some embodiments, the M^{M} does not interfere of compete with the antibody for binding to the Primary Target when the Prodrug is in a cleaved state.

In some embodiments, the antibody has a dissociation constant of about 100 nM or less for binding to its Primary Target.

In some embodiments, the antibody has a dissociation constant of about 10 nM or less for binding to its Primary Target.

In some embodiments, the antibody has a dissociation constant of about 1 nM or less for binding to its Primary Target.

In some embodiments, the coupling of the M^{M} reduces the ability of the antibody to bind its Primary Target such that the dissociation constant (K_{d}) of the antibody when coupled to the M^{M} towards its Primary Target is at least 20 times greater than the K_{d} of the antibody when not coupled to the M^{M} towards its Primary Target.

In some embodiments, the coupling of the M^{M} reduces the ability of the antibody to bind its Primary Target such that the K_{d} of the antibody when coupled to the M^{M} towards its Primary Target is at least 40 times greater than the K_{d} of the antibody when not coupled to the M^{M} towards its Primary Target.

In some embodiments, the coupling of the M^{M} reduces the ability of the antibody to bind its Primary Target such that the K_{d} of the antibody when coupled to the M^{M} towards its Primary Target is at least 100 times greater than the K_{d} of the antibody when not coupled to the M^{M} towards its Primary Target.

In some embodiments, the coupling of the M^{M} reduces the ability of the antibody to bind its Primary Target such that the K_{d} of the antibody when coupled to the M^{M} towards its Primary Target is at least 1,000 times greater than the K_{d} of the antibody when not coupled to the M^{M} towards its Primary Target.

In some embodiments, the coupling of the M^{M} reduces the ability of the antibody to bind its Primary Target such that the K_{d} of the antibody when coupled to the M^{M} towards its Primary Target is at least 10,000 times greater than the K_{d} of the antibody when not coupled to the M^{M} towards its Primary Target.

In some embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 100,000 times greater than the K_{d} of the antibody when not coupled to the M^{M} towards its Primary Target.

In some embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 1,000,000 times greater than the K_{d} of the antibody when not coupled to the M^{M} towards its Primary Target.

In some embodiments, for example when using a non-binding steric M^{M} as defined below, the coupling of the M^{M} reduces the ability of the antibody to bind its Primary Target such that the K_{d} of the antibody when coupled to the M^{M} towards its Primary Target is at least 10,000,000 times greater than the K_{d} of the antibody when not coupled to the M^{M} towards its Primary Target.

Exemplary Drugs that can be used in a Prodrug relevant to this invention using Masking Moieties include but are not limited to: antibodies, antibody derivatives, antibody fragments, proteins, aptamers, oligopeptides, oligonucleotides, oligosaccharides, carbohydrates, as well as peptides, peptoids, steroids, toxins, hormones, viruses, whole cells, phage.

In some embodiments the drugs are low to medium molecular weight compounds, preferably organic compounds (e.g. about 200 to about 2500 Da, preferably about 300 to about 1750 Da, more preferably about 300 to about 1000 Da).

In one embodiment antibodies are used as the Drug. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulins is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, camelid antibodies, minibodies, nanobodies, resurfaced antibodies, humanized antibodies, fully human antibodies, single domain antibody (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which is hereby incorporated by reference. Other embodiments use antibody mimetics as Drug, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference.

“Antibody fragment” refers to at least a portion of the variable region of the immunoglobulin that binds to its target, i.e. the antigen-binding region. Multimers may be linearly linked or may be branched and may be derived from a single vector or chemically connected, or non-covalently connected. Methods of making above listed constructs are known in the art. For the avoidance of doubt, in the context of this invention the term “antibody” is meant to encompass all of the antibody variations, fragments, derivatives, fusions, analogs and mimetics outlined in this paragraph, unless specified otherwise.

Typical drugs for which the invention is suitable include, but are not limited to: monospecific, bispecific and trispecific antibodies and antibody fragment or protein fusions, preferably bispecific and trispecific. In some embodiments the activatable antibody or derivative is formulated as part of a pro-Bispecific T Cell Engager (BITE) molecule.

Other embodiments use immunotoxins, which are a fusion or a conjugate between a toxin and an antibody. Typical toxins comprised in an immunotoxins are cholera toxin, ricin A, gelonin, saporin, bouganin, ricin, abrin, diphtheria toxin, Staphylococcal enterotoxin, Bacillus Cyt2Aa1 toxin, Pseudomonas exotoxin PE38, Pseudomonas exotoxin PE38KDEL, granule-associated serine protease granzyme B, human ribonucleases (RNase), or other pro-apoptotic human proteins. Other exemplary cytotoxic human proteins which may be incorporated into fusion constructs are caspase 3, caspase 6, and BH3-interacting domain death agonist (BID). Current immunotoxins have immunogenicity issues and toxicity issues, especially towards vascular endothelial cells. Masking the targeted toxin by a M^{M} such as a PEG or peptide and removing the M^{M} once the masked immunotoxin has bound to its target is expected to greatly reduce the toxicity and immunogenicity problems.

Other embodiments use immunocytokines, which are a fusion or a conjugate between a cytokine and an antibody. Typical cytokines used in cancer therapy include IL-2, IL-7, IL-12, IL-15, IL-21, TNF. A typical cytokine used in autoimmune diseases is the anti-inflammatory IL-10. Masking the targeted cytokine by a M^{M} such as a PEG or peptide and removing the M^{M} once the masked immunocytokine has bound to its target is expected to greatly reduce the toxicity problems.

In some embodiments the unmasked Drug is multispecific and binds to two or more same or different Primary Targets. In some embodiments the multispecific Drug comprises one or more (masked) antibodies (also referred to as binding moieties) that are designed to engage immune effector cells. In some embodiments the masked multispecific Prodrug comprises one or more (masked) antibodies that are designed to engage leukocytes. In some embodiments the masked multispecific Prodrug comprises one or more (masked) antibodies that are designed to engage T cells. In some embodiments the masked multispecific Prodrug comprises one or more (masked) antibodies that engage a surface antigen on a leukocyte such as on a T cell, natural killer (NK) cell, a myeloid mononuclear cell, a macrophage and/or another immune effector cell. In some embodiments the immune effector cell is a leukocyte, a T cell, a NK cell, or a mononuclear cell.

In an exemplary multispecific masked Prodrug the Prodrug comprises an antibody (i.e. Targeting Agent) for a cancer receptor, e.g. TAG72, a antibody for CD3 on T cells, and an antibody for CD28 on T cells, wherein either the antibody for CD3 or for CD28 or both is masked by a M^{M}. Another example is an activatable antibody that comprises an antibody for a cancer receptor, and an antibody for CD3 on T cells, wherein the antibody for CD3 is masked by a M^{M}. Another example is a Prodrug that has an antibody for a cancer receptor, and an antibody for CD28 on T cells, wherein the antibody for CD28 is masked by a M^{M}. Another example is a Prodrug that has an antibody for a cancer receptor, and an antibody for CD16a on NK cells, wherein the antibody for CD16a is masked by a M^{M}. In yet another embodiment the unmasked Drug binds two different immune cells and optionally in addition a tumor cell. Said multispecific antibody derivatives can for example be prepared by fusing or conjugating antibodies, antibody fragments such as Fab, Fabs, scFv, camel antibody heavy chain fragments and proteins.

In some preferred embodiments the M^{M} reduces the binding of the Drug to Primary Targets, equaling therapeutic targets, selected from CD3, CD28, PD-L1, PD-1, LAG-3, TIGIT, TIM-3, B7114, Vista, CTLA-4, polysialic acids and corresponding lectins. In other preferred embodiments the M^{M} masks a T-cell agonist, an NK cell agonist, an DC cell agonist.

In some embodiments of an immune effector cell engaging masked multispecific Prodrug such as a T-cell engaging multispecific activatable antibody, at least one antibody comprised in the Prodrug, preferably a Targeting Agent, binds a Primary Target that is typically an antigen present on the surface of a tumor cell or other cell type associated with disease, such as, but not limited to, EGFR, erbB2, EpCAM, PD-L1, B7113 or CD71 (transferrin receptor), and at least one other antibody comprised in the Prodrug binds Primary Target that is typically a stimulatory or inhibitory antigen present on the surface of a T-cell, natural killer (NK) cell, myeloid mononuclear cell, macrophage, and/or other immune effector cell, such as, but not limited to, B7-114, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3 or VISTA. In some embodiments it is preferred that the targeted CD3 antigen is CD3c or CD3 epsilon.

One embodiment of the disclosure is a multispecific activatable antibody that includes an antibody, preferably a Targeting Agent, directed to a tumor target and another agonist antibody, preferably a Drug, directed to a co-stimulatory receptor expressed on the surface of an activated T cell or NK cell, wherein the agonist antibody is masked. Examples of co-stimulatory receptors include but are not limited to CD27, CD137, GITR, HVEM, NKG2D, OX40. In this embodiment, once the Prodrug is tumor-bound and activated it would effectively crosslink and activate the T cell or NK cell expressed co-stimulatory receptors in a tumor dependent manner to enhance the activity of T cell or NK cells that are responding to any tumor antigen via their endogenous T cell or NK cell activating receptors. The activation dependent nature of these T cell or NK cell co-stimulatory receptors would focus the activity of the activated multispecific Prodrug to tumor specific T cells without activating all T cells independent of their antigen specificity.

One embodiment of the disclosure is a multispecific activatable antibody targeted to a disease characterized by T cell overstimulation, such as, but not limited to, an autoimmune disease or inflammatory disease microenvironment. Such a Prodrug includes an antibody, for example a IgG or scFv, directed to a target comprising a surface antigen expressed in a tissue targeted by a T cell in autoimmune or inflammatory disease and an antibody, for example IgG or scFv, directed to an inhibitory receptor expressed on the surface of a T cell or NK cell, wherein the T cell or NK cell inhibitory antibody is masked. Examples of inhibitory receptors include but are not limited to BTLA, CTLA-4, LAG3, PD-1, TIGIT, TIM3, and NK-expressed KIRs. Examples of a tissue antigen targeted by T cells in autoimmune disease include but are not limited to a surface antigen expressed on myelin or nerve cells in multiple sclerosis or a surface antigen expressed on pancreatic islet cells in Type 1 diabetes. In this embodiment, the Prodrug localizes at the tissue under autoimmune attack or inflammation, is activated by the Activator and co-engages the T-cell or NK cell inhibitory receptor to suppress the activity of autoreactive T cells responding to any disease tissue targeted antigens via their endogenous TCR or activating receptors.

Other non-limiting exemplary Primary Targets for the binding moieties comprised in Drugs of this invention are listed in the patent WO2015/013671, the contents of which are hereby incorporated by reference.

In another embodiment, the Drug is a masked vaccine, which can be unmasked at a desired time and/or selected location in the body, for example subcutaneously and/or in the proximity of lymph nodes. In another embodiment, the Drug is a masked antigen, e.g. a masked peptide, which optionally is present in a Major

Histocompatibility Complex (MHC) and which can be unmasked at a desired time and/or selected location in the body, for example subcutaneously and/or in the proximity of lymph nodes.

The Prodrug may further comprise another linked drug, which is released upon target binding, either by proteases, pH, thiols, or by catabolism. Examples are provided in the review on Antibody-drug conjugates in [Polakis, Pharmacol. Rev. 2016, 68, 3-19]. The invention further contemplates that the Prodrug can induce antibody-dependent cellular toxicity (ADCC) or complement dependent cytotoxicity (CDC) upon unmasking of one or more moieties of the Prodrug. The invention also contemplates that the Prodrug can induce antibody-dependent cellular toxicity (ADCC) or complement dependent cytotoxicity (CDC) independent of unmasking of one or more moieties of the Prodrug.

Some embodiments use as said additional drug antiproliferative/antitumor agents, antibiotics, cytokines, anti-inflammatory agents, anti-viral agents, antihypertensive agents, chemosensitizing, radiosensitizing agents, DNA damaging agents, anti-metabolites, natural products and their analogs.

It is preferred that the Drug is a protein or a antibody.

### Administration of a Prodrug

When administering the Prodrug (as further defined in the sections below) and the Activator to a living system, such as an animal or human, in preferred embodiments the Prodrug is administered first, and it will take a certain time period before the Prodrug has reached the Primary Target. This time period may differ from one application to the other and may be minutes, days or weeks. After the time period of choice has elapsed, the Activator is administered, will find and react with the Prodrug and will thus activate the Prodrug and/or afford Drug release at the Primary Target. In some preferred embodiments, the time interval between the administration of the Prodrug and the Activator is between 10 minutes and 4 weeks. In some preferred embodiments, the time interval between the administration of the Prodrug and the Activator is between 1 hour and 2 weeks, preferably between 1 and 168 hours, more preferably between 1 and 120 hours, even more preferably between 1 and 96 hours, most preferably between 3 and 72 hours.

The compositions of the invention can be administered via different routes including but not limited to intravenous or subcutaneous injection, intraperitoneal, local injection, oral administration, rectal administration and inhalation. Formulations suitable for these different types of administrations are known to the skilled person. Prodrugs or Activators according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include for example saline, buffered saline, dextrose, water, glycerol, ethanol, and combinations thereof. The formulation should suit the mode of administration.

It will be understood that the chemical entities administered, viz. the Prodrug and the Activator, can be in a modified form that does not alter the chemical functionality of said chemical entity, such as salts, hydrates, or solvates thereof.

After administration of the Prodrug, and before the administration of the Activator, it is preferred to remove excess Prodrug by means of a Clearing Agent in cases when Prodrug activation in circulation is undesired and when natural Prodrug clearance is insufficient. A Clearing Agent is an agent, compound, or moiety that is administered to a subject for the purpose of binding to, or complexing with, an administered agent (in this case the Prodrug) of which excess is to be removed from circulation. The Clearing Agent is capable of being directed to removal from circulation. The latter is generally achieved through liver receptor-based mechanisms, although other ways of secretion from circulation exist, as are known to the skilled person. In the invention, the Clearing Agent for removing circulating Prodrug, preferably comprises a dienophile moiety, e.g. as discussed above, capable of reacting to the tetrazine moiety of the Prodrug.

In other embodiments the Activator is administered first, followed by the Prodrug, wherein the time interval between the administration of the two components ranges from 1 minute to 1 week, preferably from 10 minutes to 3 days.

In other embodiments, the Prodrug and Activator are administered at the same time. either as two separate administrations or as a co-administration.

In yet another embodiment, the Prodrug and Activator are reacted with one another prior to administration and the resulting reaction mixture is then adminstered, wherein the time interval between start of the reaction and the administration varies from 1 minute to 3 days, preferably 1 minute to 1 day, more preferably from 1 minute to 3 hours.

### Therapeutic Use

In some embodiments, the kits of the invention are for use as a medicament. Alternatively, the kits of the invention are used in a method for treating patients, said method comprising administering the compounds comprised in the kits of the invention to a subject.

### Embodiments

The invention is hereinbelow presented in exemplary Embodiments.

Embodiment 1. A kit comprising a tetrazine and a dienophile, wherein the tetrazine satisfies any one of the Formulae (1), (2), (3), (4), (5), (6), (7), or (8):

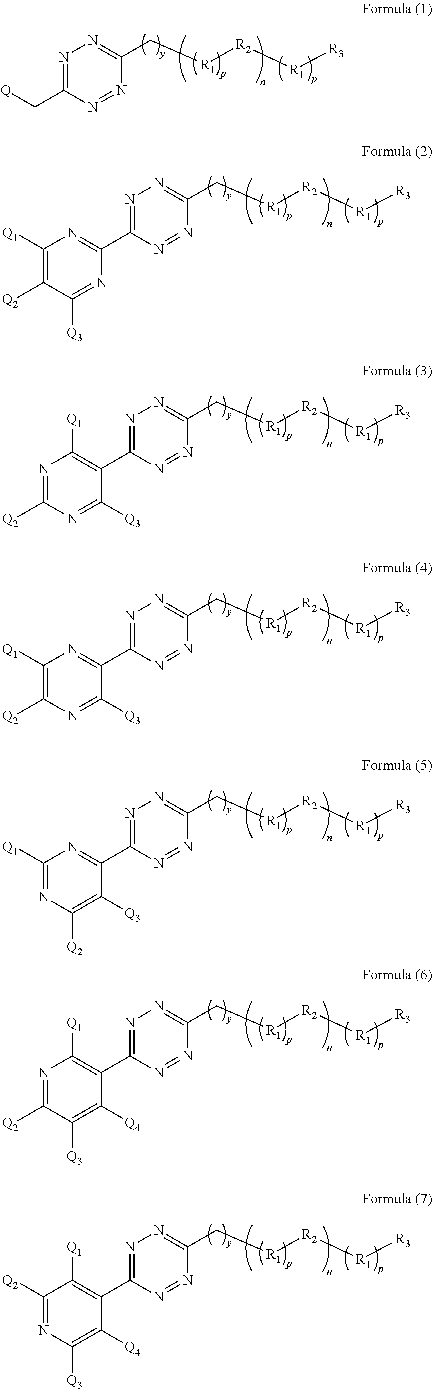

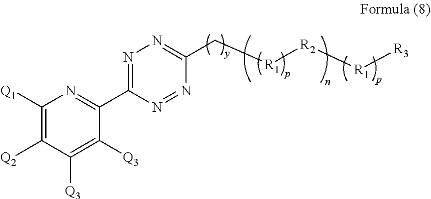

wherein each moiety Q, Q₁, Q₂, Q₃, and Q₄ is independently selected from the group consisting of hydrogen, and moieties according to Formula (9):

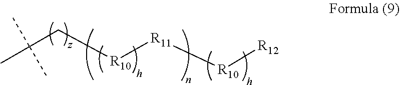

wherein the dashed line indicates a bond to the remaining part of the molecules satisfying any of the Formulae (1), (2), (3), (4), (5), (6), (7), or (8),
wherein each n is an integer independently selected from a range of from 0 to 24,
wherein each p is independently 0 or 1,
wherein y is an integer in a range of from 1 to 12,
wherein z is an integer in a range of from 0 to 12,
wherein each h is independently 0 or 1,
wherein each R₁ and R₁₀ are independently selected from the group consisting of —O—, —S—, —SS—, —NR₄—, —N(R₄)₂_{⁺}—, —N═N—, —C(O)—, —C(S)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, SC(S)—, —C(S)S—, —SC(S)NR₄—, —NR₄C(S)S—, —C(S)O—, —OC(S)O—, —OC(S)NR₄—, —NR₄C(S)—, —NR₄C(S)O—, —NR₄C(S)—, —C(S)NR₄—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —S(O)₂NR₄—, NR₄S(O)₂—, —OP(O)(R₄)₂—, —SP(O)(R₄)₂—, —NR₄P(O)(R₄)₂—,
wherein R₂ and R₁₁ are independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups,
wherein R₃ and R₁₂ are independently selected from the group consisting of hydrogen, —OH, —NH₂, —N₃, —Cl, —Br, —F, —I, and a chelating moiety,
wherein each R₄ is independently selected from the group consisting of hydrogen, C₁-C₂₄ alkyl groups, C₂-C₂₄ alkenyl groups, C₂-C₂₄ alkynyl groups, C₆-C₂₄ aryl, C₂-C₂₄ heteroaryl, C₃-C₂₄ cycloalkyl groups, C₅-C₂₄ cycloalkenyl groups, C₁₂-C₂₄ cycloalkynyl groups,
wherein in Formulae (1), (2), (3), (4), (5), (6), (7) and (8) at least one moiety selected from the group consisting of Q, Q₁, Q₂, Q₃, Q₄, and —(CH₂)_{y}—((R₁)ₚ—R₂)ₙ—(R₄)ₚ)—R₃ has a molecular weight in a range of from 100 Da to 3000 Da,
wherein in Formulae (1), (2), (3), (4), (5), (6), (7) and (8) moieties selected from the group consisting of Q, Q₁, Q₂, Q₃, Q₄, and —(CH₂)_{y}—((R₁)ₚ—R₂)ₙ—(R₁)ₚ)—R₃ have a molecular weight of at most 3000 Da,
wherein in Formula (1) when Q is not H, z is 0, n belonging to Q is at least 1, and at least one h is 1, then y is at least 2,
wherein in Formula (1) when Q is not H, y is 1, n belonging to —(CH₂)_{y}—((R₄)ₚ—R₂)ₙ—(R₄)ₚ)—R₃ is at least 1, and at least one p is 1, then z is at least 1,
wherein in Formula (8) when Q₁, Q₂, Q₃, and Q₄ are hydrogen, then y is not 1,
wherein in Formula (8) when y is 1, all p are 0, n belonging to —(CH₂)_{y}—((R₁)ₚ—R₂)ₙ—(R₁)₄))—R₃ is 0, R₃ is hydrogen, Q₄ is hydrogen, Q₃ is hydrogen, Q₄ is hydrogen, and Q₂ is not hydrogen, then z is at least 1,
wherein the R₂ groups, the R₁₁ groups, and the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized,
wherein the R₂ groups, the R₁₁ groups, and the R₄ groups not being hydrogen, are optionally further substituted with one or more substituents selected from the group consisting of —Cl, —F, —Br, —I, —OH, —NH₉, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups, wherein the substituents optionally contain one or more heteroatoms selected from the group consisting of O, S, NR₅, P, and Si, wherein the N, S, and P atoms are optionally oxidized, wherein the N atoms are optionally quaternized,
wherein each R₅ is independently selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the R₅ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

Embodiment 2. A kit according to any one of the preceding Embodiments, wherein the compound according to Formulae (1), (2), (3), (4), (5), (6), (7) or (8) has a Log P value of at most 3.0, preferably at most 2.0.

Embodiment 3. A kit according to any one of the preceding Embodiments, wherein R₃ is a chelator moiety selected from the group consisting of

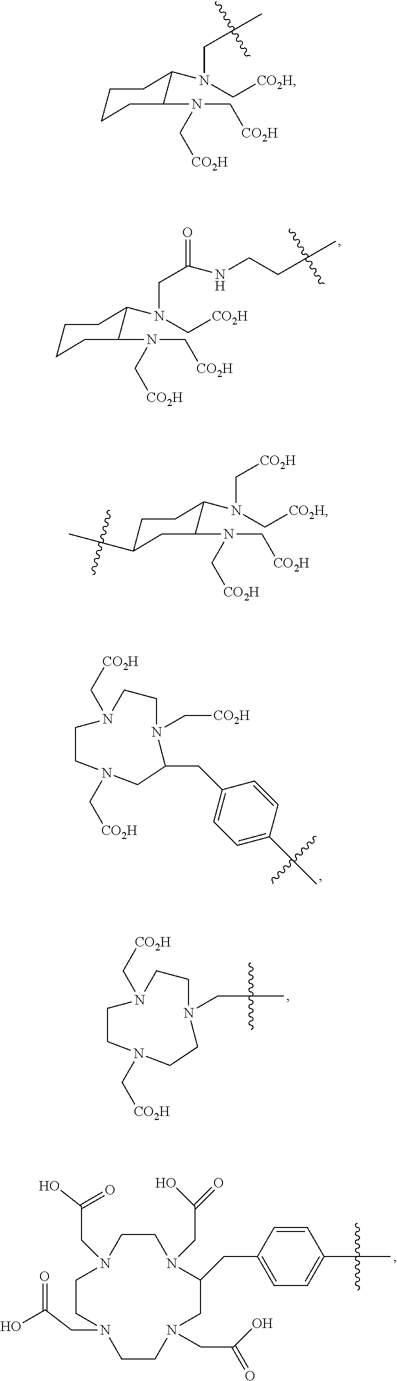 [SMILES: *CN(CC(=O)O)C1CCCCC1N(CC(=O)O)CC(=O)O ; *CCNC(=O)CN(CC(=O)O)C1CCCCC1N(CC(=O)O)CC(=O)O ; *C1CCC(N(CC(=O)O)CC(=O)O)C(N(CC(=O)O)CC(=O)O)C1 ; *c1ccc(CC2CN(CC(=O)O)CCN(CC(=O)O)CCN2CC(=O)O)cc1 ; *CN1CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1]

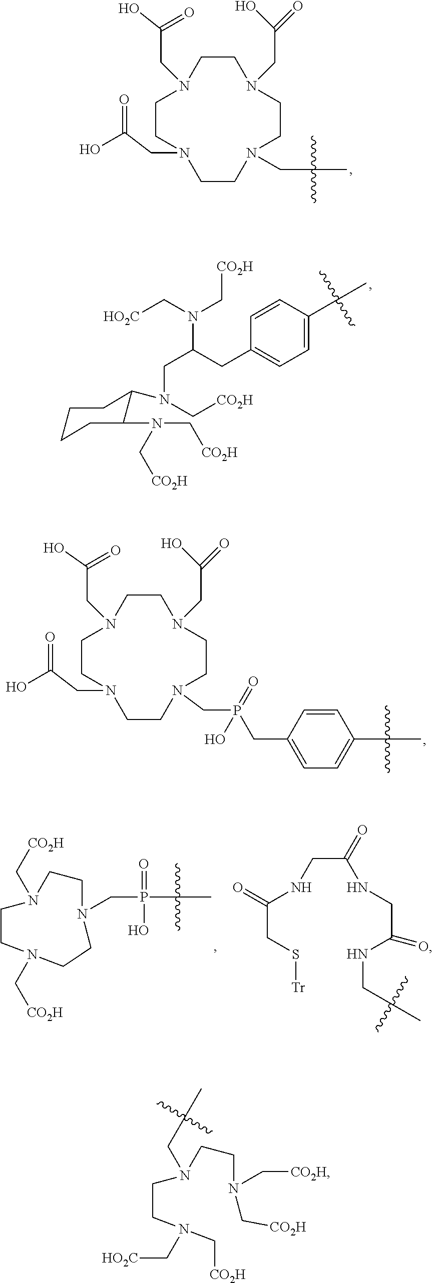

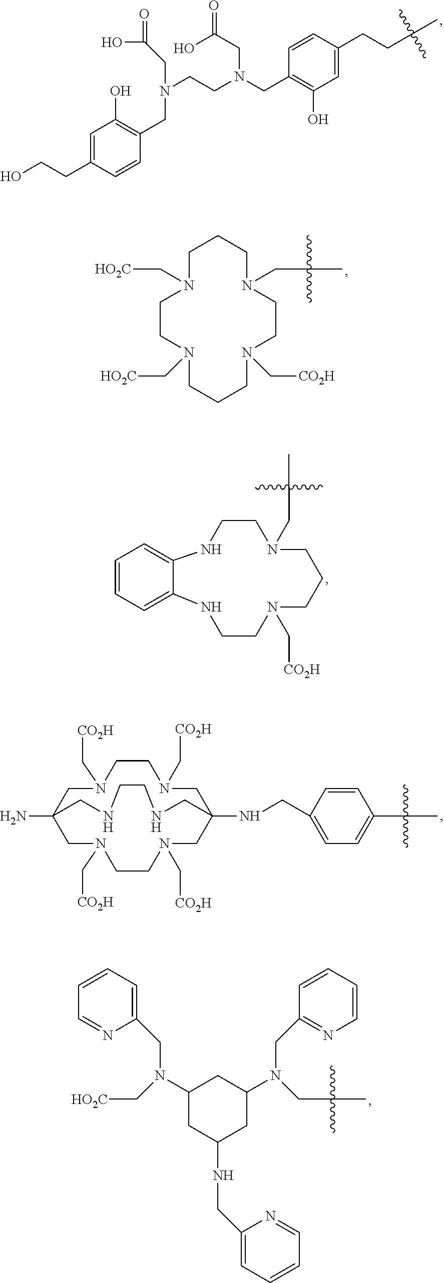 [SMILES: *CCc1ccc(CN(CCN(CC(=O)O)Cc2ccc(CCO)cc2O)CC(=O)O)c(O)c1 ; *CN1CCCN(CC(=O)O)CCN(CC(=O)O)CCCN(CC(=O)O)CC1 ; *CN1CCCN(CC(=O)O)CCNc2ccccc2NCC1 ; *c1ccc(CNC23CNCCNCC(N)(CN(CC(=O)O)CCN(CC(=O)O)C2)CN(CC(=O)O)CCN(CC(=O)O)C3)cc1 ; *CN(Cc1ccccn1)C1CC(NCc2ccccn2)CC(N(CC(=O)O)Cc2ccccn2)C1]

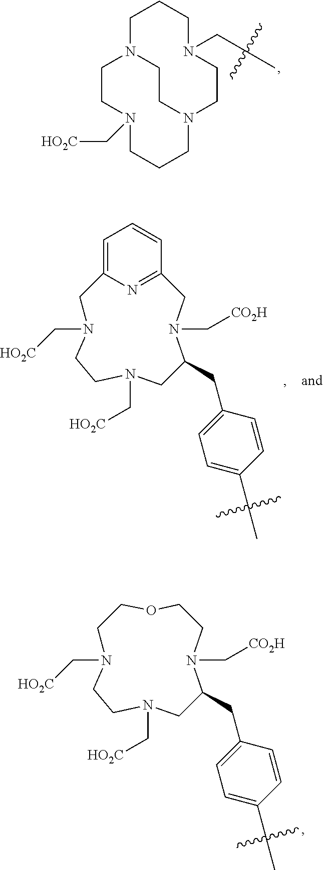 [SMILES: *CN1CCCN2CCN(CCCN(CC(=O)O)CC2)CC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1]

wherein the wiggly line denotes a bond to the remaining part of the molecule, optionally bound via —C(O)NH—, wherein the chelator moieties according to said group optionally chelate a metal.

Embodiment 4. A kit according to any one of the preceding Embodiments, wherein the chelator moiety chelates a metal ion.

Embodiment 5. A kit according to any one of the preceding Embodiments, wherein the chelator moiety chelates an isotope selected from the group consisting of ⁶²Cu, ⁶⁴Cu, ⁶⁶Ga, ⁶⁷Ga, ⁶⁷Cu, ⁶⁸Ga, ⁸⁶Y ⁸⁹Zr, ⁹⁰Y, ^{99m}Tc, ¹¹¹In, ¹⁶⁶Ho, ¹⁷⁷Lu, ¹⁸⁶Re, ¹⁸⁸Re, ²¹¹Bi, ²¹²Bi, ²¹²Pb, ²¹³Bi, ²¹⁴Bi, and ²²⁵Ac.

Embodiment 6. A kit according to any one of the preceding Embodiments, wherein the tetrazine satisfies any one of Formulae (11), (12), (13), (14), (15), (16), (17), or (18):

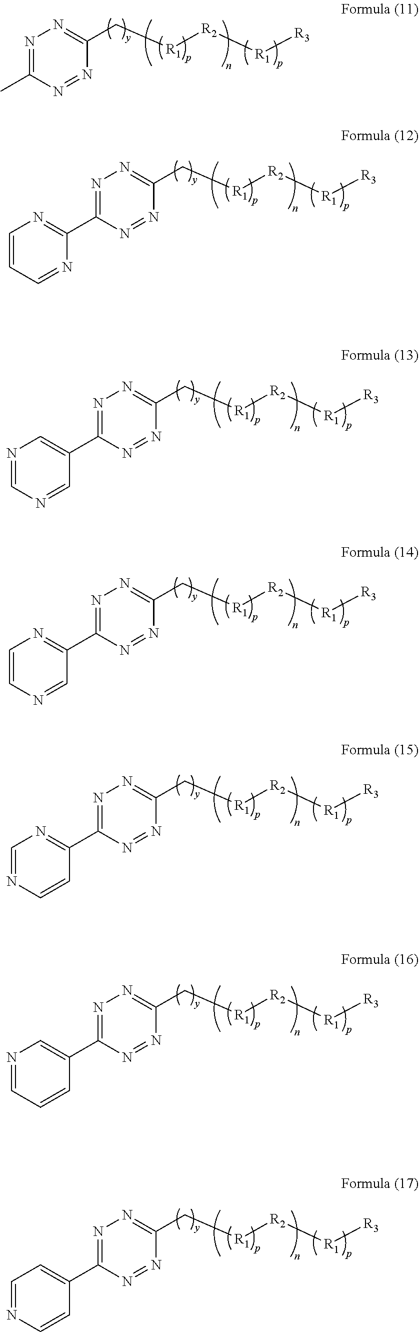

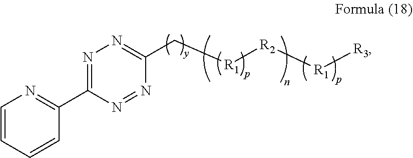

wherein n, p, y, R₁, R₂, and R₃ are as defined in Embodiment 1 for Formulae (1), (2), (3), (4), (5), (6), (7), and (8),
wherein in Formulae (11), (12), (13), (14), (15), (16), (17), and (18) the moiety —(CH₂)_{y}—((R₁)ₚ—R₂)ₙ—(R₁)ₚ)—R₃ has a molecular weight in a range of from 100 Da to 3000 Da,
wherein in Formula (18) y is not 1.

Embodiment 7. A kit according to Embodiment 6, wherein the compounds according to Formulae (11), (12), (13), (14), (15), (16), (17), or (18) have a Log P value of at most 3.0, preferably at most 2.0.

Embodiment 8. A kit according to any one of the preceding Embodiments, wherein the dienophile satisfies Formula (19):

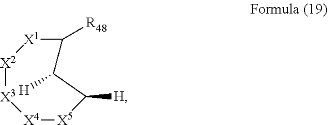

wherein R₄₈ is selected from the group consisting of —OH,
—OC(O)Cl, —OC(O)O—N-succinimidyl, —OC(O)O-4-nitrophenyl, —OC(O)O— tetrafluorophenyl, —OC(O)O-pentafluorophenyl, —OC(O)—C^{A}, —OC(S)—C^{A}, —O-(L^{C}(C^{A})ₛ(C^{A})ₛ((S^{P})ᵢ—C^{B})ⱼ)ᵣ—C^{A}, and —C^{A},
wherein r is an integer in range of from 0 to 2,
wherein each s is independently 0 or 1,
wherein i is an integer in a range of from 0 to 4,
wherein j is 0 or 1,
wherein L^{C} is a self-immolative linker,
wherein C^{A} denotes a Construct A, wherein said Construct A is selected from the group consisting of drugs and masking moieties,
wherein C^{B} denotes a Construct B, wherein said Construct B is selected from the group consisting of masking moieties and targeting agents,
wherein, when C^{B} is a targeting agent or a masking moiety, then C^{A} is a drug,
wherein, when C^{B} is a drug, then C^{A} is a masking moiety wherein, when R₄₈ is —OC(O)—C^{A} or —OC(S)—C^{A}, C^{A} is bound to the —OC(O)— or —OC(S)— of R₄₈ via an atom selected from the group consisting of O, C, S, and N, preferably a secondary or a tertiary N, wherein this atom is part of C^{A},
wherein, when R₄₈ is —O-(L^{C}(C^{A})ₛ(C^{A})ₛ((S^{P})ᵢ—C^{B})ⱼ)ᵣ—C^{A} and r is 0, C^{A} is bound to the —O— moiety of R₄₈ on the allylic position of the trans-cyclooctene ring of Formula (19) via a group selected from the group consisting of —C(O)—, and —C(S)—, wherein this group is part of C^{A},
wherein, when R₄₈ is —O-(L^{C}-(C^{A})ₛ(C^{A})ₛ((S^{P})ᵢ—C^{B})ⱼ)ᵣ—C^{A} and r is 1, L^{C} is bound to the —O— moiety on the allylic position of the trans-cyclooctene ring of Formula (19) via a group selected from the group consisting of —C(Y^{C2})Y^{C1}—, and a carbon atom, preferably an aromatic carbon, wherein this group is part of L^{C},
wherein Y^{C1} is selected from the group consisting of —O—, —S—, and —NR₃₆—,
wherein Y^{C2} is selected from the group consisting of O and S,
wherein, when R₄₈ is —O-(L^{C}(C^{A})ₛ(C^{A})ₛ((S^{P})ᵢ—C^{B})ⱼ)ᵣ—C^{A}, and n is 1, then C^{A} is bound to L^{C} via a moiety selected from the group consisting of —O—, —S—, and —N—, preferably a secondary or a tertiary N, wherein said moiety is part of C^{A},
wherein, when R₄₈ is —C^{A}, then C^{A} is bound to the allylic position of the trans-cyclooctene of Formula (19) via an —O— atom, wherein this atom is part of C^{A},
wherein R₃₆ is selected from the group consisting of hydrogen and C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein X⁵ is —C(R₄₇)₂—,
wherein each X¹, X², X³, X⁴ is independently selected from the group consisting of —C(R₄₇)₂—, —NR₃₇—, —O—, such that at most two of X′, X², X³, X⁴ are not —C(R₄₇)₂—, and with the proviso that no sets consisting of adjacent atoms are present selected from the group consisting of —O—O—, —O—N—, —C(O)—O—, N—N—, and —C(O)—C(O)—,
wherein each R₄₇ is independently selected from the group consisting of hydrogen, —F, —Cl, —Br, —I, —OH, —NH₂, —SO₃_{^{−}}, —PO₃_{^{−}}, —NO₂, —CF₃, —SH, —(S^{P})ᵢ—C^{B}, C₁-C₈ alkyl groups, C₂-C₈ alkenyl groups, C₂-C₈ alkynyl groups, C₆-C₁₂ aryl groups, C₂-C₁₂ heteroaryl groups, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the alkyl groups, alkenyl groups, alkynyl groups, aryl, heteroaryl, cycloalkyl groups, cycloalkenyl groups, alkyl(hetero)aryl groups, (hetero)arylalkyl groups, alkylcycloalkyl groups, cycloalkylalkyl groups, cycloalkyl(hetero)aryl groups and (hetero)arylcycloalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized,
wherein two R₄₇ are optionally comprised in a ring,
wherein two R₄₇ are optionally comprised in a ring so as to form a ring fused to the eight-membered trans-ring,
wherein each R₃₇ is independently selected from the group consisting of hydrogen, —(S^{P})ᵢ—C^{B}, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, GI-Cu alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the R₃₇ groups not being hydrogen are optionally substituted with a moiety selected from the group consisting of —Cl, —F, —Br, —I, —OH, —NH₂, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized
wherein S^{P} is a spacer,
wherein at most one C^{B} is comprised in the structure of Formula (19).

Embodiment 9. A kit according to Embodiment 8, wherein each S^{P} is independently selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₁-C₁₂ alkylcycloalkylene groups, C₄-C₁₂ cycloalkylalkylene groups, wherein for S^{P} the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,

wherein each R′ is independently selected from the group consisting of hydrogen, C₁-C₆ alkylene groups, C₂-C₆ alkenylene groups, C₂-C₆ alkynylene groups, C₆ arylene, C₄-C₅ heteroarylene, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein for R′ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups are optionally substituted with a moiety selected from the group consisting of —Cl, —F, —Br, —I, —OH, —NH₂, ═O, —SH, —SO₃H, —PO₃H, —PO₄H₂, —NO₂, and optionally contain one or more heteroatoms selected from the group consisting of —O—, —S—, —NH—, —P—, and —Si, wherein the N, S, and P atoms are optionally oxidized.

Embodiment 10. A kit according to any one of Embodiments 8 to 9, wherein L^{C} is selected from the group consisting of linkers according to Group I, Group II, and Group III,

wherein linkers according to Group I are

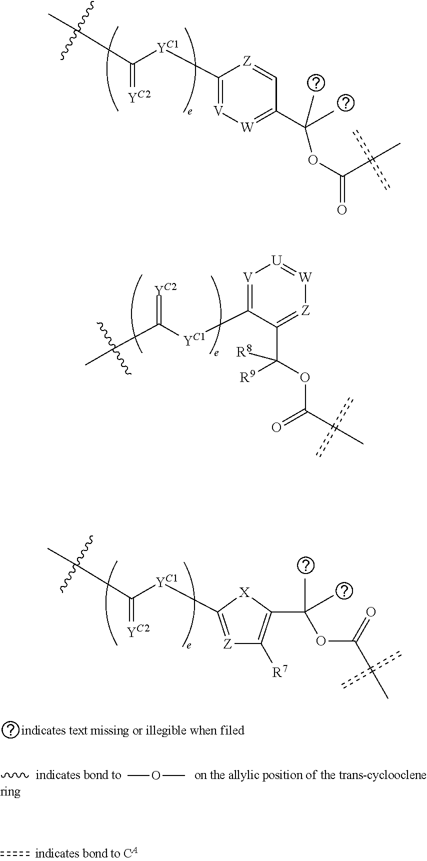

wherein U, V, W, Z are each independently selected from the group consisting of —CR⁷—, and —N—,
wherein e is either 0 or 1,
wherein X is selected from the group consisting of —O—, —S— and —NR⁶—,
wherein each R⁸ and R⁹ are independently selected from the group consisting of hydrogen, C₁-C₄ alkyl groups, C₂-C₄ alkenyl groups, and C₄₋₆ (hetero)aryl groups,
wherein for R⁸ and R⁹ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein for linkers according to Group I C^{A} is linked to L^{C} via a moiety selected from the group consisting of —O—, —N—, —C—, and —S—, preferably from the group consisting of secondary amines and tertiary amines, wherein said moieties are part of C^{A},
wherein the linker according to Group II is

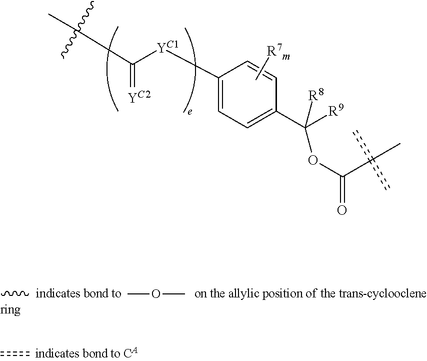

wherein m is an integer between 0 and 2, preferably m is 0,
wherein e is either 0 or 1,
wherein for linkers according to Group II C^{A} is linked to L^{C} via a moiety selected from the group consisting of —O—, —N—, —C—, and —S—, preferably from the group consisting of secondary amines and tertiary amines, wherein said moieties are part of C^{A},
wherein linkers according to Group III are

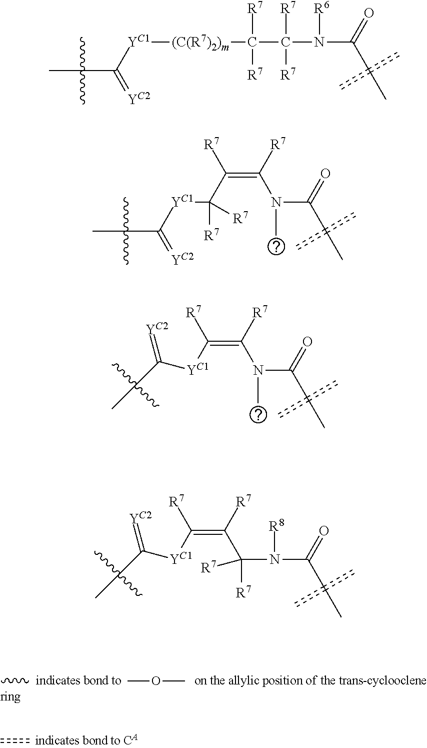

wherein for linkers according to Group III C^{A} is linked to L^{C} via a moiety selected from the group consisting of —O— and —S—, preferably —O— or —S— bound to a C₄₋₆ (hetero)aryl group, wherein said moieties are part of C^{A},
wherein each R⁶ is independently selected from the group consisting of hydrogen, C₁-C₄ alkyl groups, C₂-C₄ alkenyl groups, and C₄₋₆ (hetero)aryl groups,
wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein each R⁷ is independently selected from the group consisting of hydrogen and C₁-C₃ alkyl groups, C₂-C₃ alkenyl groups, and C₄₋₆; (hetero)aryl groups,
wherein for R⁷ the alkyl groups, alkenyl groups, and (hetero)aryl groups are optionally substituted with a moiety selected from the group consisting of —Cl, —F, —Br, —I, —OH, —NH₂, ═O, ═NH, —N(CH₃)₂, —S(O)₂CH₃, and —SH, and are optionally interrupted by at most one heteroatom selected from the group consisting of —O—, —S—, —NH—, —P—, and —Si—, wherein the N, S, and P atoms are optionally oxidized, wherein the N atoms are optionally quaternized,
wherein R⁷ is preferably selected from the group consisting of hydrogen, methyl, —CH₂—CH₂—N(CH₃)₂, and —CH₂—CH₂—S(O)₂—CH₃,
wherein R⁶, R⁷, R⁸, R⁹ comprised in said Group I, II and III, can optionally also be —(S^{P})ᵢ—C^{B},
wherein for all linkers according to Group I and Group II Y^{C1} is selected from the group consisting of —O—, —S—, and —NR⁶—, preferably —NR⁶—,
wherein for all linkers according to Group III, Y^{C1} is —NR⁶—,
wherein for all linkers according to Group I, Group II, and Group III, Y^{C2} is selected from the group consisting of O and S, preferably O,
wherein when n as defined in Embodiment 1 is two, then the L^{C} attached to the —O— at the allylic position of the trans-cyclooctene is selected from the group consisting of linkers according to Group I and Group II, and the L^{C} between the L^{C} attached to the —O— at the allylic position of the trans-cyclooctene and C^{A} is selected from Group III, and that the wiggly line in the structures of Group III then denotes a bond to the L^{C} attached to the —O— at the allylic position of the trans-cyclooctene instead of a bond to the allylic —O— on the trans-cyclooctene ring, and that the double dashed line in the structures of Groups I and II then denotes a bond to the L^{C} between the L^{C} attached to the —O— at the allylic position of the trans-cyclooctene and the C^{A} instead of a bond to C^{A}.

Embodiment 11. A kit according to any one of Embodiments 8 to 10, wherein L^{C} is selected from the group consisting of linkers according to Group IV, Group V, Group VI, and Group VII, wherein linkers according to Group IV are

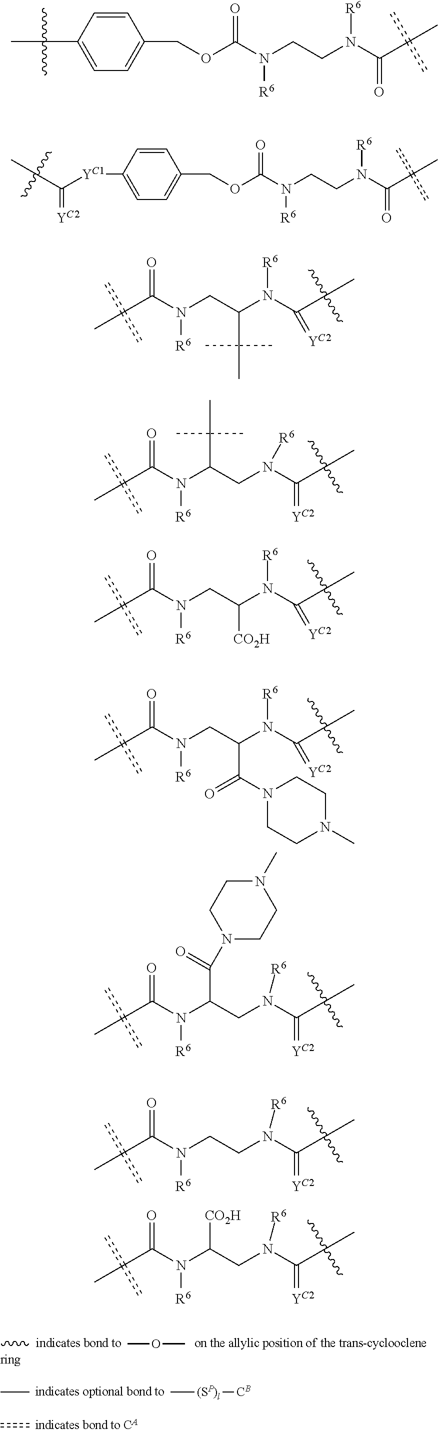

wherein C^{A} is linked to L^{C} via a moiety selected from the group consisting of —O— and —S—, preferably from the group consisting of —O—C₅₋₈-arylene- and —S—C₅₋₈-arylene-, wherein said moieties are part of C^{A},
wherein linkers according to Group V are

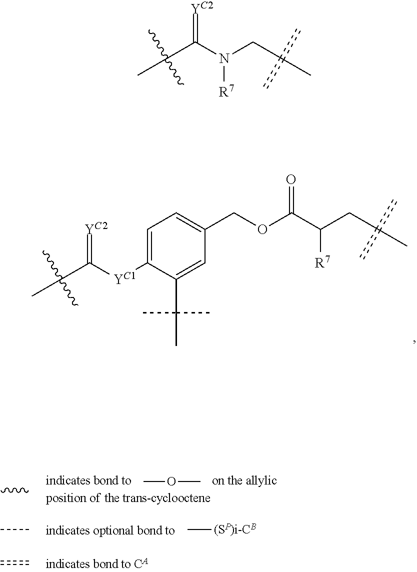

wherein C^{A} is linked to L^{C} via a moiety selected from the group consisting of —O— and —S—, wherein said moieties are part of C^{A},
wherein linkers according to Group III are

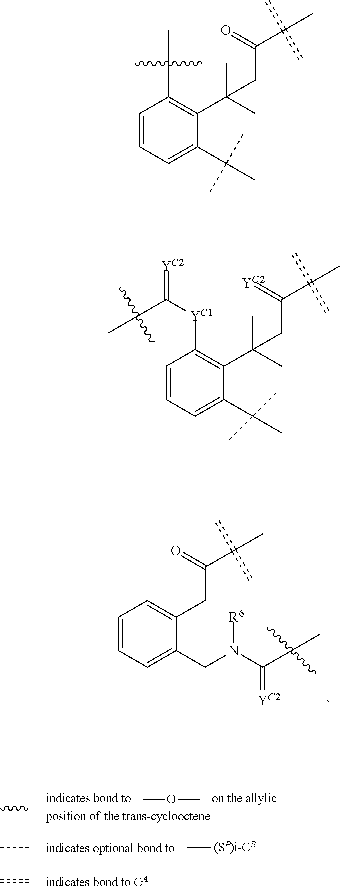

wherein C^{A} is linked to L^{C} via a moiety selected from the group consisting of —O—, —N—, and —S—, preferably a secondary or a tertiary amine, wherein said moieties are part of C^{A},
wherein linkers according to Group VI are

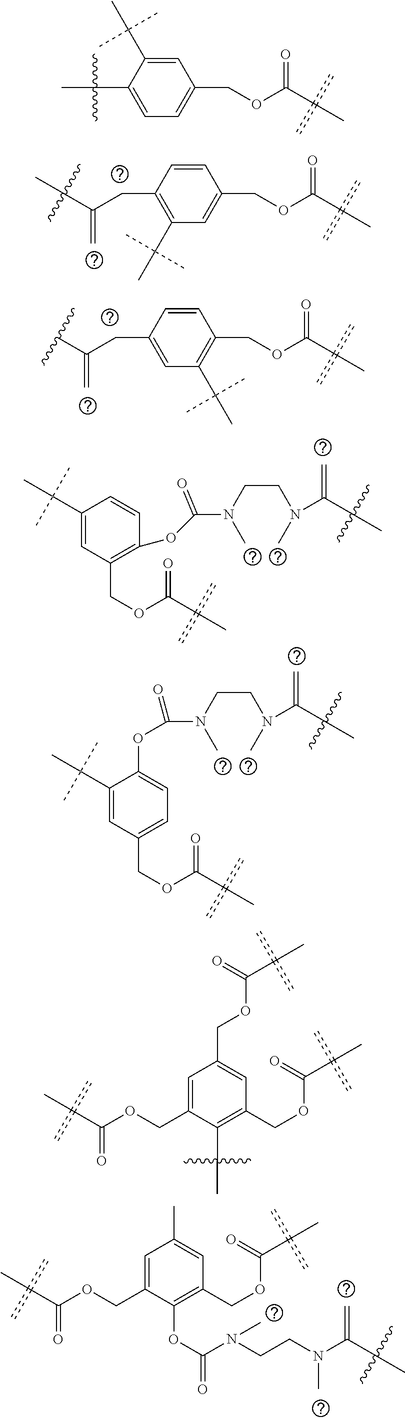

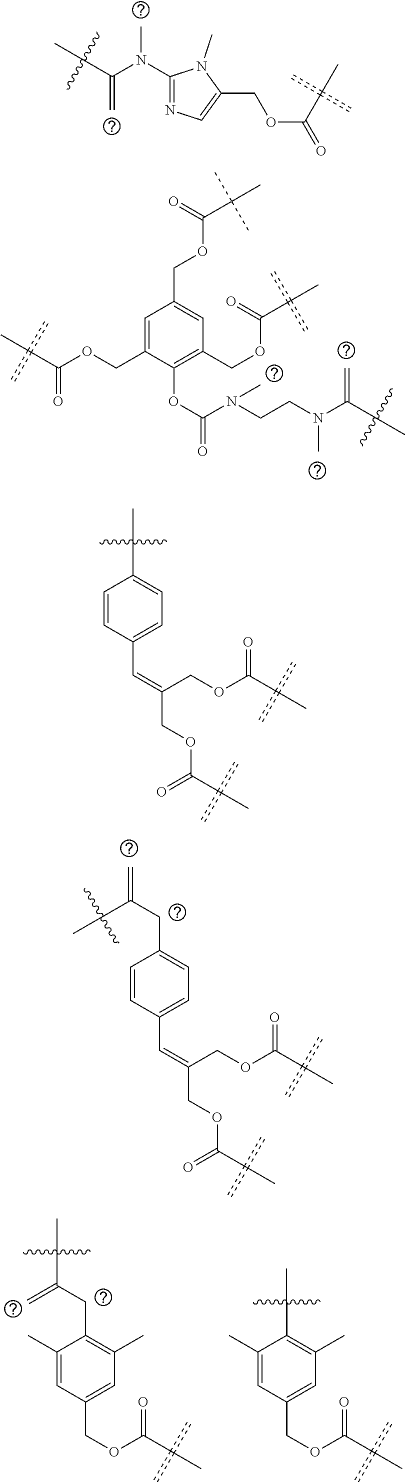

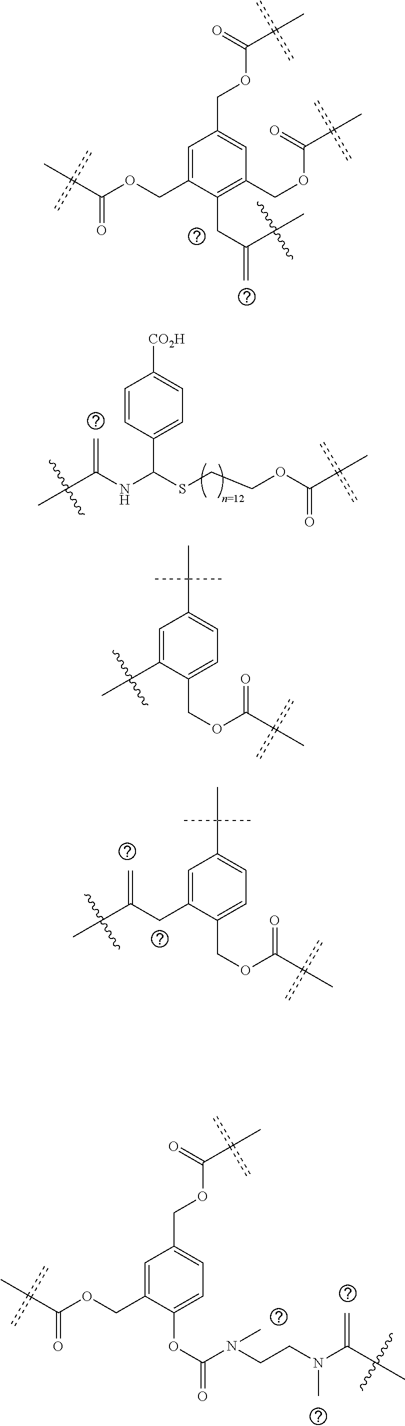

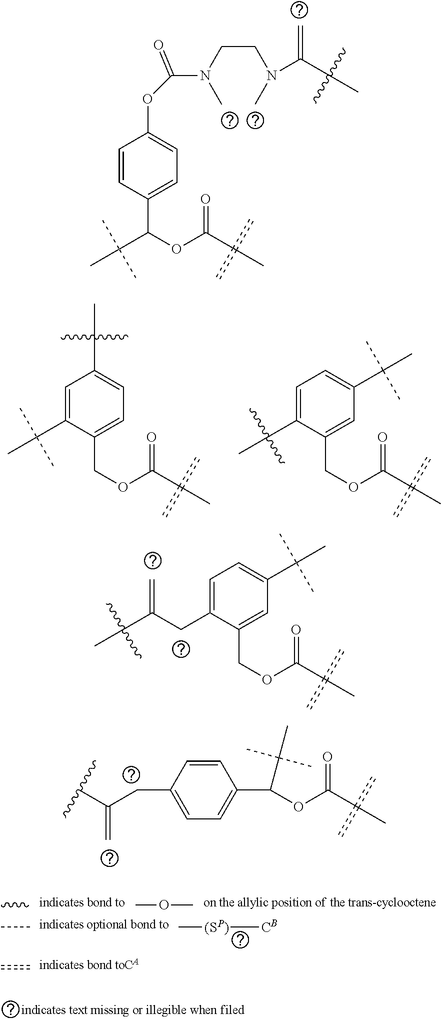

wherein C^{A} is linked to L^{C} via a moiety selected from the group consisting of —O—, —N—, and —S—, preferably from the group consisting of secondary amines and tertiary amines, wherein said moieties are part of C^{A}, wherein when multiple double dashed lines are shown within one L^{C}, each C^{A} moiety is independently selected,
wherein for all linkers according to Group IV, Group V, Group VI, and Group VII, Y^{C1} is selected from the group consisting of —O—, —S—, and —NR⁶—,
wherein C^{B} is selected from the group consisting of drugs, targeting agents, and masking moieties,
wherein R⁶, R⁷, i and j are as defined in Embodiment 10,
wherein when i is 0, then C^{B} is linked to the remaining part of L^{C} via a moiety selected from the group consisting of —O—, —C(R⁶)₂—, —NR⁶—, and —S—, wherein said moieties are part of C^{B},
wherein when i is at least 1, then C^{B} is linked to S^{P} via a moiety selected from the group consisting of —O—, —C(R⁶)₂—, —NR⁶—, and —S—, wherein said moieties are part of C^{B}, and S^{P} is linked to the remaining part of L^{C} via a moiety selected from the group consisting of —O—, —C(R⁶)₂—, —NR⁶—, and —S—, wherein said moieties are part of S^{P}.

Embodiment 12. A kit according to any one of the Embodiments 8 to 11, wherein all X in Formula (19) are —C(R₄₇)₂—.

Embodiment 13. A kit according to any one of the Embodiments 8 to 12, wherein at most three R₄₇ in Formula (19) are not H.

Embodiment 14. A kit according to any one of the Embodiments 8 to 13, wherein R₄₈ is in the axial position.

Embodiment 15. A kit according to any one of the preceding Embodiments, wherein the dienophile satisfies Formula (20)

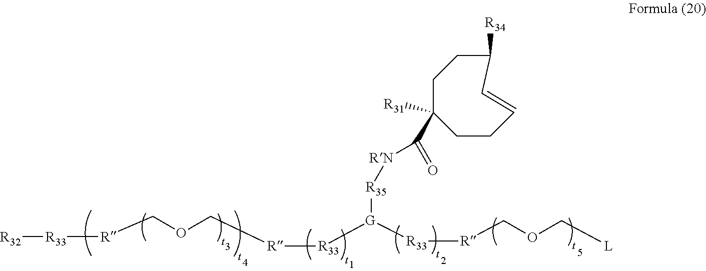

wherein t₁ is 0 or 1,
wherein t₂ is 0 or 1,
wherein t₃ is an integer in a range of from 1 to 12,
wherein t₄ is 0 or 1,
wherein t₅ is an integer in a range of from 6 to 48,
wherein L is selected from the group consisting of —CH₂—OCH₃, —CH₂—OH, —CH₂—C(O)OH, —C(O)OH, wherein L is preferably —CH₂—OCH₃,
wherein when at least one of t₁ or t₂ is 0, then G is selected from the group consisting of CR′, C₅-C₆ arenetriyl, C₄-C₅ heteroarenetriyl, C₃-C₆ cycloalkanetriyl, and C₄-C₆ cycloalkenetriyl, wherein when both t₁ and t₂ are 1, then G is selected from the group consisting of CR′, N, C₅-C₆ arenetriyl, C₄-C₅ heteroarenetriyl, C₃-C₆ cycloalkanetriyl, and C₄-C₆ cycloalkenetriyl,
wherein for G, the arenetriyl, heteroarenetriyl, cycloalkanetriyl, and cycloalkenetriyl are optionally further substituted with groups selected from the group consisting of —Cl, —F, —Br, —I, —OR′, —N(R′)₂, —SR′, —SO₃H, —PO₃H, —PO₄H₂, —NO₂, —CF₃ and —R₃₁, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein R₃₁ is selected from the group consisting of hydrogen, C₁-C₆ alkyl groups, C₆ aryl groups, C₄-C₅ heteroaryl groups, C₃-C₆ cycloalkyl groups, C₅-C₁₂ alkyl(hetero)aryl groups, C₅-C₁₂ (hetero)arylalkyl groups, C₄-C₁₂ alkylcycloalkyl groups, —N(R′)₂, —OR′, —SR′, —SO₃H, —C(O)OR′, and Si(R′)₃,
wherein for R₃₁ the alkyl groups, (hetero)aryl groups, cycloalkyl groups, alkyl(hetero)aryl groups, (hetero)arylalkyl groups, alkylcycloalkyl groups are optionally substituted with a moiety selected from the group consisting of —Cl, —F, —Br, —I, NO₂, SO₃H, PO₃H, —PO₄H₂, —OR′, —N(R′)₂, —CF₃, ═O, ═NR′, —SR′, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein R₃₂ is selected from the group consisting of N-maleimidyl groups, halogenated N-alkylamido groups, sulfonyloxy N-alkylamido groups, vinyl sulfone groups, activated carboxylic acids, benzenesulfonyl halides, ester groups, carbonate groups, sulfonyl halide groups, thiol groups or derivatives thereof, C₂₋₆ alkenyl groups, C₂₋₆ alkynyl groups, C₇₋₁₈ cycloalkynyl groups, C₅₋₁₈ heterocycloalkynyl groups, bicyclo[6.1.0]non-4-yn-9-yl] groups, C4.12 cycloalkenyl groups, azido groups, phosphine groups, nitrile oxide groups, nitrone groups, nitrile imine groups, isonitrile groups, diazo groups, ketone groups, (O-alkyl)hydroxylamino groups, hydrazine groups, halogenated N-maleimidyl groups, aryloxymaleimides, dithiophenolmaleimides, bromo- and dibromopyridazinediones, 2,5-dibromohexanediamide groups, alkynone groups, 3-arylpropionitrile groups, 1,1-bis(sulfonylmethyl)-methylcarbonyl groups or elimination derivatives thereof, carbonyl halide groups, allenamide groups, 1,2-quinone groups, isothiocyanate groups, aldehyde groups, triazine groups, squaric acids, 2-imino-2-methoxyethyl groups, (oxa)norbornene groups, (imino)sydnones, methylsulfonyl phenyloxadiazole groups, aminooxy groups, 2-amino benzamidoxime groups, groups reactive in the Pictet Spengler ligation and hydrazino-Pictet Spengler (HIPS) ligation,
wherein each individual R₃₃ is selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein each individual R₃₅ is selected from the group consisting of C₁-C₈ alkylene groups, C₂-C₈ alkenylene groups, C₂-C₈ alkynylene groups, C₆ arylene groups, C₄-C₅ heteroarylene groups, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₁₁-C₁₂ cycloalkylalkylene groups,
wherein for R₃₃ and R₃₅ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein each R′ is independently selected from the group consisting of hydrogen, C₁-C₆ alkylene groups, C₂-C₆ alkenylene groups, C₂-C₆ alkynylene groups, C₆ arylene, C₄-C₅ heteroarylene, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₁-C₁₂ cycloalkylalkylene groups,
wherein for R′ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups are optionally substituted with a moiety selected from the group consisting of —Cl, —F, —Br, —I, —OH, —NH₂, ═O, —SH, —SO₃H, —PO₃H, —PO₄H₂, —NO₂, and optionally contain one or more heteroatoms selected from the group consisting of —O—, —S—, —NH—, —P—, and —Si, wherein the N, S, and P atoms are optionally oxidized,
wherein each R″ is independently selected from the group consisting of

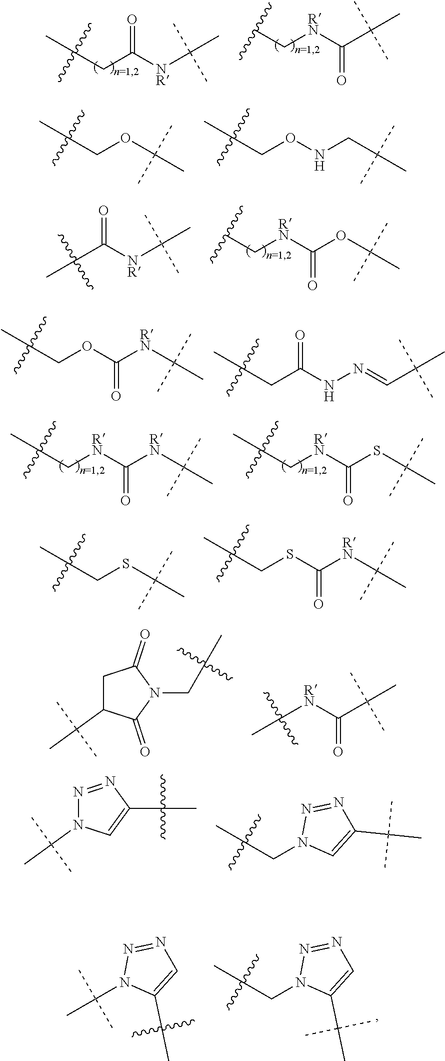

wherein the wiggly line depicts a bond to an ethylene glycol group or optionally to the R₃₃ adjacent to R₃₂ when t₄ is 0, and the dashed line depicts a bond to R₃₃ or G,
wherein R₃₄ is selected from the group consisting of —OH, —OC(O)Cl, —OC(O)O—N-succinimidyl, —OC(O)O-4-nitrophenyl, —OC(O)O-tetrafluorophenyl, —OC(O)O-pentafluorophenyl, —OC(O)—C^{A}, —OC(S)—C^{A}, —O-(L^{C}(C^{A})ₛ(C^{A})ₛ)ᵣ—C^{A}, and —C^{A},
wherein r is an integer in range of from 0 to 2,
wherein each s is independently 0 or 1,
wherein, when R₃₄ is —OC(O)—C^{A} or —OC(S)—C^{A}, C^{A} is bound to the —OC(O)— or —OC(S)— of R₃₁ via an atom selected from the group consisting of O, S, and N, preferably a secondary or a tertiary N, wherein this atom is part of C^{A},
wherein, when R₃₄ is —O-(L^{C}(C^{A})ₛ(C^{A})ₛ)ᵣ—C^{A} and n is 0, C^{A} is bound to the —O-moiety of R₃₄ on the allylic position of the trans-cyclooctene ring of Formula (20) via a group selected from the group consisting of —C(O)—, and —C(S)—, wherein this group is part of C^{A},
wherein, when R₃₄ is —O-(L^{C}(C^{A})ₛ(C^{A})ₛ)ᵣ—C_{A} and n is 1, L^{C} is bound to the —O-moiety on the allylic position of the trans-cyclooctene ring of Formula (20) via a group selected from the group consisting of —C(Y^{C2})Y^{C1}—, and a carbon atom, preferably an aromatic carbon, wherein this group is part of L^{C},
wherein Y^{C1} is selected from the group consisting of —O—, —S—, and —NR₃₆—,
wherein Y^{C2} is selected from the group consisting of O and S,
wherein, when R₃₄ is —O-(L^{C}(C^{A})ₛ(C^{A})ₛ)ᵣ—C^{A}, and n is 1, then C^{A} is bound to L^{C} via a moiety selected from the group consisting of —O—, —S—, and —N—, preferably a secondary or a tertiary N,
wherein said moiety is part of C^{A},
wherein, when R₃₁ is —C^{A}, then C^{A} is bound to the allylic position of the trans-cyclooctene of Formula (20) via an —O— atom, wherein this atom is part of C^{A},
wherein R₃₆ is selected from the group consisting of hydrogen and C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized, and pharmaceutically accepted salts thereof.

Embodiment 16. A kit according to Embodiment 15, wherein R₃₂ is an N-maleimidyl group linked to the remaining part of the compound according to Formula (20) via the amine of the N-maleimidyl group.

Embodiment 17. A kit according to anyone of the preceding Embodiments, wherein said kit comprises a compound selected from the group consisting of proteins, antibodies, peptoids and peptides, modified with at least one compound according to any one of the Embodiments 15 to 16.

Embodiment 18. A kit according to Embodiment 17, wherein the compound selected from the group consisting of proteins, antibodies, peptoids and peptides comprises at least one moiety M selected from the group consisting of —OH, —NHR′, —CO₂H, —SH, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrile, (oxa)norbornene before modification with a compound according to Embodiment 15, wherein R′ is as defined in Embodiment 15, wherein the compound selected from the group consisting of proteins, peptoids antibodies, and peptides satisfies Formula (21) after modification with at least one compound according to any one of Embodiments 15 to 16:

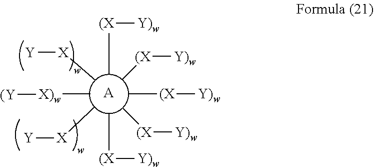

wherein moiety A is selected from the group consisting of proteins, antibodies, peptoids and peptides,
wherein each individual w is 0 or 1, wherein at least one w is 1,
wherein each moiety Y is independently selected from moieties according to Formula (22), wherein at least one moiety Y satisfies said Formula (22):

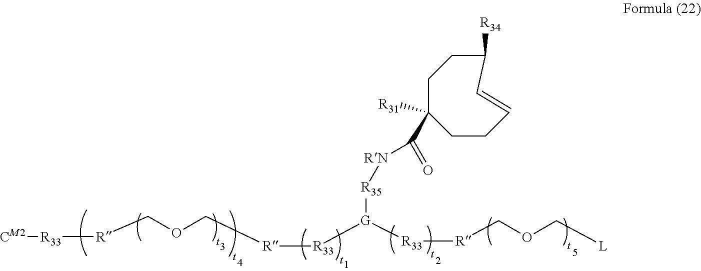

wherein n, t₁, t₂, x, y, z, G, L, R₃₁, R₃, R⁴, R⁵, R′, and R″ are as defined for Formula (20),
wherein moiety X is part of moiety A and was a moiety M before modification of moiety A,
wherein moiety C^{M2} is part of moiety Y and was a moiety R₃₂ as defined in any one of the previous Embodiments for compounds according to Formula (20) before modification of moiety A,
wherein when moiety X is —S—, then C^{M2} is selected from the group consisting of

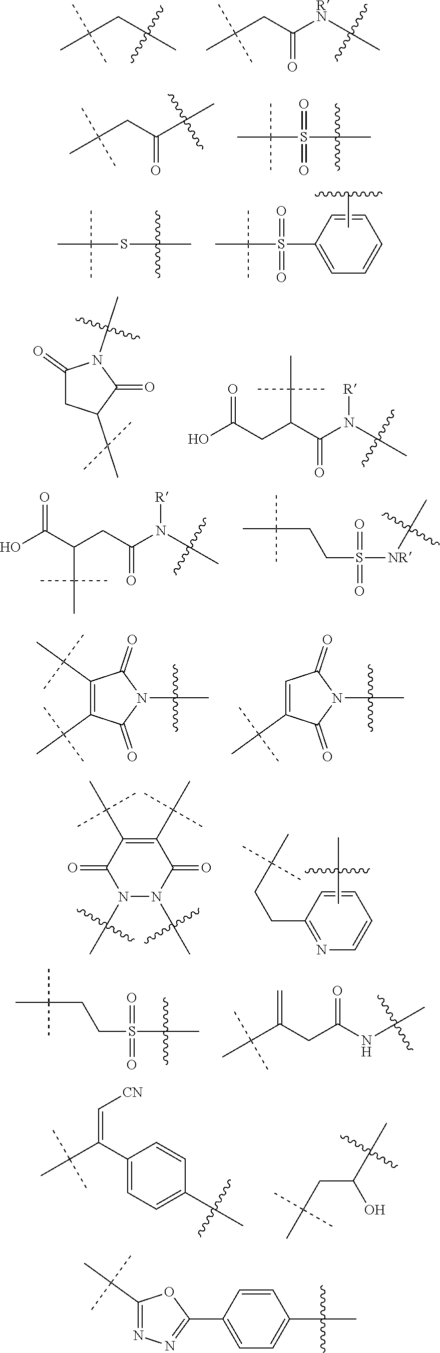

wherein the wiggly line denotes a bond to the remaining part of moiety Y, and wherein the dotted line denotes a bond to moiety X,
wherein when moiety X is —NR′—, then C^{M2} is selected from the group consisting of

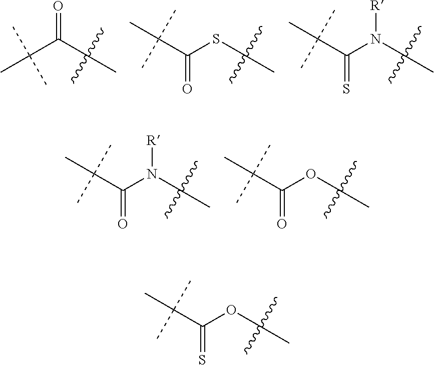

wherein the wiggly line denotes a bond to the remaining part of moiety Y, and wherein the dotted line denotes a bond to moiety X,
wherein when moiety X is —C— derived from a moiety M that was —C(O)R′ or —C(O)R′—, then C^{M2} is selected from the group consisting of

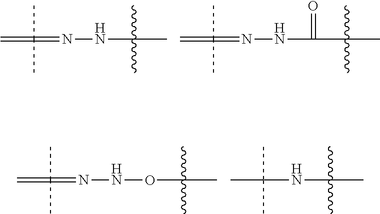

wherein the wiggly line denotes a bond to the remaining part of moiety Y, and wherein the dotted line denotes a bond to moiety X,
wherein when moiety X is —C(O)— derived from a moiety M that was —C(O)OH, then C^{M2} is selected from the group consisting of

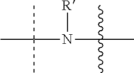

wherein the wiggly line denotes a bond to the remaining part of moiety Y, and wherein the dotted line denotes a bond to moiety X,
wherein when moiety X is —O—, then C^{M2} is selected from the group consisting of

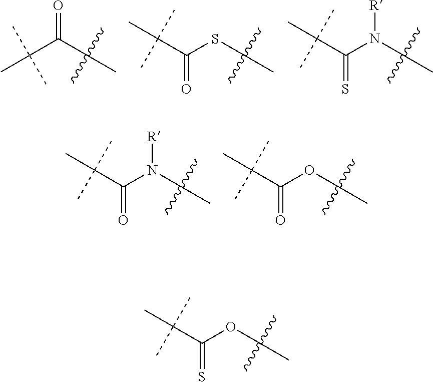

wherein the wiggly line denotes a bond to the remaining part of moiety Y, and
wherein the dotted line denotes a bond to moiety X,
wherein when moiety X is derived from a moiety M that was —N₃ and that was reacted with an R₃₂ that comprised an alkyne group, then X and CRT together form a moiety C^{X}, wherein C^{X} comprises a triazole ring.

Embodiment 19. A kit according to Embodiment 18, wherein each C^{X} is independently selected from the group consisting of

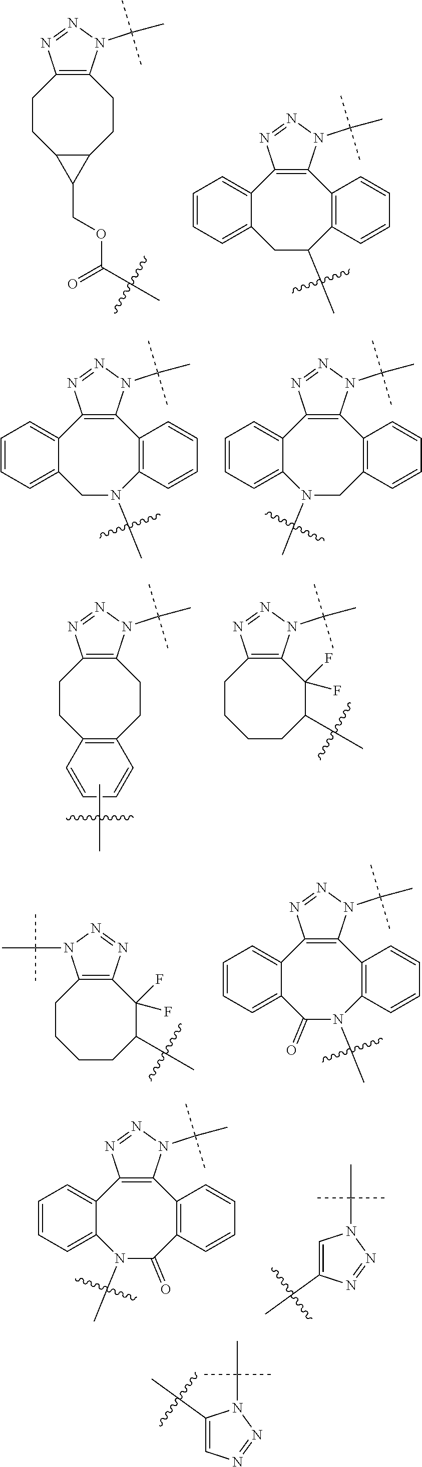

wherein the wiggly line denotes a bond to the remaining part of moiety Y, and
wherein the dotted line denotes a bond to moiety X.

Embodiment 20. A kit according to any one of the preceding Embodiments for use in the treatment of patients.

### Examples

### Example 1: Materials and Methods and General Synthetic Procedures

### Materials and Methods

All reagents, chemicals, materials and solvents were obtained from commercial sources and were used as received, including nitrile starting compounds that not have been described. All solvents were of AR quality. Moisture or oxygen-sensitive reactions were performed under an Ar atmosphere. 37-Amino-5,8,11,14,17,20,23,26,29,32,35-undecaoxa-2-azaheptatriacontanoic acid t-butyl ester, 5,8,11,14,17,20,23,26-octaoxa-2-azanonacosanedioic acid 1-t-butyl ester, 4,7,10,13,16,19,22-heptaoxapentacosanedioic acid and 3,6,9,12,15,18,21,24,27,30, 33-undecaoxatetratriacontanoic acid were obtained from PurePEG. In the synthetic procedures, equivalents (eq) are molar equivalents. Concentrations of reactants used in the synthetic procedures generally range from about 0.05 to about 3 M, and are typically and mostly in between 0.1 M and 1.0 M. Analytical thin layer chromatography was performed on Kieselgel F-254 precoated silica plates. Column chromatography was carried out on Screening Devices B.V. silica gel (flash: 40-63 μm mesh and normal: 60-200 μm mesh). ¹H-NMR, ¹³C-NMR and ¹⁹F-NMR spectra were recorded on a Bruker Avance III HD (400 MHz for ¹H-NMR, 100 MHz for ¹³C-NMR and 376 MHz for ¹⁹F-NMR) spectrometer at 298 K. Chemical shifts are reported in ppm downfield from TMS at room temperature. Abbreviations used for splitting patterns are s=singlet, d=doublet, t=triplet, q=quartet, qn=quintet, m=multiplet and br=broad. HPLC-PDA/MS was performed using a Shimadzu LC-10 AD VP series HPLC coupled to a diode array detector (Finnigan Surveyor PDA Plus detector, Thermo Electron Corporation) and an Ion-Trap (LCQ Fleet, Thermo Scientific). HPLC-analyses were performed using a Alltech Alltima HP C₁₈ 3μ column using an injection volume of 1-4 μL, a flow rate of 0.2 mL min⁻¹ and typically a gradient (5% to 100% in 10 min, held at 100% for a further 3 min) of MeCN in H₂O (both containing 0.1% formic acid) at 298 K. Preparative RP-HPLC (MeCN/H₂O with 0.1% formic acid) was performed using a Shimadzu SCL-10A VP coupled to two Shimadzu LC-8A pumps and a Shimadzu SPD-10AV VP UV-vis detector on a Phenomenex Gemini 5μ C₁₈ 110A column. Preparative RP-MPLC (MeCN/H₂O with 0.1% formic acid) was performed on a Biotage column machine using a 12 g Biotage SNAP KP-C18-HS cartridge and a flow rate of 10 mL min⁻¹.

TCO-containing ADCs used in the examples include anti-TAG72 mAb conjugate CC49-TCO-doxorubicin (DAR ca 3), the anti-TAG72 diabody conjugate AVP458-TCO-MMAE (DAR=4), and the anti-PSMA diabody conjugate AVP06-TCO-MMAE (DAR=4), and the enzymatically cleavable control ADC (AVP458-vc-MMAE, vc-ADC) and their synthesis and evaluation have been reported in respectively Rossin et al., Bioconjug. Chem., 2016, 27, 1697-1706, and Rossin et al., Nature Communications 2018, 9, 1484.

### General Procedure A—Tetrazine (TZ) Synthesis

The nitrile (or combination of two different nitriles) and zinc triflate (0.05 eq to the total nitrile content) were combined. When this did not yield a clear solution this was achieved by shortly heating the mixture at 60° C. or by the addition of a minimum amount of EtOH. When a clear solution was obtained hydrazine monohydrate (2 eq to the total nitrile content) was added at once and the mixture was stirred at 60° C. for typically 16 h, after which the volatiles were removed in vacuo.

A1. Oxidation of dihydrotetrazine precursor ([2H]-TZ) having NHBoc functionality: The crude mixture containing [2H]-TZ was divided between CHCl₃ and H₂O and the aqueous layer was extracted with CHCl₃ (3×). The organic layer was dried with Na₂SO₄, filtrated and the volatiles were removed in vacuo. The crude [2H]-TZ was dissolved in CH₂Cl₂ and PhI(OAc)₂ (1.5 eq) was added. The mixture was stirred at room temperature until HPLC-PDA/MS indicated full conversion of [2H]-TZ to TZ (typically 2 to 4 h).

A2. Oxidation of [2H]-TZ lacking NHBoc functionality: The crude mixture containing [2H]-TZ was re-dissolved in THF/AcOH (1:1) and this solution was cooled on an ice-bath. NaNO₂ (5 eq to the total nitrile content) in H₂O (5 to 10 mL per gram NaNO₂) was added dropwise (CAUTION: toxic fumes!). After stirring at room temperature for 10 min, H₂O was added and the solution was extracted with CHCl₃ until an aqueous layer was obtained that lacked the typical TZ pink (sometimes red or purple) coloration. The organic layer was dried with Na₂SO₄, filtrated and the volatiles were removed in vacuo. Traces of AcOH were removed by flushing with CHCl₃, or by performing an additional sat. NaHCO₃ wash.

A3. Alternative oxidation of [2H]-TZ lacking NHBoc functionality: To the crude mixture containing [2H]-TZ was added NaNO₂ (5 eq to the total nitrile content) in H₂O (5 to 10 mL per gram NaNO₂). On an ice-bath, 1 M HCl was added dropwise (CAUTION: toxic fumes!) until pH=3. H₂O was added and the solution was extracted with CHCl₃ until an aqueous layer was obtained that lacked the typical TZ pink (sometimes red or purple) coloration. The organic layer was dried with Na₂SO₄, filtrated and the volatiles were removed in vacuo.

### General Procedure B—N-t-Boc Deprotection

tBoc-protected TZ was dissolved in CHCl₃/TFA (1:1) and the mixture was stirred at room temperature for 30 min to 1 h. After removal of the volatiles in vacuo the product was flushed with CHCl₃ (3×).

### General Procedure C—Coupling of TZ-Amine (TFA-Salt) to PEG-Acid

TZ amine (TFA-salt), PEG-acid and PyBOP (1.1 eq) were combined in CH₂Cl₂. Upon dropwise addition of N,N-diisopropylethylamine (3 eq) the solution cleared and was further stirred at room temperature until HPLC-PDA/MS indicated full conversion (typically 1 h). CHCl₃ was added and the organic layer was sequentially washed with 0.1 M HCl (2×), sat. NaHCO₃ and brine, dried with Na₂SO₄, filtrated and the filtrate was concentrated in vacuo.

### General Procedure D—Coupling of Tz-Amine to Glutaric Acid

A solution of TZ-amine and N,N-diisopropylethylamine (4 eq) in CH₂Cl₂ was added to solid glutaric anhydride (1 eq). The solution was stirred at room temperature for 30 min and the solvent was removed in vacuo.

### General Procedure E—Coupling of Tz-Glut-COOH to Mono-Hoc-Protected PEG Diamine

TZ-glut-COOH, mono-boc-protected PEG diamine (1 eq) and N,N-diisopropylethylamine (3 eq) were combined in DMF. PyBOP (1 eq) was added as a solid and the solution was stirred at room temperature until HPLC-PDA/MS indicated full conversion (typically 1 h). DMF was removed in vacuo at 40° C. using an oil pump. CHCl₃ was added and the organic layer was sequentially washed with 0.1 M HCl, sat. NaHCO₃ and H₂O, dried with Na₂SO₄, filtrated and the filtrate was concentrated in vacuo.

### General Procedure F—Coupling of TZ-PEG-Amine (TFA-Salt) to DOTA

TZ-PEG-amine (TFA-salt) was dissolved in DMF with N,N-diisopropylethylamine (10 eq). As a solid, 1,4,7,10-tetraazacyclododecane-1,4,7,10-tetraacetic acid p-nitrophenyl ester (Mier et al., Bioconjugate Chem. 2005, 16, 237-240) (1.1 eq) was added and the solution was stirred at room temperature until HPLC-PDA/MS indicated full conversion (typically 30 min). Precipitation was performed by directly adding the reaction mixture to a stirring solution of diethyl ether and followed by centrifugation and decantation. The solid was washed once with diethyl ether after which centrifugation and decantation were repeated. The resulting solid was dried in vacuo.

### Example 2: Synthesis of 3,6-Bisalkyl TZ Precursors and Activators

The synthesis of 3,6-dimethyl-1,2,4,5-tetrazine (2.1) was reported in Versteegen et al., Angew. Chem. Int. Ed., 2013, 52, 14112-14116.

The syntheses of 3,6-dimethyl-1,2,4,5-tetrazine functional dextran (2.2) and 5-(((6-methyl-1,2,4,5-tetrazin-3-yl)methyl)amino)-5-oxopentanoic acid (2.5) were reported in Rossin et al., Bioconjug. Chem., 2016, 27, 1697-1706. The synthesis of 3-ethyleneamine-6-(2,6-pyrimidyl)-1,2,4,5-tetrazine was reported in Sarris et al., Chem. Eur. J. 2018, 24, 18075-18081.

### Synthesis (2.9), (2.10) and (2.11).

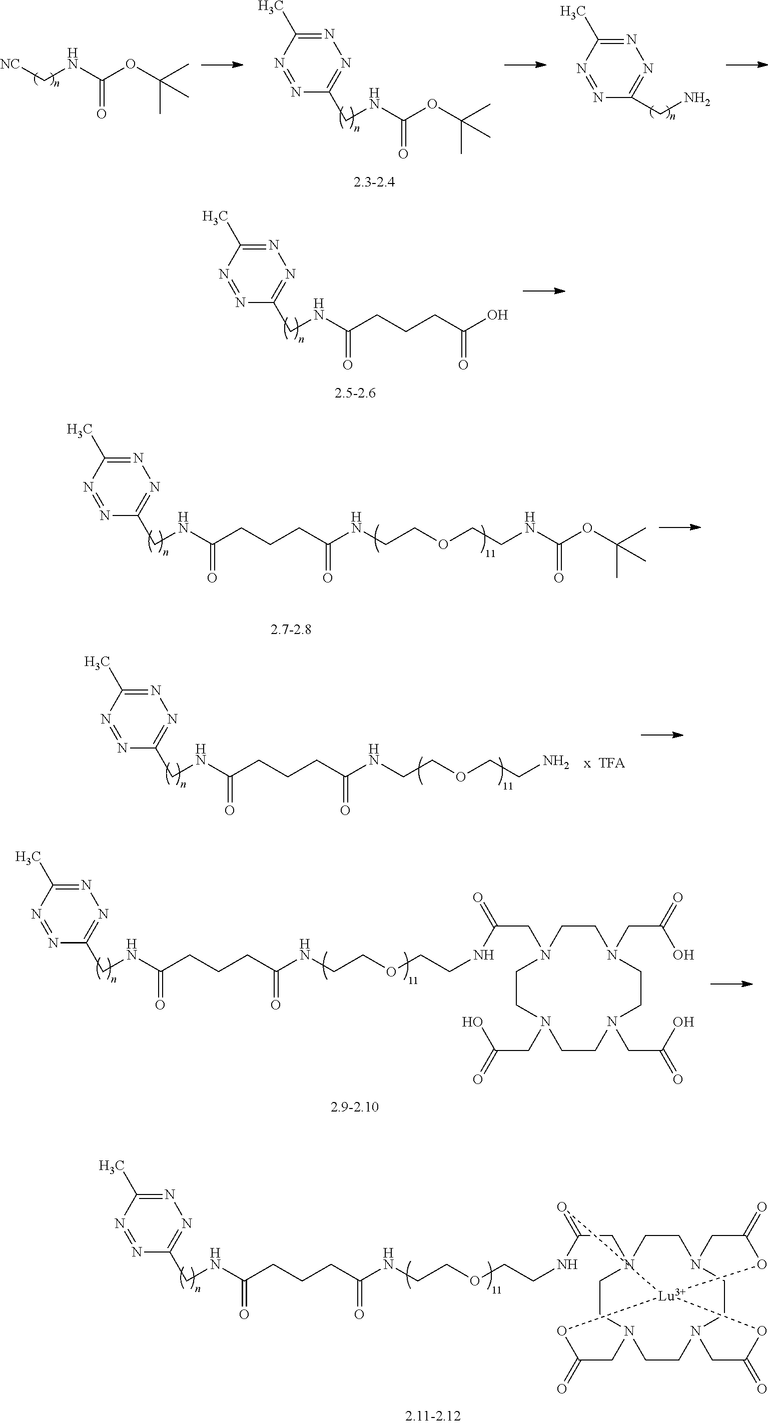

| Code | n |
|---|---|
| 2.3, 2.5, 2.7, | 1 |
| 2.9, 2.11 | |
| 2.4, 2.6, 2.8, | 3 |
| 2.10, 2.12 | |

Compound 2.4 has been prepared according to general procedure A.

This compound was prepared from 3-cyano-N-Boc-propylamine (Houssin et al., Synthesis, 1988, 1988, 259-261) and acetonitrile that were reacted in a 1:5 molar ratio. Oxidation was performed according to general procedure A2. Column chromatography (flash SiO₂) using 1:3 ethyl acetate/heptane and recrystallization from diisopropyl ether at −20° C. yielded pure 2.4. ¹H NMR (CDCl₃): δ=4.69 (br s, 1H), 3.35 (t, J=7.6 Hz, 2H), 3.28 (q, J=6.5 Hz, 2H), 2.15 (m, 2H), 1.44 (s, 9H) ppm. ¹³C NMR (CDCl₃): δ=169.50, 167.45, 155.88, 79.36, 39.71, 31.96, 28.39, 28.34, 21.10 ppm. HPLC-MS/PDA (5% to 100% in 10 min): tᵣ=5.33 min (m/z=+137.08, +154.08, +198.00, +253.92 [M+H]⁺; calcd 254.16 for C₁₁H₂₀N₅O₂: λₘₐₓ=277, 524 nm).

Compound 2.6 has been prepared according to general procedure D.

Compound 2.4 was deprotected and the reaction intermediate was reacted with glutaric anhydride in a 1:1 molar ratio. After trituration with cold diethyl ether, compound 2.6 was obtained as a pink powder. ¹H NMR (CDCl₃): δ=6.11 (br s, 1H), 3.41 (q, J=6.5 Hz, 2H), 3.35 (t, J=7.6 Hz, 2H), 3.05 (s, 3H), 2.43 (t, J=7.0 Hz, 2H), 2.31 (t, J=7.3 Hz, 2H), 2.18 (m, 2H), 1.98 (m, 2H) ppm. ¹³C NMR (CDCl₃): δ=176.83, 173.31, 169.24, 167.46, 38.68, 35.19, 33.04, 31.88, 27.57, 21.00, 20.79 ppm. HPLC-MS/PDA (5% to 100% in 10 min): tᵣ=2.72 min (m/z=+268.17 Da [M+H]⁺; calcd 268.14 for C₁₁H₁₈N₅O₃; λₘₐₓ=278, 518 nm).

The following compounds 2.7-2.8 have been prepared according to procedure E. 2.7

This compound was prepared from 2.5 and 37-amino-5,8,11,14,17,20,23,26,29,32,35-undecaoxa-2-azaheptatriacontanoic acid t-butyl ester that were reacted in a 1:1 molar ratio. Column chromatography (flash SiO₂) using an elution gradient of 0% to 8% MeOH in CH₂Cl₂ yielded pure 2.7 (2.46 g, 2.84 mmol, 95%) as a purple oil. ¹H-NMR (CDCl₃): δ=7.72 (t, 1H, NH), 7.33 (t, 1H, NH), 5.08 (d, 2H, TZCH₂), 3.74-3.39 (m, 46H, OCH₂, NHCH₂), 3.31 (q, 2H, CH₂NHBoc), 3.07 (s, 3H, TZCH₃), 2.36 (t, 2H, NHC(O)CH₂), 2.24 (t, 2H, NHC(O)CH₂), 2.03 (m, 2H, CH₂CH₂CH₂), 1.44 (s, 9H, C(CH₃)₃). ¹³C-NMR (CDCl₃): δ=173.3, 173.0, 168.3, 166.8, 156.0, 79.0, 70.5, 70.2, 69.6, 42.2, 40.3, 39.3, 34.7, 34.0, 28.4, 21.8, 21.1. ESI-MS: m/z Calc. for C₃₈H₇₁N₇O₁₅ 865.50; Obs. [M+H]⁺ 866.50, [M+Na]⁺ 888.58.

2.8

Compound 2.6 was reacted with mono-Boc-protected PEG diamine in a 1:1 molar ratio. Compound 2.8 was obtained as a pink solid, containing a trace amount of tri(pyrrolidin-1-yl) phosphine oxide.

¹H NMR (CDCl₃): δ=6.39 (s, 1H), 6.37 (s, 1H), 5.05 (br s, 1H), 3.85-3.59 (m, 40H), 3.59-3.49 (m, 4H), 3.44 (d, J=5.5 Hz, 2H), 3.41-3.23 (m, 6H), 3.04 (s, 3H), 2.27 (td, J=7.1, 2.0 Hz, 4H), 2.17 (m, 2H), 1.96 (m, 2H), 1.44 (s, 9H) ppm. ¹³C NMR (CDCl₃): δ=172.73, 172.66, 169.35, 167.48, 155.96, 70.54 (m), 70.21, 70.19, 69.67, 46.28, 46.24, 40.35, 39.21, 38.41, 35.19, 35.12, 31.97, 28.41, 27.88, 26.44, 26.36, 21.84, 21.08 ppm. HPLC-MS/PDA (5% to 100% in 10 min): tᵣ=5.05 min (m/z=+894.33 Da [M+H]⁺; calcd 894.54 for C₄₀H₇₆N₇O₁₅; λₘₐₓ=277, 523 nm).

The following compounds 2.9-2.10 have been prepared according to general procedures B and F.

2.9

Compound 2.7 was deprotected and the reaction was monitored with HPLC-MS/PDA. ESI-MS: m/z Calc. for C₃₃H₆₃N₇O₁₃ 765.45; Obs. [M+H]⁺ 766.67, [M+Na]⁺ 788.50, [M+2H]²⁺ 384.00. The intermediate was then reacted with the mono(4-nitrophenyl) ester derivative of DOTA in a 1:1.1 molar ratio. Precipitation (10 mL MeCN→200 mL diethyl ether) was followed by decantation and drying of the solid in vacuo. Purification with preparative RP-MPLC using an elution gradient of 10% to 40% MeCN in H₂O (both containing 0.1% formic acid) followed by lyophilization yielded pure 2.9 (1.15 g, 1.00 mmol, 72% over two steps) as a red sticky solid. ESI-MS: m/z Calc. for C₄₉H₈₉N₁₁O₂₀ 1151.63; Obs. [M+2H]²⁺ 577.17, [M+H]⁺ 1152.75.

2.10

Compound 2.8 was deprotected and the reaction was monitored with MS. HPLC-MS/PDA (5% to 100% in 10 min): tᵣ=3.80 min (m/z=+794.50 Da [M+H]⁺; calcd 794.49 for C₃₅H₆₈N₇O₁₃; λₘₐₓ=278, 521 nm).

The intermediate was then reacted with the mono(4-nitrophenyl) ester derivative of DOTA in a 1:1.1 molar ratio. The product was purified by column chromatography (RP silica gel, acetonitrile/0.1 v/v % aqueous formic acid=15:85), and isolated by lyophilization, to yield product 2.10 as a pink solid. ¹H NMR (D₂O): δ=4.04-3.47 (m, 54H), 3.38 (m, 14H), 3.12 (m, 8H), 3.01 (s, 3H), 2.26 (m, 4H), 2.13 (m, 2H), 1.85 (m, 2H) ppm. HPLC-MS/PDA (5% to 100% in 10 min): tᵣ=3.82 min (m/z=+1180.83 Da [M+H]⁺; calcd 1180.67 for C₅₁H₉₄N₁₁O₂₀; λₘₐₓ=276, 519 nm).

2.11

To a solution of compound 2.9 (0.159 g, 0.138 mmol) in 0.1 M aqueous sodium acetate buffer (5.5 mL, pH=5.5) was added lutetium(III) chloride hexahydrate (80.3 mg, 0.206 mmol). The solution was stirred at 20° C. for 1 h, and then the product was purified by column chromatography (RP silica gel, acetonitrile/0.1 v/v % aqueous formic acid=30:70), and isolated by lyophilization, to yield product 2.11 as a pink solid (0.170 g, 93%). ¹H NMR (D₂O): δ=5.00 (s, 2H), 3.90-3.10 (m, 60H), 3.05 (s, 3H), 2.90-2.45 (m, 12H), 2.41 (t, 2H), 2.31 (t, 2H), 1.92 (m, 2H) ppm. HPLC-MS/PDA (5% to 100% in 10 min): tᵣ=4.2 min (m/z=+663.25 [M+2H]²⁺, +1324.75 [M+H]⁺, −1323.33 [M−H]⁻, −1367.83 [M+HCOO]⁻ Da; calcd 1324.55 for C₄₉H₈₇N₁₁O₂₀Lu [M+H]⁺).

2.12

To a solution of compound 2.10 (1.94 g, 1.64 mmol) in 0.2 M aqueous sodium acetate buffer (60 mL, pH=5.5) was added lutetium(III) chloride hexahydrate (1.28 g, 3.28 mmol). The solution was stirred at 4° C. for 16 h, and then the product was purified by column chromatography (RP silica gel, acetonitrile/0.1 v/v % aqueous formic acid=20:80), and isolated by lyophilization, to yield product 2.11 as a pink solid (1.70 g, 77%). ¹H NMR (D₂O): δ=3.83 3.15 (m, 64H), 3.03 (s, 3H), 2.81 (m, 8H), 2.53 (m, 4H), 2.28 (m, 4H), 2.16 (m, 2H), 1.87 (m, 2H) ppm. ¹³C NMR (D₂O): δ=180.78, 175.99, 175.85, 175.74, 169.18, 167.52, 69.57, 69.37, 68.80, 68.55, 65.71, 55.81, 55.29, 39.67, 38.89, 38.35, 34.86, 34.82, 31.28, 26.61, 21.75, 20.05 ppm. HPLC-MS/PDA (5% to 100% in 10 min): tᵣ=3.62 min (m/z=+677.17 [M++2H]²⁺, +1352.83 [M+H]⁺, −1351.17 [M−H]⁻, −1396.00 [M+HCOO]⁻ Da; calcd 1352.58 for C₅₁H₉₁N₁₁O₂₀Lu [M+H]⁺).

### Example 3: Synthesis of 3-Alkyl-6-Pyridyl TZ Precursors and Activators

The synthesis of 3-(2-pyridyl)-6-methyl-1,2,4,5-tetrazine (3.1) was reported in Versteegen et al., Angew. Chem. Int. Ed., 2013, 52, 14112-14116.

The syntheses of 5-((6-(6-methyl-1,2,4,5-tetrazin-3-yl)pyridin-3-yl)amino)-5-oxopentanoic acid and 3-(pyridin-2-yl)-6-methyl-1,2,4,5-tetrazine functional dextran (3.2) were reported in Rossin et al., Bioconjug. Chem., 2016, 27, 1697-1706.

Synthesis of 2,2′,2″-(10-(44-((6-(6-methyl-1,2,4,5-tetrazin-3-yl)pyridin-3-yl)amino)-2,40,44-trioxo-6,9,12,15,18,21,24,27,30,33,36-undecaoxa-3,39-diazatetratetracontyl)-1,4,7,10-tetraazacyclododecane-1,4,7-triyl)triacetic acid (3.4).

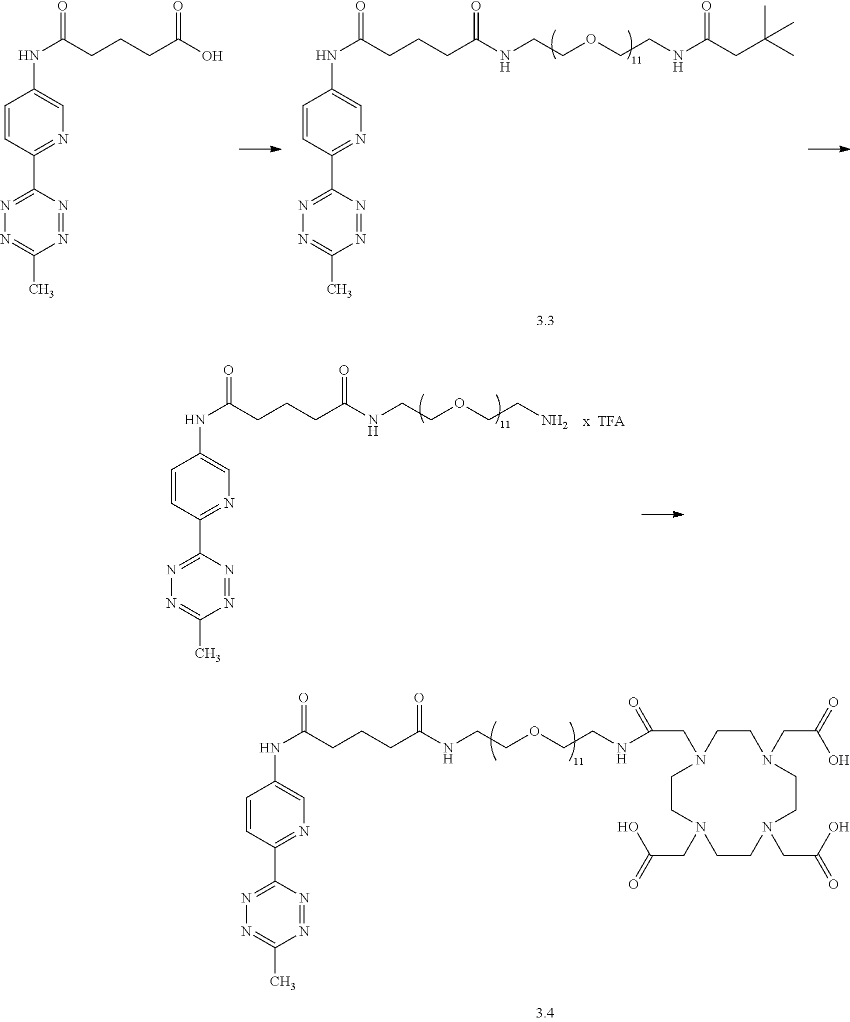

Compound 3.3 has been prepared according to general procedure E.

3.3

This compound was prepared from previously reported 5-((6-(6-methyl-1,2,4,5-tetrazin-3-yl)pyridin-3-yl)amino)-5-oxopentanoic acid (Rossin et al., Bioconjug. Chem., 2016, 27, 1697-1706) and 37-amino-5,8,11,14,17,20,23,26,29,32,35-undecaoxa-2-azaheptatriacontanoic acid t-butyl ester that were reacted in a 1:1 molar ratio. Column chromatography (flash SiO₂) using an elution gradient of 1% to 8% MeOH in CHCl₃ yielded pure 3.3 (2.60 g, 2.80 mmol, 79%) as a purple oil. ¹H-NMR (CDCl₃): δ=9.63 (s, 1H, NH), 8.95 (t, 1H, ArH), 8.61 (d, 2H, ArH), 6.81 (br, 1H, NH), 5.08 (br, 1H, NH), 3.71-3.44 (m, 46H, OCH₂, NHCH₂), 3.30 (q, 2H, CH₂NHBoc), 3.13 (s, 3H, TZCH₃), 2.56 (t, 2H, NHC(O)CH₂), 2.37 (t, 2H, NHC(O)CH₂), 2.09 (qn, 2H, CH₂CH₂CH₂), 1.44 (s, 9H, C(CH₃)₃). ¹³C-NMR (CDCl₃): δ=172.9, 172.5, 167.6, 163.1, 156.0, 144.1, 141.8, 138.4, 126.5, 124.3, 79.1, 70.5, 70.1, 69.6, 40.3, 39.3, 36.0, 35.0, 28.4, 21.3, 21.2. ESI-MS: m/z Calc. for C₄₂H₇₂N₈O₁₅ 928.51; Obs. [M+H]⁺ 929.58, [M+Na]⁺ 951.58.

Compound 3.4 has been prepared according to general procedures B and F.

3.4

Compound 3.3 was deprotected and the reaction was monitored with HPLC-MS/PDA. ESI-MS: m/z Calc. for C₃₇H₆₄N₈O₁₃ 828.46; Obs. [M+H]⁺ 829.67, [M+2H]²⁺ 415.50. The intermediate was then reacted with the mono(4-nitrophenyl) ester derivative of DOTA in a 1:1.1 molar ratio. Precipitation (7 mL MeCN→150 mL diethyl ether) was followed by decantation and drying of the solid in vacuo. Purification with preparative RP-MPLC using an elution gradient of 10% to 30% MeCN in H₂O (both containing 0.1% formic acid) followed by lyophilization yielded pure 3.4 (0.57 g, 0.47 mmol, 62% over two steps) as a red sticky solid. ESI-MS: m/z Calc. for C₅₃H₉₀N₁₂O₂₀ 1214.64; Obs. [M+3H]³⁺ 406.08, [M+2H]²⁺ 608.67, [M+H]⁺ 1215.75, [M+Na]⁺ 1237.67.

### Example 4: Synthesis of Alkyl-Pyrimidyl TZ Building Blocks and Activators

The synthesis of 3-methyl-6-(pyrimidin-2-yl)-1,2,4,5-tetrazine (4.1) was reported in Fan et al., Angew. Chem. Int. Ed. 2016, 55, 14046-14050.

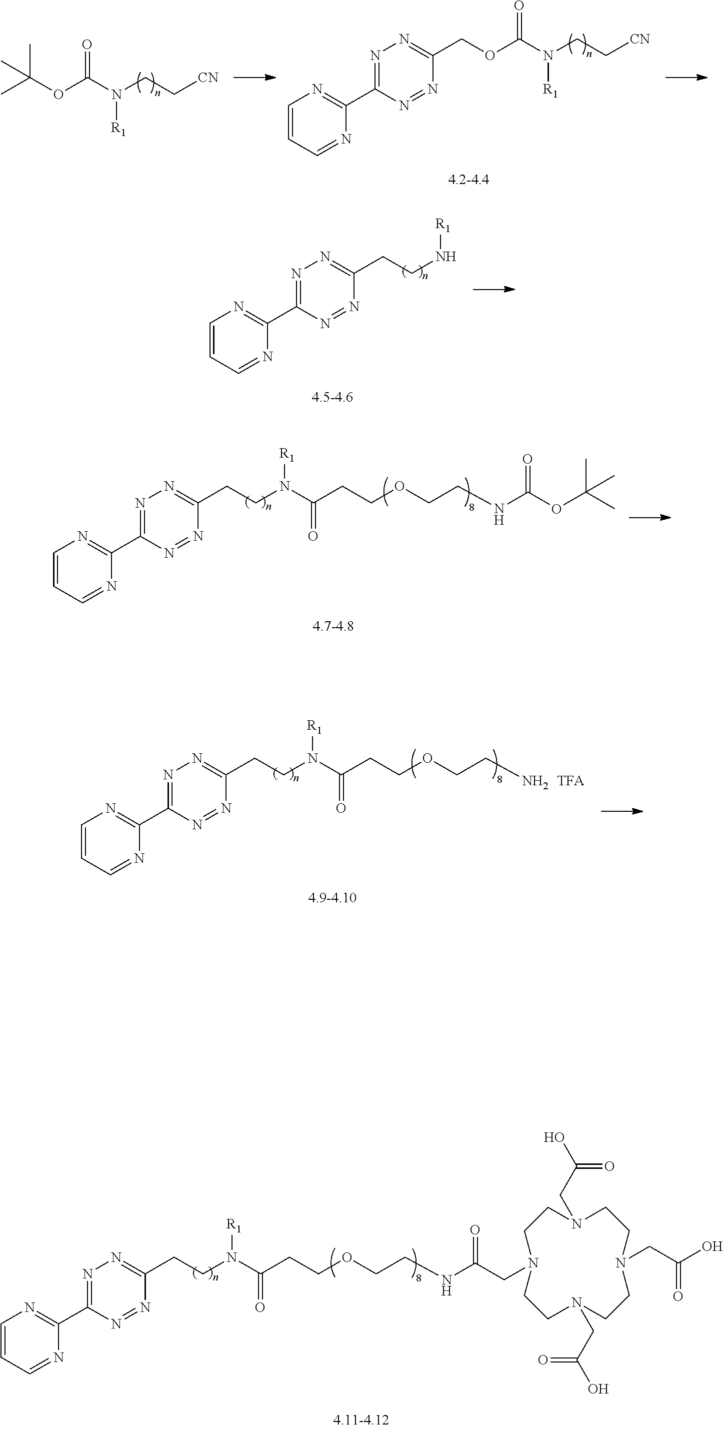

| Code | n | Ri |
|---|---|---|
| 4.2 | 1 | H |
| 4.3, 4.5, 4.7, 4.9, | 1 | Me |
| 4.11 | | |
| 4.4, 4.6, 4.8, | 2 | H |
| 4.10, 4.12 | | |

The following compounds 4.2-4.4 have been prepared according to general procedure A.

4.2

This compound was prepared from 2-pyrimidinecarbonitrile and t-butyl N-(2-cyanoethyl)carbamate that were reacted in a 3:2 molar ratio. Oxidation was performed according to general procedure A1. Column chromatography (flash SiO₂) using an elution gradient of 20% to 60% EtOAc in CHCl₃ and, in a second chromatography step (normal SiO₂), elution with 55% acetone in heptane yielded pure 4.2 (113 mg, 0.37 mmol, 22%) as a red solid. ¹H-NMR (CDCl₃): δ=9.13 (d, 2H, ArH), 7.60 (t, 1H, ArH), 5.18 (br, 1H, NH), 3.84 (q, 2H, CH₂N), 3.70 (t, 2H, TZCH₂), 1.39 (s, 9H, CH₃). ¹³C-NMR (CDCl₃): δ=169.4, 163.3, 159.4, 158.4, 155.8, 122.6, 79.4, 38.4, 35.6, 28.3. ESI-MS: m/z Calc. for C₁₃H₁₇N₇O₂ 303.14; Obs. [M−tboc+H]⁺ 204.17, [M-tbutyl+2H]⁺ 248.08, [M+Na]⁺ 326.08.

4.3

This compound was prepared from 2-pyrimidinecarbonitrile and t-butyl N-(2-cyanoethyl)-N-methylcarbamate that were reacted in a 1:1 molar ratio. Oxidation was performed according to general procedure A2. Column chromatography (flash SiO₂) using an elution gradient of 20% to 50% EtOAc in CHCl₃ and, in a second chromatography step (normal SiO₂), elution with 40% acetone in heptane yielded pure 4.3 (56 mg, 0.18 mmol, 16%) as a red solid. ¹H-NMR (CDCl₃): δ=9.12 (d, 2H, ArH), 7.59 (t, 1H, ArH), 3.86 (br, 2H, CH₂N), 3.70 (t, 2H, TZCH₂), 2.94 (s, 3H, NCH₃), 1.34 (s, 9H, C(CH₃)₃). ESI-MS: m/z Calc. for C₁₄H₁₉N₇O₂ 317.16; Obs. [M-tboc+H]⁺ 218.00, [M-tbutyl+2H]⁺ 261.92, [M+Na]⁺ 340.08.

4.4

This compound was prepared from 2-pyrimidinecarbonitrile and t-butyl N-(3-cyanopropyl)carbamate that were reacted in a 3:2 molar ratio. Oxidation was performed according to general procedure A1. Column chromatography (flash SiO₂) using an elution gradient of 20% to 60% EtOAc in CHCl₃ and, in a second chromatography step (normal SiO₂), elution with 50% acetone in heptane yielded pure 4.4 (55 mg, 0.17 mmol, 20%) as a red oil. ¹H-NMR (CDCl₃): δ=9.13 (d, 2H, ArH), 7.60 (t, 1H, ArH), 4.76 (br, 1H, NH), 3.53 (t, 2H, TZCH₂), 3.34 (q, 2H, CH₂N), 2.24 (qn, 2H, CH₂CH₂N), 1.44 (s, 9H, CH₃). ¹³C-NMR (CDCl₃): δ=171.0, 163.4, 159.6, 158.4, 155.9, 122.6, 79.4, 39.7, 32.4, 28.4 (2×). ESI-MS: m/z Calc. for C₁₄H₁₉N₇O₂ 317.16; Obs. [M-tboc+H]⁺ 218.00, [M-tbutyl+2H]⁺ 261.92, [M+Na]⁺ 340.08.

The following compounds 4.5 and 4.6 have been prepared according to general procedure B.

4.5

This compound was prepared from 4.3. Pure 4.5 was obtained as a red oil (10.4 mg, 31 μmol, 100%). ¹H-NMR (CD₃OD): δ=9.15 (d, 2H, ArH), 7.80 (t, 1H, ArH), 3.91 (t, 2H, CH₂), 3.78 (t, 2H, CH₂), 2.86 (s, 3H, NCH₃).

4.6

This compound was prepared from 4.4. Pure 4.6 was obtained as a red oil (109 mg, 0.33 mmol, 100%). ¹H-NMR (CD₃OD): δ=9.14 (d, 2H, ArH), 7.79 (t, ArH), 3.60 (t, 2H, CH₂), 3.22 (t, 2H, CH₂), 2.44 (qn, 2H, CH₂CH₂N). ¹³C-NMR (CD₃OD): δ=171.5, 164.0, 161.1 (q), 160.0, 159.7, 124.6, 117.1 (q), 40.1, 32.7, 26.0. ¹⁹F-NMR (CD₃OD): δ=−77.5. ESI-MS: m/z Calc. for C₁₁H₁₂F₃N₇O₂ 331.10; Obs. [M-TFA+H]⁺ 218.00. Note that 4.6 is highly unstable in its free base form due to intramolecular nucleophilic attack by the amine functionality.

The following compounds 4.7 and 4.8 have been prepared according to general procedure C.

4.7

This compound was prepared from 4.5 and 5,8,11,14,17,20,23,26-octaoxa-2-azanonacosanedioic acid 1-t-butyl ester that were reacted in a 1:1 molar ratio. Title compound 4.7 was obtained as a purple oil and used without further purification. The NMR spectrum indicates the presence of two carbamate rotamers. ¹H-NMR (CDCl₃): δ=9.13 (2d, 2H, ArH), 7.62 and 7.59 (2t, 1H, ArH), 5.10 (br, 1H, NH), 4.13-3.51 (m, 36H, OCH₂, TZCH₂CH₂), 3.31 (q, 2H, CH₂NH), 3.11 and 3.02 (2s, 3H, NCH₃), 2.73 and 2.57 (2t, 2H, C(O)CH₂), 1.44 (s, 9H, C(CH₃)₃). ESI-MS: m/z Calc. for C₃₃H₅₆N₈O₁₁ 740.41; Obs. [M-tboc+H]⁺ 641.33, [M+H]⁺ 741.00, [M+Na]⁺ 763.17.

4.8

This compound was prepared from 4.6 and 5,811,14,17,20,23,26-octaoxa-2-azanonacosanedioic acid 1-t-butyl ester that were reacted in a 1:1 molar ratio. Column chromatography (flash SiO₂) using an elution gradient of 1% to 6% MeOH in CHCl₃ yielded pure 4.8 (198 mg, 0.27 mmol, 81%) as a purple oil. ¹H-NMR (CDCl₃): δ=9.13 (d, 2H, ArH), 7.61 (t, 1H, ArH), 6.89 (br t, 1H, NH), 5.09 (br, 1H, NH), 3.76-3.43 (m, 36H, OCH₂, CH₂CH₂CH₂), 3.31 (q, 2H, OCH₂CH₂NH), 2.49 (t, 2H, C(O)CH₂), 2.26 (qn, 2H, CH₂CH₂CH₂), 1.44 (s, 9H, CH₃). ESI-MS: m/z Calc. for C₃₃H₅₆N₈O₁₁ 740.41; Obs. [M-tboc+H]⁺ 641.42, [M+Na]⁺ 763.33.

The following compounds 4.9 and 4.10 have been prepared according to general procedure B.

4.9

This compound was prepared from 4.7. Precipitation (0.5 mL CHCl₃→25 mL diethyl ether) followed by centrifugation, decantation and drying of the solid in vacuo yielded pure 4.9 (55 mg, 0.17 mmol, 80%) as a red oil. The NMR spectrum indicates the presence of two carbamate rotamers. ¹H-NMR (CDCl₃): δ=9.14 and 9.12 (2d, 2H, ArH), 7.62 and 7.60 (2t, 1H, ArH), 4.12-3.52 (m, 36H, OCH₂, TZCH₂CH₂), 3.18 (m, 2H, CH₂NH₂), 3.11 and 3.01 (2s, 3H, NCH₃), 2.73 and 2.56 (2t, 2H, C(O)CH₂). ESI-MS: m/z Calc. for C₃₀H₄₉F₃N₈O₁₁ 754.35; Obs. [M-TFA+H]⁺ 641.33.

4.10

This compound was prepared from 4.8. Pure 4.10 was obtained as a red oil (202 mg, 0.27 mmol, 100%). ¹H-NMR (CDCl₃): δ=9.19 (d, 2H, ArH), 7.70 (t, 1H, ArH), 3.81-3.46 (m, 36H, OCH₂, CH₂CH₂CH₂), 3.15 (m, 2H, CH₂NH₂), 2.63 (t, 2H, C(O)CH₂), 2.29 (qn, 2H, CH₂CH₂CH₂). ¹⁹F-NMR (CDCl₃): δ=−76.0. ESI-MS: m/z Calc. for C₃₀H₄₉F₃N₈O₁₁ 754.35; Obs. [M-TFA+H]⁺ 641.50.

The following compounds 4.11 and 4.12 have been prepared according to general procedure F.

4.11

This compound was prepared from 4.9. Purification with preparative RP-HPLC using an elution gradient of 15% to 17 MeCN in H₂O (both containing 0.1% formic acid) followed by lyophilization yielded pure 4.11 (35 mg, 34 μmol, 38%) as a red solid. ESI-MS: m/z Calc. for C₄₄H₇₄N₁₂O₁₆ 1026.53; Obs. [M+2H]²⁺ 514.42, [M+H]⁺ 1027.42.

4.12

This compound was prepared from 4.10. Purification with preparative RP-HPLC using an elution gradient of 14% to 18% MeCN in H₂O (both containing 0.1% formic acid) followed by lyophilization yielded pure 4.12 (148 mg, 0.14 mmol, 54%) as a red solid. ESI-MS: m/z Calc. for C₄₄H₇₄N₁₂O₁₆ 1026.53; Obs. [M+2H]²⁺ 514.50, [M+H]⁺ 1027.67.

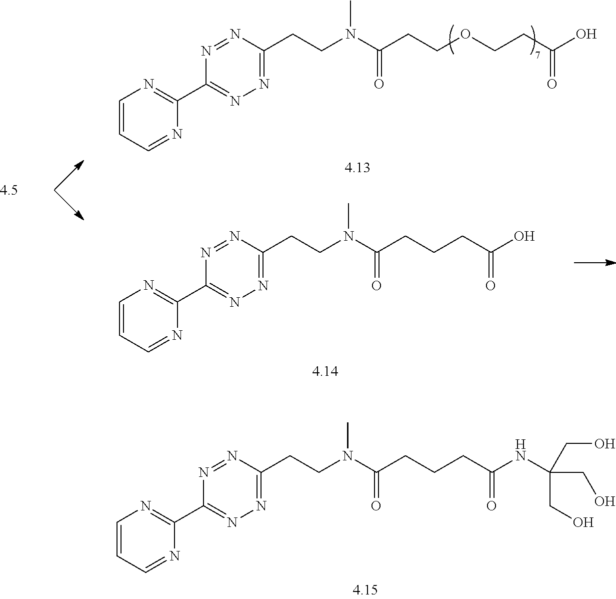

4.13

This compound has been prepared according to general procedure C from 4.5 and 4,7,10,13,16,19,22-heptaoxapentacosanedioic acid that were reacted in a 1:6 molar ratio. PyBOP was added to a mixture of TZ amine (TFA-salt), PEG-acid and N,N-diisopropylethylamine (16 eq) in CH₂Cl₂. During work-up, the sat. NaHCO₃ wash was omitted. Precipitation (0.5 mL CHCl₃→20 mL diethyl ether) was promoted at −20° C. for 40 h followed by centrifugation, decantation and drying of the solid in vacuo. Purification with preparative RP-HPLC using an elution gradient of 20% to 25% MeCN in H₂O (both containing 0.1% formic acid) followed by lyophilization yielded pure 4.13 (10.4 mg, 17 μmol, 30%) as a red oil. The NMR spectrum indicates the presence of two carbamate rotamers. ¹H-NMR (CDCl₃): δ=9.14 (2d, 2H, ArH), 7.62 and 7.59 (2t, 1H, ArH), 4.13-3.55 (m, 32H, OCH₂, TZCH₂CH₂), 3.12 and 3.03 (2s, 3H, NCH₃), 2.74 and 2.59 and 2.57 (3t, 4H, C(O)CH₂). ESI-MS: m/z Calc. for C₂₇H₄₃N₇O₁₀ 625.31; Obs. [M+H]⁺ 626.33, [M+Na]⁺ 648.17.

4.14

To a 5 mL test tube containing glutaric anhydride (6.1 mg, 54 μmol, 1 eq), a solution of 4.5 (17.7 mg, 53 μmol) and N,N-diisopropylethylamine (37 μL, 0.21 mmol, 4 eq) in CH₂Cl₂ (1 mL) was added. The solution was stirred at room temperature for 30 min and the solvent was removed in vacuo. Column chromatography (flash SiO₂) using an elution gradient of 4% to 16% MeOH in CHCl₃ yielded the N,N-diisopropylethylamine salt of 4.14 (24 mg, 52 μmol, 97%) as a pink oil. The NMR spectrum indicates the presence of two carbamate rotamers. ¹H-NMR (CD₃OD): δ=9.12 (2d, 2H, ArH), 7.78 and 7.77 (2t, 1H, ArH), 4.13 and 3.95 (2t, 2H, CH₂), 3.80-3.64 (m, 4H, CH₂, dipea-CH), 3.23 (q, 2H, dipea-CH₂), 3.19 and 3.02 (2s, 3H, NCH₃), 2.48 and 2.34 (2t, 2H, C(O)CH₂), 2.31 and 2.23 (2t, 2H, C(O)CH₂), 1.86 and 1.69 (2qn, 2H, CH₂CH₂CH₂), 1.37 (m, 15H, dipea-CH₃). ESI-MS: m/z Calc. for C₁₄H₁₇N₇O₃ 331.14; Obs. [M+H]⁺ 332.08, [2M+Na]⁺ 684.92.

4.15

The N,N-diisopropylethylamine salt of 4.14 (24 mg, 52 μmol), 2-amino-2-(hydroxymethyl)propane-1,3-diol (6.9 mg, 57 μmol, 1.1 eq) and N,N-diisopropylethylamine (29 μL, 0.16 mmol, 3 eq) were combined in DMF (800 μL). PyBOP (29 mg, 56 μmol, 1.1 eq) was added and the mixture was stirred at room temperature for 30 min. After removal of the solvent in vacuo, precipitation (1 mL DMF→25 mL diethyl ether) was followed by centrifugation and decantation. Purification with preparative RP-HPLC using an elution gradient of 12% to 14% MeCN in H₂O (both containing 0.1% formic acid) followed by lyophilization yielded pure 4.15 (7.8 mg, 18 μmol, 34%) as a pink fluffy solid. The NMR spectrum indicates the presence of two carbamate rotamers. ¹H-NMR (CD₃CN+2 drops D₂O): δ=9.08 (d, 2H, ArH), 7.70 (t, 1H, ArH), 6.91 and 6.77 (2br, 1H, NH), 4.00 and 3.85 (2t, 2H, CH₂), 3.72-3.55 (m, 8H, CH₂, CH₂OH), 3.07 and 2.93 (2s, 3H, NCH₃), 2.38 and 2.23 (2t, 2H, C(O)CH₂), 2.21 and 2.07 (2t, 2H, C(O)CH₂), 1.79 and 1.61 (2qn, 2H, CH₂CH₂CH₂). ESI-MS: m/z Calc. for C₁₈H₂₆N₈O₅ 434.20; Obs. [M+H]⁺ 435.17, [M+Na]⁺ 457.17.

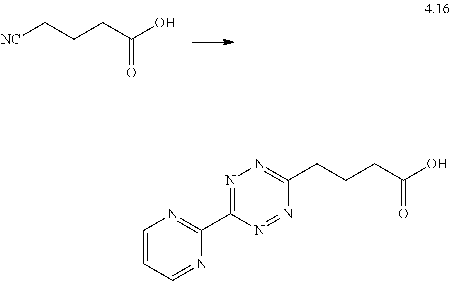 [SMILES: N#CCCCC(=O)O ; O=C(O)CCCc1nnc(-c2ncccn2)nn1]

The following compound 4.16 has been prepared according to general procedure A.

4.16

This compound was prepared from 2-pyrimidinecarbonitrile and 4-cyanobutanoic acid that were reacted in a 1:1 molar ratio. Oxidation was performed according to general procedure A2. Column chromatography (flash SiO₂) using an elution gradient of 1% to 3% MeOH in CHCl₃ and, in a second chromatography step (normal SiO₂), elution with 50% acetone in heptane yielded pure 4.16 (38 mg, 0.15 mmol, 7%) as a red solid. ¹H-NMR (CDCl₃): δ=9.13 (d, 2H, ArH), 7.62 (t, 1H, ArH), 3.57 (t, 2H, TZCH₂), 2.55 (t, OH, CH₂C(O)), 2.37 (qn, 2H, CH₂CH₂CH₂).

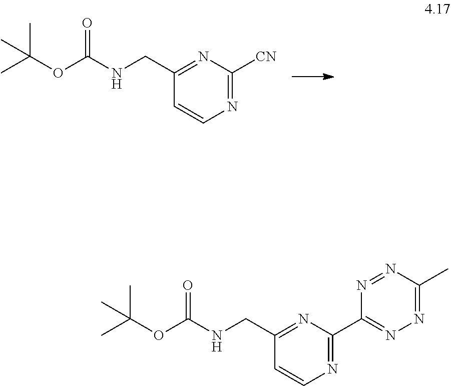 [SMILES: CC(C)(C)OC(=O)NCc1ccnc(C#N)n1 ; Cc1nnc(-c2nccc(CNC(=O)OC(C)(C)C)n2)nn1]

The following compound 4.17 has been prepared according to general procedure A.

4.17

This compound was prepared from t-butyl N-((2-cyano-4-pyrimidinyl)methyl)carbamate (Sweeney et al., ACS Med. Chem. Lett. 2014, 5, 937-941) and acetonitrile that were reacted in a 1:6 molar ratio. Oxidation was performed according to general procedure A1. Column chromatography (flash SiO₂) using an elution gradient of 0% to 70% EtOAc in CHCl₃ and, in a second chromatography step (normal SiO₂), elution with 40% acetone in heptane yielded pure 4.17 (33 mg, 0.11 mmol, 19%) as a purple solid. ¹H-NMR (CDCl₃): δ=9.04 (d, 1H, ArH), 7.59 (d, 1H, ArH), 5.54 (br, 1H, NH), 4.64 (d, 2H, CH₂NH), 3.21 (s, 3H, TZCH₃), 1.48 (s, 9H, C(CH₃)₃). ESI-MS: m/z Calc. for C₁₃H₁₇N₇O₂ 303.14; Obs. [M-tboc+H]⁺ 204.17, [M-tbutyl+2H]⁺ 248.08, [M+Na]⁺ 326.17.

The following compounds 4.18, 4.19 and 4.20 have been prepared according to general procedure A.

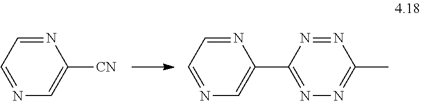

Compound 4.18 was prepared from pyrazine-2-carbonitrile and acetonitrile that were reacted in a 2:3 molar ratio. Oxidation was performed according to general procedure A3. Column chromatography (flash SiO₂) using an elution gradient of 10% to 40% EtOAc in CHCl₃ and, in a second chromatography step (normal SiO₂), elution with 40% acetone in heptane yielded pure 4.18 (70 mg, 0.40 mmol, 12%) as a pink solid. ¹H-NMR (CDCl₃): δ=9.85 (d, 1H, ArH), 8.91 (m, 1H, ArH), 8.87 (d, 1H, ArH), 3.21 (s, 3H, CH₃). ¹³C-NMR (CDCl₃): δ=168.6, 162.8, 147.3, 146.0, 145.1, 145.0, 21.5. ESI-MS: m/z Calc. for C₇H₆N₆ 174.07; Obs. [M+H]⁺ 175.08.

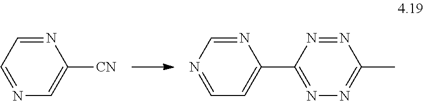

Compound 4.19 was prepared from pyrimidine-4-carbonitrile and acetonitrile that were reacted in a 2:3 molar ratio. Oxidation was performed according to general procedure A2. Column chromatography (flash SiO₂) using an elution gradient of 10% to 40% EtOAc in CHCl₃ and, in a second chromatography step (normal SiO₂), elution with 40% acetone in heptane yielded pure 4.19 (28 mg, 0.16 mmol, 6%) as a pink solid. ¹H-NMR (CDCl₃): δ=9.58 (d, 1H, ArH), 9.11 (d, 1H, ArH), 8.59 (dd, 1H, ArH), 3.22 (s, 3H, CH₃). ¹³C-NMR (CDCl₃): δ=169.2, 162.9, 159.9, 159.2, 157.4, 119.8, 21.6. ESI-MS: m/z Calc. for C₇H₆N₆ 174.07; Obs. [M+H]⁺ 175.08.

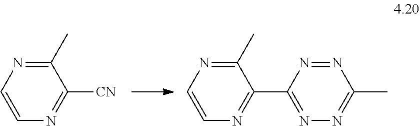

Compound 4.20 was prepared from 3-methylpyrazine-2-carbonitrile and acetonitrile that were reacted in a 2:3 molar ratio. Oxidation was performed according to general procedure A2. Column chromatography (flash SiO₂) using an elution gradient of 10% to 20% EtOAc in CHCl₃ and, in a second chromatography step (normal SiO₂), elution with 38% acetone in heptane yielded pure 4.20 (33 mg, 0.18 mmol, 9%) as a pink oil. ¹H-NMR (CDCl₃): δ=8.72 (2d, 2H, ArH), 3.20 (s, 3H, CH₃), 2.88 (s, 3H, CH₃). ¹³C-NMR (CDCl₃): δ=167.7, 165.3, 154.7, 145.7, 145.5, 142.4, 23.2, 21.5. ESI-MS: m/z Calc. for C₈H₈N₆ 188.08; Obs. [M+H]⁺ 189.08.

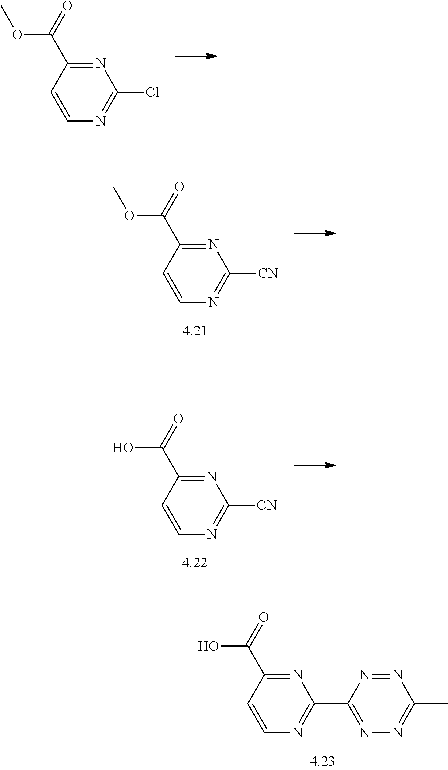 [SMILES: COC(=O)c1ccnc(Cl)n1 ; COC(=O)c1ccnc(C#N)n1 ; N#Cc1nccc(C(=O)O)n1 ; Cc1nnc(-c2nccc(C(=O)O)n2)nn1]

4.21:

Methyl 2-chloropyrimidine-4-carboxylate (0.80 g, 4.5 mmol), Zn(CN)₂ (0.56 g, 4.7 mmol, 1.04 eq) and Pd(PPh₃)₄ (0.52 g, 0.44 mmol, 0.1 eq) were combined in DMF (4 mL) and the mixture was stirred at 100° C. for 2 h. After cooling to room temperature, the solvent was removed in vacuo (oil pump, 44° C.) and the resulting purple paste was triturated with CHCl₃ (18 mL). The suspension was filtrated, the solid was washed with CHCl₃ (2×4 mL) and the filtrate was evaporated to dryness yielding a purple oil. The oil was purified with column chromatography (flash SiO₂) using an elution gradient of pentane/CHCl₃ 1:2 to CHCl₃ to 15% EtOAc in CHCl₃. Finally, precipitation (2 mL CHCl₃→60 mL pentane), filtration and drying the solid in vacuo yielded pure 4.21 (0.64 g, 3.9 mmol, 87%) as a white solid. ¹H-NMR (CDCl₃): δ=9.11 (d, 1H, ArH), 8.20 (d, 1H, ArH), 4.08 (s, 3H, CH₃).

4.22:

4.21 (0.40 g, 2.5 mmol) was dissolved in 1,2-dichloroethane (14 mL). Me₃SnOH (1.36 g, 7.4 mmol, 3 eq) was added as a solid and the mixture was stirred at 70° C. for PA h. The volatiles were removed in vacuo and the mixture was redissolved in EtOAc (100 mL). The organic layer was washed with 1 M HCl (30 mL), dried using Na₂SO₄ and the solvent was removed in vacuo. Trituration in hot CHCl₃ (10 mL), filtration and drying the solid in vacuo yielded pure 4.22 (0.27 g, 1.8 mmol, 73%) as a white solid. ¹H-NMR (MeOD): δ=9.16 (d, 1H, ArH), 8.26 (d, 1H, ArH). ESI-MS: m/z Calc. for C₆H₃N₃O₂ 149.02; Obs. [M−H]⁻ 148.08.

Compound 4.23 was prepared according to general procedure A from 4.22 and acetonitrile that were reacted in a 1:3 molar ratio. Water was added as a co-solvent during the [2H]-TZ synthesis. Oxidation was performed according to general procedure A3 except that 1 M HCl was added up to pH=1. Column chromatography (flash SiO₂) using an elution gradient of 20% to 80% EtOAc in CHCl₃ followed by 4% to 8% MeOH in CHCl₃ yielded 4.23 as a red solid. ¹H-NMR (MeOD): δ=9.33 (d, 1H, ArH), 8.31 (d, 1H, ArH), 3.16 (s, 3H, CH₃). ESI-MS: m/z Calc. for C₈H₆N₆O₂ 218.06; Obs. [M+H]⁺ 219.17.

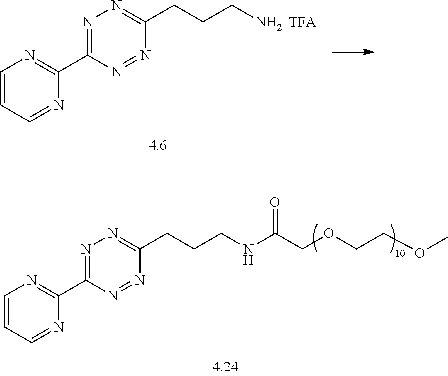

Compound 4.22 was prepared according to general procedure C from 4.6 and 3,6, 9,12,15,18,21,24,27,30,33-undecaoxatetratriacontanoic acid that were reacted in a 1:1 molar ratio. Column chromatography (flash SiO₂) using an elution gradient of 3% to 5% MeOH in CHCl₃ yielded pure 4.24 (32 mg, 44 μmol, 58%) as a pink oil. ¹H-NMR (CDCl₃): δ=9.13 (d, 2H, Aril), 7.60 (t, 1H, ArH), 7.26 (br t, 1H, NH), 4.00 (s, 2H, C(O)CH₂), 3.71-3.49 (m, 44H, OCH₂, CH₂CH₂CH₂), 3.38 (s, 3H, OCH₃), 2.29 (qn, 2H, CH₂CH₂CH₂). ESI-MS: m/z Calc. for C₃₂H₅₅N₇O₁₂ 729.39; Obs. [M+H]⁺ 730.50, [M+Na]⁺ 752.42.

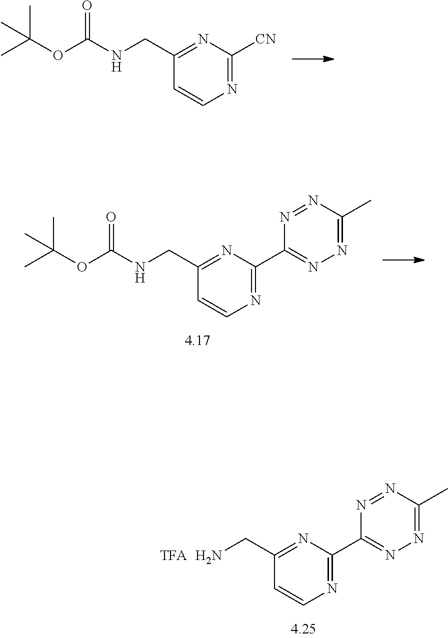 [SMILES: CC(C)(C)OC(=O)NCc1ccnc(C#N)n1 ; Cc1nnc(-c2nccc(CNC(=O)OC(C)(C)C)n2)nn1 ; Cc1nnc(-c2nccc(CN)n2)nn1 ; "TFA"]

Compound 4.25 was prepared according to general procedure B from 4.17. Pure 4.25 was obtained as a pink oil (35 mg, 0.11 mmol, 100%). ¹H-NMR (CD₃OD): δ=9.13 (d, 1H, ArH), 7.81 (d, 1H, ArH), 4.54 (s, 2H, CH₂), 3.18 (s, 3H, CH₃). ¹³C-NMR (CD₃OD): δ=170.6, 164.7, 163.7, 161.4 (q), 160.11, 160.08, 122.4, 117.3 (q), 43.5, 21.5. ¹⁹F-NMR (CD₃OD): δ=−77.4. ESI-MS: m/z Calc. for C₁₀H₁₀F₃N₇O₂ 317.08; Obs. [M-TFA+H]⁺ 204.17.

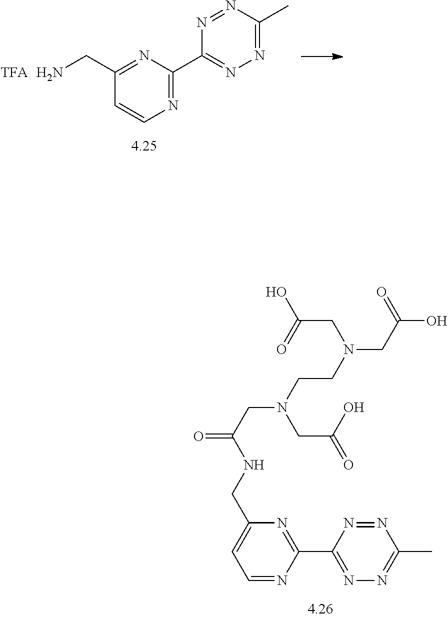 [SMILES: Cc1nnc(-c2nccc(CN)n2)nn1 ; "TFA" ; Cc1nnc(-c2nccc(CNC(=O)CN(CCN(CC(=O)O)CC(=O)O)CC(=O)O)n2)nn1]

4.26

A solution of 4.25 (35 mg, 0.11 mmol) in dry DMSO (2 mL) was added to a solution of ethylenediaminetetraacetic dianhydride (341 mg, 1.30 mmol, 12 eq) in dry DMSO (3 mL) under Ar. N,N′-diisopropylethylamine (230 μL, 1.30 mmol, 12 eq) was added dropwise over 5 min and the resulting mixture was stirred at room temperature for 1 h. Precipitation was induced by the addition of CHCl₃ (5 mL) and diisopropylether (ca. 30 mL) yielding a red solid that was filtrated, washed with diisopropylether and dried in vacuo. Purification was achieved with repeated RP-MPLC using an elution gradient of 2% to 12% MeCN in H₂O (both containing 0.1 v/v % formic acid). Lyophilization yielded pure 4.26 as a pink, fluffy solid (14.5 mg, 30 μmol, 28%). ¹H-NMR (D₂O): δ=9.04 (d, 1H, ArH), 7.79 (d, 1H, ArH), 3.93 (s, 4H, CH₂COOH), 3.83 (s, 2H, NCH₂), 3.76 (s, 2H, NCH₂), 3.51 (t, 2H, CH₂CH₂), 3.32 (t, 2H, CH₂CH₂), 3.19 (s, 3H, CH₃). The TZCH₂ signal is not observed since it overlaps with the residual H₂O peak at δ=4.79. ¹³C-NMR (D₂O): δ=173.2, 171.7, 170.5, 169.1, 168.4, 162.1, 158.6, 157.8, 120.7, 57.0, 56.0, 55.5, 52.3, 50.2, 43.9, 20.5. ESI-MS: m/z Calc. for C₁₈H₂₃N₉O₇ 477.17; Obs. [M+H]⁺ 478.25.

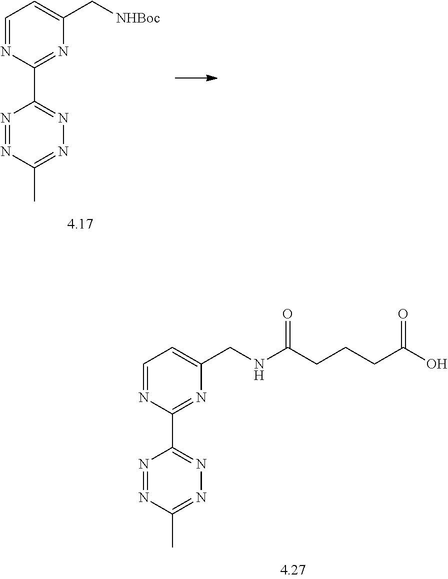 [SMILES: Cc1nnc(-c2nccc(CNC(=O)OC(C)(C)C)n2)nn1 ; Cc1nnc(-c2nccc(CNC(=O)CCCC(=O)O)n2)nn1]

Compound 4.27 was prepared from 4.17 according to general procedure B followed by general procedure D. Preparative RP-HPLC purification using an elution gradient of 5% to 40% MeCN in H₂O (both containing 0.1% TFA) followed by lyophilization yielded pure 4.27 (60 mg, 189 μmol, 26%) as a pink fluffy solid. ¹H NMR (400 MHz, CD₃OD): δ 9.04 (d, 1H, ArH), 7.70 (d, 1H, Aril) 4.68 (d, 2H, CH₂NH), 3.17 (s, 3H, TZCH₃), 2.44 (t, 2H, CH₂C(O)OH), 2.38 (t, 2H, NHC(O)CH₂), 1.97 (qn, 2H, CH₂CH₂CH₂) ppm. ESI-MS: m/z Calc, for C₁₃H₁₅N₇O₃ 317.12; Obs. [M−H]⁻ 316.08, [2M−H]⁻ 632.72, [M+H]⁺ 317.84, [2M+H]⁺ 634.36.

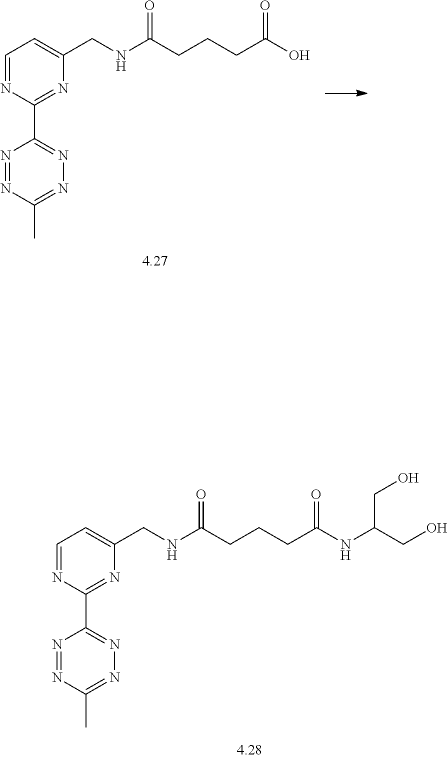 [SMILES: Cc1nnc(-c2nccc(CNC(=O)CCCC(=O)O)n2)nn1 ; Cc1nnc(-c2nccc(CNC(=O)CCCC(=O)NC(CO)CO)n2)nn1]

4.28:
4.27 (10 mg, 31.5 μmol), serinol (4.3 mg, 47.3 μmol), PyBOP (24.6 mg, 47.3 mmol) and DiPEA (22 μL, 126.1 μmol) were stirred in DMF (0.5 mL) for 45 minutes at room temperature. The reaction mixture was diluted with H₂O/formic acid (99:1) followed by preparative RP-HPLC purification using an elution gradient of 5% to 40% MeCN in H₂O (both containing 0.1 (N) TFA). Lyophilization yielded pure 4.28 (6.0 mg, 15.4 μmol, 49%) as a pink solid. 1H NMR (400 MHz, CD₃OD): δ 9.04 (d, 1H, ArH), 7.72 (d, 1H, ArH) 4.69 (d, 2H, CH₂NH), 3.55-3.64 (m, 4H, CH(CH₂OH)₂), 3.25 (qn, 1H, NHCH(CH₂OH)₂), 3.17 (s, 3H, TZCH₃), 2.41 (t, 2H, CH₂C(O)NH), 2.38 (t, 2H, NHC(O)CH₂), 2.00 (qn, 2H, CH₂CH₂CH₂) ppm. ESI-MS: m/z Calc, for C₁₆H₂₂N₈O₄ 390.18; Obs. [M+H]⁺ 390.76, [2M+H]⁺ 780.16.

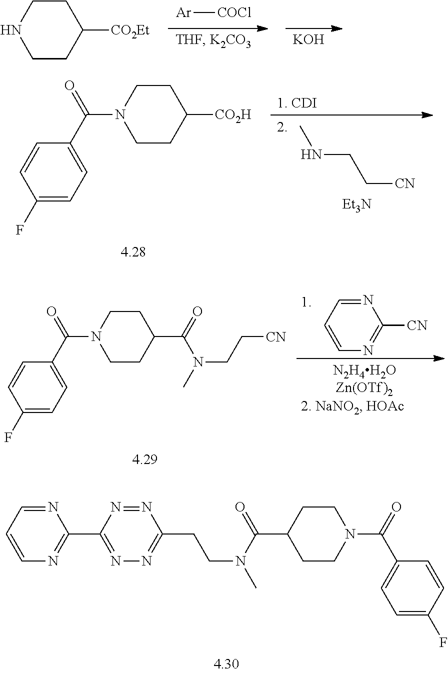

### 1-(4-fluorobenzoyl)piperidine-4-carboxylic Acid (4.28)

A solution of ethyl 4-isonipecotate (22.60 g, 0.144 mol) in 100 mL THF was cooled in ice. Potassium carbonate (37.18 g, 0.269 mol) was added, followed by 4-fluorobenzoyl chloride (25.80 g, 0.163 mol). The mixture was stirred for 2 h in the ice-bath, then 50 mL water was added over a 30 min period. The mixture was stirred at rt for 3 d, 25 mL 30% sodium hydroxide solution was added and the mixture was heated under reflux for 1½ h. Most of the THF was removed by rotary evaporation and the remainder was diluted with 75 mL water. The resulting solution was extracted with 2×150 mL toluene. The successive toluene layers were washed with 30 mL water. The combined aqueous layers were treated with 40 mL 37% hydrochloric acid, then with 40 g citric acid. The resulting suspension was extracted with 3×150 mL dichloromethane. Drying, rotary evaporation and heating under high vacuum at 80° C. left a residue of 37.5 g, which was used as such in the next step. ¹H-NMR (CDCl₃): δ 7.45 (m, 2H), 7.1 (m, 2H), 4.2-4.7 (broad s, 1H), 3.6-4.0 (broad s, 1H), 3.1 (m, 2H), 2.6 (m, 1H), 2.0 (broad s, 2H), 1.7 (broad s, 2H).

### N-(2-cyanoethyl)-1-(4-fluorobenzoyl)-N-methylpiperidine-4-carboxamide (4.29)

The crude acid of above (8.18 g, 32.5 mmol) was mixed with 40 mL dichloromethane, 1,1′-carbonyldiimidazole (9.05 g, 70 wt %, 39.1 mmol) was added and the mixture was heated under reflux for 30 min. 3-methylaminopropionitrile (5.0 g, 58.7 mmol) was added and the mixture was heated under reflux for 1 h, then stirred at 30° C. overnight. The solution was diluted with 60 mL dichloromethane and washed with a solution of 9.2 g citric acid in 50 mL water and with 2×50 mL water. The successive aqueous layers were extracted with 50 mL dichloromethane. Drying and rotary evaporation gave a residue which was chromatographed on silica, using heptane containing increasing amounts of ethyl acetate as the eluent, and finally with methanol in the eluent. Two product fractions were obtained, 5.39 g and 3.06 g (after heating under high vacuum at 90° C.). The 3.06 g portion contained a small amount of the amide, formed from the residual 4-fluorobenzoic acid and 3-methylaminopropionitrile. ¹H-NMR (CDCl₃): δ 7.4 (m, 2H), 7.1 (m, 2H), 4.4-4.8 (broad s, 1H), 3.7-4.1 (broad s, 1H), 3.6 (broad s, 2H), 3.2 (s, 3H), 3.0 (broad s, 2H), 2.8 (m, 1H), 2.65 (m, 2H), 1.8 (broad s, 4H).

### 1-(4-fluorobenzoyl)-N-methyl-N-(2-(6-(pyrimidin-2-yl)-1,2,4,5-tetrazin-3-yl)ethyl)piperidine-4-carboxamide (4.30)

A mixture of 2-cyanopyrimidine (4.05 g, 38.53 mmol) and 10 ml ethanol was cooled. 37% hydrochloric acid (3.76 g, 38.11 mmol) was added dropwise at <18° C., followed by 5 mL ethanol and then 10 mL hydrazine hydrate at <24° C. The amide (4.20 g, 13.24 mmol was added as a solution in 5 mL ethanol, followed by another 7 mL hydrazine hydrate (total 17 mL, 350 mmol). Zinc triflate (1.02 g, 2.81 mmol) was added and the mixture was brought to 62° C. over a 4 h period, stirred for 24h, and rotary evaporated. 50 mL water was added, the solution was partly evaporated and the residue was diluted with 50 mL ice-water. The mixture was extracted with dichloromethane. Drying and rotary evaporation gave a residue, which was dissolved in a mixture of 40 mL acetic acid and 10 mL THF. The mixture was cooled and sodium nitrite (3.07 g, 44.5 mmol) was added in portions at <5° C. The mixture was stirred for 1 h in ice, then 75 mL ice-water was added, and the product was extracted with dichloromethane. Drying and rotary evaporation gave a residue which was chromatographed on silica with heptane containing increasing amounts of acetone. The product fractions were combined and stirred with TBME until a homogeneous suspension was obtained. Filtration and washing gave 393 mg of product. ¹H-NMR (CDCl₃): δ 9.1 (d, 2H), 7.6 (m, 1H), 7.4 (m, 2H), 7.05 (m, 2H), 4.4-4.8 (broad s, 1H), 3.95 (broad s, 2H), 3.5-3.9 (broad s) and 3.65 (t) (3H), 3.15 (s, 3H), 2.8-3.1 (broad m, 2H), 2.7 (m, 1H), 1.5-2.0 (broad in, 4H). MS: 451.2 (M+1), 449.0 (M−1).

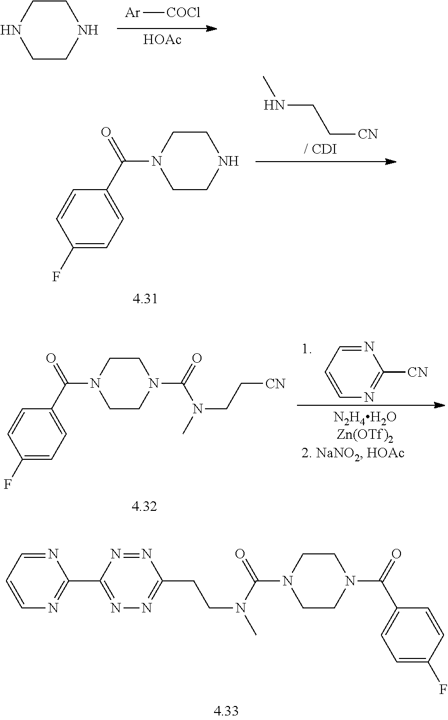

### (4-fluorophenyl)(piperazin-1-yl)methanone (4.31)

Piperazine (10.06 g, 117 mmol) was added in portions to 100 mL acetic acid. The solution was stirred for 1 h at 60° C., then 4-fluorobenzoyl chloride (18.19 g, 0.115 mol) was added. The resulting suspension was heated for 3 h at 66° C., rotary evaporated, then 100 mL dichloromethane was added, followed by 100 mL ice-water and then 30% sodium hydroxide solution until the water layer was basic. The layers were separated and the aqueous layer was extracted with 150 mL dichloromethane. Drying and rotary evaporation left a residue, which was stirred with a mixture of 100 mL TBME and 20 mL ethyl acetate until homogeneous. Filtration and washing gave a filtrate which was evaporated and the residue (15.15 g) was used as such in the next step. ¹H-NMR (CDCl₃): δ 7.40 (m, 2H), 7.08 (m, 2H), 3.3-3.9 (broad in, 4H), 2.87 (broad s, 4H).

### N-(2-cyanoethyl)-4-(4-fluorobenzoyl)-N-methylpiperazine-1-carboxamide (4.32)

1,1′-carbonyldiimidazole (2.45 g, 70 wt %, 10.6 mmol) was added to a solution of 3-methylaminopropionitrile (890 mg, 10.6 mmol) in 25 mL dichloromethane. The solution was stirred under reflux for 1 h, cooled to rt and amide 4.31 (1.80 g, 8.65 mmol) and toluene was added and the mixture was heated for 3 d at 80° C. The mixture was rotary evaporated and 100 mL toluene was added to the residue. The mixture was washed with 2×25 mL water, then dried and rotary evaporated to give the product (2.64 g) which was used as such in the next step. ¹H-NMR (CDCl₃): δ 7.4 (m, 2H), 7.1 (m, 2H), 3.2-3.8 (broad m) and 3.45 (t) (10H), 3.0 (s, 3H), 2.65 (t, 2H).

### 4-(4-fluorobenzoyl)-N-methyl-N-(2-(6-(pyrimidin-2-yl)-1,2,4,5-tetrazin-3-yl)ethyl)piperazine-1-carboxamide (4.33)

2-cyanopyrimidine (3.57 g, 34.0 mmol) was mixed with 5 mL ethanol, and 37% hydrochloric acid (3.33 g, 33.8 mmol) was added. The solution was cooled and 4.32 (2.64 g, 8.29 mmol) in 10 mL ethanol, was added dropwise at 15° C. followed by 12.5 mL hydrazine hydrate (257 mmol) at <27° C. Zinc triflate (0.485 g, 1.33 mmol) was added and the cooling bath was removed. The mixture was stirred for 15 min, then brought to 62° C. over 1 h. It was stirred at that temperature for 20 h, then rotary evaporated. The residue was oxidized by stirring with a mixture of 50 mL acetic acid and 16 mL THF and adding sodium nitrite (4.96 g, 0.179 mol) in portions at <9° C. The mixture was stirred for 1½ h in ice, then 100 mL ice-water was added, and the mixture was extracted with 2×100 mL dichloromethane. Drying and rotary evaporation gave a residue which was chromatographed on 55 g silica, with heptane containing increasing amounts of ethyl acetate, then with heptane containing 3% methanol affording impure product, which was purified on 40 g silica, using heptane-acetone, and then acetone. The product fractions were heated for 4 h with 40 mL TBME, followed by stirring at rt until a homogeneous suspension was obtained. Filtration and washing gave 220 mg product (0.49 mmol, 6%). ¹H-NMR (CDCl₃): δ 9.1 (d, 2H), 7.6 (m, 1H), 7.4 (m, 2E1), 7.1 (m, 2H), 3.3-3.9 (broad in) and 3.85 (t) and 3.75 (t) (8H), 3.1 (broad s, 4H), 3.0 (s, 3H).

MS: 452.2 (M+1), 450.1 (M−1).

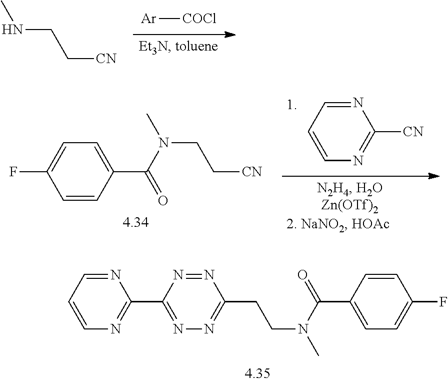

### N-(2-cyanoethyl)-4-fluoro-N-methylbenzamide (4.34)

4-fluorobenzoyl chloride (10.05 g, 63.4 mmol) in 10 mL toluene was added dropwise to a water-cooled mixture of 3-methylaminopropionitrile (5.16 g, 61.3 mmol), triethylamine (10.1 g, 100 mmol) and 70 mL toluene. The mixture was stirred for 1½ h at rt, then 25 mL water was added and the mixture was stirred for 10 min. The layers were separated and the organic layer was washed with 2×25 mL water, then dried and rotary evaporated. The residue (11.59 g, 56.2 mmol, 92%) was used as such in the next step. ¹H-NMR (CDCl₃): δ 7.45 (m, 2H), 7.1 (m, 211), 3.75 (broad s, 2H), 3.15 (s, 3H), 2.8 (broads, 2H).

### 4-fluoro-N-methyl-N-(2-(6-(pyrimidin-2-yl)-1,2,4,5-tetrazin-3-yl)ethyl)benzamide (4.35)

A mixture of 2-cyanopyrimidine (10.0 g, 95.1 mmol) and 25 ml ethanol was cooled. 37% hydrochloric acid (9.40 g, 95.3 mmol) was added dropwise at <14° C., followed by 5 mL ethanol and then 20 mL hydrazine hydrate at <25° C. The amide 4.34 (9.78 g, 47.43 mmol) was added, followed by another 15 mL hydrazine hydrate (total 35 mL, 0.72 mmol) and 5 mL ethanol. Zinc triflate (2.0 g, 5.50 mmol) was added and the mixture was brought to 62° C. over a 4 h period. It was stirred at that temperature for 18h, then rotary evaporated. 50 mL water was added, the solution was partly rotary evaporated and diluted with 75 mL water. The mixture was extracted with 3×75 mL dichloromethane. Drying and rotary evaporation gave 16.0 g residue, which was dissolved in a mixture of 60 mL acetic acid and 25 mL THF. The mixture was cooled and sodium nitrite (8.5 g, 0.123 mol) was added in portions over a 30 min period at <7° C. The mixture was stirred for 1½ h in ice, then 100 mL ice-water was added, and the mixture was extracted with 2×75 mL dichloromethane. Drying and rotary evaporation gave ca. 10 g residue which was chromatographed on 74 g silica. Elution was done with heptane containing increasing amounts of ethyl acetate. The product fractions were combined and stirred with 50 mL TBME until a homogeneous suspension was obtained. Filtration and washing gave 4.20 g product (12.38 mmol, 26%). ¹H-NMR (CDCl₃): δ 9.1 (d, 2H), 7.6 (m, 1H), 7.4 (m, 2H), 7.05 (m, 2H), 4.1 (broad s, 2H), 3.8 (broad s, 2H), 3.05 (broad s, 3H). MS: 340.1 (M+1), 338.0 (M−1).

### Example 5: Other TZ Derivatives

The synthesis of 2,2′,2″-(10-(2,40,44-Trioxo-44-((6-(6-(pyridine-2-yl)-1,2,4,5-tetrazin-3-yl)pyridine-3-yl)amino)-6,9,12,15,18,21,24,27,30,33,36-undecaoxa-3,39-diazatetratetracontyl)-1,4,7,10-tetraazacyclododecane-1,4,7-triyl)triacetic acid (5.1) was reported in Rossin et al., Angew. Chem. Int. Ed. 2010, 49, 3375-3378. Compounds 1,1′-(1,2,4,5-tetrazine-3,6-diyl)bis(3-oxo-5,8,11,14,17,20-hexaoxa-2-azadocosan-22-oic acid) (5.2) and Tz-bis-DOTA (5.3) were synthesized following general procedures described in Example 1.

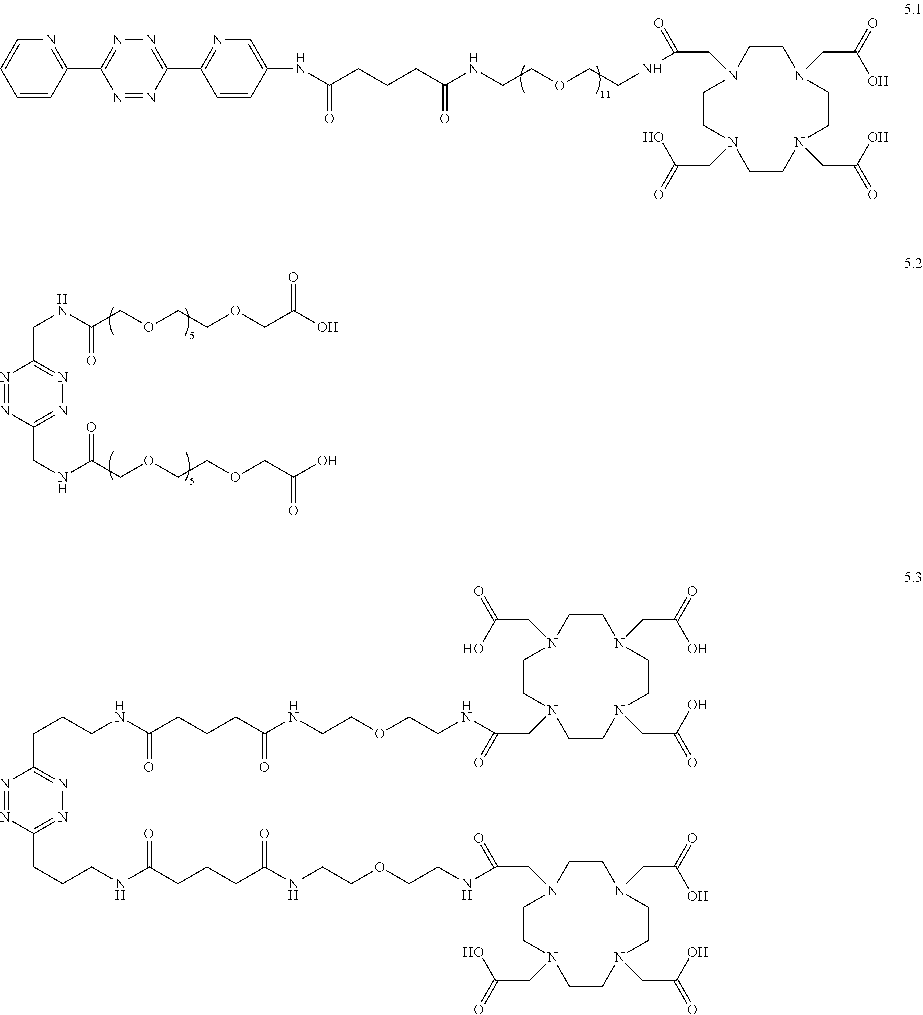

Synthesis routes to additional alkyl-pyrimidyl TZ building blocks and activators:

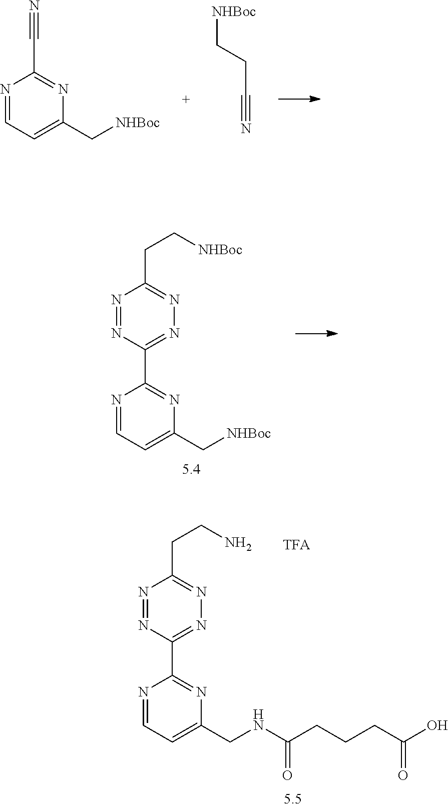

Compound 5.4 can be prepared from t-butyl N-((2-cyano-4-pyrimidinyl)methyl)carbamate (Sweeney et al, ACS Med. Chem. Lett. 2014, 5, 937-941) and tert-butyl N-(2-cyanoethyl)carbamate that are reacted in a 10:1 molar ratio. Oxidation is performed according to general procedure A1. Column chromatography (flash SiO2) using an elution gradient of EtOAc in CHCl₃ and, in a second chromatography step (normal SiO2), elution with acetone in heptane yields 5.4.

Compound 5.5 can be prepared from compound 5.4 generated according to general procedure B and D. After removal of the boc protecting groups, the tetrazine intermediate is reacted with 1.0 eq. glutaric anhydride. Purification with preparative RP-HPLC using an elution gradient MeCN in H2O (both containing 0.1% formic acid) to achieve separation of 5.5 from the tetrazine that is functionalized on the ethyl amine, that is functionalized on both amines, or that is functionalized on neither amine. Lyophilization yields 5.5.

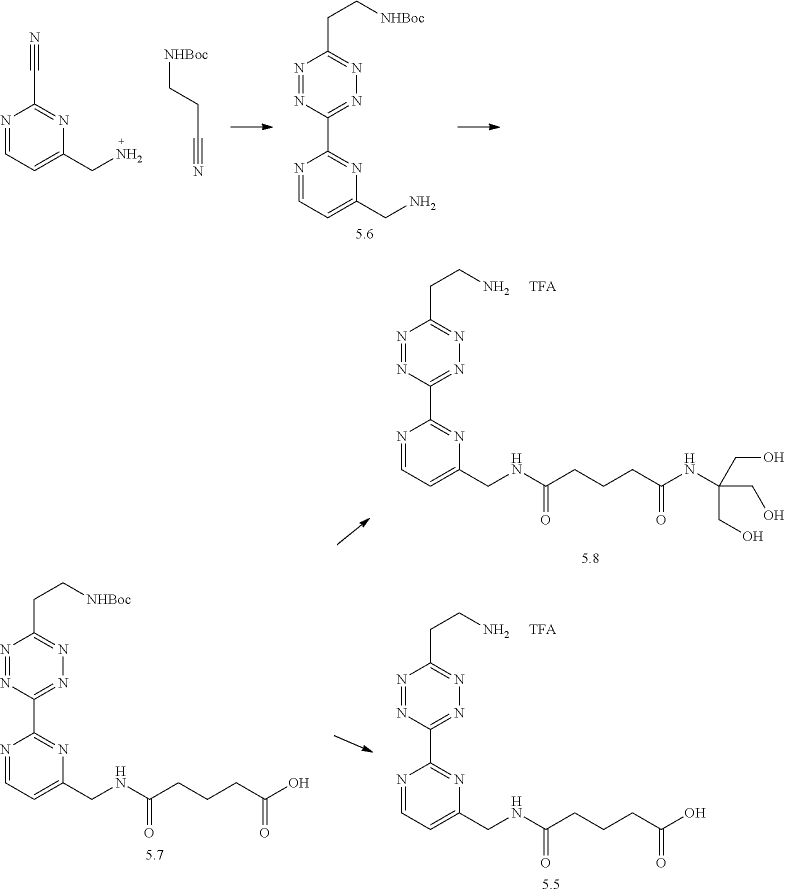

In an alternative synthesis, compound 5.5 can be generated via intermediate 5.6 and intermediate 5.7.

Compound 5.6 can be prepared according to general procedure A from 4-(aminomethyl)pyrimidine-2-carbonitrile and tert-butyl N-(2-cyanoethyl)carbamate that are reacted in a 10:1 molar ratio. Oxidation is performed according to general procedure A1. Column chromatography (flash SiO2) using an elution gradient of EtOAc in CHCl₃ and, in a second chromatography step (normal SiO2), elution with acetone in heptane yields 5.6.

Compound 5.7 can be generated according to general procedure D from compound 5.6. Column chromatography (flash SiO2) using an elution gradient of MeOH in CHCl₃ yields 5.7.

Compound 5.5 can be generated according to general procedure B from compound 5.7. Purification with preparative RP-HPLC using an elution gradient MeCN in H2O (both containing 0.1% formic acid) yields 5.5. An alternative method of purification, uses column chromatography (flash SiO2) with an elution gradient of EtOAc in CHCl₃ and, in a second chromatography step (normal SiO2), elution with acetone in heptane yielding 5.5.

Compound 5.8 can be generated from compound 5.5 that is reacted with 2-amino-2-(hydroxymethyl)propane-1,3-diol (1.1 eq.) and DiPEA (3 eq.) and PyBOP (1.1 eq.) in DMF. The mixture is stirred at room temperature for 30 min. After removal of the solvent in vacuo, the boc protecting group is removed according to general procedure B. Purification with preparative RP-HPLC using an elution gradient of MeCN in 120 (both containing 0.1% formic acid) followed by lyophilization yields 5.8.

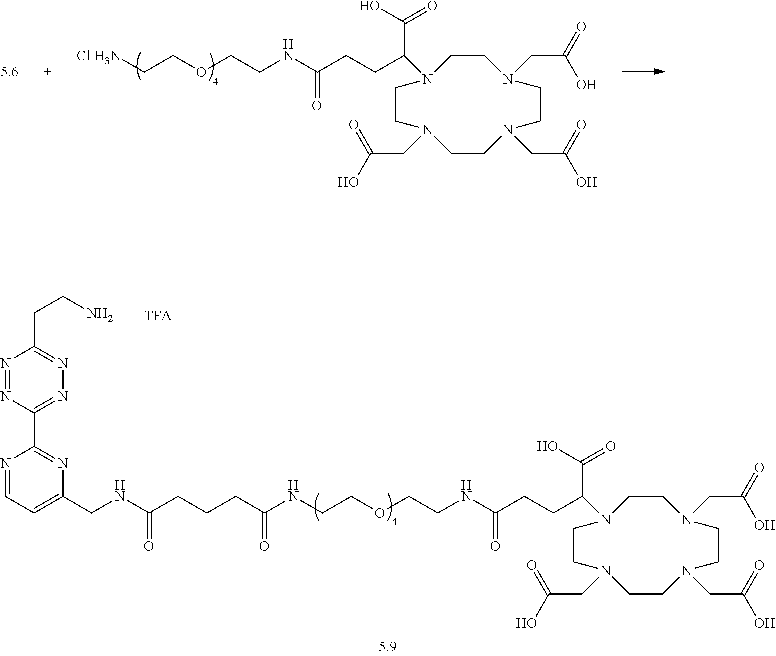

Compound 5.9 can be generated from compound 5.6 that is reacted with DiPEA (3 eq.) and PyBOP (1.1 eq.) in DMF and stirred for 15 minutes. A solution of 2,2′,2″-(10-(1-amino-19-carboxy-16-oxo-3,6,9,12-tetraoxa-15-azanonadecan-19-yl)-1,4,7,10-tetraazacyclododecane-1,4,7-triyl)triacetic acid (1 eq.) and DiPEA (5 eq.) in DMF is added and after stirring for 30 minutes the solvent is removed in vacuo. The boc protecting group is removed according to general procedure B. Purification with preparative RP-HPLC using an elution gradient of MeCN in H₂O (both containing 0.1% formic acid) followed by lyophilization yields 5.9.

Synthesis routes to additional 3,6-bisalkyl TZ building blocks and activators

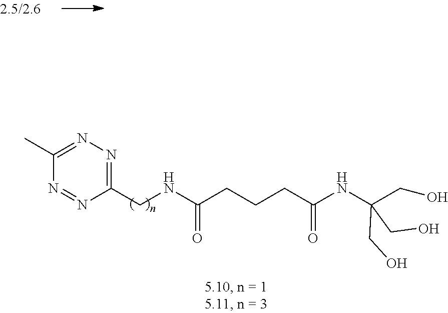

Compounds 5.10 and 5.11 can be generated from compounds 2.5 and 2.6 that are reacted with 2-amino-2-(hydroxymethyl)propane-1,3-diol (1.1 eq.) and DiPEA (3 eq.) and PyBOP (1.1 eq.) in DMF, the mixture stirred at rt for 30 min. After removal of the solvent in vacuo, purification with preparative RP-HPLC using an elution gradient of MeCN in H₂O (both containing 0.1% formic acid) followed by lyophilization affords 5.10 and 5.11.

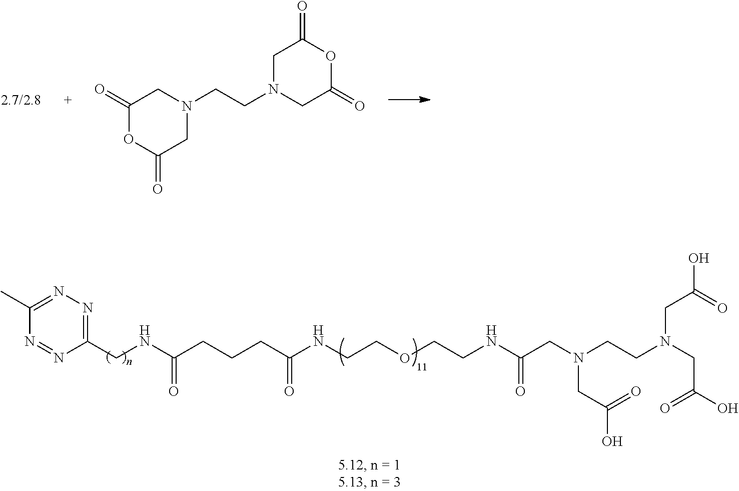

Compounds 5.12 and 5.13 can be generated from compounds 2.7 and 2.8. The boc protecting group is removed according to general procedure B. The resulting intermediates are reacted with DiPEA (4 eq.) and 4,4′-Ethylenebis(2,6-morpholinedione) (12 eq.) in DMSO and stirred for 30 minutes. The reaction mixtures are diluted with 0.1M HCl and purification with preparative RP-HPLC using an elution gradient of MeCN in H₂O (both containing 0.1% formic acid) followed by lyophilization affords 5.12 and 5.13.

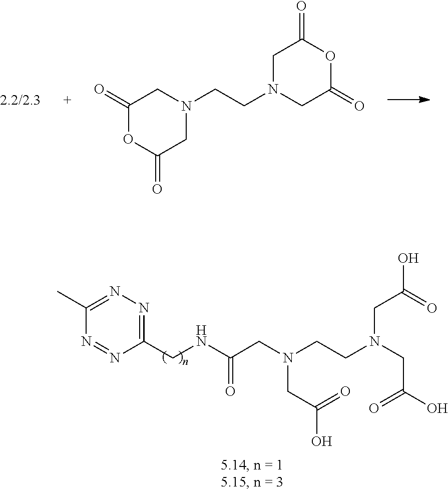

Compound 5.14 and 5.15 can be generated from compounds 2.3 and 2.4. The boc protecting group are removed according to general procedure B. The resulting intermediates are reacted with DiPEA (4 eq.) and 4,4′-Ethylenebis(2,6-morpholinedione) (12 eq.) in DMSO and stirred for 30 minutes. The reaction mixtures are diluted with 0.1M HCl and purification with preparative RP-HPLC using an elution gradient of MeCN in H₂O (both containing 0.1% formic acid) followed by lyophilization yields 5.14 and 5.15.

Synthesis routes to additional 3-alkyl-6-pyridyl TZ precursors and activators

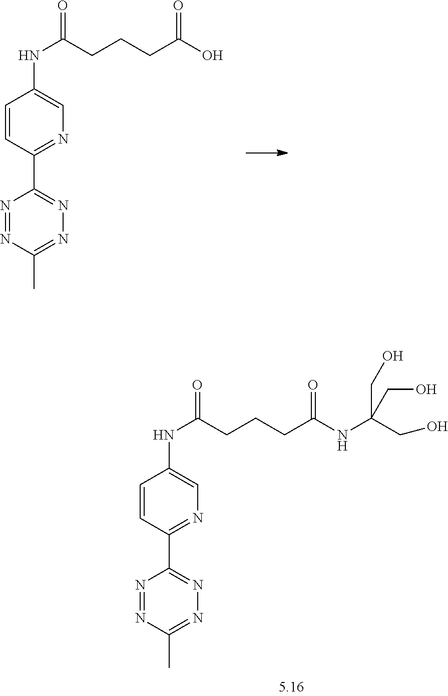 [SMILES: Cc1nnc(-c2ccc(NC(=O)CCCC(=O)O)cn2)nn1 ; Cc1nnc(-c2ccc(NC(=O)CCCC(=O)NC(CO)(CO)CO)cn2)nn1]

Compound 5.16 can be generated from previously reported 5-((6-(6-methyl-1,2,4,5-tetrazin-3-yl)pyridin-3-yl)amino)-5-oxopentanoic acid (Rossin et al., Bioconjug. Chem., 2016, 27, 1697-1706) that is reacted with 2-amino-2-(hydroxymethyl)propane-1,3-diol (1.1 eq.) and DiPEA (3 eq.) and PyBOP (1.1 eq.) in DMF. The mixture is stirred at room temperature for 30 min. After removal of the solvent in vacuo, purification with preparative RP-HPLC using an elution gradient of MeCN in H₂O (both containing 0.1% formic acid) followed by lyophilization yields 5.16.

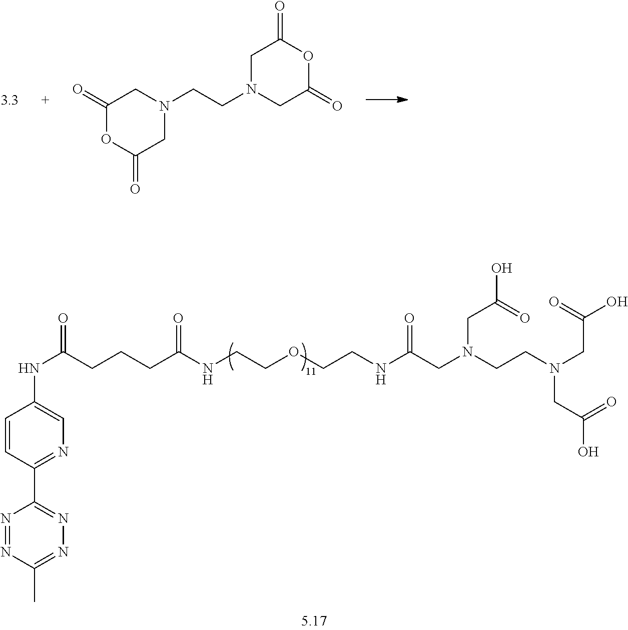

Compound 5.17 can be generated from compound 3.3. The boc protecting group is removed according to general procedure B. The resulting intermediate is reacted with DiPEA (4 eq.) and 4,4′-Ethylenebis(2,6-morpholinedione) (12 eq.) in DMSO and stirred for 30 minutes. The reaction mixture is diluted with 0.1M HCl and purification with preparative RP-HPLC using an elution gradient of MeCN in H₂O (both containing 0.1% formic acid) followed by lyophilization affords 5.17.

### Example 6: In Vitro Microsomal Stability of TZ Activators

The in vitro stability of activators 4.11, 4.13, and 4.15 was tested in the presence of human, mouse, rat and cynomolgus microsomes after 30 min incubation (normalized to 100% at t=0). Control samples were incubated with the same concentration of BSA. The activators (5 μM) were incubated in 20 mM phosphate buffer pH 7.4 containing the microsomes (0.5 mg/mL), 1 mM NADP, 5 mM glucose-6-phosphate, 1 U/mL glucose-6-phosphate dehydrogenase, and 2 mM MgCl₂ at 37° C. and the measurements were performed by LC-MS. The amount of activator in solution was quantified using calibration curves.

TABLE 2
In vitro microsomal stability of TZ
activators after 30 min incubation at
37° C.

| Compound | Human | Mouse | Rat | Cynomolgus | BSA |
|---|---|---|---|---|---|
| 4.11 | 101.6 ± | 89.9 ± | 79.8 ± | 103 ± 8.2% | 100.5 ± |
| | 3.1% | 2.1% | 4.6% | | 6.2% |
| 4.13 | 86.5 ± | 73.3 ± | 82.6 ± 3.6 | 79.3 ± 2.2 | 99.2 ± 5.2 |
| | 3.3% | 2.6% | | | |
| 4.15 | 87.7 ± | 81.2 ± | 91.1 ± | 85.6 ± 0.5% | 87.5 ± |
| | 2.6% | 5.7% | 11.8% | | 7.2 % |

### Example 7: In Vitro Stability and Reactivity of TZ Activators

The stability of tetrazines was evaluated by dissolving them in 10% MeCN/PBS at 37° C. and following the decrease of the tetrazine UV absorbance at 520 nm in time (Table 4).

The second-order reaction rate constant of the reaction of axial (E)-cyclooct-2-en-1-yl N-methyl benzylcarbamate with a range of tetrazines was determined in 25% MeCN/PBS at 20° C. by UV spectroscopy. A cuvette was filled with 3 mL of a 83 μNI solution of the appropriate TZ in 25% MeCN/PBS (0.25 μmol), and equilibrated at 20° C. Subsequently, a stock-solution of the TCO derivative was added (10 μL 25 mM in DMSO; 0.25 μmol). The second-order reaction rate constants were calculated from the rate at which the absorption at 520 nm (specific for the TZ moiety) decreased (Table 4).

The reaction kinetics between a diabody-based TCO-linked ADC according to this invention (AVP0458-TCO-MMAE) and activators 2.12 and 4.11 were determined as published in Rossin et al. Angew. Chem. 2010, 49, 3375-3378. Compounds 2.10 and 4.11 were radiolabeled with no-carrier added lutetium-177 and indium-111, respectively. The obtained radioactive activators were then reacted with increasing concentrations of AVP0458-TCO-MMAE (DAR=4) in PBS at 37° C. in pseudo-first order conditions. The obtained [¹⁷⁷Lu]Lu-2.12 ca. 0.2 μM was reacted with 0.6-1.8 μM ADC while [¹¹¹In]In-4.11 (ca. 93 nM) was reacted with 0.2-0.8 μM ADC. In these conditions k₂ values of 54.7±2.2 M⁻¹s⁻¹, and 375.9±43.2 M⁻¹-s⁻¹ were calculated for activators 2.12, and 4.11 respectively.

### Example 8: In Vitro Drug Release from ADC Upon Activation

Doxorubicin (Dox) release from the TCO-linked ADC CC49-TCO-Dox with various TZ activators in PBS and 50% mouse serum at 37° C. was evaluated in vitro as described in Rossin et al. Bioconjug. Chem., 2016, 27, 1697-1706. The results of these experiments are depicted in Tables 3 (PBS) and 4 (mouse serum). The release reactions were performed in triplicate unless otherwise stated in the result table.

TABLE 3
In vitro Dox release (%) from CC49-Dox
at 37° C. in PBS at various times

| Compound | 1 h | 2 h | 3 h | 24 h | 48 h |
|---|---|---|---|---|---|
| 2.1 | 76.1 ± | 78.5 ± | 78.8 ± | 80.6 ± | 81.5 ± |
| | 4.4 | 4.8 | 6.1 | 3.9 | 0.4 |
| 2.2 | 44.9 ± | 51.3 ± | 54.6 ± | 62.3 ± | |
| | 2.7 | 3.9 | 3.2 | 4.2 | |
| 2.10^{a} | 59.1 | 64.5 | 65.2 | 75.8 | 83.7 |
| 2.11^{a} | 51.0 | 57.6 | 59.1 | 68.7 | 72.8 |
| 3.1 | 55.1 ± | 55.9 ± | 58.9 ± | 59.6 ± | |
| | 0.5 | 0.7 | 2.2 | 1.1 | |
| 3.2 | 44.3 ± | 44.7 ± | 47.5 ± | 45.0 ± | |
| | 0.9 | 0.4 | 0.4 | 0.6 | |
| 5.1 | 14.2 ± | 20.4 ± | 24.3 ± | 48.1 ± | 54.9 ± |
| | 0.4 | 0.6 | 1.4 | 1.6 | 3.6 |
| 5.3^{b} | 50.5 ± | 59.3 ± | 62.5 ± | 80.3 ± | 91.2 ± |
| | 5.2 | 3.9 | 3.7 | 3.2 | 4.3 |

^{a}n = 1;
^{b}n = 2.

TABLE 4
In vitro Dox release (%) from CC49-Dox
at 37° C. in 50% mouse serum at
various times

| Compound | 1 h | 2 h | 3 h | 6 h |
|---|---|---|---|---|
| 2.1 | 64.6 ± 1.0 | 68.7 ± 1.1 | 69.7 ± 1.0 | |
| 2.2 | 34.0 ± 1.2 | 43.4 ± 1.5 | 48.6 ± 1.2 | |
| 2.6^{a} | 60.1 | 66.9 | 69.2 | 69.4 |
| 3.1 | 53.8 ± 1.7 | 57.1 ± 1.8 | 59.6 ± 2.0 | |
| 3.2 | 36.1 ± 0.1 | 39.8 ± 0.2 | 42.0 ± 0.4 | |
| 5.1 | 13.7 ± 0.7 | 15.2 ± 1.4 | 14.8 ± 0.8 | |
| 5.3 | 43.6 ± 3.5 | 54.9 ± 6.0 | 64.4 ± 3.3 | 67.5 ± 8.3 |

^{a}n = 1;
^{b}n = 2.

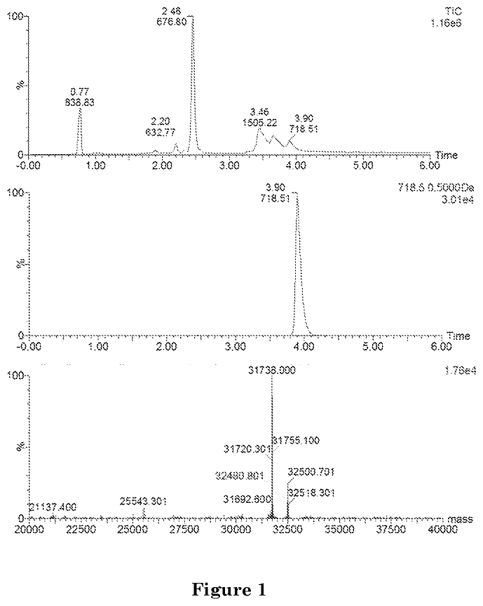

Monomethylauristatin E (MMAE) release from diabody ADCs (AVP0458-TCO-MMAE) and AVP06-TCO-MMAE with activator 2.12 was tested in PBS and 50% mouse serum at 37° C., as described in Rossin et al., Nature Communications 2018, 9, 1484. An aliquot of ADC solution (10 μL 2 μg/μL in phosphate buffer pH 6.8 containing 2 mM EDTA (EDTA-PB) and 5% DMSO) was diluted with PBS (90 μL), mixed with activator 2.12 (5 μL 2.5 mM in PBS; 1.25×10⁻⁸ mol) and incubated at 37° C. for 1 h. Subsequent HPLC-QTOF-MS analysis demonstrated the formation of free MMAE (m/z=+718.51 Da) and the diabody reaction products without MMAE (FIG. 1, ca. 90% release after 1h incubation). Similar results were obtained for the analogous experiment with AVP06-TCO-MMAE.

An aliquot of ADC solution (2 μg/μL in 5% DMSO/EDTA-PB) was ten-fold diluted with PBS. Subsequently 50 μL of this solution was two-fold diluted with mouse serum and activator 2.12 was added (6.5 μL, 5 mM) followed by incubation at 37° C. After 10 min, 1 h, and 20 h aliquots of the solution were taken and proteins were precipitated by adding two parts of ice-cold acetonitrile. After vortexing, 10 min standing at −20° C. and centrifugation, the supernatants were separated from the protein pellets, diluted with five parts of PBS and analysed by HPLC-QTOF-MS (FIG. 1). The reactions were performed in triplicate. MMAE recovery was quantified using calibration curves in 50% mouse serum. With this procedure, 26±3%, 51±3%, and 80±2% MMAE release was observed at 10 min, 1 h and 20 h, respectively.

In an alternative method MMAE release from diabody ADC (AVP0458-TCO-MMAE) was determined by using a deuterated internal standard D8-MMAE. ADC (1.1×10⁻¹⁰ moles bound MMAE) and D8-MMAE (1.1×10⁻¹⁰ moles) and 10 equiv of tetrazine were incubated in 100 uL PBS/plasma (1/1) at 37° C. After 24 h the samples (n=3) were process as above and the ratio MMAE/D8-MMAE was measured with LC-SIM-MS, affording the release yields (Table 5).

TABLE 5
In vitro tetrazine stability
(t_{1/2} in 10% MeCN/PBS at
37° C.), reactivity (k₂
(M⁻¹ s⁻¹) in 25% MeCN/PBS
at 20° C.), and induced MMAE
release from AVP0458-
TCO-MMAE at 37° C. after 24 h.

| Compound | stability (h) | reactivity(k₂) | release (%) |
|---|---|---|---|
| 2.1 | 14 | 14 | 95 ± 0.4 |
| 2.11 | n.d. | n.d. | 72 ± 0.5 |
| 2.12 | n.d. | n.d. | 88 ± 0.1 |
| 3.4 | n.d. | n.d. | 47 ± 0.3 |
| 4.1 | 10 | 250 | 69 ± 0.0 |
| 4.2 | 14 | n.d. | 56 ± 0.2 |
| 4.3 | 18 | 275 | 62 ± 0.5 |
| 4.4 | 15 | n.d. | 61 ± 0.4 |
| 4.11 | n.d. | n.d. | 56 ± 0.3 |
| 4.12 | n.d. | n.d. | 67 ± 0.4 |
| 4.13 | n.d. | n.d. | 61 ± 0.1 |
| 4.15 | n.d. | n.d. | 55 ± 0.4 |
| 4.17 | 12 | 412 | 70 ± 0.1 |
| 4.18 | 16 | 150 | 55 ± 0.8 |
| 4.19 | 1 | 290 | 60 ± 0.1 |
| 4.20 | 15 | 28 | 59 ± 0.3 |
| 4.23 | 6 | 246 | 69 ± 0.5 |
| 4.24 | 13 | n.d. | n.d. |
| 4.26 | 13 | 135 | 59 ± 0.6 |
| 4.27 | n.d. | n.d. | 66 ± 0.5 |
| 4.33 | n.d. | n.d. | 53 ± 0.6 |
| 4.35 | n.d. | n.d. | 67 ± 0.1 |
| 5.5 | n.d. | n.d. | 81 ± 0.4 |
| 5.8 | n.d. | n.d. | 80 ± 0.5 |

### Example 9: In Vivo Reactivity of TZ Activators—Tumor Blocking Experiments

The animal studies were performed in accordance with the principles established by the revised Dutch Act on Animal Experimentation (1997) and were approved by the institutional Animal Welfare Committee of the Maastricht University and Radboud University Nijmegen. The colorectal cancer (LS174T) mouse model was reported in Rossin et al. Bioconjug. Chem., 2016, 27, 1697-1706.

To assess the in vivo reaction of TZ activators towards TCO-containing ADCs, tumor blocking experiments were performed as described in Rossin et al., Bioconjug. Chem. 2016, 27, 1697-1706. Briefly, the highly reactive probe 5.1 was used as a reporter to show the presence of residual (unreacted) TCO moieties in the tumors of mice treated with a TCO-containing ADC followed by an activator, in comparison to mice that did not receive any activator.

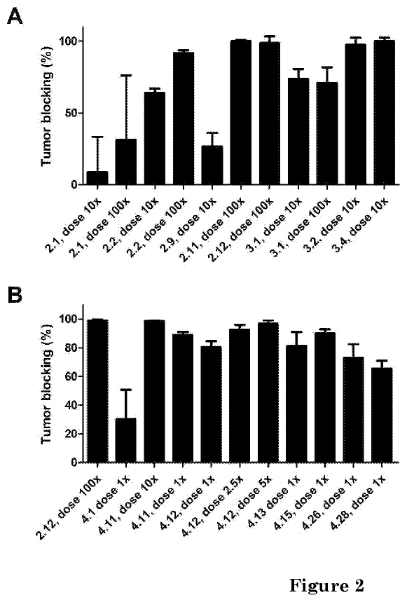

A series of 3,6-bisalkyl TZ activators (2.1, 2.2, 2.9, 2.11, 2.12) and 3-alkyl-6-pyridyl TZ activators (3.1, 3.2, 3.4) was tested in tumor-bearing mice (n=3-4) pretreated with an IgG-based ADC (CC49-TCO-Dox, DAR ca. 2) at a 5 mg/kg dose. A clearing agent (galactose-albumin-TZ, Rossin et al., J. Nucl. Med. 2013, 54, 1989-1995) was administered to the mice 24 post-ADC injection followed by the TZ activator (dose 10×: ca. 0.033 mmol/kg; close 100×: ca. 0.335 mmol/kg) 2 h later. One-hour after activation the mice were administered the highly reactive radiolabeled probe [¹⁷⁷Lu]Lu-5.1 (ca. 0.335 μmol/kg, ca. 1.5 MBq/mouse) and were euthanized 3 h later. All injections were performed intravenously. Tumors were harvested and the radioactivity was measured by γ-counting along with standards to determine the % injected dose per gram (% ID/g). The [¹⁷⁷Lu]Lu-5.1 uptake in tumor was corrected for non-TCO-specific retention (uptake in tumors of non-ADC-pretreated mice) and normalized to the maximum (uptake in tumors of ADC-pretreated mice that did not receive any activator). The tumor blocking capacity of TZ activators (signifying in vivo reaction between activator and tumor-bound TCO) at the administered dose was estimated from the difference between the maximum and the tumor uptake in each experimental group (FIG. 2A) with the formula:

$$Tumor blocking ( \% ) = ( 100 - \frac{Tumor uptake}{Tumor {uptake}_{Max}} \times 100 )$$

A series of 3-pyrimidyl-6-alkyl TZ activators (4.1, 4.11, 4.13, 4.15, 4.26, 4.28) was then compared to compound 4.12 in LS174T tumor-bearing mice (n=3-5) pretreated with a diabody-based ADC (AVP0458-TCO-MMAE; DAR=4). The mice were injected the ADC at a ca. 2 mg/kg dose followed 48 h later by the activator (dose 1×: ca. 3.35 μmol/kg; dose 2.5×: ca. 8.37 mol/kg; dose 5×: ca. 0.017 mmol/kg; dose 10×: ca. 0.033 mmol/kg; dose 100×: ca. 0.335 mmol/kg) and, 1 h post-activator, by the [¹¹¹In]In-5.1 probe. Three hours (4.1, 4.11, 4.12, 4.3, 4.15) or 24 h (4.26, 4.28) post-probe injection the mice were euthanized and the tumor blocking capacity of the various activators at the administered dose (FIG. 2B) was calculated as reported above.

From FIGS. 2A and 2B it becomes clear TZ's 2.1 and 4.1 perform much worse that the other TZ's that comprise a moiety with a mass of at least 100 Da

### Example 10: Drug Concentration in Tumors Upon In Vivo ADC Activation

Groups of LS174T tumor-bearing mice (n=3) were injected a diabody-based ADC (AVP0458-TCO-MMAE; ca. 2 mg/kg) followed 48 h later by compound 2.12 (0.335 mmol/kg dose) or vehicle and were euthanized 72 or 96 h post-ADC injection. One extra group of mice was injected with a diabody-ADC containing the valine-citrulline enzymatically cleavable linker (AVP0458-vc-MMAE (vc-ADC); ca. 2 mg/kg) and were euthanized 24 h later. Tumor, liver and plasma samples were harvested from all mice and added with an internal standard (d8-MMAE) and MMAE concentration in the samples was determined as described in Burke et al., Mol. Cancer Ther. 2017, 16, 116-123. The sample extracts were then analysed by LC-QTOF-MS to quantify the amount of free MMAE. Tumour, liver and plasma samples from non-treated mice added with ADC anchor d8-MMAE were used as controls. The limit of detection for MMAE in this assay was 0.2 nM.

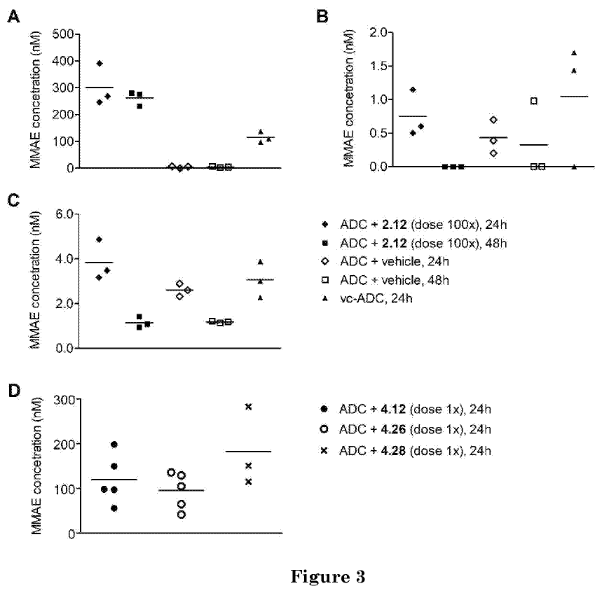

The activation of tumor-bound TCO-ADC gave high and sustained MMAE tumor levels 24 h and 48 h after injection of 2.12, indicating that tumor washout of MMAE, if any, is minimal (FIG. 3A). In comparison, a 2-3 fold lower MMAE concentration was detected in the tumors of mice 24 h after the administration of the enzymatically cleavable vc-ADC. Furthermore, the MMAE levels were more than 100-fold lower in plasma (FIG. 3B) and liver (FIG. 3C) and in tumors that only received the ADC and not 2.12, underlining the very favorable biodistribution of the ADC, its stability and its TZ-dependent release.

Three more groups of LS174T tumor bearing mice (n=3-5) were pre-treated with AVP0458-TCO-MMAE (ca. 2 mg/kg) followed 48 h later by a low close of activator 4.12, 4.26 or 4.28 (ca. 3.35 μmol/kg). Twenty-four hours post-activation the tumors were harvested and the content of free MMAE was determined as described above. Despite the 100-fold lower dose of activator used in these experiments, a high amount of free MMAE was found in tumors (100-180 nM, FIG. 3D).

### Example 11 Cytotoxicity of ADCs with Range of Tetrazines

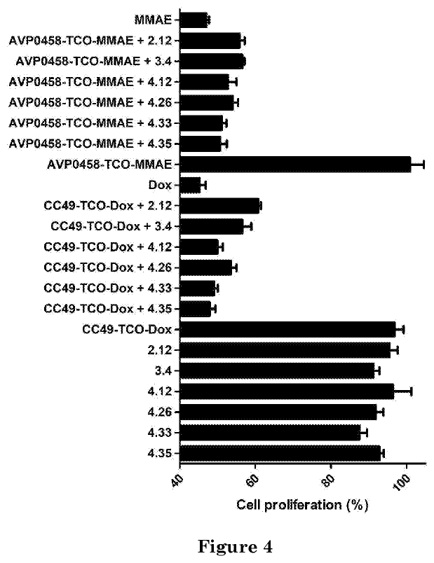

LS174T human colon carcinoma cells were plated at a 5000 cells/well density in RPMI-1640 medium containing 2 mM glutamine and 10% FCS in 96-well plates 24 h prior to the experiment. The wells (n=4) were then added with CC49-TCO-Dox (0.1 μM, DAR=1.9) or AVP0458-TCO-MMAE (1 nM, DAR=4), alone or in combination with TZ activators 2.12, 3.4, 4.12, 4.26, 4.33 and 4.35 (1 μM). Control experiments were performed with the activators alone, Dox (0.19 μM) and MMAE (4 nM). Cell proliferation was assessed after a 3-day incubation by means of an MTT assay and was expressed as the % of that obtained without treatment. The results of this assay (FIG. 4) showed minimal cell growth inhibition in the wells added with the ADCs or with the activators alone, while in the wells treated with a combination of ADC and activator the growth inhibition approached that achieved with the corresponding amount of free drug, signifying effective drug release in the experimental conditions.

### Example 12 ADC Therapy with Activator 4.12

One group of LS174T tumor bearing mice (n=8) was treated with 4 cycles (one every 4 clays) of AVP0458-TCO-MMAE (ca. 3 mg/kg) followed by activator 4.12 (ca. 0.017 mmol/kg) 48 h later. Three more groups of mice (n=8-10) were treated with ADC alone, activator alone or vehicle. The mice were monitored daily and body weight and tumor sizes were recorder at least twice per week up to 50 days from the beginning of the treatment or until a humane end point was reached (>1.5 gr tumor, >20% weight loss, discomfort, etc). Blood samples were collected from 4 mice per group before (day −1) and after the treatment (day 14) and haemoglobin, thrombocytes and leukocytes levels were measured.

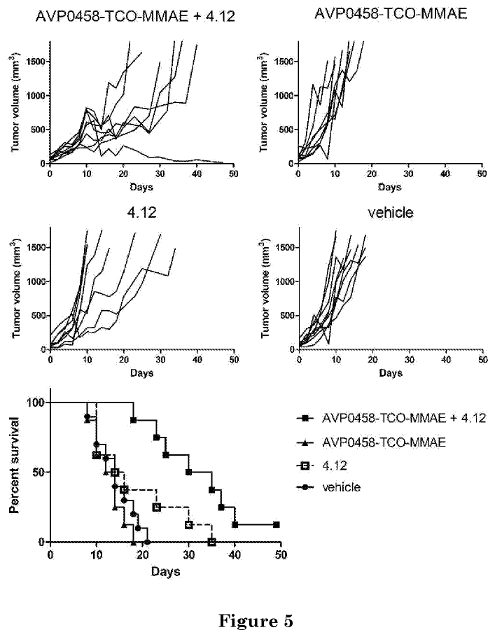

Most of the mice treated with ADC or activator alone were euthanized immediately before or shortly after completion of the fourth treatment cycle due to rapid tumor growth, similar to the group that received the vehicle (13-15 days median survival). On the contrary, despite heterogeneous tumor growth, the mice treated with four cycles of AVP0458-TCO-MMAE and activator 4.12 showed a pronounced response to therapy with a 32.5-day median survival (FIG. 5). Overall ADC and activator were well tolerated by the mice.

### Example 13: Synthesis of TCO-Bound TLR Agonists

The synthesis of rel-(1R,4E,6R,pS)-2,5-dioxopyrrolidin-1-yl-6-((((2,5-dioxopyrrolidin-1-yl)oxy)carbonyl)oxy)-1-methylcyclooct-4-ene-1-carboxylate (axial isomer) (6.1) was reported in Rossin et al. Bioconjug. Chem. 2016, 27, 1697-1706.

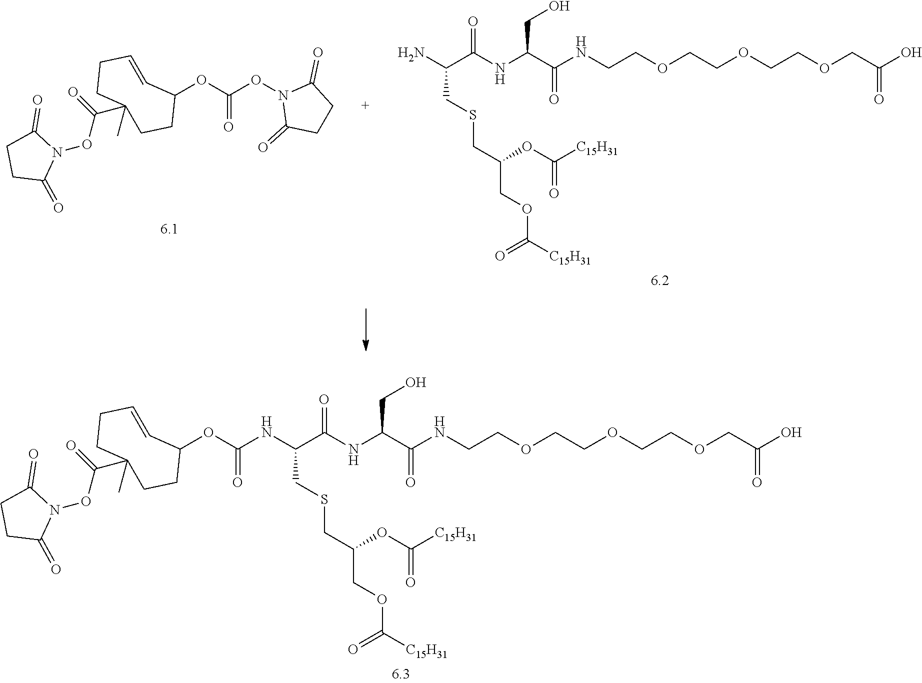

Compound 6.1 (10 μma 9.5 mg) was dissolved in dry DMF (200 μL) in an eppendorf tube. TLR-2/6 agonist 6.2 (12.5 μmol, 5.3 mg) and DiPEA (20 μmol, 3.5 μL) were added. The tube was wrapped in aluminum foil and shaken for 20 h at room temperature after which LCMS (5090, diphenyl column, TFA) indicated depletion of the starting material. The crude reaction mixture was used as is for subsequent conjugation reactions. ESI-MS: m/z Calc. for C₆₄H₁₁₀N₄O₁₈S 1254.75; Obs. [M+H]⁺ 1255.17, [M+Na]⁺ 1277.74.

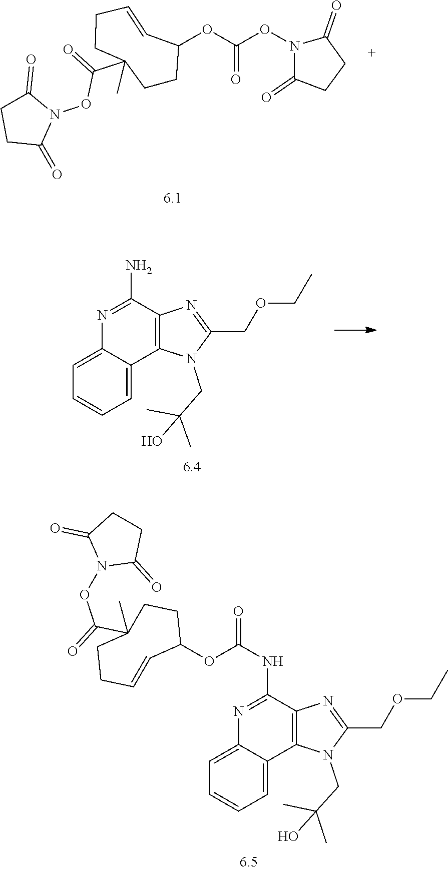 [SMILES: CC1(C(=O)ON2C(=O)CCC2=O)CC/C=C/C(OC(=O)ON2C(=O)CCC2=O)CC1 ; CCOCc1nc2c(N)nc3ccccc3c2n1CC(C)(C)O ; CCOCc1nc2c(NC(=O)OC3/C=C/CCC(C)(C(=O)ON4C(=O)CCC4=O)CC3)nc3ccccc3c2n1CC(C)(C)O]

TLR7/8 agonist 6.4 (Resiquimod, R848, 32 μmol, 10 mg) was dissolved in dry DMF (200 μL) in an eppendorf tube. DiPEA (153 μmol, 28 μL) and compound 6.1 (40 μmol, 17 mg) were added and the tube was shaken for 10 days. LCMS analysis indicated >50% conversion of the starting material. Water was added to the reaction mixture and the product was extracted with DCM (3×). The organic layers were combined, dried (MgSO₄) and concentrated in vacuo. The product was purified using silica gel column chromatography (50% to 80% EtOAc in pentane) yielding compound 6.5 (7.8 μmol, 3.6 mg). ESI-MS: m/z Calc. for C₃₂H₃₉N₅O₈ 621.28; Obs. [M+H]⁺ 622.07. ¹H-NMR (CDCl₃): δ=8.15 (t, 2H, ArH), 7.61 (t, 1H, ArH), 7.48 (t, 1H, ArH), 6.20 (m, 1H, NH), 5.70 (m, 1H, NHCOOCH), 5.48 (s, 1H, trans-alkene H), 5.34 (s, 1H, trans-alkene H), 4.95 (s, 2H, CCH₂N), 4.81 (s, 2H, CCH₂O), 3.68 (q, 2H, OCH₂CH₃), 2.83 (d, 4H, CCH₂CH₂C), 2.50-0.77 (m, 21H, aliphatic protons).

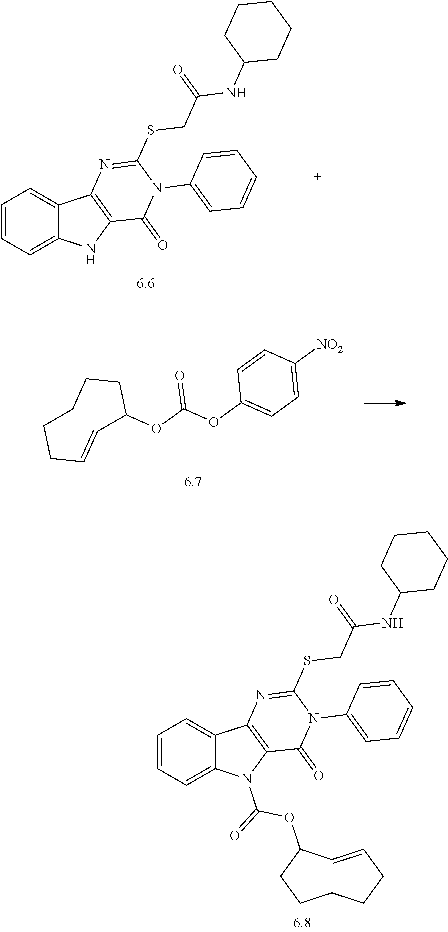 [SMILES: O=C(CSc1nc2c([nH]c3ccccc32)c(=O)n1-c1ccccc1)NC1CCCCC1 ; O=C(Oc1ccc([N+](=O)[O-])cc1)OC1/C=C/CCCCC1 ; O=C(CSc1nc2c3ccccc3n(C(=O)OC3/C=C/CCCCC3)c2c(=O)n1-c1ccccc1)NC1CCCCC1]

TLR 4 agonist, N-Cyclohexyl-2-((4-oxo-3-phenyl-4,5-dihydro-3H-pyrimido[5,4-b]indol-2-yl)thio)acetamide (6.6, 1 equiv.) was suspended in dry THF (0.1 M) under a nitrogen atmosphere. NaH (5 equiv.) was added to the suspension upon which it turned into a yellow solution. After 30 min stirring at room temperature, compound 6.7 (1.2 equiv.) was added to the reaction mixture. After 10 min, LCMS indicated near quantitative conversion of the starting material. H₂O was added to the mixture after which the product was extracted using DCM (5×). The organic layers were combined, dried (MgSO₄) and concentrated in vacuo. The product (6.8) was purified using silica gel column chromatography (0 to 5% acetonitrile in DCM). ESI-MS: m/z Calc. for C₃₃H₃₆N₄O₄S 584.25; Obs. [M+H]⁺ 585.17.

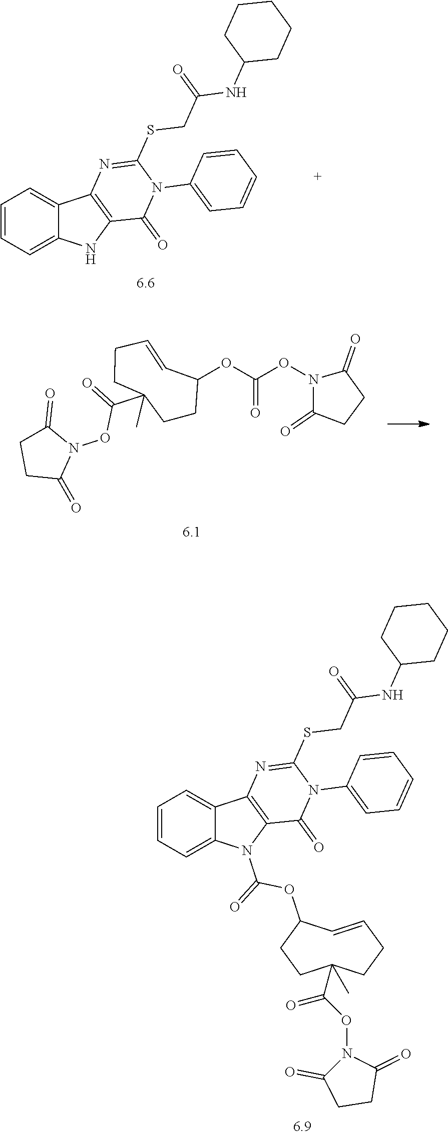 [SMILES: O=C(CSc1nc2c([nH]c3ccccc32)c(=O)n1-c1ccccc1)NC1CCCCC1 ; CC1(C(=O)ON2C(=O)CCC2=O)CC/C=C/C(OC(=O)ON2C(=O)CCC2=O)CC1 ; CC1(C(=O)ON2C(=O)CCC2=O)CC/C=C/C(OC(=O)n2c3ccccc3c3nc(SCC(=O)NC4CCCCC4)n(-c4ccccc4)c(=O)c32)CC1]

Compound 6.9 was prepared like 6.8 using 6.1 as reagent. ESI-MS: m/z Calc. for C₃₉H₄₁N₅O₈S 739.27; Obs. [M+H]+ 740.08.

### Example 14 mAb Conjugation of TCO-Linked TLR Agonist and Evaluation

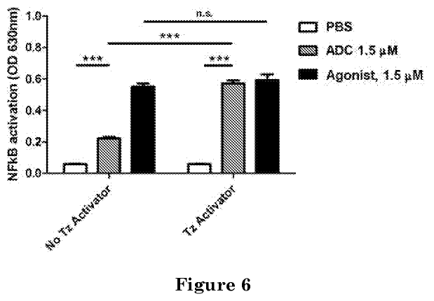

The TA99-TCO-R848 construct was obtained by reacting the anti-glycoprotein 75 (gp-75) mAb TA99 with TCO-linked TLR7/8 agonist 6.5 (80 equiv.) following the conjugation procedure described for CC49-TCO-Dox in Rossin et al., Bioconjug. Chem. 2016, 27, 1697-1706. The construct was purified by PD-10 and a DAR of ca. 1.7 was measured with a tetrazine titration followed by SDS-PAGE analysis. In vitro R848 release from the conjugate was tested in a cell assay. TA99-TCO-R848 (3 μM, 100 μl) was incubated with or without activator 4.12 (30 μM) for 3.5 h in PBS at 37° C., then 10⁵ THP1-Dual cells (Invivogen) were added to the wells in culture medium (100 ul). Free R848 was used as positive control. THP1-Dual cells express secreted embryonic alkaline phosphatase (SEAP) upon NF-kappaB triggering by bioactive R848. After overnight culture, absorbance measurements at 630 nm confirmed increase SEAP production when the cells were incubated in the presence of TA99-TCO-R848 and activator 4.12 with respect to ADC alone (FIG. 6).

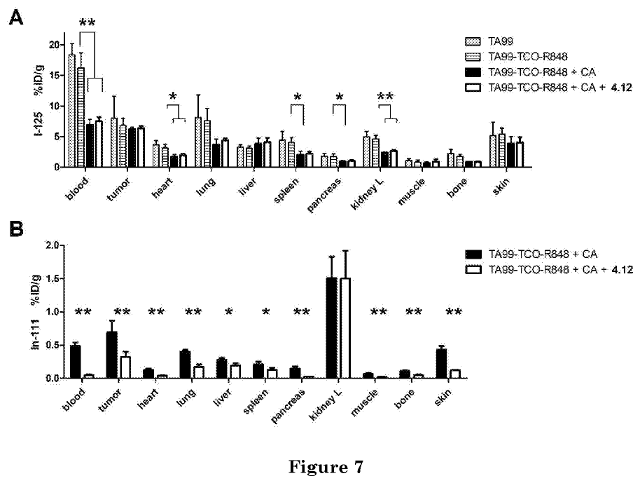

Native TA99 and the TA99-TCO-R848 construct were radiolabeled with ¹²⁵I with the Bolton-Hunter method and the in vivo behavior was evaluated in female C57BL/6 mice bearing subcutaneous B16-F10 melanoma. Two groups of tumor-bearing mice (n=4) were injected with TA99 and TA99-TCO-R848 (ca. 5 mg/kg, ca. 0.3 MBq/mouse) and were euthanized 48 h post-mAb injection. Two more groups of mice were pretreated with the same dose of TA99-TCO-R848 followed 48 h later by a clearing agent (CA, galactose-albumin-TZ, Rossin et al., J. Nucl. Med. 2013, 54, 1989-1995; ca. 10 mg/kg) and, in one group, by activator 4.12 (ca. 0.017 mmol/kg) 50 h post-mAb injection. Both groups were then injected the [¹¹¹In]In-5.1 probe (ca. 0.335 μmol/kg, ca. 1 MBq/mouse) 51 h post-mAb injection and were euthanized 3 h later. The biodistribution of TA99 and TA99-TCO-R848 (I-125) and probe (In-111) is depicted in FIG. 6. The results confirmed retention of target affinity and in vivo behavior after TA99 conjugation to TCO-R848. TA99-TCO-R848 exhibited long retention in blood (16.26±2.43% ID/g 48 h p.i.) and blood-rich organs (e.g. heart and lung) and high uptake in gp-75 positive melanoma and skin (FIG. 7A). Following administration of a clearing agent the mAb uptake in blood and blood rich organs was significantly reduced (one-way ANOVA with Bonferroni post-test, *: p<0.05; **p<0.01) while that in melanoma and skin was maintained, proving target-specific accumulation in these tissues. In vivo reaction between activator 4.12 and the TCO on the mAb construct was confirmed by using the ¹¹¹In-labeled 5.1 probe, following the approach of Example 9. In fact, in mice treated with TA99-TCO-R848 followed by activator, the probe uptake in all tissues (beside kidney due to probe elimination) was significantly lower (Student's t-test) than that in mice that did not receive the activator (FIG. 7B).

### Example 15 ADC Therapy with Activator 2.12

OVCAR-3 tumor bearing mice were administered 4 cycles of ADC (AVP0458-TCO-MMAE) followed by activator (or vehicle) 48 h later. The cycle was repeated every 4 days. Two groups of OVCAR-3 tumor bearing mice (n=8) received 4 cycles of ADC, or the non-binding control AVP06-TCO-MMAE (nb-ADC) at a 3.75 mg/kg dose followed by activator 2.12 (0.335 mmol/kg). Two groups of mice (n=8) were injected either with an analogous enzymatically cleavable vc-ADC or AVP0458-TCO-MMAE at the same dose followed by vehicle and, finally, two more groups of mice (n=8) received either 2.12 or vehicle only.

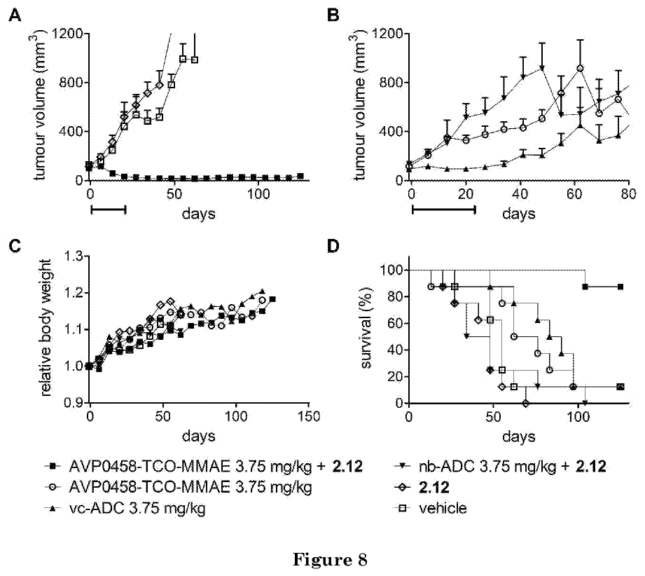

The group of mice that received AVP0458-TCO-MMAE and 2.12 showed significant tumor regression in the first weeks after treatment (117±46 mm³ and 18±9 mm³ tumor volumes at 6 and 34 days, respectively; P=0.0004) followed by 3 months with barely palpable residual tumor masses (FIG. 8A). On the contrary, most of the mice that received vehicle, 2.12 or nb-ADC developed significantly larger tumors (P<0.05 at day 20; FIGS. 8A and 8B) and were removed from the study within two months (41-55 days median survival. Four cycles of ADC alone or vc-ADC followed by vehicle produced very heterogeneous tumor response with significantly larger mean tumor sizes in the second half of the study. Despite the partial therapeutic effect, these groups of mice exhibited a limited median survival (72-86 days) and only one mouse per group reached the end of the study. Overall, repeated doses of ADC and activator were well tolerated by the mice and only one mouse was removed from the study during the last month because of poor health. On the contrary, 4/8 mice treated with vc-ADC were euthanized in the second half of the study due to poor general health or extreme weight losses.

1. A kit comprising a tetrazine and a dienophile, wherein the tetrazine satisfies any one of the Formulae (1), (2), (3), (4), (5), (6), (7), or (8): 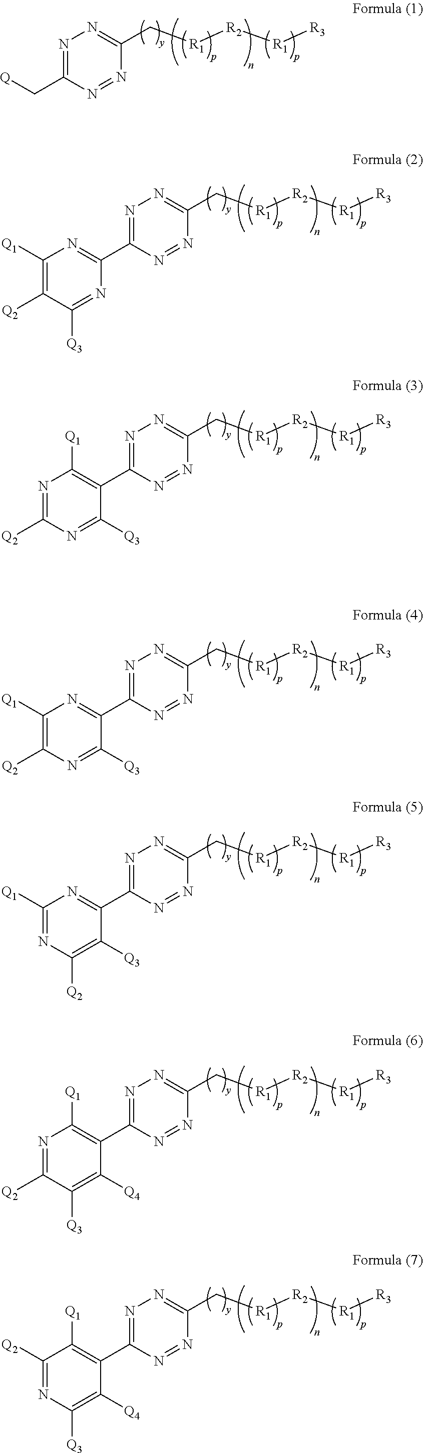 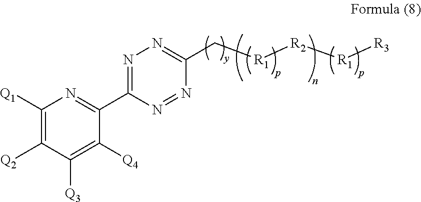
wherein each moiety Q, Q₁, Q₂, Q₃, and Q₄ is independently selected from the group consisting of hydrogen, and moieties according to Formula (9): 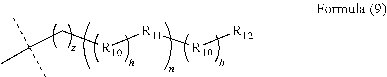
wherein the dashed line indicates a bond to the remaining part of the molecules satisfying any of the Formulae (1), (2), (3), (4), (5), (6), (7), or (8),
wherein each n is an integer independently selected from a range of from 0 to 24,
wherein each p is independently 0 or 1,
wherein y is an integer in a range of from 1 to 12,
wherein z is an integer in a range of from 0 to 12,
wherein each h is independently 0 or 1,
wherein each R₁ and R₁₀ is independently-selected from the group consisting of —O—, —S—, —SS—, —NR₄—, —N(R₄)₂_{⁺}—, —N═N—, —C(O)—, —C(S)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, SC(S)—, —C(S)S—, —SC(S)NR₄—, —NR₄C(S)S—, —C(S)O—, —OC(S)O—, —OC(S)NR₄—, —NR₄C(S)—, —NR₄C(S)O—, —NR₄C(S)—, —C(S)NR₄—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —S(O)₂NR₄—, NR₄S(O)₂—, —OP(O)(R₄)₂—, —SP(O)(R₄)₂—, —NR₄P(O)(R₄)₂—,
wherein R₂ and R₁₁ are independently selected from the group consisting of C₁-C₂₄ alkylene groups, C₂-C₂₄ alkenylene groups, C₂-C₂₄ alkynylene groups, C₆-C₂₄ arylene, C₂-C₂₄ heteroarylene, C₃-C₂₄ cycloalkylene groups, C₅-C₂₄ cycloalkenylene groups, and C₁₂-C₂₄ cycloalkynylene groups,
wherein R₃ and R₁₂ are independently selected from the group consisting of hydrogen, —OH, —NH₂, —N3, —Cl, —Br, —F, —I, and a chelating moiety,
wherein each R₄ is independently-selected from the group consisting of hydrogen, C₁-C₂₄ alkyl groups, C₂-C₂₄ alkenyl groups, C₂-C₂₄ alkynyl groups, C₆-C₂₄ aryl, C₂-C₂₄ heteroaryl, C₃-C₂₄ cycloalkyl groups, C₅-C₂₄ cycloalkenyl groups, C₁₂-C₂₄ cycloalkynyl groups,
wherein in Formulae (1), (2), (3), (4), (5), (6), (7) and (8) at least one moiety selected from the group consisting of Q, Q₁, Q₂, Q₃, Q₄, and —(CH₂)_{y}—((R₁)ₚ—R₂)ₙ—(R₁)ₚ—R₃ has a molecular weight in a range of from 100 Da to 3000 Da,
wherein in Formulae (1), (2), (3), (4), (5), (6), (7) and (8) moieties selected from the group consisting of Q, Q₁, Q₂, Q₃, Q₄, and —(CH₂)_{y}—((R₁)ₚ—R₂)ₙ—(R₁)ₚ—R₃ have a molecular weight of at most 3000 Da,
wherein in Formula (1) when Q is not H, z is 0, n belonging to Q is at least 1, and at least one h is 1, then y is at least 2,
wherein in Formula (1) when Q is not H, y is 1, n belonging to —(CH₂)_{y}—((R₁)ₚ—R₂)ₙ—(R₁)ₚ—R₃ is at least 1, and at least one p is 1, then z is at least 1,
wherein in Formula (8) when Q₁, Q₂, Q₃, and Q₄ are hydrogen, then y is not 1,
wherein in Formula (8) when y is 1, each p is 0, n belonging to —(CH₂)_{y}—((R₁)ₚ—R₂)ₙ—(R₁)ₚ—R₃ is 0, R₃ is hydrogen, Q₁ is hydrogen, Q₃ is hydrogen, Q₄ is hydrogen, and Q₂ is not hydrogen, then z is at least 1,
wherein the R₂ groups, the R₁₁ groups, and the R₄ groups not being hydrogen, optionally contain one or more heteroatoms selected from the group consisting of O, S, NR₅, P, and Si, wherein the N, S, and P atoms are optionally oxidized, wherein the N atoms are optionally quaternized,
wherein the R₂ groups, the R₁₁ groups, and the R₄ groups not being hydrogen, are optionally further substituted with one or more substituents selected from the group consisting of —Cl, —F, —Br, —I, —OH, —NH₂, —SO₃H, —PO₃H, —PO₄H₂, —NO₂, —CF₃, ═O, ═NR₅, —SR₅, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ alkyl(hetero)aryl groups, C₃-C₂₄ (hetero)arylalkyl groups, C₄-C₂₄ (hetero)arylalkenyl groups, C₄-C₂₄ (hetero)arylalkynyl groups, C₄-C₂₄ alkenyl(hetero)aryl groups, C₄-C₂₄ alkynyl(hetero)aryl groups, C₄-C₂₄ alkylcycloalkyl groups, C₆-C₂₄ alkylcycloalkenyl groups, C₁₃-C₂₄ alkylcycloalkynyl groups, C₄-C₂₄ cycloalkylalkyl groups, C₆-C₂₄ cycloalkenylalkyl groups, C₁₃-C₂₄ cycloalkynylalkyl groups, C₅-C₂₄ alkenylcycloalkyl groups, C₇-C₂₄ alkenylcycloalkenyl groups, C₁₄-C₂₄ alkenylcycloalkynyl groups, C₅-C₂₄ cycloalkylalkenyl groups, C₇-C₂₄ cycloalkenylalkenyl groups, C₁₄-C₂₄ cycloalkynylalkenyl groups, C₅-C₂₄ alkynylcycloalkyl groups, C₇-C₂₄ alkynylcycloalkenyl groups, C₁₄-C₂₄ alkynylcycloalkynyl groups, C₅-C₂₄ cycloalkylalkynyl groups, C₇-C₂₄ cycloalkenylalkynyl groups, C₁₄-C₂₄ cycloalkynylalkynyl groups, C₅-C₂₄ cycloalkyl(hetero)aryl groups, C₇-C₂₄ cycloalkenyl(hetero)aryl groups, C₁₄-C₂₄ cycloalkynyl(hetero)aryl groups, C₅-C₂₄ (hetero)arylcycloalkyl groups, C₇-C₂₄ (hetero)arylcycloalkenyl groups, and C₁₄-C₂₄ (hetero)arylcycloalkynyl groups,
wherein the substituents optionally contain one or more heteroatoms selected from the group consisting of O, S, NR₅, P, and Si, wherein the N, S, and P atoms are optionally oxidized, wherein the N atoms are optionally quaternized,
wherein each R₅ is independently selected from the group consisting of hydrogen, C₁-C₈ alkyl groups, C₂-C₈ alkenyl groups, C₂-C₈ alkynyl groups, C₆-C₁₂ aryl, C₂-C₁₂ heteroaryl, C₃-C₈ cycloalkyl groups, C₅-C₈ cycloalkenyl groups, C₃-C₁₂ alkyl(hetero)aryl groups, C₃-C₁₂ (hetero)arylalkyl groups, C₄-C₁₂ alkylcycloalkyl groups, C₄-C₁₂ cycloalkylalkyl groups, C₅-C₁₂ cycloalkyl(hetero)aryl groups and C₅-C₁₂ (hetero)arylcycloalkyl groups,
wherein the R₅ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized;
and optionally including pharmaceutically acceptable salts thereof.

2. The kit according to claim 1,
wherein the compound according to Formulae (1), (2), (3), (4), (5), (6), (7) or (8) has a Log P value of at most 3.0.

3. The kit according to claim 1, wherein R₃ is a chelator moiety selected from the group consisting of 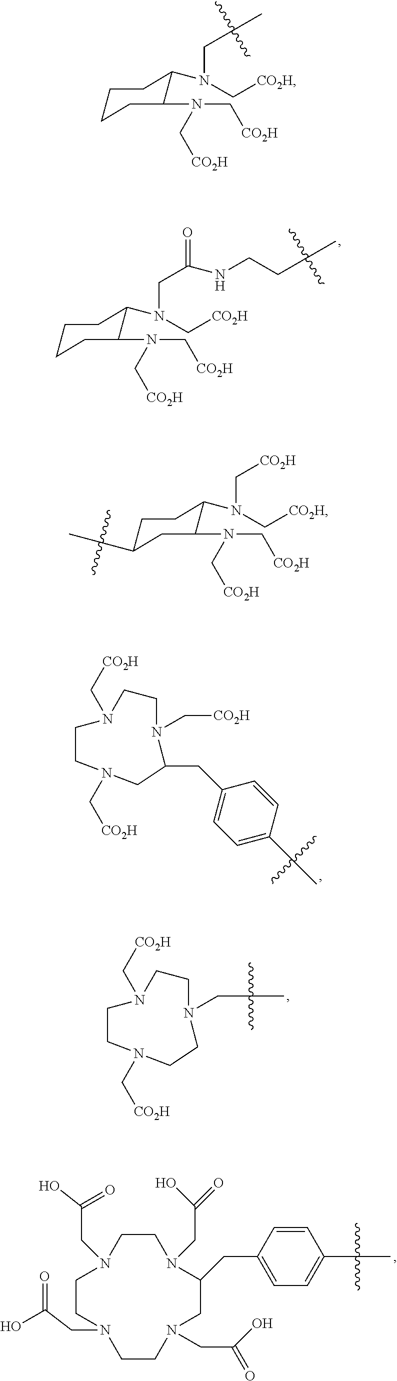 [SMILES: *CN(CC(=O)O)C1CCCCC1N(CC(=O)O)CC(=O)O ; *CCNC(=O)CN(CC(=O)O)C1CCCCC1N(CC(=O)O)CC(=O)O ; *C1CCC(N(CC(=O)O)CC(=O)O)C(N(CC(=O)O)CC(=O)O)C1 ; *c1ccc(CC2CN(CC(=O)O)CCN(CC(=O)O)CCN2CC(=O)O)cc1 ; *CN1CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1] 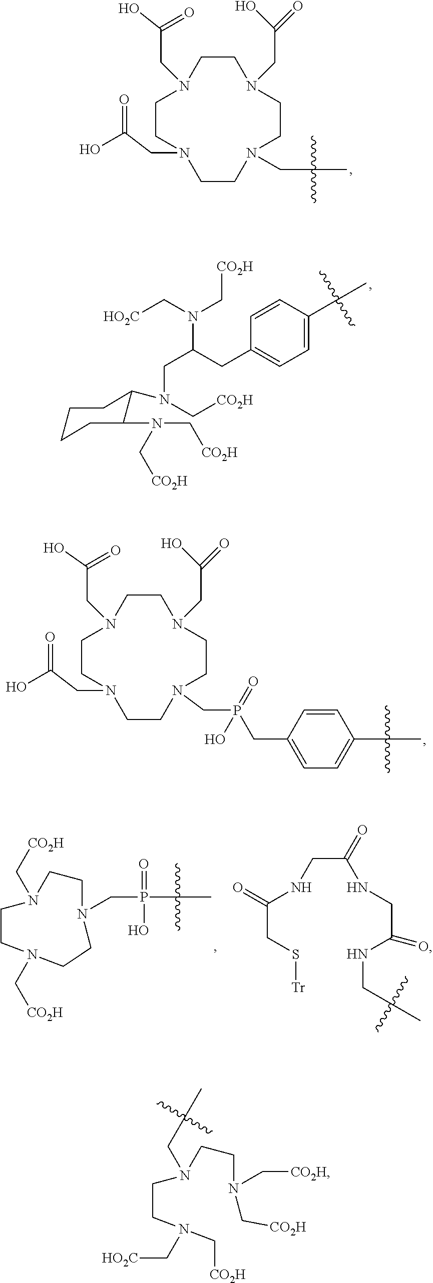 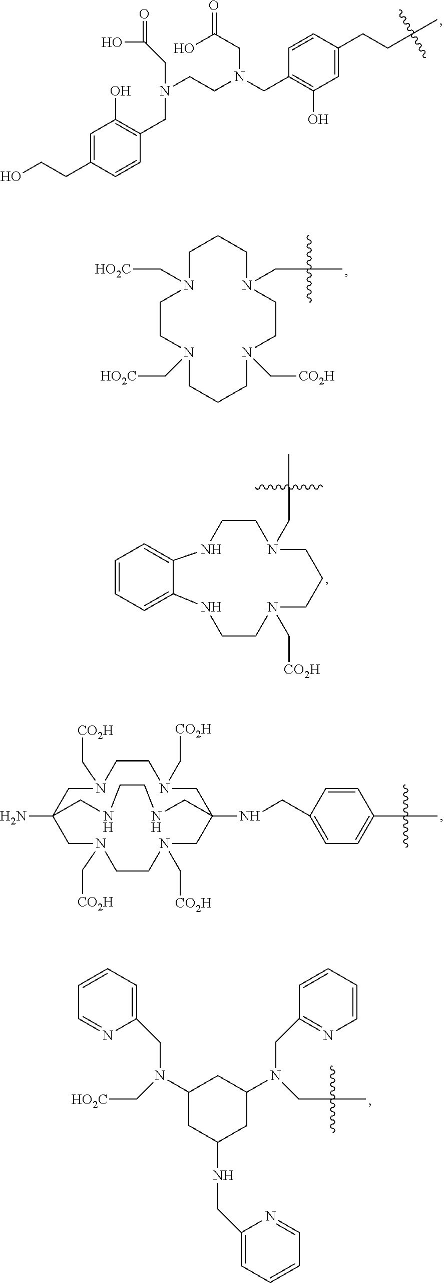 [SMILES: *CCc1ccc(CN(CCN(CC(=O)O)Cc2ccc(CCO)cc2O)CC(=O)O)c(O)c1 ; *CN1CCCN(CC(=O)O)CCN(CC(=O)O)CCCN(CC(=O)O)CC1 ; *CN1CCCN(CC(=O)O)CCNc2ccccc2NCC1 ; *c1ccc(CNC23CNCCNCC(N)(CN(CC(=O)O)CCN(CC(=O)O)C2)CN(CC(=O)O)CCN(CC(=O)O)C3)cc1 ; *CN(Cc1ccccn1)C1CC(NCc2ccccn2)CC(N(CC(=O)O)Cc2ccccn2)C1] 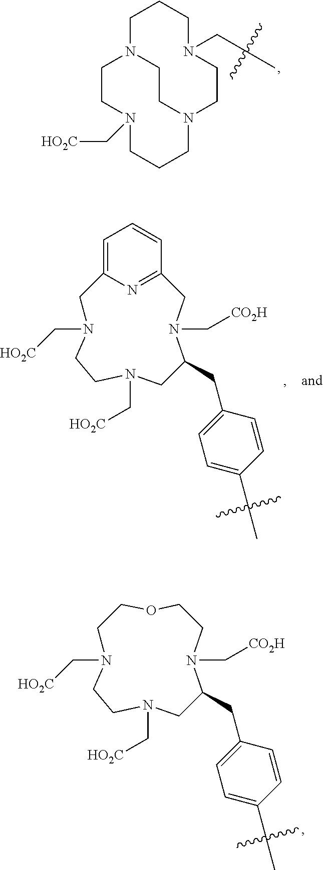 [SMILES: *CN1CCCN2CCN(CCCN(CC(=O)O)CC2)CC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1]
wherein the wiggly line denotes a bond to the remaining part of the molecule, optionally bound via —C(O)NH—,
wherein the chelator moieties according to said group optionally chelate a metal.

4. The kit according to claim 1, wherein the chelator moiety chelates a metal ion.

5. The kit according to claim 1, wherein the chelator moiety chelates an isotope selected from the group consisting of ⁶²Cu, ⁶⁴Cu, ⁶⁶Ga, ⁶⁷Ga, ⁶⁷Cu, ⁶⁸Ga, ⁸⁶Y, ⁸⁹Zr, ⁹⁰Y, ^{99m}Tc, ¹¹¹In, ¹⁶⁶Ho, ¹⁷⁷Lu, ¹⁸⁶Re, ¹⁸⁸Re, ²¹¹Bi, ²¹²Bi, ²¹²Pb, ²¹³Bi, ²¹⁴Bi, and ²²⁵Ac.

6. The kit according to claim 1, wherein the tetrazine satisfies any one of Formulae (11), (12), (13), (14), (15), (16), (17), or (18): 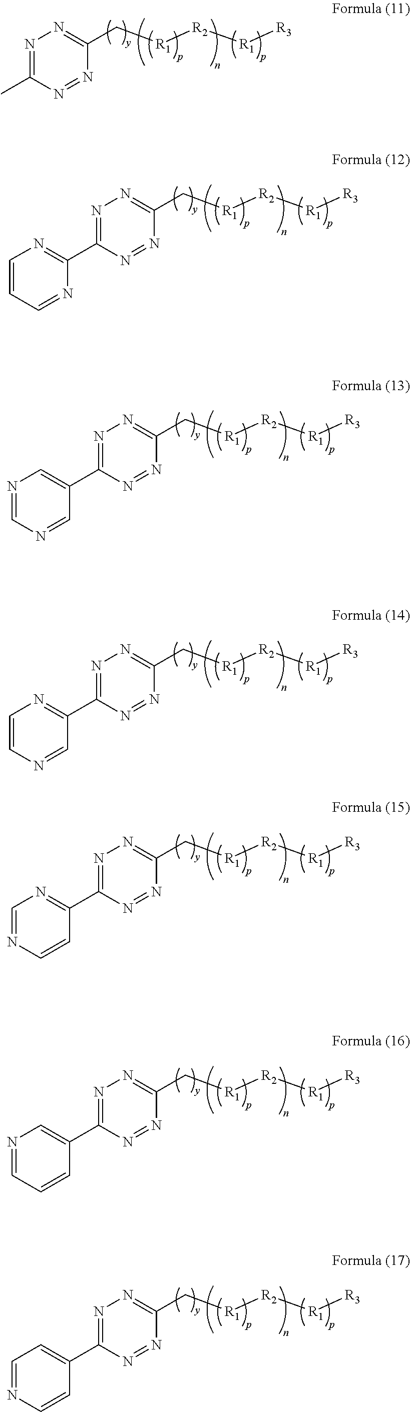 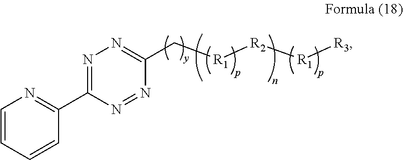
wherein n, p, y, R₁, R₂, and R₃ are as defined in claim 1 for Formulae (1), (2), (3), (4), (5), (6), (7), and (8),
wherein in Formulae (11), (12), (13), (14), (15), (16), (17), and (18) the moiety —(CH₂)_{y}—((R₁)ₚ—R₂)ₙ—(R₁)ₚ—R₃ has a molecular weight in a range of from 100 Da to 3000 Da, and
wherein in Formula (18) y is not 1.

7. The kit according to claim 6, wherein the compounds according to Formulae (11), (12), (13), (14), (15), (16), (17), or (18) have a Log P value of at most 3.0.

8. The kit according to claim 1, wherein the dienophile satisfies Formula (19a): 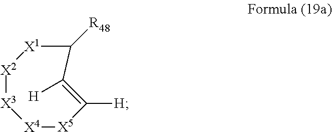 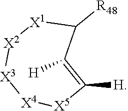
and including pharmaceutically acceptable salts thereof,
wherein R₄₈ is selected from the group consisting of —OH,
—OC(O)Cl, —OC(O)O—N-succinimidyl, —OC(O)O-4-nitrophenyl, —OC(O)O-tetrafluorophenyl, —OC(O)O-pentafluorophenyl, —OC(O)—C^{A}, —OC(S)—C^{A},
—O-(L^{C}(C^{A})ₛ(C^{A})ₛ((S^{P})ᵢ—C^{B})ⱼ)ᵣ—C^{A}, and —C^{A},
wherein r is an integer in range of from 0 to 2,
wherein each s is independently 0 or 1,
wherein i is an integer in a range of from 0 to 4,
wherein j is 0 or 1,
wherein L^{C} is a self-immolative linker,
wherein C^{A} denotes a Construct A, wherein said Construct A is selected from the group consisting of drugs, targeting agents and masking moieties,
wherein C^{B} denotes a Construct B, wherein said Construct B is selected from the group consisting of masking moieties, drugs and targeting agents,
wherein, when C^{B} is a targeting agent or a masking moiety, then C^{A} is a drug,
wherein, when C^{B} is a drug, then C^{A} is a masking moiety or a targeting agent,
wherein, when R₄₈ is —OC(O)—C^{A} or —OC(S)—C^{A}, C^{A} is bound to the —OC(O)— or —OC(S)— of R₄₈ via an atom selected from the group consisting of O, C, S, and N, preferably a secondary or a tertiary N, wherein this atom is part of C^{A},
wherein, when R₄₈ is —O-(L^{C}(C^{A})ₛ(C^{A})ₛ((S^{P})ᵢ—C^{B})ⱼ)ᵣ—C^{A} and r is 0, C^{A} is bound to the —O— moiety of R₄₈ on the allylic position of the trans-cyclooctene ring of Formula (19) via a group selected from the group consisting of —C(O)—, and —C(S)—, wherein this group is part of C^{A},
wherein, when R₄₈ is —O-(L^{C}(C^{A})ₛ(C^{A})ₛ((S^{P})ᵢ—C¹³)ⱼ)ᵣ—C^{A} and r is 1, L^{C} is bound to the —O— moiety on the allylic position of the trans-cyclooctene ring of Formula (19) via a group selected from the group consisting of —C(Y^{C2})Y^{C1}—, and a carbon atom, wherein this group is part of L^{C},
wherein Y^{C1} is selected from the group consisting of —O—, —S—, and —NR₃₆—,
wherein Y^{C2} is selected from the group consisting of O and S,
wherein, when R₄₈ is —O-(L^{C}(C^{A})ₛ(C^{A})ₛ((S^{P})ᵢ—C^{B})ⱼ)ᵣ—C^{A}, and r is 1, then C^{A} is bound to L^{C} via a moiety selected from the group consisting of —O—, —S—, and —N—, wherein said moiety is part of C^{A},
wherein, when R₄₈ is —C^{A}, then C^{A} is bound to the allylic position of the trans-cyclooctene of Formula (19) via an —O— atom, wherein this atom is part of C^{A},
wherein R₃₆ is selected from the group consisting of hydrogen and C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein X⁵ is —C(R₄₇)₂— or —CHR₄₈,
wherein each X¹, X², X³, X⁴ is independently selected from the group consisting of —C(R₄₇)₂—, —NR₃₇—, —C(O)—, —O—, such that at most two of X¹, X², X³, X⁴ are not —C(R₄₇)₂—, and with the proviso that no sets consisting of adjacent atoms are present selected from the group consisting of —O—O—, —O—N—, —C(O)—O—, N—N—, and —C(O)—C(O)—,
wherein each R₄₇ is independently selected from the group consisting of hydrogen, —(S^{P})ᵢ—C^{B} with i being an integer in a range of from 0 to 4, —F, —Cl, —Br, —I, —OR₃₇, —N(R₃₇)₂, —SO₃, —PO₃⁻, —NO₂, —CF₃, —SR₃₇, S(═O)₂N(R₃₇)₂, OC(═O)R₃₇, SC(═O) R₃₇, OC(═S)R₃₇, SC(═S)R₃₇, NR₃₇C(═O)—R₃₇, NR₃₇C(═S)—R₃₇, NR₃₇C(═O)O—R₃₇, NR₃₇C(═S)O—R₃₇, NR₃₇C(═O)S—R₃₇, NR₃₇C(═S)S—R₃₇, OC(═O)N(R₃₇)₂, SC(═O)N(R₃₇)₂, OC(═S)N(R₃₇)₂, SC(═S)N(R₃₇)₂, NR₃₇C(═O)N(R₃₇)₂, NR₃₇C(═S)N(R₃₇)₂, C(═O)R₃₇, C(═S)R₃₇, C(═O)N(R₃₇)₂, C(═S)N(R₃₇)₂, C(═O)O—R₃₇, C(═O)S—R₃₇, C(═S)O—R₃₇, C(═S)S—R₃₇, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups;
wherein the alkyl groups, alkenyl groups, alkynyl groups, aryl, heteroaryl, cycloalkyl groups, cycloalkenyl groups, cycloalkynyl groups, (cyclo)alkyl(hetero)aryl groups, (hetero)aryl(cyclo)alkyl groups, (cyclo)alkenyl(hetero)aryl groups, (hetero)aryl(cyclo)alkenyl groups, (cyclo)alkynyl(hetero)aryl groups, (hetero)aryl(cyclo)alkynyl groups, alkylcycloalkyl groups, cycloalkylalkyl groups are optionally substituted with a moiety selected from the group consisting of —Cl, —F, —Br, —I, —OR₃₇, —N(R₃₇)₂, —SO₃R₃₇, —PO₃(R₃₇)₂, —PO₄(R₃₇)₂, —NO₂, —CF₃, ═O, ═NR₃₇, and —SR₃₇, and optionally contain one or more heteroatoms selected from the group consisting of O, S, NR₃₇, P, and Si, wherein the N, S, and P atoms are optionally oxidized, wherein the N atoms are optionally quaternized,
wherein two R₄₇ are optionally comprised in a ring,
wherein each R₃₇ is independently selected from the group consisting of hydrogen, —(S^{P})ᵢ—C^{B} with i being an integer in a range of from 0 to 4, C₁-C₂₄ alkyl groups, C₂-C₂₄ alkenyl groups, C₂-C₂₄ alkynyl groups, C₆-C₂₄ aryl groups, C₂-C₂₄ heteroaryl groups, C₃-C₂₄ cycloalkyl groups, C₅-C₂₄ cycloalkenyl groups, C₁₂-C₂₄ cycloalkynyl groups, C₃-C₂₄ (cyclo)alkyl(hetero)aryl groups, C₃-C₂₄ (hetero)aryl(cyclo)alkyl, C₄-C₂₄ (cyclo)alkenyl(hetero)aryl groups, C₄-C₂₄ (hetero)aryl(cyclo)alkenyl groups, C₄-C₂₄ (cyclo)alkynyl(hetero)aryl groups, C₄-C₂₄ (hetero)aryl(cyclo)alkynyl groups, C₄-C₂₄ alkylcycloalkyl groups, and C₄-C₂₄ cycloalkylalkyl groups;
wherein the R₃₇ groups not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized,
wherein S^{P} is a spacer.

9. The kit according to claim 8, wherein each S^{P} is selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups, wherein for S^{P} the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein each R′ is independently selected from the group consisting of hydrogen, C₁-C₆ alkylene groups, C₂-C₆ alkenylene groups, C₂-C₆ alkynylene groups, C₆ arylene, C₄-C₅ heteroarylene, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein for R′ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups are optionally substituted with a moiety selected from the group consisting of —Cl, —F, —Br, —I, —OH, —NH₂, ═O, —SH, —SO₃H, —PO₃H, —PO₄H₂, —NO₂, and optionally contain one or more heteroatoms selected from the group consisting of —O—, —S—, —NH—, —P—, and —Si, wherein the N, S, and P atoms are optionally oxidized.

10. The kit according to claim 8, wherein L^{C} is selected from the group consisting of linkers according to Group I, Group II, and Group III, wherein linkers according to Group I are 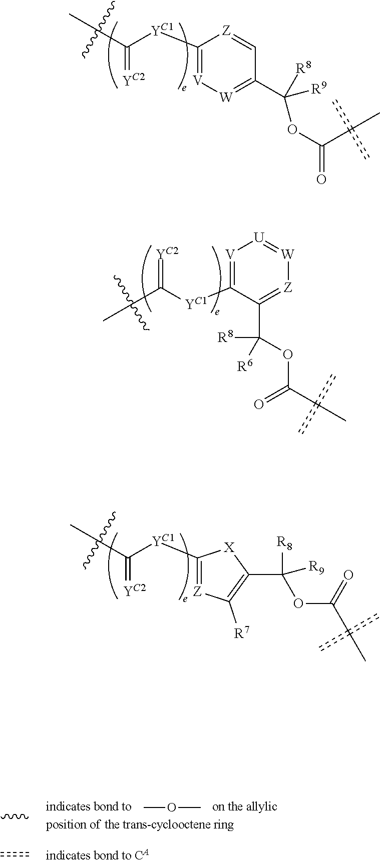
wherein U, V, W, Z are each selected from the group consisting of —CR⁷—, and —N—,
wherein e is either 0 or 1,
wherein X is selected from the group consisting of —O—, —S— and —NR⁶—,
wherein for all Groups I, II, and III, each R⁸ and R⁹ are* selected from the group consisting of hydrogen, C₁-C₄ alkyl groups, C₂-C₄ alkenyl groups, and C₄₋₆ (hetero)aryl groups,
wherein for R⁸ and R⁹ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein for linkers according to Group I C^{A} is linked to L^{C} via a moiety selected from the group consisting of —O—, —N—, —C—, and —S—, wherein said moieties are part of C^{A},
wherein the linker according to Group II is 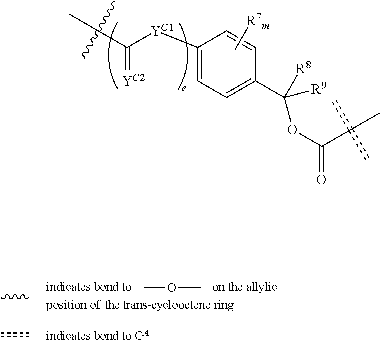
wherein m is an integer between 0 and 2,
wherein e is either 0 or 1,
wherein for linkers according to Group II C^{A} is linked to L^{C} via a moiety selected from the group consisting of —O—, —N—, —C—, and —S—, wherein said moieties are part of C^{A},
wherein linkers according to Group III are 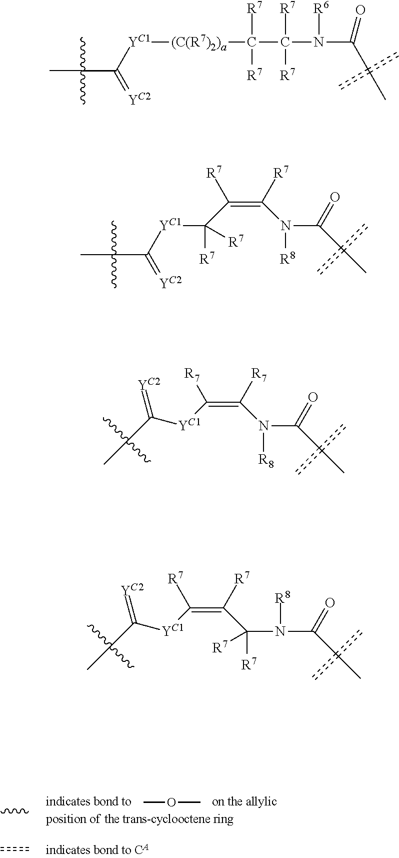
wherein for linkers according to Group III C^{A} is linked to L^{C} via a moiety selected from the group consisting of —O— and —S—, wherein said moieties are part of C^{A},
wherein for all Groups I, II, and III, each R⁶ is selected from the group consisting of hydrogen, C₁-C₄ alkyl groups, C₂-C₄ alkenyl groups, and C₄₋₆ (hetero)aryl groups,
wherein for R⁶ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
wherein for all Groups I, II, and III, each R⁷ is independently selected from the group consisting of hydrogen and C₁-C₃ alkyl groups, C₂-C₃ alkenyl groups, and C₄₋₆ (hetero)aryl groups,
wherein for R⁷ the alkyl groups, alkenyl groups, and (hetero)aryl groups are optionally substituted with a moiety selected from the group consisting of —Cl, —F, —Br, —I, —OH, —NH₂, ═O, ═NH, —N(CH₃)₂, —S(O)₂CH₃, and —SH, and are optionally interrupted by at most one heteroatom selected from the group consisting of —O—, —S—, —NH—, —P—, and —Si—, wherein the N, S, and P atoms are optionally oxidized, wherein the N atoms are optionally quaternized,
wherein R⁶, R⁷, R⁸, R⁹ comprised in said Group I, II and III, can optionally also be —(S^{P})ᵢ—C^{B},
wherein for all linkers according to Group I and Group II Y^{C1} is selected from the group consisting of —O—, —S—, and —NR⁶,
wherein for all linkers according to Group III, Y^{C1} is —NR⁶—,
wherein for all linkers according to Group I, Group II, and Group III, Y^{C2} is selected from the group consisting of O and S,
wherein when r as defined in claim 1 is two, then the L^{C} attached to the —O— at the allylic position of the trans-cyclooctene is selected from the group consisting of linkers according to Group I and Group II, and the L^{C} between the L^{C} attached to the —O— at the allylic position of the trans-cyclooctene and C^{A} is selected from Group III, and that the wiggly line in the structures of Group III then denotes a bond to the L^{C} attached to the —O— at the allylic position of the trans-cyclooctene instead of a bond to the allylic —O— on the trans-cyclooctene ring, and that the double dashed line in the structures of Groups I and II then denotes a bond to the L^{C} between the L^{C} attached to the —O— at the allylic position of the trans-cyclooctene and the C^{A} instead of a bond to C^{A}.

11. The kit according to claim 8, wherein L^{C} is selected from the group consisting of linkers according to Group IV, Group V, Group VI, and Group VII, wherein linkers according to Group IV are 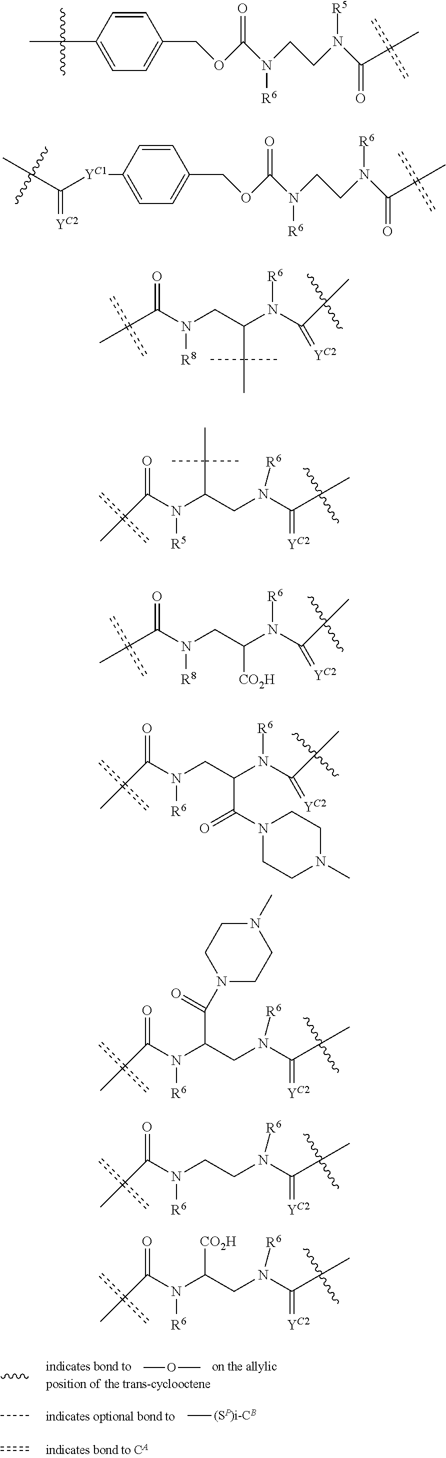
wherein C^{A} is linked to L^{C} via a moiety selected from the group consisting of —O— and —S—, wherein said moieties are part of C^{A},
wherein linkers according to Group V are 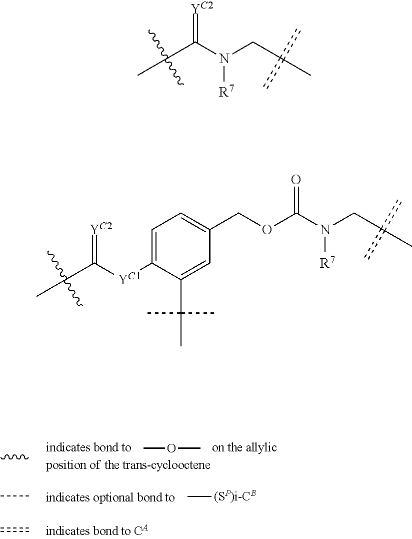
wherein C^{A} is linked to L^{C} via a moiety selected from the group consisting of —O— and —S—, wherein said moieties are part of C^{A},
wherein linkers according to Group VI are 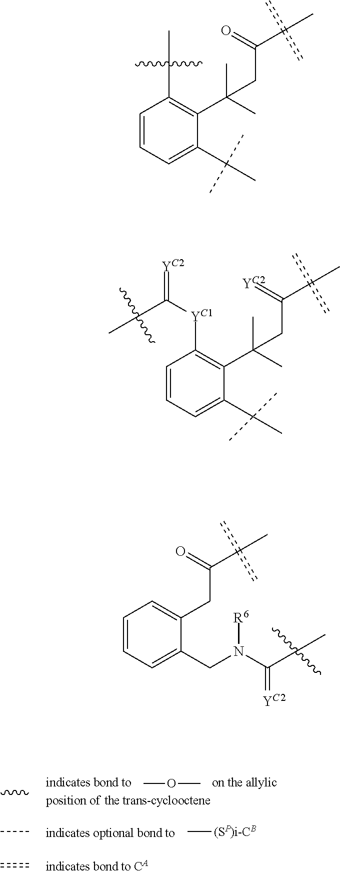
wherein C^{A} is linked to L^{C} via a moiety selected from the group consisting of —O—, —N—, and —S—, wherein said moieties are part of C^{A},
wherein linkers according to Group VII are 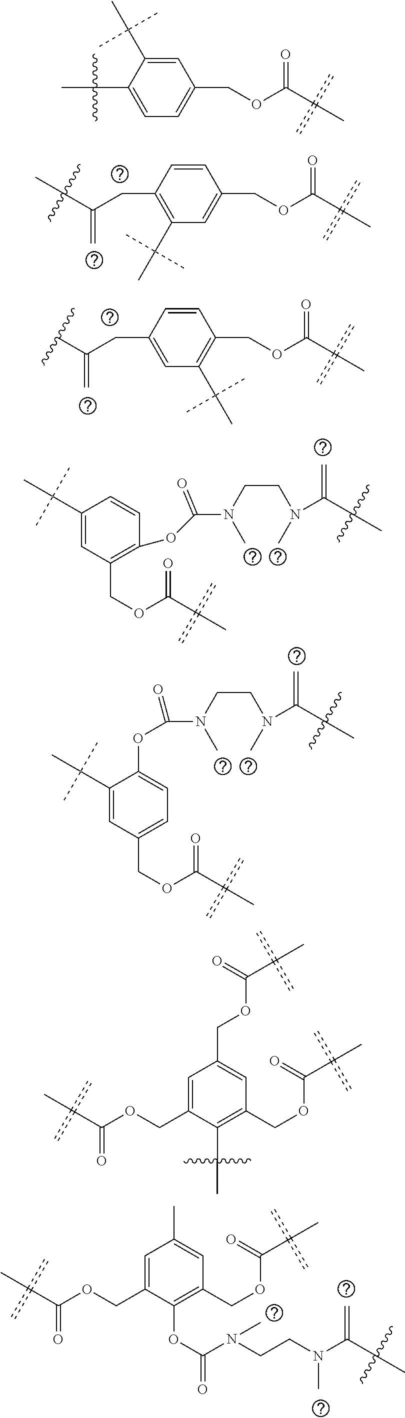 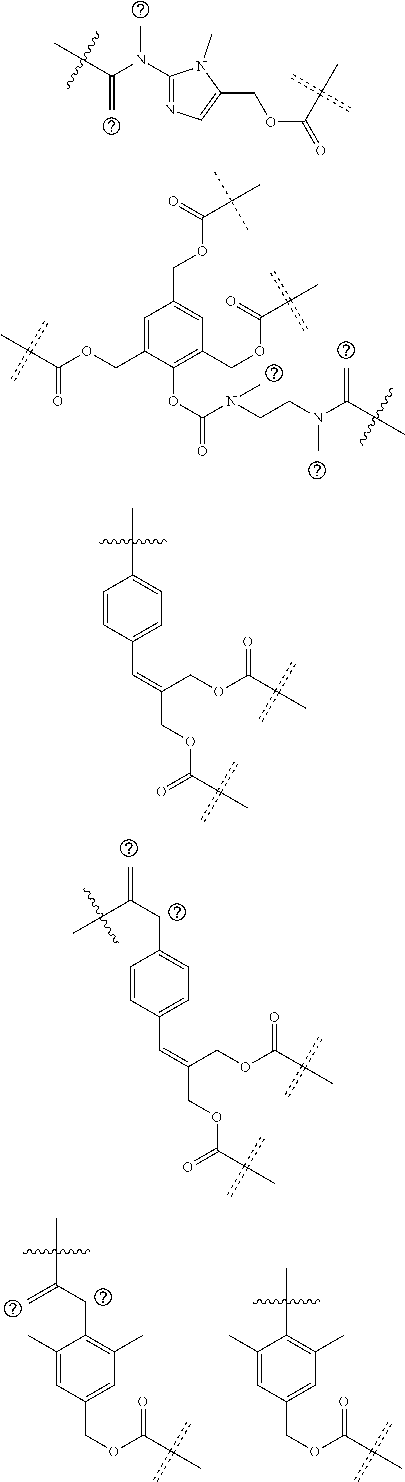 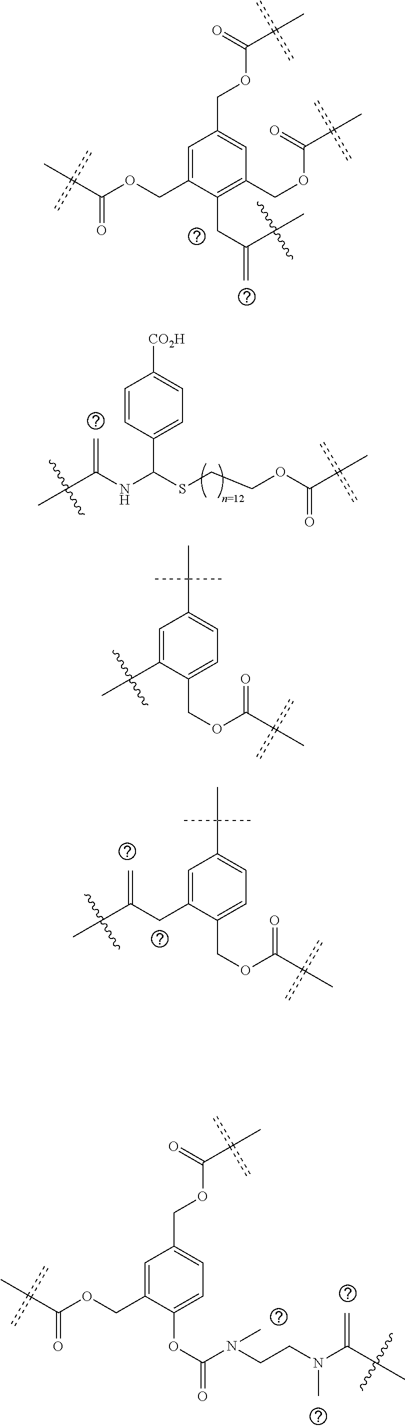 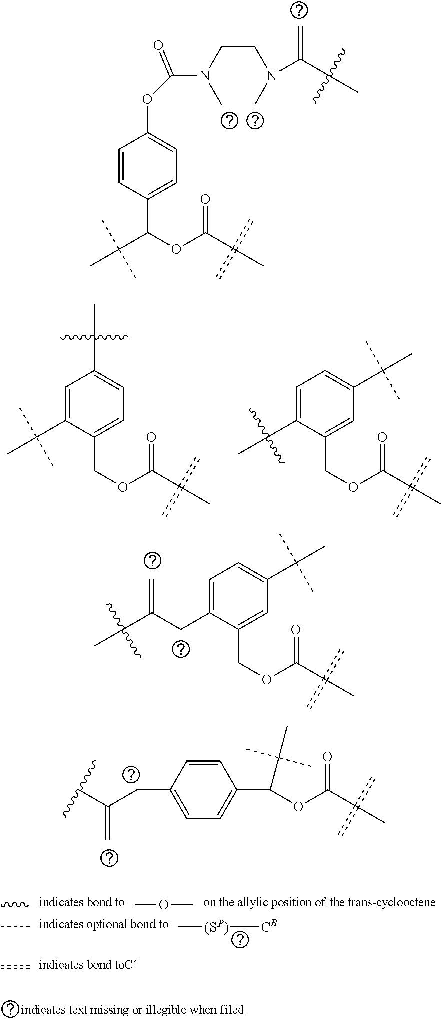
wherein C^{A} is linked to L^{C} via a moiety selected from the group consisting of —O—, —N—, and —S—, wherein said moieties are part of C^{A}, wherein when multiple double dashed lines are shown within one L^{C}, each C^{A} moiety is independently-selected,
wherein for all linkers according to Group IV, Group V, Group VI, and Group VII, Y^{C1} is selected from the group consisting of —O—, —S—, and —NR⁶—,
wherein C^{B} is selected from the group consisting of drugs, targeting agents, and masking moieties,
wherein R⁶, and Ware as defined in claim 10.

12. The kit according to claim 8, wherein each X in Formula (19) is —C(R₄₇)₂—.

13. The kit according to claim 8, wherein at most three R₄₇ in Formula (19) are not H.

14. The kit according to claim 8, wherein R₄₈ is in the axial position.

15. The kit according to claim 8, wherein the dienophile satisfies Formula (20) 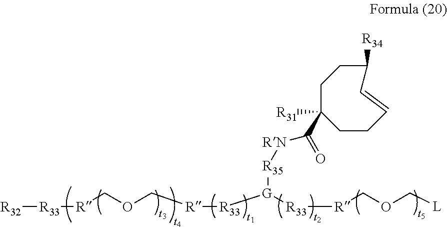
wherein t₁ is 0 or 1,
wherein t₂ is 0 or 1,
wherein t₃ is an integer in a range of from 1 to 12,
wherein t₄ is 0 or 1,
wherein t₅ is an integer in a range of from 6 to 48,
wherein L is selected from the group consisting of —CH₂—OCH₃, —CH₂—OH, —CH₂—C(O)OH, —C(O)OH,
wherein when at least one of ti or t₂ is 0, then G is selected from the group consisting of CR′, C₅-C₆ arenetriyl, C₄-C₅ heteroarenetriyl, C₃-C₆ cycloalkanetriyl, and C₄-C₆ cycloalkenetriyl, wherein when both t₁ and t₂ are 1, then G is selected from the group consisting of CR′, N, C₅-C₆ arenetriyl, C₄-C₅ heteroarenetriyl, C₃-C₆ cycloalkanetriyl, and C₄-C₆ cycloalkenetriyl,
wherein for G, the arenetriyl, heteroarenetriyl, cycloalkanetriyl, and cycloalkenetriyl are optionally further substituted with groups selected from the group consisting of —Cl, —F, —Br, —I, —OR′, —N(R′)₂, —SR′, —SO₃H, —PO₃H, —PO₄H₂, —NO₂, —CF₃ and —R₃₁, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein R₃₁ is selected from the group consisting of hydrogen, C₁-C₆ alkyl groups, C₆ aryl groups, C₄-C₅ heteroaryl groups, C₃-C₆ cycloalkyl groups, C₅-C₁₂ alkyl(hetero)aryl groups, C₅-C₁₂ (hetero)arylalkyl groups, C₄-C₁₂ alkylcycloalkyl groups, —N(R′)₂, —OR′, —SR′, —SO₃H, —C(O)OR′, and Si(R′)₃,
wherein for R₃₁ the alkyl groups, (hetero)aryl groups, cycloalkyl groups, alkyl(hetero)aryl groups, (hetero)arylalkyl groups, alkylcycloalkyl groups are optionally substituted with a moiety selected from the group consisting of —Cl, —F, —Br, —I, NO₂, SO₃H, POSH, —PO₄H₂, —OR′, —N(R′)₂, —CF₃, ═O, ═NR′, —SR′, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein R₃₂ is selected from the group consisting of N-maleimidyl groups, halogenated N-alkylamido groups, sulfonyloxy N-alkylamido groups, vinyl sulfone groups, activated carboxylic acids, benzenesulfonyl halides, ester groups, carbonate groups, sulfonyl halide groups, thiol groups or derivatives thereof, C₂₋₆ alkenyl groups, C₂₋₆ alkynyl groups, C₇₋₁₈ cycloalkynyl groups, C₅₋₁₈ heterocycloalkynyl groups, bicyclo[6.1.0]non-4-yn-9-yl] groups, C₄₋₁₂ cycloalkenyl groups, azido groups, phosphine groups, nitrile oxide groups, nitrone groups, nitrile imine groups, isonitrile groups, diazo groups, ketone groups, (O-alkyl)hydroxylamino groups, hydrazine groups, halogenated N-maleimidyl groups, aryloxymaleimides, dithiophenolmaleimides, bromo- and dibromopyridazinediones, 2,5-dibromohexanediamide groups, alkynone groups, 3-arylpropionitrile groups, 1,1-bis(sulfonylmethyl)-methylcarbonyl groups or elimination derivatives thereof, carbonyl halide groups, allenamide groups, 1,2-quinone groups, isothiocyanate groups, aldehyde groups, triazine groups, squaric acids, 2-imino-2-methoxyethyl groups, (oxa)norbornene groups, (imino)sydnones, methylsulfonyl phenyloxadiazole groups, aminooxy groups, 2-amino benzamidoxime groups, groups reactive in the Pictet Spengler ligation and hydrazino-Pictet Spengler (HIPS) ligation,
wherein each individual R₃₃ is selected from the group consisting of C₁-C₁₂ alkylene groups, C₂-C₁₂ alkenylene groups, C₂-C₁₂ alkynylene groups, C₆ arylene groups, C₄-C₅ heteroarylene groups, C₃-C₈ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein each individual R₃₅ is selected from the group consisting of C₁-C₈ alkylene groups, C₂-C₈ alkenylene groups, C₂-C₈ alkynylene groups, C₆ arylene groups, C₄-C₅ heteroarylene groups, C₃-C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein for R₃₃ and R₃₅ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups, are optionally substituted with a moiety selected from the group consisting of —Cl, —F, —Br, —I, —OR′, —N(R′)₂, ═O, ═NR′, —SR′, —SO₃H, —PO₃H, —PO₄H₂, —NO₂ and —Si(R′)₃, and optionally contain one or more heteroatoms selected from the group consisting of —O—, —S—, —NR′—, —P—, and —Si—, wherein the N, S, and P atoms are optionally oxidized, wherein the N atoms are optionally quaternized,
wherein each R′ is independently selected from the group consisting of hydrogen, C₁-C₆ alkylene groups, C₂-C₆ alkenylene groups, C₂-C₆ alkynylene groups, C₆ arylene, C₄-C₅ heteroarylene, C₃—C₆ cycloalkylene groups, C₅-C₈ cycloalkenylene groups, C₅-C₁₂ alkyl(hetero)arylene groups, C₅-C₁₂ (hetero)arylalkylene groups, C₄-C₁₂ alkylcycloalkylene groups, C₄-C₁₂ cycloalkylalkylene groups,
wherein for R′ the alkylene groups, alkenylene groups, alkynylene groups, (hetero)arylene groups, cycloalkylene groups, cycloalkenylene groups, alkyl(hetero)arylene groups, (hetero)arylalkylene groups, alkylcycloalkylene groups, cycloalkylalkylene groups are optionally substituted with a moiety selected from the group consisting of —Cl, —F, —Br, —I, —OH, —NH₂, ═O, —SH, —SO₃H, —PO₃H, —PO₄H₂, —NO₂, and optionally contain one or more heteroatoms selected from the group consisting of —O—, —S—, —NH—, —P—, and —Si, wherein the N, S, and P atoms are optionally oxidized,
wherein each R″ is selected from the group consisting of 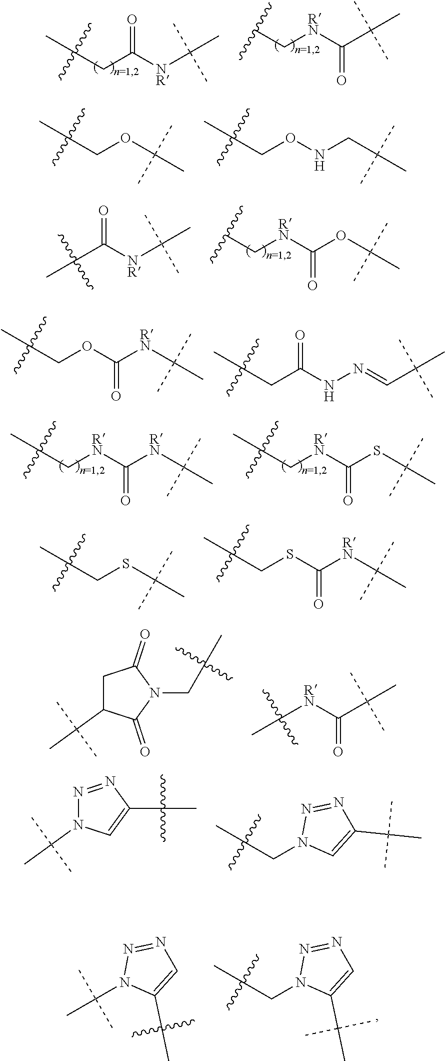
wherein the wiggly line depicts a bond to an ethylene glycol group or optionally to the R₃₃ adjacent to R₃₂ when t₄ is 0, and the dashed line depicts a bond to R₃₃ or G,
wherein R₃₄ is selected from the group consisting of —OH, —OC(O)Cl, —OC(O)O—N-succinimidyl, —OC(O)O-4-nitrophenyl, —OC(O)O-tetrafluorophenyl, —OC(O)O— pentafluorophenyl, —OC(O)—C^{A}, —OC(S)—C^{A}, —O-(L^{C}(C^{A})ₛ(C^{A})ₛ)ᵣ—C^{A}, and —C^{A},
wherein r is an integer in range of from 0 to 2,
wherein each s is independently 0 or 1,
wherein, when R₃₄ is —OC(O)—C^{A} or —OC(S)—C^{A}, C^{A} is bound to the —OC(O)— or —OC(S)— of R₃₄ via an atom selected from the group consisting of O, S, and N, wherein this atom is part of C^{A},
wherein, when R₃₄ is —O-(L^{C}(C^{A})ₛ(C^{A})ₛ)ᵣ—C^{A} and r is 0, C^{A} is bound to the —O— moiety of R₃₄ on the allylic position of the trans-cyclooctene ring of Formula (20) via a group selected from the group consisting of —C(O)—, and —C(S)—, wherein this group is part of C^{A},
wherein, when R₃₄ is —O-(L^{C}(C^{A})ₛ(C^{A})ₛ)ᵣ—C^{A} and r is 1, L^{C} is bound to the —O— moiety on the allylic position of the trans-cyclooctene ring of Formula (20) via a group selected from the group consisting of —C(Y^{C2})Y^{C1}—, and a carbon atom,
wherein Y^{C1} is selected from the group consisting of —O—, —S—, and —NR₃₆—,
wherein Y^{C2} is selected from the group consisting of O and S,
wherein, when R₃₄ is —O-(L^{C}(C^{A})ₛ(C^{A})ₛ)ᵣ—C^{A}, and r is 1, then C^{A} is bound to L^{C} via a moiety selected from the group consisting of —O—, —S—, and —N—, wherein said moiety is part of C^{A},
wherein, when R₃₄ is —C^{A}, then C^{A} is bound to the allylic position of the trans-cyclooctene of Formula (20) via an —O— atom, wherein this atom is part of C^{A},
wherein R₃₆ is selected from the group consisting of hydrogen and C₁-C₄ alkyl groups, C₂-C₄ alkenyl groups, and C₄₋₆ (hetero)aryl groups,
wherein for R₃₆ the alkyl groups, alkenyl groups, and (hetero)aryl groups are optionally substituted with a moiety selected from the group consisting of —Cl, —F, —Br, —I, —OH, —NH₂, ═O, —SH, —SO₃H, —PO₃H, —PO₄H₂ and —NO₂ and optionally contain at most two heteroatoms selected from the group consisting of —O—, —S—, —NH—, —P—, and —Si—, wherein the N, S, and P atoms are optionally oxidized,
and pharmaceutically accepted salts thereof.

16. The kit according to claim 15, wherein R₃₂ is an N-maleimidyl group linked to the remaining part of the compound according to Formula (20) via the amine of the N-maleimidyl group.

17. The kit according to claim 15, wherein said kit comprises a compound selected from the group consisting of proteins, antibodies, peptoids and peptides, modified with at least one compound according to any one of the claims 15 to 16.

18. The kit according to claim 17, wherein the compound selected from the group consisting of proteins, antibodies, peptoids and peptides comprises at least one moiety M selected from the group consisting of —OH, —NHR′, —CO₂H, —SH, —S—S—, —N₃, terminal alkynyl, terminal alkenyl, —C(O)R′, —C(O)R′—, C₈-C₁₂ (hetero)cycloalkynyl, nitrone, nitrile oxide, (imino)sydnone, isonitrile, (oxa)norbornene before modification with a compound according to claim 15, wherein R′ is as defined in claim 15, wherein the compound selected from the group consisting of proteins, peptoids antibodies, and peptides satisfies Formula (21) after modification with at least one compound according to any one of claims 15 to 16: 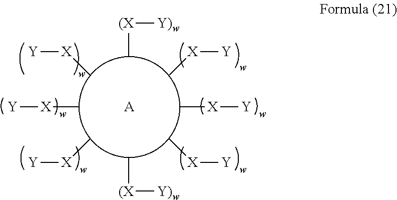
wherein moiety A is selected from the group consisting of proteins, antibodies, peptoids and peptides,
wherein each individual w is 0 or 1, wherein at least one w is 1,
wherein each moiety Y is selected from moieties according to Formula (22),
wherein at least one moiety Y satisfies said Formula (22): 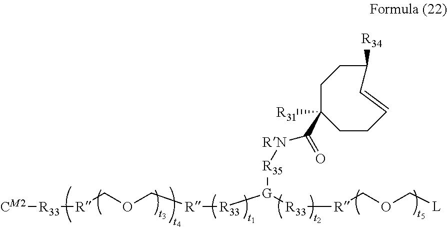
wherein n, t₁, t₂, x, y, z, G, L, R₃₁, R³, R⁴, R⁵, R′, and R″ are as defined for Formula (20),
wherein moiety X is part of moiety A and was a moiety M before modification of moiety A, wherein moiety C^{M2} is part of moiety Y and was a moiety R₃₂ as defined in claim 15 for compounds according to Formula (20) before modification of moiety A,
wherein when moiety X is —S—, then C^{M2} is selected from the group consisting of 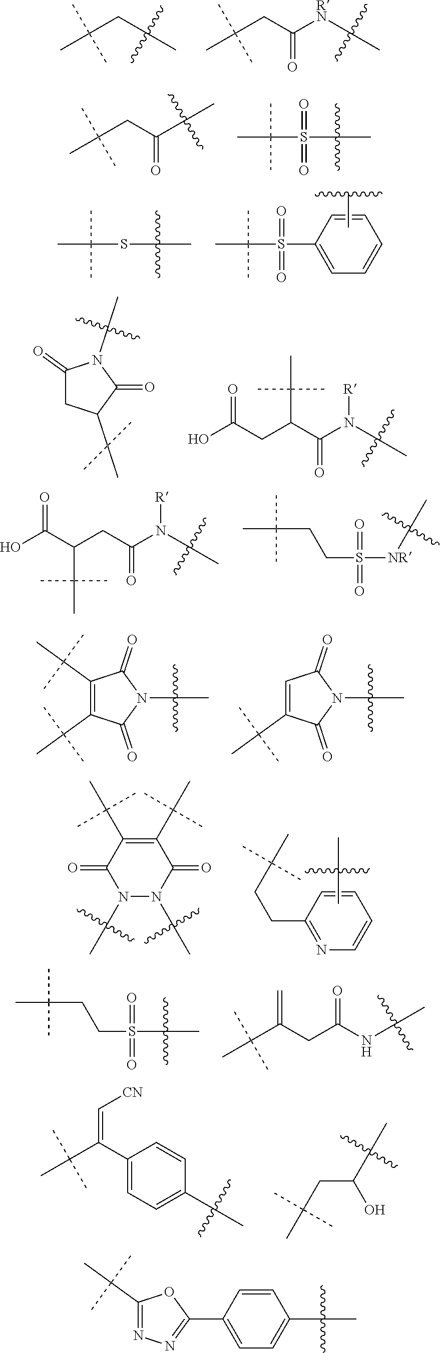
wherein the wiggly line denotes a bond to the remaining part of moiety Y, and wherein the dotted line denotes a bond to moiety X,
wherein when moiety X is —NR′—, then C^{M2} is selected from the group consisting of 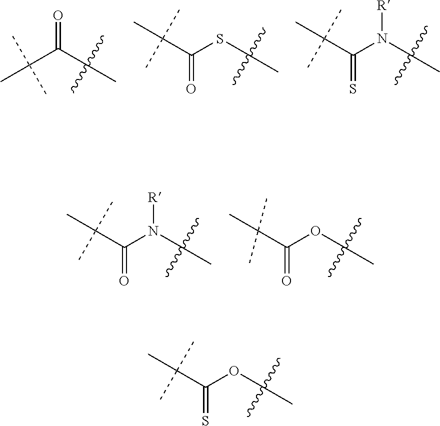
wherein the wiggly line denotes a bond to the remaining part of moiety Y, and wherein the dotted line denotes a bond to moiety X,
wherein when moiety X is —C— derived from a moiety M that was —C(O)R′ or —C(O)R′—, then C^{M2} is selected from the group consisting of 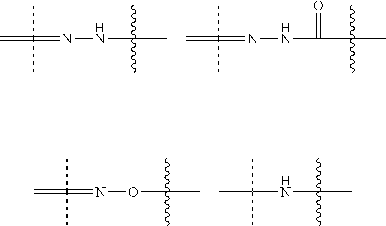
wherein the wiggly line denotes a bond to the remaining part of moiety Y, and wherein the dotted line denotes a bond to moiety X,
wherein when moiety X is —C(O)— derived from a moiety M that was —C(O)OH, then C^{M2} is selected from the group consisting of 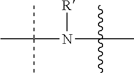
wherein the wiggly line denotes a bond to the remaining part of moiety Y, and wherein the dotted line denotes a bond to moiety X,
wherein when moiety X is —O—, then C^{M2} is selected from the group consisting of 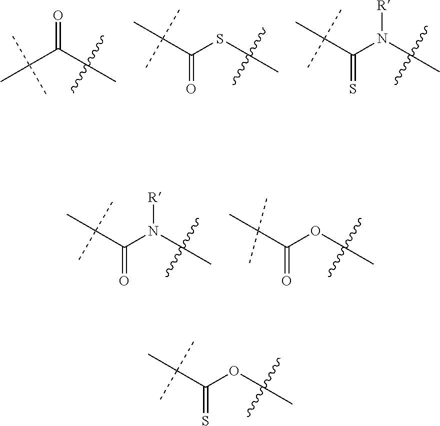
wherein the wiggly line denotes a bond to the remaining part of moiety Y, and wherein the dotted line denotes a bond to moiety X,
wherein when moiety X is derived from a moiety M that was —N₃ and that was reacted with an R₃₂ that comprised an alkyne group, then X and C^{M2} together form a moiety C^{X}, wherein C^{X} comprises a triazole ring.

19. The kit according to claim 18, wherein each C^{X} is selected from the group consisting of 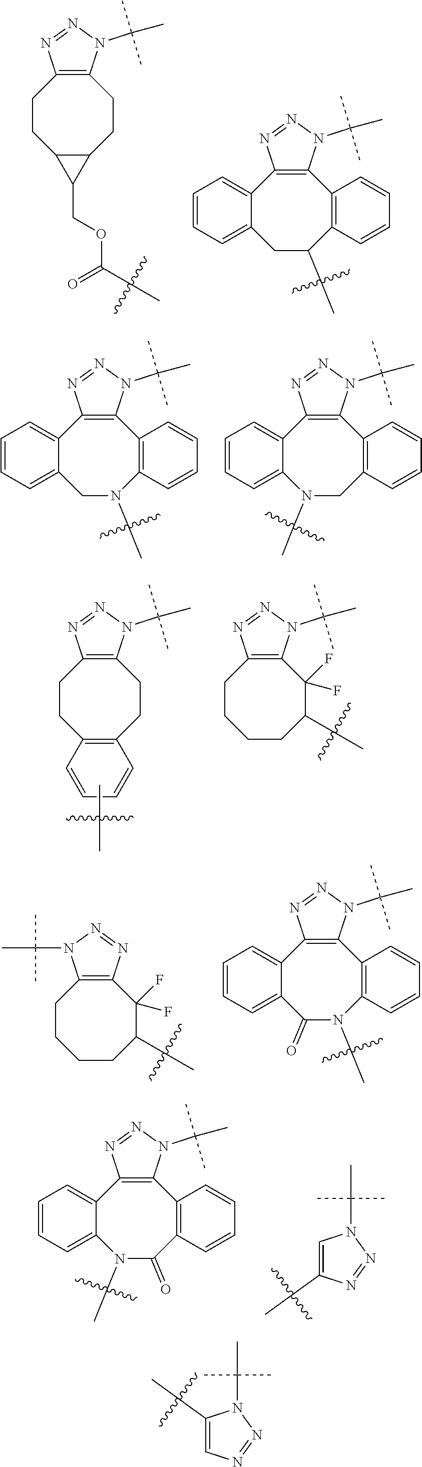
wherein the wiggly line denotes a bond to the remaining part of moiety Y, and wherein the dotted line denotes a bond to moiety X.

20. (canceled)
